(19) United States
(12) Patent Application Publication
(10) Pub. No.: US 2005/0227929 A1
(43) Pub. Date: Oct. 13, 2005

(54) Combination therapy comprising a Cox-2 inhibitor and an antineoplastic agent

(76) Inventor: Jaime L. Masferrer, Ballwin, MO (US)

Correspondence Address: Harness, Dickey & Pierce, P.L.C., Suite 400, 7700 Bonhomme, St. Louis, MO, 63105, US

(21) Appl. No.: 10/989,192

(22) Filed: Nov. 15, 2004

Related U.S. Application Data
(60) Provisional application No. 60/519,701, filed on Nov. 13, 2003.

(51) Int. Cl. A61K 31/7072; A61K 31/7048; A61K 31/5377; A61K 31/427; A61K 38/08; A61K 31/4152; A61K 31/365; A61K 31/175

(52) U.S. Cl. 514/27; 514/43; 514/234.2; 514/365; 514/406; 514/449; 514/471; 514/602; 514/49; 514/591; 514/17

(57) ABSTRACT

A method for treating or preventing neoplasia or a neoplasia-related disorder in a subject is provided, the method comprising administering to the subject an effective amount of a combination comprising a Cox-2 inhibitor and an antineoplastic agent.

### CROSS REFERENCE TO RELATED APPLICATIONS

This application claims the benefit of U.S. provisional application Ser. No. 60/519,701, filed on Nov. 13, 2003, the disclosure of which in its entirety is incorporated by reference herein.

### FIELD OF THE INVENTION

The present invention relates to therapeutic combinations and methods for use thereof for treatment or prevention of neoplasia disorders.

### BACKGROUND OF THE INVENTION

More than 1.2 million Americans develop cancer each year, making cancer the second leading cause of death in the United States. In 2000, cancer accounted for 23% of all deaths in the United States. U.S. Dept. of Health and Human Services, National Center for Health Statistics, National Vital Statistics Report, Vol. 50, No. 16 (2002). Consequently, novel treatment therapies are needed to counter the growing threat of cancer.

Cancer is a disorder arising from one or more genetic mutations that ultimately give rise to development of neoplasia. It is known that exposure of a cell to carcinogens, such as certain viruses, chemicals and radiation, can lead to DNA alteration that either inactivates a “suppressive” gene or activates an “oncogene”.

“Suppressive” genes are growth regulatory genes, which upon mutation can no longer control cell growth. “Oncogenes” are initially normal genes (protooncogenes) that by mutation or altered context of expression become transforming genes. The protein products of transforming genes cause inappropriate cell growth. This occurs through activation of several intracellular signaling pathways, including the protein kinase C/mitogen-activated protein kinase (PKC/MAPK) pathway and the Ras/Raf/MEK 1/2/ERK ½ pathway. Transformed cells differ from normal cells in many ways, including cell morphology, cell-to-cell interactions, membrane content, cytoskeletal structure, protein secretion, gene expression and loss of apoptosis.

Oncogene transformed cells and cells that have lost suppressive gene regulation undergo uncontrolled proliferation, modified control of apoptosis, and initiation of angiogenesis. All three of these effects are characteristic for development of neoplasia and neoplasms.

Neoplasia is an abnormal, unregulated and disorganized proliferation of cell growth that is distinguished from normal cells by autonomous growth and somatic mutations. As neoplastic cells grow and divide they pass on their genetic mutations and proliferative characteristics to progeny cells. A neoplasm, or tumor, is an accumulation of neoplastic cells. A neoplasm can be benign or malignant.

Although several advances have been made in detection and therapy of cancer, no universally successful method for prevention or treatment is currently available. Cancer therapy currently relies on a combination of early diagnosis and aggressive treatment, which can include surgery, chemotherapy, radiation therapy and/or hormone therapy.

Surgery involves bulk removal of neoplasms. While surgery is sometimes effective in removing tumors located at certain sites, for example in the breast, colon or skin, it cannot be used in treatment of tumors located in other areas, such as the backbone, nor in treatment of disseminated neoplastic conditions such as leukemia. Moreover, surgical treatments are generally successful only if the cancer is detected at an early stage and before the cancer has metastasized to major organs, thus making surgery non-feasible.

Chemotherapy involves disruption of cell replication and/or cell metabolism. It is used most often in treatment of breast, lung and testicular cancer. The adverse effects of systemic chemotherapy used in treatment of neoplastic disease is problematic for patients undergoing cancer treatment. Of these adverse effects nausea and vomiting are the most common and severe side effects. Other adverse side effects include cytopenia, infection, cachexia, mucositis in patients receiving high doses of chemotherapy with bone marrow rescue or radiation therapy, alopecia (hair loss), cutaneous complications including pruritus, urticaria and angioedema, and neurological, pulmonary, cardiac, reproductive and endocrine complications. See Abeloff et al. (1992) Alopecia and Cutaneous Complications, in Abeloff et al. (ed.) Clinical Oncology, pp. 755-756. New York: Churchill Livingston.

The adverse side effects induced by chemotherapeutic agents and radiation therapy have become of major importance to the clinical management of cancer patients.

Chemotherapy-induced side effects significantly impact quality of life of the patient and can dramatically influence patient compliance with treatment. Additionally, adverse side effects associated with chemotherapeutic agents are generally the major dose-limiting toxicity (DLT) in the administration of these drugs. For example, mucositis is a major DLT for several anticancer agents, including the antimetabolite cytotoxic agents 5-FU (5-fluorouracil), methotrexate and antitumor antibiotics such as doxorubicin. Many of these chemotherapy-induced side effects, if severe, can lead to hospitalization, or require treatment with analgesics for management of pain.

Likewise, radiation therapy is not without side effects such as nausea, fatigue and fever.

Novel cancer treatment strategies that eliminate need for surgical intervention and/or reduce chemotherapy-induced or radiation-induced side effects would, therefore, benefit many cancer sufferers.

Due to the high incidence and high mortality rate associated with cancer, a wealth of research is going on in this field. Of particular interest is the recent discovery that use of nonsteroidal anti-inflammatory drugs (NSAIDs) has been associated with prevention and treatment of several types of cancer. Thun et al. (2002) J. National Cancer Inst. 94(4), 252-266. Historically, physicians have treated inflammation-related disorders with a regimen of NSAIDs such as, for example, aspirin and ibuprofen. Undesirably, however, some NSAIDs are known to cause gastrointestinal (GI) bleeding or ulcers in patients undergoing consistent long term regimens of NSAID therapy. Henry et al. (1991) Lancet 337, 730.

A reduction of unwanted side effects of common NSAIDs was made possible by the discovery that two cyclooxygenases are involved in transformation of arachidonic acid as the first step in the prostaglandin synthesis pathway. These enzymes exist in two forms and have been termed cyclooxygenase-1 (Cox-1) and cyclooxygenase-2 (Cox-2). Needleman et al. (1997) J. Rheumatol. 24, Suppl. 49, 6-8.

Cox-1 is a constitutive enzyme responsible for biosynthesis of prostaglandins in the gastric mucosa and in the kidney. Cox-2 is an enzyme that is produced by an inducible gene that is responsible for biosynthesis of prostaglandins in inflammatory cells. Inflammation causes induction of Cox-2, leading to release of prostanoids (prostaglandin E2), which sensitize peripheral nociceptor terminals and produce localized pain hypersensitivity, inflammation and edema. Samad et al. (2001) Nature 410(6827), 471-475.

Many common NSAIDs are now known to be inhibitors of both Cox-1 and Cox-2. Accordingly, when administered in sufficiently high levels, these NSAIDs not only alleviate the inflammatory consequences of Cox-2 activity, but also inhibit the beneficial gastric maintenance activities of Cox-1.

Research into the area of arachidonic acid metabolism has resulted in the discovery of compounds that selectively inhibit the Cox-2 enzyme to a greater extent than they inhibit Cox-1. These Cox-2 selective inhibitors are believed to offer advantages that include the capacity to prevent or reduce inflammation while avoiding harmful side effects associated with the inhibition of Cox-1. Thus, Cox-2 selective inhibitors have shown great promise for use in therapies, especially in therapies that require maintenance administration, such as for pain and inflammation control.

Of particular importance for the present invention is that overexpression of Cox-2 has been documented in several premalignant and malignant tissues. Subbaramaiah & Dannenberg (2003) Trends Pharmacol. Sci. 24, 96-102. This increase in expression is thought to be a product of stimulation of PKC signaling, which stimulates activity of MAPK, enhancing transcription of Cox-2 by nuclear factors. Additionally, enhanced stability of Cox-2 mRNA transcripts in cancer cells due to augmented binding of the RNA-binding protein HuR, as well as activation of extracellular signal related kinase 1/2 (ERK 1/2) and p38, contributes to increased expression of Cox-2. Id.

Recently, several new chemotherapeutic agents have been reported to be efficacious in treating or preventing neoplasia-related disorders. Nevertheless, even with the multitude of chemotherapeutic agents that are now available or in clinical trials, neoplasia is still a disorder that defies most attempts at eradication. At best, remission of an existing neoplasia disorder is the only available prognosis. In addition, conventional chemotherapeutic agents have the marked disadvantage of causing a wide array of debilitating side effects.

From the foregoing, it can be seen that a need exists for improved methods and therapeutic compositions to treat neoplasia and neoplasia-related disorders. It would also be useful to provide an improved method and composition for reducing the symptoms associated with neoplasia. Likewise, methods and compositions that improve patient outcomes following radiation and chemotherapy treatment regimens for neoplasms would also be desirable. Also, methods and compositions that reduce dosages or reduce unwanted side effects in conventional treatments for neoplasia or neoplasia-related disorders are desirable. Finally, methods and compositions that improve the efficacy of treating neoplasia or a neoplasia-related disorder that is considered resistant or intractable to known methods of therapy alone would also be desirable.

Combination therapies comprising a Cox-2 inhibitor and an antineoplastic agent for treatment or prevention of neoplasia are disclosed in U.S. Pat. No. 5,972,986, incorporated herein in its entirety by reference.

Combination therapies comprising a Cox-2 inhibitor and an antineoplastic agent for treatment or prevention of angiogenic disorders are disclosed in U.S. Pat. No. 6,025,353, incorporated herein in its entirety by reference.

Combination therapies comprising a substituted benzopyran derivative Cox-2 inhibitor and an antineoplastic agent for treatment of neoplasia are disclosed in U.S. Pat. No. 6,034,256, incorporated herein in its entirety by reference.

Combination therapies comprising a Cox-2 inhibitor and an antineoplastic agent for treatment or prevention of neoplasia are disclosed in International Patent Publication No. WO 00/38730, incorporated herein in its entirety by reference.

### SUMMARY OF THE INVENTION

Briefly, the present invention is directed to a combination comprising a Cox-2 inhibitor and an antineoplastic agent selected from a group defined hereinbelow, in amounts effective when used in combination therapy for treatment or prevention of neoplasia or a neoplasia-related disorder.

The invention is also directed to a method for treating or preventing neoplasia or a neoplasia-related disorder in a subject, the method comprising administering in combination therapy to the subject a Cox-2 inhibitor and an antineoplastic agent selected from a group defined hereinbelow, in amounts effective when used in said combination therapy for treatment or prevention of the neoplasia or neoplasia-related disorder.

The present invention is further directed to a method for treating or preventing a pathological condition or physiological disorder characterized by or associated with neoplasia in a subject that is in need of such prevention or treatment, the method comprising administering to the subject a Cox-2 inhibitor in combination with an antineoplastic agent selected from a group defined hereinbelow.

An “antineoplastic agent” herein can be an agent administrable to a subject by any method or route known in the art for treatment or prevention of neoplasia, a neoplasia-related disorder, or a pathological condition or physiological disorder characterized by or associated with neoplasia. Such an agent can illustratively be an antineoplastic (including anti-angiogenic) drug, an adjunctive agent, an immunotherapeutic agent, a vaccine or a radiotherapeutic agent, and can be administrable by means of a pharmaceutical dosage form or otherwise.

The invention is still further directed to a kit comprising a first dosage form that comprises a Cox-2 inhibitor in a first amount and a second dosage form that comprises an antineoplastic agent, selected from a group defined hereinbelow, in a second amount; wherein said antineoplastic agent is administrable in a dosage form; and wherein said first and second amounts are effective when used in combination therapy for treating or preventing neoplasia or a neoplasia-related disorder.

The invention is yet further directed to a pharmaceutical composition comprising a combination as defined herein.

In all of the above embodiments, the antineoplastic agent can be selected from agents listed in Tables 3-17 herein, and more particularly from the group consisting of:
(1) polyglutamic acid-paclitaxel;
(2) BMS-184476;
(3) Paclimer microspheres with encapsulated paclitaxel;
(4) taxane (IV) of Bayer;
(5) BMS-188797;
(6) epothilone B and analogs thereof including BMS-247550;
(7) ILX-651;
(8) N-[3-[(aminocarbonyl)amino]-4-methoxyphenyl]-2,3,4,5,6-pentafluorobenzenesulfonamide;
(9) T-900607;
(10) BAY 59-8862;
(11) T-138067;
(12) N,N-dimethyl-L-valyl-L-valyl-N-methyl-L-valyl-L-prolyl-N-(1,1-dimethylethyl)-L-prolinamide;
(13) benzoylphenylurea;
(14) trimetrexate glucuronate;
(15) 5-aza-2′-deoxycytidine;
(16) tocladesine;
(17) imatinib;
(18) PTK-787;
(19) BAY-439006;
(20) N-[4-[(3-chloro-4-fluorophenyl)amino]-7-[3-(4-morpholinyl)propoxy]-6-quinazolinyl]-2-propenamide;
(21) GW-572016;
(22) EKB-569;
(23) CP 609754;
(24) CI-1033;
(25) CCI-779;
(26) BMS-214662;
(27) (R)-2,3,4,5-tetrahydro-1-(1H-imidazol-4-ylmethyl)-3-(phenylmethyl)-4-(2-thienylsulfonyl)-1H-1,4-benzodiazepine-7-carbonitrile;
(28) cilengitide;
(29) bevacizumab;
(30) PK-412;
(31) IMC-1C11;
(32) 1-(2-chloroethyl)-2-[(methylamino)]carbonyl}-2-(methylsulfonyl) hydrazide;
(33) VNP-40101M;
(34) camptothecin glycoconjugate;
(35) liposome lurtotecan;
(36) gallium maltolate;
(37) N-[(3S,4E)-3-hydroxy-7-mercapto-1-oxo-4-heptenyl]-D-valyl-D-cysteinyl-(2Z)-2-amino-2-butenoyl-L-valine (4-1)-lactone cyclic (1-2) disulfide;
(38) buthionine sulfoximine;
(39) BMS-275291;
(40) phenylacetate;
(41) MS-275;
(42) chloroquinoxaline sulfonamide;
(43) INX-3280;
(44) phosphorothioate antisense oligonucleotide;
(45) GTI-2501;
(46) GTI-2040;
(47) K-ras protein vaccine;
(48) K-ras antisense oligonucleotide;
(49) MG-98;
(50) liposome C-raf antisense oligonucleotide;
(51) liposome raf-1 antisense oligonucleotide;
(52) SPD-424;
(53) Abarelix-depot;
(54) ERA-923;
(55) GTx-006;
(56) ILX 23-7553;
(57) 2B1 bispecific MAb;
(58) 3A1 MAb;
(59) SS1(dsFv)-PE38;
(60) chimeric TNT 1/B labeled with I-131;
(61) MAb Hum291;
(62) MEDI-507;
(63) HumaRad-HN;
(64) HumaRad-OV;
(65) MAb humanized CD3;
(66) Mylotarg;
(67) MAb-CTLA-4;
(68) cetuximab;
(69) BEC2;
(70) chimeric MAb 14.18;
(71) anti-transferrin receptor MAb;
(72) epratuzumab;
(73) MGS rCEA;
(74) INGN-241;
(75) CV-787;
(76) peripheral blood lymphocytes transduced with a gene encoding a chimeric T-cell receptor;
(77) BCI Immune Activator;
(78) Interferon-alpha gene therapy;
(79) Xcellerate;
(80) interleukin-2+staphylococcal enterotoxin B;
(81) NBI-3001;
(82) beta-alethine;
(83) APC-8020;
(84) interleukin-2/superantigen B gene combination;
(85) Melacine vaccine;
(86) SD/01;
(87) ALVAC B7.1 vaccine;
(88) APC-8024;
(89) GnRH Pharmaccine vaccine;
(90) rV-MUC-1;
(91) HPV 16 E6 and E7 peptide vaccine;
(92) allogeneic colon cancer vaccine;
(93) allogeneic glioma vaccine;
(94) autologous vaccine;
(95) VHL peptide vaccine;
(96) myeloma-derived idiotypic antigen vaccine;
(97) CaPVax;
(98) idiotype KLH lymphoma vaccine;
(99) LHRH immunotherapeutic (synthetic peptide vaccine);
(100) MAGE-12:170-178 peptide vaccine;
(101) MART-1 melanoma vaccine;
(102) MART-1 with gp100;
(103) rF-tyrosine vaccine;
(104) ESO-1:157-165 peptide vaccine;
(105) fowlpox-CEA(6D) tricom and vaccinia-CEA(6D) tricom vaccine;
(106) fowlpox gp100:ES 209-217 (2m) vaccine;
(107) RAS 5-17 peptide vaccine;
(108) proteinase-3 peptide vaccine;
(109) canarypox CEA;
(110) Helicobacter pylori vaccine;
(111) P53 and RAS vaccine;
(112) BAM-002;
(113) MedPulser in combination with bleomycin;
(114) lasofoxifene;
(115) Filmix;
(116) L-377202;
(117) T4N5 Liposome Lotion;
(118) Egr-1+TNF-alpha;
(119) aprepitant;
(120) skeletal targeted radiotherapy;
(121) combretastatin;
(122) CDC-501;
(123) taurolidine;
(124) Oramed;
(125) nystatin;
(126) Dynepo gene activated EPO;
(127) NC-100150;
(128) NC-100100;
(129) CDC-801;
(130) atrasentan;
(131) Aranesp;
(132) RK-0202;
(133) SB-251353;
(134) rasburicase;
(135) AFP-scan;
(136) Lymphoscan;
(137) ADL 8-2698;
(138) carboxypeptidase G2;
(139) metoclopromide nasal;
(140) dalteparin;
(141) MK-869;
(142) monomethyl arginine;
(143) repifermin;
(144) rH TPO;
(145) SR-29142;
(146) ancestin;
(147) CP-461;
(148) Bexxar;
   and combinations thereof.

Among several advantages found to be achieved by the present invention, therefore, may be noted the provision, in certain embodiments, of combinations, methods, kits and compositions that are directed to preventing or treating neoplasia, for example cancers such as colon cancer, lung cancer, prostate cancer and breast cancer, in a subject that is in need of such prevention or treatment. Also provided in certain embodiments are improved combinations, methods, kits and compositions for reducing symptoms, including inflammation and pain, associated with neoplasia. Further, according to certain embodiments, combinations, methods, kits and compositions are provided that improve patient outcomes following radiation and chemotherapy treatment regimens for neoplasms and acute neoplasia episodes. Still further, according to certain embodiments, combinations, methods, kits and compositions are provided that reduce dosages or reduce unwanted side effects in conventional treatments for neoplasia or neoplasia-related disorders. Still further, according to certain embodiments, combinations, methods, kits and compositions are provided that improve the efficacy of treating neoplasia or a neoplasia-related disorder that is considered resistant or intractable to known methods of therapy alone.

### DETAILED DESCRIPTION OF THE INVENTION

In some embodiments, administration of a Cox-2 inhibitor in combination with an antineoplastic agent as described herein for prevention or treatment of neoplasia or a neoplasia-related disorder can be unexpectedly superior to the use of either agent alone. Therefore, according to such embodiments, treatment or prevention of neoplasia can be accomplished by administering to a subject suffering from or needing prevention of neoplasia or a neoplasia-related disorder a combination therapy comprising a Cox-2 inhibitor and an antineoplastic agent as described herein.

In certain of such embodiments, the dosage amount of one or both components of the combination can be reduced without sacrificing therapeutic efficacy. Use of low doses of certain antineoplastic agents can reduce incidence and/or severity of undesirable side effects.

Moreover, in certain of such embodiments, a combination therapy demonstrates synergistic efficacy for treating and preventing neoplasia or a neoplasia-related disorder, wherein the efficacy is greater than would be expected from simply combining the two component monotherapies.

As used herein, the term “neoplasia” refers to new cell growth that results from a loss of responsiveness to normal growth controls, e.g., “neoplastic” cell growth. For purposes of the present invention, cancer is one subtype of neoplasia. As used herein, the term “neoplasia-related disorder” encompasses neoplasia, but also encompasses other cellular abnormalities, such as hyperplasia, metaplasia and dysplasia. The terms neoplasia, metaplasia, dysplasia and hyperplasia collectively refer generally to cells experiencing abnormal cell growth.

Both neoplasia and neoplasia-related disorders can involve a neoplasm or tumor, which can be benign, premalignant, metastatic or malignant. The present invention thus encompasses methods and compositions useful for treating or preventing benign, premalignant, metastatic and malignant neoplasias, and benign, premalignant, metastatic and malignant tumors. Tumors are generally known in the art to be formed from a mass of neoplastic cells. It is to be understood, however, that even one neoplastic cell is considered, for purposes of the present invention, to be a neoplasm or alternatively, neoplasia.

The phrase “combination therapy” or “co-therapy” describes administration of two or more therapeutic agents, in the present instance a Cox-2 inhibitor and an antineoplastic agent, as part of a treatment regimen intended to provide a beneficial effect from co-action of these therapeutic agents. Such beneficial effect of the combination includes, but is not limited to, pharmacokinetic or pharmacodynamic co-action.

Combination therapy generally does not encompass administration of two or more therapeutic agents as part of separate monotherapy regimens that are incidental to one another.

Combination therapy embraces administration of therapeutic agents in a sequential manner, that is, wherein each therapeutic agent is administered at a different time. Sequential administration can occur within any time period that allows for co-action, for example within about 1 day, or about 6 hours, or about 3 hours, or about 1 hour, or about 30 minutes, or about 10 minutes.

Combination therapy also embraces administration of therapeutic agents in a substantially simultaneous manner. Substantially simultaneous administration can be accomplished, for example, by administering to the subject a single dosage form, such as a capsule, having a fixed ratio of the therapeutic agents, or in a plurality of individual dosage forms each containing one of the therapeutic agents.

Sequential or substantially simultaneous administration of therapeutic agents can be effected by any appropriate route including, but not limited to, oral, intravenous, intramuscular and subcutaneous routes and direct absorption through mucous membrane tissues. The therapeutic agents can be administered by the same route or by different routes. For example, a Cox-2 inhibitor can be administered orally and an antineoplastic agent parenterally, for example by intravenous injection or infusion. The sequence in which the therapeutic agents are administered is not narrowly critical.

Combination therapy can also embrace administration of the therapeutic agents as described herein in further combination with one or more other agents, for example a second and different antineoplastic agent or a non-drug therapy, for example surgery or radiation treatment. Where the combination therapy further comprises radiation treatment, the radiation treatment can be conducted at any suitable time. In one embodiment, the timing of administration of the combination of the invention and of radiation treatment are such as to enable a beneficial effect from co-action of the combination of the therapeutic agents and the radiation treatment. Such a beneficial effect can be achieved in some cases when the radiation treatment is temporally removed from the administration of the therapeutic agents, for example by days or even weeks.

The phrases “low dose” or “low dose amount”, in characterizing a therapeutically effective amount of a Cox-2 inhibitor or antineoplastic agent, defines a quantity that is capable of having a preventive or ameliorating effect on neoplasia or a neoplasia-related disorder while reducing or avoiding one or more side effects, such as myelosupression, cardiac toxicity, alopecia, nausea or vomiting.

The phrase “adjunctive therapy” describes treatment of a subject with agents that reduce or avoid side effects associated with cancer therapy, including, but not limited to, agents that reduce the toxic effect of anticancer drugs (e.g., bone resorption inhibitors and cardioprotective agents), prevent or reduce incidence of nausea and vomiting associated with chemotherapy, radiotherapy or surgery, or reduce the incidence of infection associated, for example, with administration of myelosuppressive anticancer drugs.

An “immunotherapeutic agent” is an agent used to transfer the immunity of an immune donor, e.g., another person or an animal, to a host by inoculation. Examples of use of immunotherapeutic agents are serum or gamma globulin containing preformed antibodies produced by another individual or an animal; nonspecific systemic stimulation; adjuvants; active specific immunotherapy; and adoptive immunotherapy. Adoptive immunotherapy refers to treatment of a disease by therapy or agents that include host inoculation of sensitized lymphocytes, transfer factor, immune RNA, or antibodies in serum or gamma globulin.

“Vaccines” herein include agents that induce a subject's immune system to mount an immune response against a tumor by attacking cells that express tumor associated antigens (TAAs).

The phrase “radiotherapeutic agent” refers to the use of electromagnetic or particulate radiation in treatment of neoplasia.

The amount or dosage of a combination therapy comprising a Cox-2 inhibitor and an antineoplastic agent is one that provides a therapeutically effective amount of the combination. Respective amounts of the Cox-2 inhibitor and of the antineoplastic agent are such as to provide such a therapeutically effective amount of the combination.

The term “therapy” herein refers to administration of agent(s) to a subject for purposes of prevention of occurrence of a condition or disorder and/or treatment of an existing condition or disorder. “Therapeutic” and “therapeutically effective” likewise embrace prevention as well as treatment.

Therapeutic effectiveness can include one or more of the following: (1) reduction in number of cancer cells; (2) reduction in tumor size; (3) inhibition (i.e., slowing or stopping) of cancer cell infiltration into peripheral organs; (4) inhibition of tumor metastasis; (5) inhibition of tumor growth; (6) relieving or reducing to some extent one or more symptoms associated with the neoplasia or neoplasia-related disorder; and (7) relieving or reducing side effects associated with administration of anticancer agents.

In one embodiment, a combination of the present invention is administered for prevention of neoplasia or a neoplasia-related disorder. As used herein, the term “prevention” refers to any reduction, no matter how slight, of a subject's predisposition or risk for developing a neoplasia or neoplasia-related disorder. For purposes of prevention herein, the subject is one that is at some degree of risk for, or is to some degree predisposed to, developing a neoplasia or a neoplasia-related disorder.

As used herein, a subject that is “predisposed to” or “at risk for” developing neoplasia or a neoplasia-related disorder or condition includes any subject having an increased chance or statistical probability for such development. Such increased chance or probability can be due to various factors, including genetic predisposition, diet, age, exposure to neoplasia causing agents, physiological factors such as anatomical and biochemical abnormalities and certain autoimmune diseases, and the like.

In another embodiment, a combination of the present invention is administered for treating an existing neoplasia or neoplasia-related disorder.

The terms “treat”, “treating” and “treatment” include alleviating symptoms, eliminating the causation of symptoms, either on a temporary or permanent basis, or altering or slowing the appearance of symptoms.

In still another embodiment, the present invention provides a method for preventing or treating neoplasia or a neoplasia-related disorder in a subject that is in need of such prevention or treatment, the method comprising administering to the subject a combination comprising a Cox-2 inhibitor and an antineoplastic agent as described herein, in further combination with radiation therapy, for example conventional radiation therapy. Thus in one embodiment a three-way combination of a Cox-2 inhibitor, an antineoplastic agent as described herein and radiation therapy is administered to a subject in need thereof.

As used herein, the term “alkyl”, alone or in combination, means an alkyl radical, linear, cyclic or branched, which, unless otherwise noted, typically contains 1 to about 10 carbon atoms, and more typically 1 to about 6 carbon atoms. Examples of such radicals include methyl, ethyl, n-propyl, isopropyl, n-butyl, isobutyl, sec-butyl, tert-butyl, pentyl, iso-amyl, hexyl, octyl and the like. Cyclic alkyl (“cycloalkyl”) radicals contain 3 to about 7 carbon atoms, typically 3 to 6 carbon atoms, for example cyclopropyl, cyclobutyl, cyclopentyl, cyclohexyl and cycloheptyl. The term “cycloalkyl” additionally encompasses spiro systems wherein the cycloalkyl ring has a carbon ring atom in common with the seven-membered heterocyclic ring of benzothiepine.

Alkyl radicals can optionally be substituted with substituent groups as defined below. Examples of such substituted alkyl radicals include chloroethyl, hydroxyethyl, trifluoromethyl, cyanobutyl, aminopentyl, and the like.

The term “alkenyl” refers to an unsaturated, hydrocarbon radical, linear, cyclic or branched, that contains at least one double bond. Unless otherwise noted, such radicals typically contain 2 to about 6 carbon atoms, more typically 2 to 4 carbon atoms, for example 2 to 3 carbon atoms. Cyclic alkenyl (“cycloalkenyl”) radicals have 3 to about 10 carbon atoms, and include cyclopropenyl, cyclobutenyl, cyclopentenyl, cyclohexenyl and cycloheptenyl. Alkenyl radicals can optionally be substituted with substituent groups as defined below. Examples of suitable alkenyl radicals include propenyl, 2-chloropropenyl, buten-1-yl, isobutenyl, penten-1-yl, 2-methylbuten-1-yl, 3-methylbuten-1-yl, hexen-1-yl, 3-hydroxyhexen-1-yl, hepten-1-yl, octen-1-yl, and the like.

The term “hydrido” denotes a single hydrogen atom (H). A hydrido radical can be attached, for example, to an oxygen atom to form a hydroxyl radical or two hydrido radicals may be attached to a carbon atom to form a methylene (—CH₂—) radical.

The term “halo” means a halogen group such as fluoro, chloro, bromo or iodo radicals. The term “haloalkyl” describes alkyl radicals that is substituted with a halo group as defined above. Specifically embraced are monohaloalkyl, dihaloalkyl and polyhaloalkyl radicals. A monohaloalkyl radical, for example, can have either a bromo, chloro or fluoro group attached to the alkyl radical. Dihalo radicals can have two or more of the same halo group or a combination of different halo groups, and polyhaloalkyl radicals can have more than two of the same halo group or a combination of different halo groups.

The term “hydroxyalkyl” describes a linear or branched alkyl radical having 1 to about 10 carbon atoms, any one of which can be substituted with one or more hydroxyl radicals.

The terms “alkoxy” and “alkoxyalkyl” describe linear or branched oxy-containing radicals each having alkyl portions of 1 to about 10 carbon atoms, such as a methoxy radical. The term “alkoxyalkyl” describes alkyl radicals having one or more alkoxy radicals attached thereto, to form for example a monoalkoxyalkyl or dialkoxyalkyl radical. Alkoxy or alkoxyalkyl radicals can be further substituted with one or more halo atoms, such as fluoro, chloro or bromo, to provide “haloalkoxy” or “haloalkoxyalkyl” radicals. Examples of alkoxy and haloalkoxy radicals include methoxy, ethoxy, propoxy, isopropoxy, isobutoxy, fluoromethoxy, chloromethoxy, trifluoromethoxy, difluoromethoxy, trifluoroethoxy, fluoroethoxy, tetrafluoroethoxy, pentafluoroethoxy and fluoropropoxy.

The term “aryl”, alone or in combination, means a carbocyclic aromatic system containing one, two or three rings wherein such rings may be attached together in a pendent manner or may be fused. The term “aryl” includes aromatic radicals such as phenyl, naphthyl, tetrahydronapthyl, indane and biphenyl.

The term “heterocyclyl” or “heterocyclic” means a saturated or unsaturated mono- or multi-ring carbocycle wherein one or more carbon atoms is replaced by N, S, P, or O. This includes, for example, structures such as 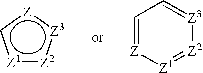
wherein Z, Z¹, Z² and Z³ are C, S, P, O or N, with the proviso that at least one of Z, Z¹, Z² and Z³ is other than carbon, but is not O or S when attached to another Z atom by a double bond or when attached to another O or S atom. Furthermore, optional substituents are understood to be attached to Z, Z¹, Z² or Z³ only when the Z atom is C. Heterocyclic radicals can be saturated, partially saturated or unsaturated heteroatom-containing ring-shaped radicals, where the heteroatoms are selected from N, S and O. Examples of saturated heterocyclic radicals include piperazinyl, dioxanyl, tetrahydrofuranyl, oxiranyl, aziridinyl, morpholinyl, pyrrolidinyl, piperidinyl, thiazolidinyl, and others. Examples of unsaturated heterocyclic radicals, also termed “heteroaryl” radicals, include thienyl, pyrryl, furyl, pyridyl, pyrimidyl, pyrazinyl, pyrazolyl, oxazolyl, isoxazolyl, imidazolyl, thiazolyl, pyranyl, quinolinyl, isoquinolinyl, benzothienyl, indolyl and tetrazolyl. Also included are radicals where a heterocyclic ring is fused with an aryl ring. Examples of fused bicyclic radicals include benzofuran, benzothiophene, and the like.

The term “sulfonyl”, whether used alone or linked to other terms as in “alkylsulfonyl”, denotes the divalent radical —SO₂—. “Alkylsulfonyl” denotes an alkyl radical attached to a sulfonyl radical, where alkyl is defined as above. The term “arylsulfonyl” denotes a sulfonyl radical substituted with an aryl radical. The terms “sulfamyl” or “sulfonamidyl”, whether alone or linked to other terms as in “N-alkylsulfamyl”, “N-arylsulfamyl”, “N,N-dialkylsulfamyl” and “N-alkyl-N-arylsulfamyl”, denote a sulfonyl radical substituted with an amine radical, forming a sulfonamide (—SO₂NH₂). The terms “N-alkylsulfamyl” and “N,N-dialkylsulfamyl” denote sulfamyl radicals substituted with 1 to 2 alkyl radicals or a cycloalkyl ring. The terms “N-arylsulfamyl” and “N-alkyl-N-arylsulfamyl” denote sulfamyl radicals substituted, respectively, with one aryl radical, or with one alkyl and one aryl radical.

The terms “carboxy” or “carboxyl”, whether used alone or linked to other terms, as in “carboxyalkyl”, denote —CO₂H. The term “carboxyalkyl” denotes a carboxy radical as defined above, attached to an alkyl radical.

The term “carbonyl”, whether used alone or linked to other terms, as in “alkylcarbonyl”, denotes —(C═O)—. The term “alkylcarbonyl” denotes a carbonyl radical substituted with an alkyl radical, for example CH₃—(C═O)—. “Alkylcarbonylalkyl” denotes an alkyl radical substituted with an alkylcarbonyl radical. The term “alkoxycarbonyl” means a radical containing an alkoxy group, attached via an oxygen atom to a carbonyl radical, for example (CH₃)₃CO—C(═O)— or —(O═)C—OCH₃. The term “alkoxycarbonylalkyl” denotes a radical having alkoxycarbonyl, as defined above, attached to an alkyl radical. Examples of such alkoxycarbonylalkyl radicals include (CH₃)₃CO—C(═O)(CH₂)₂— and —(CH₂)₂(═O)C—OCH₃.

The term “amido” when used by itself or linked to other terms as in “amidoalkyl”, “N-monoalkylamido”, “N-monoarylamido”, “N,N-dialkylamido”, “N-alkyl-N-arylamido”, “N-alkyl-N-hydroxyamido” and “N-alkyl-N-hydroxyamidoalkyl”, denotes a carbonyl radical substituted with an amino radical. The terms “N-alkylamido” and “N,N-dialkylamido” denote amido groups which have been substituted with one or two alkyl radicals, respectively. The terms “N-monoarylamido” and “N-alkyl-N-arylamido” denote amido radicals substituted, respectively, with one aryl radical, or with one alkyl and one aryl radical. The term “N-alkyl-N-hydroxyamido” denotes an amido radical substituted with a hydroxyl radical and with an alkyl radical. The term “N-alkyl-N-hydroxyamidoalkyl” denotes an alkyl radical substituted with an N-alkyl-N-hydroxyamido radical. The term “amidoalkyl” denotes an alkyl radical substituted with one or more amido radicals. The term “aminoalkyl” denotes an alkyl radical substituted with one or more amino radicals. The term “alkylaminoalkyl” denotes an aminoalkyl radical having the nitrogen atom of the amino group substituted with an alkyl radical. The term “amidino” denotes a —C(═NH)—NH₂ radical. The term “cyanoamidino” denotes a —C(═N—CN)—NH₂ radical.

The term “heterocycloalkyl” denotes a heterocyclic-substituted alkyl radical such as pyridylmethyl or thienylmethyl.

The term “aralkyl” denotes an aryl-substituted alkyl radical such as benzyl, diphenylmethyl, triphenylmethyl, phenethyl or diphenethyl. The terms benzyl and phenylmethyl are interchangeable.

The term “alkylthio” denotes a radical containing a linear or branched alkyl radical of 1 to about 10 carbon atoms, attached to a divalent sulfur atom. An example is methylthio, (CH₃—S—). The term “alkylsulfinyl” denotes a radical containing a linear or branched alkyl radical of 1 to about 10 carbon atoms, attached to a divalent —S(═O)-group. The term “alkylthioalkyl” denotes an alkylthio radical attached to an alkyl group, an example being methylthiomethyl.

The terms “N-alkylamino” and “N,N-dialkylamino” denote amino groups which have been substituted with one alkyl radical or with two alkyl radicals, respectively.

The term “acyl”, whether used alone or within a term such as “acylamino”, denotes a radical provided by the residue after removal of hydroxyl from an organic acid. The term “acylamino” denotes an amino radical substituted with an acyl group, an example being acetylamine (CH₃C(═O)—NH—).

In either heterocyclyl or heteroaryl rings, the point of attachment to the molecule of interest can be at the heteroatom or elsewhere within the ring.

The term “oxo” means a doubly-bonded oxygen.

As used herein, “organic halide” means a compound having fluorine, chlorine, bromine, iodine or astatine covalently coupled with an alkyl, alkenyl, alkynyl, alkoxy, aralkyl, aryl, carbonyl, cycloalkyl, benzyl, phenyl, alicyclic or heterocyclic group.

As used herein, the term “carbamoyl” refers to a carbonyl group covalently bonded at the oxo carbon to an amino group.

As used herein, the term “hydroxamate” refers to a carbonyl group covalently bonded at the oxo carbon to an amino group, wherein the amino group is in turn bonded to a hydroxyl group.

The term “oxime” means a radical comprising ═NOH.

The terms “cyclooxygenase-2 inhibitor” and “Cox-2 inhibitor”, which can be used interchangeably herein, denote compounds which inhibit the cyclooxygenase-2 enzyme (Cox-2) regardless of the degree of inhibition of the cyclooxygenase-1 enzyme (Cox-1), and include pharmaceutically acceptable racemates, enantiomers, tautomers, salts, esters and prodrugs of those compounds. Thus, for purposes of the present invention, a compound is considered a Cox-2 inhibitor although the compound inhibits Cox-2 to an equal, greater, or lesser degree than it inhibits Cox-1. Cox-2 inhibitors herein therefore encompass many traditional non-selective NSAIDs (non-steroidal anti-inflammatory drugs).

Suitable NSAIDs include ibuprofen, naproxen, benoxaprofen, flurbiprofen, fenoprofen, fenbufen, ketoprofen, indoprofen, pirprofen, carprofen, oxaprozin, prapoprofen, miroprofen, tioxaprofen, suprofen, alminoprofen, tiaprofenic acid, fluprofen, bucloxic acid, indomethacin, sulindac, tolmetin, zomepirac, diclofenac, fenclofenac, alclofenac, ibufenac, isoxepac, furofenac, tiopinac, zidometacin, acetyl salicylic acid, indomethacin, piroxicam, tenoxicam, nabumetone, ketorolac, azapropazone, mefenamic acid, tolfenamic acid, diflunisal, podophyllotoxin derivatives, acemetacin, droxicam, floctafenine, oxyphenbutazone, phenylbutazone, proglumetacin, acemetacin, fentiazac, clidanac, oxipinac, mefenamic acid, meclofenamic acid, flufenamic acid, niflumic acid, flufenisal, sudoxicam, etodolac, piprofen, salicylic acid, choline magnesium trisalicylate, salicylate, benorylate, fentiazac, clopinac, feprazone, isoxicam, 2-fluoro-a-methyl[1,1′-biphenyl]-4-acetic acid, 4-(nitrooxy)butyl ester (See Wenk et al. (2002) Europ. J. Pharmacol. 453, 319-324, incorporated herein by reference) and mixtures thereof.

Particular NSAIDs of interest include ibuprofen, naproxen, sulindac, ketoprofen, fenoprofen, tiaprofenic acid, suprofen, etodolac, carprofen, ketorolac, piprofen, indoprofen, salicylic acid, flurbiprofen and mixtures thereof.

Further Cox-2 inhibitors useful according to embodiments of the present invention are agents and compounds that selectively or preferentially inhibit Cox-2 to a greater degree than they inhibit Cox-1. Such agents and compounds are termed “Cox-2 selective inhibitors” herein.

In practice, in a test for selectivity of a Cox-2 selective inhibitor, the observed selectivity varies depending upon the conditions under which the test is performed and on the compound being tested. However, for the present purpose, selectivity of a Cox-2 inhibitor can be measured as a ratio of the in vitro or in vivo IC₅₀ value for inhibition of Cox-1, divided by the corresponding IC₅₀ value for inhibition of Cox-2 (Cox-1 IC₅₀/Cox-2 IC₅₀). A Cox-2 selective inhibitor herein is thus any inhibitor for which Cox-1 IC₅₀/Cox-2 IC₅₀ is greater than 1. In various embodiments this ratio is greater than about 2, greater than about 5, greater than about 10, greater than about 50, or greater than about 100.

The term “IC₅₀” with respect to a Cox-2 inhibitor refers to the concentration of a compound that is required to produce 50% inhibition of activity of Cox-1 or Cox-2. In various embodiments, Cox-2 selective inhibitors useful in the present invention can have a Cox-2 IC₅₀ of less than about 1 μM, less than about 0.5 μM, or less than about 0.2 μM. Cox-2 selective inhibitors useful in the present invention can have a Cox-1 IC₅₀ of greater than about 1 μM, for example greater than about 20 μM.

Cox-2 inhibitors exhibiting a high degree of selectivity for Cox-2 over Cox-1 inhibition can indicate ability to reduce incidence of common NSAID-induced side effects.

A Cox-2 selective inhibitor can be used in a form of a prodrug thereof. In the present context, a “prodrug” is a compound that can be converted into an active Cox-2 selective inhibitor by metabolic or simple chemical processes within the body of the subject. One example of a prodrug for a Cox-2 selective inhibitor is parecoxib, for example in a form of a salt such as parecoxib sodium, which is a therapeutically effective prodrug of the tricyclic Cox-2 selective inhibitor valdecoxib. A class of prodrugs of Cox-2 selective inhibitors is described in U.S. Pat. No. 5,932,598, incorporated herein by reference.

In one embodiment the Cox-2 selective inhibitor is meloxicam or a pharmaceutically acceptable salt or prodrug thereof.

In another embodiment the Cox-2 selective inhibitor is RS 57067 (6-[[5-(4-chlorobenzoyl)-1,4-dimethyl-1H-pyrrol-2-yl]methyl]-3(2H)-pyridazinone) or a pharmaceutically acceptable salt or prodrug thereof.

In another embodiment the Cox-2 selective inhibitor is of the chromene or chroman structural class that is a substituted benzopyran or a substituted benzopyran analog, for example selected from the group consisting of substituted benzothiopyrans, dihydroquinolines and dihydronaphthalenes. These compounds can have a structure as shown in any of formulas (I), (II), (III), (IV), (V) and (VI) below, and as illustrated in Table 1, and can be diastereomers, enantiomers, racemates, tautomers, salts, esters, amides and prodrugs of such compounds.

Benzopyrans that can serve as a COX-2 selective inhibitor of the present invention include substituted benzopyran derivatives that are described in U.S. Pat. No. 6,271,253, incorporated herein by reference. One such class of compounds is defined by the general formula shown below in formula (I): 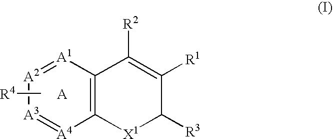
wherein:
X¹ is selected from O, S, CR^{c}R^{b} and NR^{a}, where R^{a} is selected from hydrido, C₁-C₃ alkyl, (optionally substituted phenyl)-C₁-C₃ alkyl, acyl and carboxy-C₁-C₆ alkyl; and where each of R^{b} and R^{c} is independently selected from hydrido, C₁-C₃ alkyl, phenyl-C₁-C₃ alkyl, C₁-C₃ perfluoroalkyl, chloro, C₁-C₆ alkylthio, C₁-C₆ alkoxy, nitro, cyano and cyano-C₁-C₃ alkyl; or where CR^{b}R^{c} forms a 3-6 membered cycloalkyl ring;
R¹ is selected from carboxyl, aminocarbonyl, C₁-C₆ alkylsulfonylaminocarbonyl and C₁-C₆ alkoxycarbonyl;
R² is selected from hydrido, phenyl, thienyl, C₁-C₆ alkyl and C₂-C₆ alkenyl;
R³ is selected from C₁-C₃ perfluoroalkyl, chloro, C₁-C₆ alkylthio, C₁-C₆ alkoxy, nitro, cyano and cyano-C₁-C₃ alkyl;
R⁴ is one or more radicals independently selected from hydrido, halo, C₁-C₆ alkyl, C₂-C₆ alkenyl, C₂-C₆ alkynyl, halo-C₂-C₆ alkynyl, aryl-C₁-C₃ alkyl, aryl-C₂-C₆ alkynyl, aryl-C₂-C₆ alkenyl, C₁-C₆ alkoxy, methylenedioxy, C₁-C₆ alkylthio, C₁-C₆ alkylsulfinyl, aryloxy, arylthio, arylsulfinyl, heteroaryloxy, C₁-C₆ alkoxy-C₁-C₆ alkyl, aryl-C₁-C₆ alkyloxy, heteroaryl-C₁-C₆ alkyloxy, aryl-C₁-C₆ alkoxy-C₁-C₆ alkyl, C₁-C₆ haloalkyl, C₁-C₆ haloalkoxy, C₁-C₆ haloalkylthio, C₁-C₆ haloalkylsulfinyl, C₁-C₆ haloalkylsulfonyl, C₁-C₃ haloalkyl-C₁-C₃ hydroxyalkyl, C₁-C₆ hydroxyalkyl, hydroxyimino-C₁-C₆ alkyl, C₁-C₆ alkylamino, arylamino, aryl-C₁-C₆ alkylamino, heteroarylamino, heteroaryl-C₁-C₆ alkylamino, nitro, cyano, amino, aminosulfonyl, C₁-C₆ alkylaminosulfonyl, arylaminosulfonyl, heteroarylaminosulfonyl, aryl-C₁-C₆ alkylaminosulfonyl, heteroaryl-C₁-C₆ alkylaminosulfonyl, heterocyclylsulfonyl, C₁-C₆ alkylsulfonyl, aryl-C₁-C₆ alkylsulfonyl, optionally substituted aryl, optionally substituted heteroaryl, aryl-C₁-C₆ alkylcarbonyl, heteroaryl-C₁-C₆ alkylcarbonyl, heteroarylcarbonyl, arylcarbonyl, aminocarbonyl, C₁-C₁ alkoxycarbonyl, formyl, C₁-C₆ haloalkylcarbonyl and C₁-C₆ alkylcarbonyl; and
the A ring atoms A¹, A², A³ and A⁴ are independently selected from carbon and nitrogen with the proviso that at least two of A¹, A², A³ and A⁴ are carbon; or
R⁴ together with ring A forms a radical selected from naphthyl, quinolinyl, isoquinolinyl, quinolizinyl, quinoxalinyl and dibenzofuryl;
   or an isomer or pharmaceutically acceptable salt thereof.

Another class of benzopyran derivatives that can serve as the COX-2 selective inhibitor of the present invention includes a compound having the structure of formula (II): 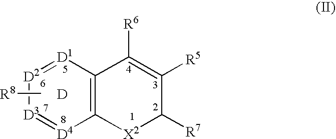
wherein:
X² is selected from O, S, CR^{c}R^{b} and NR^{a}; where R^{a} is selected from hydrido, C₁-C₃ alkyl, (optionally substituted phenyl)-C₁-C₃ alkyl, alkylsulfonyl, phenylsulfonyl, benzylsulfonyl, acyl and carboxy-C₁-C₆ alkyl; and where each of R^{b} and R^{c} is independently selected from hydrido, C₁-C₃ alkyl, phenyl-C₁-C₃ alkyl, C₁-C₃ perfluoroalkyl, chloro, C₁-C₆ alkylthio, C₁-C₆ alkoxy, nitro, cyano and cyano-C₁-C₃ alkyl; or where CR^{c}R^{b} form a cyclopropyl ring;
R⁵ is selected from carboxyl, aminocarbonyl, C₁-C₆ alkylsulfonylaminocarbonyl and C₁-C₆ alkoxycarbonyl;
R⁶ is selected from hydrido, phenyl, thienyl, C₂-C₆ alkynyl and C₂-C₆ alkenyl;
R⁷ is selected from C₁-C₃ perfluoroalkyl, chloro, C₁-C₆ alkylthio, C₁-C₆ alkoxy, nitro, cyano and cyano-C₁-C₃ alkyl;
R⁸ is one or more radicals independently selected from hydrido, halo, C₁-C₆ alkyl, C₂-C₆ alkenyl, C₂-C₆ alkynyl, halo-C₂-C₆ alkynyl, aryl-C₁-C₃ alkyl, aryl-C₂-C₆ alkynyl, aryl-C₂-C₆ alkenyl, C₁-C₆ alkoxy, methylenedioxy, C₁-C₆ alkylthio, C₁-C₆ alkylsulfinyl, —O(CF₂)₂O—, aryloxy, arylthio, arylsulfinyl, heteroaryloxy, C₁-C₆ alkoxy-C₁-C₆ alkyl, aryl-C₁-C₆ alkyloxy, heteroaryl-C₁-C₆ alkyloxy, aryl-C₁-C₆ alkoxy-C₁-C₆ alkyl, C₁-C₆ haloalkyl, C₁-C₆ haloalkoxy, C₁-C₆ haloalkylthio, C₁-C₆ haloalkylsulfinyl, C₁-C₆ haloalkylsulfonyl, C₁-C₃ haloalkyl-C₁-C₃ hydroxyalkyl, C₁-C₆ hydroxyalkyl, hydroxyimino-C₁-C₆ alkyl, C₁-C₆ alkylamino, arylamino, aryl-C₁-C₆ alkylamino, heteroarylamino, heteroaryl-C₁-C₆ alkylamino, nitro, cyano, amino, aminosulfonyl, C₁-C₆ alkylaminosulfonyl, arylaminosulfonyl, heteroarylaminosulfonyl, aryl-C₁-C₆ alkylaminosulfonyl, heteroaryl-C₁-C₆ alkylaminosulfonyl, heterocyclylsulfonyl, C₁-C₆ alkylsulfonyl, aryl-C₁-C₆ alkylsulfonyl, optionally substituted aryl, optionally substituted heteroaryl, aryl-C₁-C₆ alkylcarbonyl, heteroaryl-C₁-C₆ alkylcarbonyl, heteroarylcarbonyl, arylcarbonyl, aminocarbonyl, C₁-C₆ alkoxycarbonyl, formyl, C₁-C₆ haloalkylcarbonyl and C₁-C₆ alkylcarbonyl; and
the D ring atoms D¹, D², D³ and D⁴ are independently selected from carbon and nitrogen with the proviso that at least two of D¹, D², D³ and D⁴ are carbon; or
R⁸ together with ring D forms a radical selected from naphthyl, quinolinyl, isoquinolinyl, quinolizinyl, quinoxalinyl and dibenzofuryl;
   or an isomer or pharmaceutically acceptable salt thereof.

Other benzopyran COX-2 selective inhibitors useful in the practice of the present invention are described in U.S. Pat. Nos. 6,034,256 and 6,077,850, incorporated herein by reference. The general formula for these compounds is shown in formula (III): 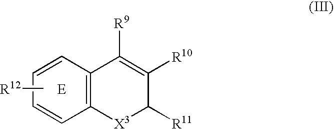
wherein:
X³ is selected from the group consisting of O or S or NR^{a} where R^{a} is alkyl;
R⁹ is selected from the group consisting of H and aryl;
R¹⁰ is selected from the group consisting of carboxyl, aminocarbonyl, alkylsulfonylaminocarbonyl and alkoxycarbonyl;
R¹¹ is selected from the group consisting of haloalkyl, alkyl, aralkyl, cycloalkyl and aryl optionally substituted with one or more radicals selected from alkylthio, nitro and alkylsulfonyl; and
R¹² is selected from the group consisting of one or more radicals selected from H, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, hydroxyarylcarbonyl, nitroaryl, optionally substituted aryl, optionally substituted heteroaryl, aralkylcarbonyl, heteroarylcarbonyl, arylcarbonyl, aminocarbonyl, and alkylcarbonyl; or
R¹² together with ring E forms a naphthyl radical;
   or an isomer or pharmaceutically acceptable salt thereof; and including diastereomers, enantiomers, racemates, tautomers, salts, esters, amides and prodrugs thereof.

A related class of compounds useful as COX-2 selective inhibitors in the present invention is described by formulas (IV) and (V): 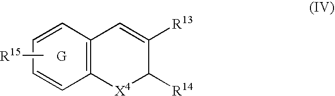
wherein:
X⁴ is selected from O or S or NR^{a} where R^{a} is alkyl;
R¹³ is selected from carboxyl, aminocarbonyl, alkylsulfonylaminocarbonyl and alkoxycarbonyl;
R¹⁴ is selected from haloalkyl, alkyl, aralkyl, cycloalkyl and aryl optionally substituted with one or more radicals selected from alkylthio, nitro and alkylsulfonyl; and
R¹⁵ is one or more radicals selected from hydrido, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, optionally substituted aryl, optionally substituted heteroaryl, aralkylcarbonyl, heteroarylcarbonyl, arylcarbonyl, aminocarbonyl, and alkylcarbonyl; or
R¹⁵ together with ring G forms a naphthyl radical;
   or an isomer or pharmaceutically acceptable salt thereof.

Formula (V) is: 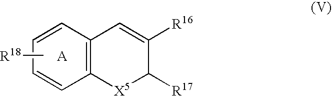
wherein:
X⁵ is selected from the group consisting of O or S or NR^{b} where R^{b} is alkyl;
R¹⁶ is selected from the group consisting of carboxyl, aminocarbonyl, alkylsulfonylaminocarbonyl and alkoxycarbonyl;
R¹⁷ is selected from the group consisting of haloalkyl, alkyl, aralkyl, cycloalkyl and aryl, wherein haloalkyl, alkyl, aralkyl, cycloalkyl, and aryl each is independently optionally substituted with one or more radicals selected from the group consisting of alkylthio, nitro and alkylsulfonyl; and
R¹⁸ is one or more radicals selected from the group consisting of hydrido, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, optionally substituted aryl, optionally substituted heteroaryl, aralkylcarbonyl, heteroarylcarbonyl, arylcarbonyl, aminocarbonyl and alkylcarbonyl; or
wherein R¹⁸ together with ring A forms a naphthyl radical;
   or an isomer or pharmaceutically acceptable salt thereof.

The COX-2 selective inhibitor can be a compound of Formula (V), wherein:
X⁵ is selected from the group consisting of oxygen and sulfur;
R¹⁶ is selected from the group consisting of carboxyl, lower alkyl, lower aralkyl and lower alkoxycarbonyl;
R¹⁷ is selected from the group consisting of lower haloalkyl, lower cycloalkyl and phenyl; and
R¹⁸ is one or more radicals selected from the group of consisting of hydrido, halo, lower alkyl, lower alkoxy, lower haloalkyl, lower haloalkoxy, lower alkylamino, nitro, amino, aminosulfonyl, lower alkylaminosulfonyl, 5-membered heteroarylalkylaminosulfonyl, 6-membered heteroarylalkylaminosulfonyl, lower aralkylaminosulfonyl, 5-membered nitrogen-containing heterocyclosulfonyl, 6-membered-nitrogen containing heterocyclosulfonyl, lower alkylsulfonyl, optionally substituted phenyl, lower aralkylcarbonyl, and lower alkylcarbonyl; or
R¹⁸ together with ring A forms a naphthyl radical;
   or an isomer or pharmaceutically acceptable salt thereof.

The COX-2 selective inhibitor can be a compound of Formula (V), wherein:
X⁵ is selected from the group consisting of oxygen and sulfur;
R¹⁶ is carboxyl;
R¹⁷ is lower haloalkyl; and
R¹⁸ is one or more radicals selected from the group consisting of hydrido, halo, lower alkyl, lower haloalkyl, lower haloalkoxy, lower alkylamino, amino, aminosulfonyl, lower alkylaminosulfonyl, 5-membered heteroarylalkylaminosulfonyl, 6-membered heteroarylalkylaminosulfonyl, lower aralkylaminosulfonyl, lower alkylsulfonyl, 6-membered nitrogen-containing heterocyclosulfonyl, optionally substituted phenyl, lower aralkylcarbonyl, and lower alkylcarbonyl; or
R¹⁸ together with ring A forms a naphthyl radical;
   or an isomer or pharmaceutically acceptable salt thereof.

The COX-2 selective inhibitor can be a compound of Formula (V), wherein:
X⁵ is selected from the group consisting of oxygen and sulfur;
R¹⁶ is selected from the group consisting of carboxyl, lower alkyl, lower aralkyl and lower alkoxycarbonyl;
R¹⁷ is selected from the group consisting of fluoromethyl, chloromethyl, dichloromethyl, trichloromethyl, pentafluoroethyl, heptafluoropropyl, difluoroethyl, difluoropropyl, dichloroethyl, dichloropropyl, difluoromethyl and trifluoromethyl; and
R¹⁸ is one or more radicals selected from the group consisting of hydrido, chloro, fluoro, bromo, iodo, methyl, ethyl, isopropyl, tert-butyl, butyl, isobutyl, pentyl, hexyl, methoxy, ethoxy, isopropyloxy, tertbutyloxy, trifluoromethyl, difluoromethyl, trifluoromethoxy, amino, N,N-dimethylamino, N,N-diethylamino, N-phenylmethylaminosulfonyl, N-phenylethylaminosulfonyl, N-(2-furylmethyl)aminosulfonyl, nitro, N,N-dimethylaminosulfonyl, aminosulfonyl, N-methylaminosulfonyl, N-ethylsulfonyl, 2,2-dimethylethylaminosulfonyl, N,N-dimethylaminosulfonyl, N-(2-methylpropyl)aminosulfonyl, N-morpholinosulfonyl, methylsulfonyl, benzylcarbonyl, 2,2-dimethylpropylcarbonyl, phenylacetyl and phenyl; or
R¹⁸ together with ring A forms a naphthyl radical;
   or an isomer or pharmaceutically acceptable salt thereof.

The COX-2 selective inhibitor can be a compound of Formula (V), wherein:
X⁵ is selected from the group consisting of oxygen and sulfur;
R¹⁶ is selected from the group consisting of carboxyl, lower alkyl, lower aralkyl and lower alkoxycarbonyl;
R¹⁷ is selected from the group consisting trifluoromethyl and pentafluoroethyl; and
R¹⁸ is one or more radicals selected from the group consisting of hydrido, chloro, fluoro, bromo, iodo, methyl, ethyl, isopropyl, tert-butyl, methoxy, trifluoromethyl, trifluoromethoxy, N-phenylmethylaminosulfonyl, N-phenylethylaminosulfonyl, N-(2-furylmethyl)aminosulfonyl, N,N-dimethylaminosulfonyl, N-methylaminosulfonyl, N-(2,2-dimethylethyl)aminosulfonyl, dimethylaminosulfonyl, 2-methylpropylaminosulfonyl, N-morpholinosulfonyl, methylsulfonyl, benzylcarbonyl and phenyl; or
R¹⁸ together with ring A forms a naphthyl radical;
   or an isomer or prodrug thereof.

Another class of benzopyran derivatives that can serve as the COX-2 selective inhibitor of the present invention includes a compound having the structure of formula (VI): 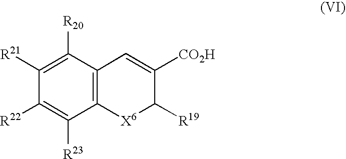
wherein:
X⁶ is selected from the group consisting of O and S;
R¹⁹ is lower haloalkyl;
R²⁰ is selected from the group consisting of hydrido and halo;
R²¹ is selected from the group consisting of hydrido, halo, lower alkyl, lower haloalkoxy, lower alkoxy, lower aralkylcarbonyl, lower dialkylaminosulfonyl, lower alkylaminosulfonyl, lower aralkylaminosulfonyl, lower heteroaralkylaminosulfonyl, 5-membered nitrogen-containing heterocyclosulfonyl, and 6-membered nitrogen-containing heterocyclosulfonyl;
R²² is selected from the group consisting of hydrido, lower alkyl, halo, lower alkoxy and aryl; and
R²³ is selected from the group consisting of the group consisting of hydrido, halo, lower alkyl, lower alkoxy, and aryl;
   or an isomer or prodrug thereof.

The COX-2 selective inhibitor can be a compound of Formula (VI), wherein:
X⁶ is selected from the group consisting of O and S;
R¹⁹ is selected from the group consisting of trifluoromethyl and pentafluoroethyl;
R²⁰ is selected from the group consisting of hydrido, chloro and fluoro;
R²¹ is selected from the group consisting of hydrido, chloro, bromo, fluoro, iodo, methyl, tert-butyl, trifluoromethoxy, methoxy, benzylcarbonyl, dimethylaminosulfonyl, isopropylaminosulfonyl, methylaminosulfonyl, benzylaminosulfonyl, phenylethylaminosulfonyl, methylpropylaminosulfonyl, methylsulfonyl and morpholinosulfonyl;
R²² is selected from the group consisting of hydrido, methyl, ethyl, isopropyl, tert-butyl, chloro, methoxy, diethylamino and phenyl; and
R²³ is selected from the group consisting of hydrido, chloro, bromo, fluoro, methyl, ethyl, tert-butyl, methoxy and phenyl;

or an isomer or prodrug thereof.

TABLE 1
Examples of chromene Cox-2 selective inhibitors

| No. | Structural formula and name |
|---|---|
| B-3 | 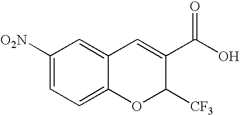 |
| | 6-Nitro-2-trifluoromethyl-2H-1- |
| | benzopyran-3-carboxylicc acid |
| B-4 | 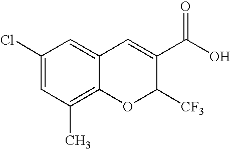 |
| | 6-Chloro-8-methyl-2-trifluoromethyl- |
| | 2H-1-benzopyran-3-carboxylic acid |
| B-5 | 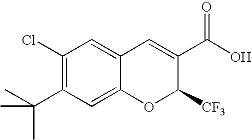 |
| | ((S)-5-Chloro-7-(1,1-dimethylethyl)-2-(tri- |
| | fluoromethyl-2H-1-benzopyran-3-carboxylic acid |
| B-6 | 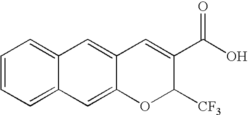 |
| | 2-Trifluoromethyl-2H-nephtho[2,3- |
| | b]pyran-3-carboxylic acid |
| B-7 | 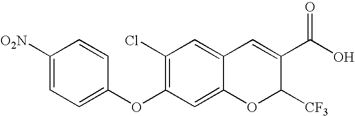 |
| | 6-Chloro-7-(4-nitrophenoxy)-2-(trifluoromethyl)-2H- |
| | 1-benzopyran-3-carboxylic acid |
| B-8 | 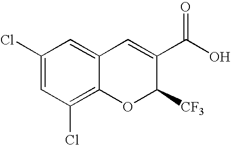 |
| | ((S)-6,8-Dichloro-2-(trifluoromethyl)- |
| | 2H-1-benzopyran-3-carboxylic acid |
| B-9 | 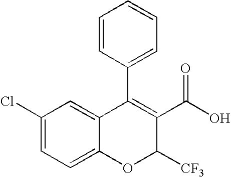 |
| | 6-Chloro-2-(trifluoromethyl)-4-phenyl-2H- |
| | 1-benzopyran-3-carboxylic acid |
| B-10 | 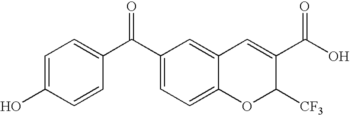 |
| | 6-(4-Hydroxybenzoyl)-2-(trifluoromethyl)- |
| | 2H-1-benzopyran-3-carboxylic acid |
| B-11 | 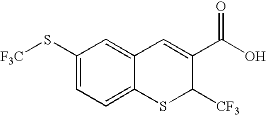 |
| | 2-(Trifluoromethyl)-6-[(trifluoromeethyl)thio]- |
| | 2H-1-benzothiopyran-3-carboxylic acid |
| B-12 | 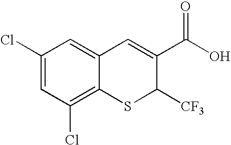 |
| | 6,8-Dichloro-2-trifluoromethyl-2H-1- |
| | benzothiopyran-3-carboxylicc acid |
| B-13 | 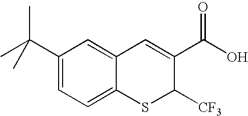 |
| | 6-(1,1-Dimethylethyl)-2-(trifluoromethyl)- |
| | 2H-1-benzothiopyran-3-carboxylic acid |
| B-14 | 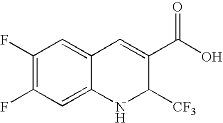 |
| | 6,7-Dichloro-1,2-dihydro-2-(trifluoro- |
| | methyl)-3-quinolinecarboxylic acid |
| B-15 | 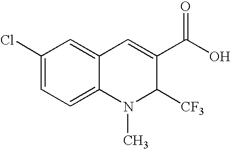 |
| | 6-Chloro-1,2-dihydro-1-methyl-2-(trifluoro- |
| | methyl)-3-quinolinecarboxylic acid |
| B-16 | 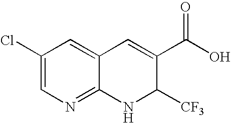 |
| | 6-Chloro-2-(trifluoromethyl)-1,2-dihydro- |
| | [1,8]naphthyridine-3-carboxylic acid |
| B-17 | 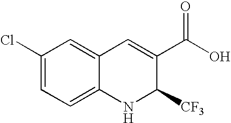 |
| | ((S)-6-Chloro-1,2-dihydro-2-(trifluoro- |
| | methyl)-3-quinolinecarboxylic acid |

In other embodiments the COX-2 selective inhibitor can be selected from the class of tricyclic COX-2 selective inhibitors represented by the general structure of formula (VII): 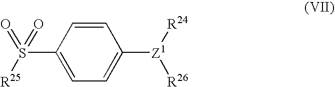
wherein:
Z¹ is selected from the group consisting of partially unsaturated or unsaturated heterocyclyl and partially unsaturated or unsaturated carbocyclic rings;
R²⁴ is selected from the group consisting of heterocyclyl, cycloalkyl, cycloalkenyl and aryl, wherein R²⁴ is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;
R²⁵ is selected from the group consisting of methyl and amino; and
R²⁶ is selected from the group consisting of a radical selected from H, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl and N-alkyl-N-arylaminosulfonyl;
   and pharmaceutically acceptable salts and prodrugs thereof.

The COX-2 selective inhibitor of formula (VII) can be selected from the group of compounds illustrated in Table 2, which includes celecoxib (B-18), valdecoxib (B-19), deracoxib (B-20), rofecoxib (B-21), etoricoxib or MK-663 (B-22) and JTE-522 (B-23), and pharmaceutically acceptable salts and prodrugs thereof.

Additional information about these COX-2 selective inhibitors can be found in patents individually cited below and incorporated herein by reference.

U.S. Pat. No. 5,466,823.

U.S. Pat. No. 5,840,924.

International Patent Publication No. WO 00/25779.

International Patent Publication No. WO 98/03484.

TABLE 2
Examples of tricyclic Cox-2 selective inhibitors

| No. | Structural formula |
|---|---|
| B-18 | 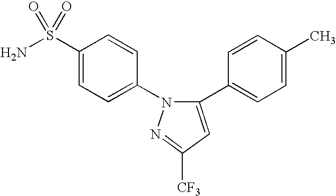 |
| B-19 | 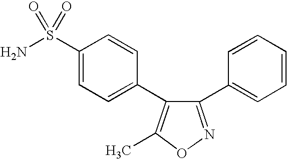 |
| B-20 | 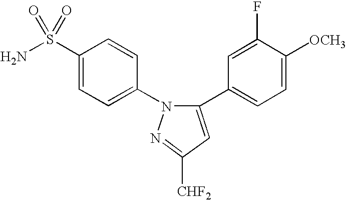 |
| B-21 | 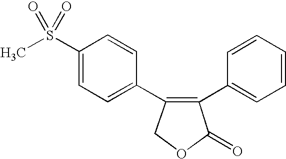 |
| B-22 | 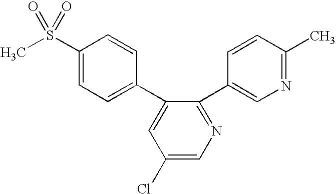 |
| B-23 | 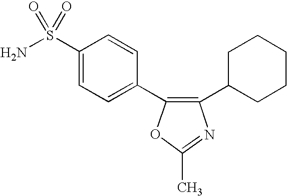 |

In certain embodiments of the invention, the Cox-2 selective inhibitor is selected from the group consisting of celecoxib, rofecoxib and etoricoxib.

In one embodiment of the invention, parecoxib (see, e.g., U.S. Pat. No. 5,932,598), which is a therapeutically effective prodrug of the tricyclic Cox-2 selective inhibitor valdecoxib, B-19 (see, e.g., U.S. Pat. No. 5,633,272), may be advantageously employed as a source of a Cox-2 inhibitor.

Parecoxib can be used as a salt, for example parecoxib sodium.

In another embodiment of the invention, the compound ABT-963 having the formula: 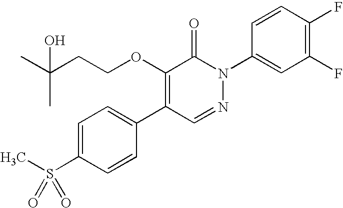
previously described in International Patent Publication No. WO 00/24719, is another tricyclic COX-2 selective inhibitor which can be advantageously employed.

Examples of specific compounds that are useful as the COX-2 selective inhibitor include, without limitation:
8-acetyl-3-(4-fluorophenyl)-2-(4-methylsulfonyl)phenyl-imidazo(1,2-a)pyridine;
5,5-dimethyl-4-(4-methylsulfonyl)phenyl-3-phenyl-2-(5H)-furanone;
5-(4-fluorophenyl)-1-[4-(methylsulfonyl)phenyl]-3-(trifluoromethyl)pyrazole;
4-(4-fluorophenyl)-5-[4-(methylsulfonyl)phenyl]-1-phenyl-3-(trifluoromethyl) pyrazole;
4-(5-(4-chlorophenyl)-3-(4-methoxyphenyl)-1H-pyrazol-1-yl)benzenesulfonamide;
4-(3,5-bis(4-methylphenyl)-1H-pyrazol-1-yl)benzenesulfonamide;
4-(5-(4-chlorophenyl)-3-phenyl-1H-pyrazol-1-yl)benzenesulfonamide;
4-(3,5-bis(4-methoxyphenyl)-1H-pyrazol-1-yl)benzenesulfonamide;
4-(5-(4-chlorophenyl)-3-(4-methylphenyl)-1H-pyrazol-1-yl)benzenesulfonamide;
4-(5-(4-chlorophenyl)-3-(4-nitrophenyl)-1H-pyrazol-1-yl)benzenesulfonamide;
4-(5-(4-chlorophenyl)-3-(5-chloro-2-thienyl)-1H-pyrazol-1-yl)benzenesulfonamide;
4-(4-chloro-3,5-diphenyl-1H-pyrazol-1-yl)benzenesulfonamide;
4-[5-(4-chlorophenyl)-3-(trifluoromethyl)-1H-pyrazol-1-yl]benzenesulfonamide;
4-[5-phenyl-3-(trifluoromethyl)-1H-pyrazol-1-yl]benzenesulfonamide;
4-[5-(4-fluorophenyl)-3-(trifluoromethyl)-1H-pyrazol-1-yl]benzenesulfonamide;
4-[5-(4-methoxyphenyl)-3-(trifluoromethyl)-1H-pyrazol-1-yl]benzenesulfonamide;
4-[5-(4-chlorophenyl)-3-(difluoromethyl)-1H-pyrazol-1-yl]benzenesulfonamide;
4-[5-(4-methylphenyl)-3-(trifluoromethyl)-1H-pyrazol-1-yl]benzenesulfonamide;
4-[4-chloro-5-(4-chlorophenyl)-3-(trifluoromethyl)-1H-pyrazol-1-yl]benzenesulfonamide;
4-[3-(difluoromethyl)-5-(4-methylphenyl)-1H-pyrazol-1-yl]benzenesulfonamide;
4-[3-(difluoromethyl)-5-phenyl-1H-pyrazol-1-yl]benzenesulfonamide;
4-[3-(difluoromethyl)-5-(4-methoxyphenyl)-1H-pyrazol-1-yl]benzenesulfonamide;
4-[3-cyano-5-(4-fluorophenyl)-1H-pyrazol-1-yl]benzenesulfonamide;
4-[3-(difluoromethyl)-5-(3-fluoro-4-methoxyphenyl)-1H-pyrazol-1-yl]benzenesulfonamide;
4-[5-(3-fluoro-4-methoxyphenyl)-3-(trifluoromethyl)-1H-pyrazol-1-yl]benzenesulfonamide;
4-[4-chloro-5-phenyl-1H-pyrazol-1-yl]benzenesulfonamide;
4-[5-(4-chlorophenyl)-3-(hydroxymethyl)-1H-pyrazol-1-yl]benzenesulfonamide;
4-[5-(4-(N,N-dimethylamino)phenyl)-3-(trifluoromethyl)-1H-pyrazol-1-yl]benzenesulfonamide;
5-(4-fluorophenyl)-6-[4-(methylsulfonyl)phenyl]spiro[2.4]hept-5-ene;
4-[6-(4-fluorophenyl)spiro[2.4]hept-5-en-5-yl]benzenesulfonamide;
6-(4-fluorophenyl)-7-[4-(methylsulfonyl)phenyl]spiro[3.4]oct-6-ene;
5-(3-chloro-4-methoxyphenyl)-6-[4-(methylsulfonyl)phenyl]spiro[2.4]hept-5-ene;
4-[6-(3-chloro-4-methoxyphenyl)spiro[2.4]hept-5-en-5-yl]benzenesulfonamide;
5-(3,5-dichloro-4-methoxyphenyl)-6-[4-(methylsulfonyl)phenyl]spiro[2.4]hept-5-ene;
5-(3-chloro-4-fluorophenyl)-6-[4-(methylsulfonyl)phenyl]spiro[2.4]hept-5-ene;
4-[6-(3,4-dichlorophenyl)spiro[2.4]hept-5-en-5-yl]benzenesulfonamide;
2-(3-chloro-4-fluorophenyl)-4-(4-fluorophenyl)-5-(4-methylsulfonylphenyl) thiazole;
2-(2-chlorophenyl)-4-(4-fluorophenyl)-5-(4-methylsulfonylphenyl)thiazole;
5-(4-fluorophenyl)-4-(4-methylsulfonylphenyl)-2-methylthiazole;
4-(4-fluorophenyl)-5-(4-methylsulfonylphenyl)-2-trifluoromethylthiazole;
4-(4-fluorophenyl)-5-(4-methylsulfonylphenyl)-2-(2-thienyl)thiazole;
4-(4-fluorophenyl)-5-(4-methylsulfonylphenyl)-2-benzylaminothiazole;
4-(4-fluorophenyl)-5-(4-methylsulfonylphenyl)-2-(1-propylamino)thiazole;
2-[(3,5-dichlorophenoxy)methyl)-4-(4-fluorophenyl)-5-[4-(methylsulfonyl) phenyl]thiazole;
5-(4-fluorophenyl)-4-(4-methylsulfonylphenyl)-2-trifluoromethylthiazole;
1-methylsulfonyl-4-[1,1-dimethyl-4-(4-fluorophenyl)cyclopenta-2,4-dien-3-yl]benzene;
4-[4-(4-fluorophenyl)-1,1-dimethylcyclopenta-2,4-dien-3-yl]benzenesulfonamide;
5-(4-fluorophenyl)-6-[4-(methylsulfonyl)phenyl]spiro[2.4]hepta-4,6-diene;
4-[6-(4-fluorophenyl)spiro[2.4]hepta-4,6-dien-5-yl]benzenesulfonamide;
6-(4-fluorophenyl)-2-methoxy-5-[4-(methylsulfonyl)phenyl]-pyridine-3-carbonitrile;
2-bromo-6-(4-fluorophenyl)-5-[4-(methylsulfonyl)phenyl]-pyridine-3-carbonitrile;
6-(4-fluorophenyl)-5-[4-(methylsulfonyl)phenyl]-2-phenyl-pyridine-3-carbonitrile;
4-[2-(4-methylpyridin-2-yl)-4-(trifluoromethyl)-1H-imidazol-1-yl]benzenesulfonamide;
4-[2-(5-methylpyridin-3-yl)-4-(trifluoromethyl)-1H-imidazol-1-yl]benzenesulfonamide;
4-[2-(2-methylpyridin-3-yl)-4-(trifluoromethyl)-1H-imidazol-1-yl]benzenesulfonamide;
3-[1-[4-(methylsulfonyl)phenyl]-4-(trifluoromethyl)-1H-imidazol-2-yl]pyridine;
2-[1-[4-(methylsulfonyl)phenyl-4-(trifluoromethyl)-1H-imidazol-2-yl]pyridine;
2-methyl-4-[1-[4-(methylsulfonyl)phenyl-4-(trifluoromethyl)-1H-imidazol-2-yl]pyridine;
2-methyl-6-[1-[4-(methylsulfonyl)phenyl-4-(trifluoromethyl)-1H-imidazol-2-yl]pyridine;
4-[2-(6-methylpyridin-3-yl)-4-(trifluoromethyl)-1H-imidazol-1-yl]benzenesulfonamide;
2-(3,4-difluorophenyl)-1-[4-(methylsulfonyl)phenyl]-4-(trifluoromethyl)-1H-imidazole;
4-[2-(4-methylphenyl)-4-(trifluoromethyl)-1H-imidazol-1-yl]benzenesulfonamide;
2-(4-chlorophenyl)-1-[4-(methylsulfonyl)phenyl]-4-methyl-1H-imidazole;
2-(4-chlorophenyl)-1-[4-(methylsulfonyl)phenyl]-4-phenyl-1H-imidazole;
2-(4-chlorophenyl)-4-(4-fluorophenyl)-1-[4-(methylsulfonyl)phenyl]-1H-imidazole;
2-(3-fluoro-4-methoxyphenyl)-1-[4-(methylsulfonyl)phenyl-4-(trifluoromethyl)-1H-imidazole;
1-[4-(methylsulfonyl)phenyl]-2-phenyl-4-trifluoromethyl-1H-imidazole;
2-(4-methylphenyl)-1-[4-(methylsulfonyl)phenyl]-4-trifluoromethyl-1H-imidazole;
4-[2-(3-chloro-4-methylphenyl)-4-(trifluoromethyl)-1H-imidazol-1-yl]benzenesulfonamide;
2-(3-fluoro-5-methylphenyl)-1-[4-(methylsulfonyl)phenyl]-4-(trifluoromethyl)-1H-imidazole;
4-[2-(3-fluoro-5-methylphenyl)-4-(trifluoromethyl)-1H-imidazol-1-yl]benzenesulfonamide;
2-(3-methylphenyl)-1-[4-(methylsulfonyl)phenyl]-4-trifluoromethyl-1H-imidazole;
4-[2-(3-methylphenyl)-4-trifluoromethyl-1H-imidazol-1-yl]benzenesulfonamide;
1-[4-(methylsulfonyl)phenyl]-2-(3-chlorophenyl)-4-trifluoromethyl-1H-imidazole;
4-[2-(3-chlorophenyl)-4-trifluoromethyl-1H-imidazol-1-yl]benzenesulfonamide;
4-[2-phenyl-4-trifluoromethyl-1H-imidazol-1-yl]benzenesulfonamide;
4-[2-(4-methoxy-3-chlorophenyl)-4-trifluoromethyl-1H-imidazol-1-yl]benzenesulfonamide;
1-allyl-4-(4-fluorophenyl)-3-[4-(methylsulfonyl)phenyl]-5-(trifluoromethyl)-1H-pyrazole;
4-[1-ethyl-4-(4-fluorophenyl)-5-(trifluoromethyl)-1H-pyrazol-3-yl]benzenesulfonamide;
N-phenyl-[4-(4-luorophenyl)-3-[4-(methylsulfonyl)phenyl]-5-(trifluoromethyl)-1H-pyrazol-1-yl]acetamide;
ethyl[4-(4-fluorophenyl)-3-[4-(methylsulfonyl)phenyl]-5-(trifluoromethyl)-1H-pyrazol-1-yl]acetate;
4-(4-fluorophenyl)-3-[4-(methylsulfonyl)phenyl]-1-(2-phenylethyl)-1H-pyrazole;
4-(4-fluorophenyl)-3-[4-(methylsulfonyl)phenyl]-1-(2-phenylethyl)-5-(trifluoromethyl)pyrazole;
1-ethyl-4-(4-fluorophenyl)-3-[4-(methylsulfonyl)phenyl]-5-(trifluoromethyl)-1H-pyrazole;
5-(4-fluorophenyl)-4-(4-methylsulfonylphenyl)-2-trifluoromethyl-1H-imidazole;
4-[4-(methylsulfonyl)phenyl]-5-(2-thiophenyl)-2-(trifluoromethyl)-1H-imidazole;
5-(4-fluorophenyl)-2-methoxy-4-[4-(methylsulfonyl)phenyl]-6-(trifluoromethyl) pyridine;
2-ethoxy-5-(4-fluorophenyl)-4-[4-(methylsulfonyl)phenyl]-6-(trifluoromethyl) pyridine;
5-(4-fluorophenyl)-4-[4-(methylsulfonyl)phenyl]-2-(2-propynyloxy)-6-(trifluoromethyl)pyridine;
2-bromo-5-(4-fluorophenyl)-4-[4-(methylsulfonyl)phenyl]-6-(trifluoromethyl) pyridine;
4-[2-(3-chloro-4-methoxyphenyl)-4,5-difluorophenyl]benzenesulfonamide;
1-(4-fluorophenyl)-2-[4-(methylsulfonyl)phenyl]benzene;
5-difluoromethyl-4-(4-methylsulfonylphenyl)-3-phenylisoxazole;
4-[3-ethyl-5-phenylisoxazol-4-yl]benzenesulfonamide;
4-[5-difluoromethyl-3-phenylisoxazol-4-yl]benzenesulfonamide;
4-[5-hydroxymethyl-3-phenylisoxazol-4-yl]benzenesulfonamide;
4-[5-methyl-3-phenyl-isoxazol-4-yl]benzenesulfonamide;
1-[2-(4-fluorophenyl)cyclopenten-1-yl]-4-(methylsulfonyl)benzene;
1-[2-(4-fluoro-2-methylphenyl)cyclopenten-1-yl]-4-(methylsulfonyl)benzene;
1-[2-(4-chlorophenyl)cyclopenten-1-yl]-4-(methylsulfonyl)benzene;
1-[2-(2,4-dichlorophenyl)cyclopenten-1-yl]-4-(methylsulfonyl)benzene;
1-[2-(4-trifluoromethylphenyl)cyclopenten-1-yl]-4-(methylsulfonyl)benzene;
1-[2-(4-methylthiophenyl)cyclopenten-1-yl]-4-(methylsulfonyl)benzene;
1-[2-(4-fluorophenyl)-4,4-dimethylcyclopenten-1-yl]-4-(methylsulfonyl)benzene;
4-[2-(4-fluorophenyl)-4,4-dimethylcyclopenten-1-yl]benzenesulfonamide;
1-[2-(4-chlorophenyl)-4,4-dimethylcyclopenten-1-yl]-4-(methylsulfonyl)benzene;
4-[2-(4-chlorophenyl)-4,4-dimethylcyclopenten-1-yl]benzenesulfonamide;
4-[2-(4-fluorophenyl)cyclopenten-1-yl]benzenesulfonamide;
4-[2-(4-chlorophenyl)cyclopenten-1-yl]benzenesulfonamide;
1-[2-(4-methoxyphenyl)cyclopenten-1-yl]-4-(methylsulfonyl)benzene;
1-[2-(2,3-difluorophenyl)cyclopenten-1-yl]-4-(methylsulfonyl)benzene;
4-[2-(3-fluoro-4-methoxyphenyl)cyclopenten-1-yl]benzenesulfonamide;
1-[2-(3-chloro-4-methoxyphenyl)cyclopenten-1-yl]-4-(methylsulfonyl)benzene;
4-[2-(3-chloro-4-fluorophenyl)cyclopenten-1-yl]benzenesulfonamide;
4-[2-(2-methylpyridin-5-yl)cyclopenten-1-yl]benzenesulfonamide;
ethyl 2-[4-(4-fluorophenyl)-5-[4-(methylsulfonyl)phenyl]oxazol-2-yl]-2-benzylacetate;
2-[4-(4-fluorophenyl)-5-[4-(methylsulfonyl)phenyl]oxazol-2-yl]acetic acid;
2-(tert-butyl)-4-(4-fluorophenyl)-5-[4-(methylsulfonyl)phenyl]oxazole;
4-(4-fluorophenyl)-5-[4-(methylsulfonyl)phenyl]-2-phenyloxazole;
4-(4-fluorophenyl)-2-methyl-5-[4-(methylsulfonyl)phenyl]oxazole;
4-[5-(3-fluoro-4-methoxyphenyl)-2-trifluoromethyl-4-oxazolyl]benzenesulfonamide;
6-chloro-2-trifluoromethyl-2H-1-benzopyran-3-carboxylic acid;
6-chloro-7-methyl-2-trifluoromethyl-2H-1-benzopyran-3-carboxylic acid;
8-(1-methylethyl)-2-trifluoromethyl-2H-1-benzopyran-3-carboxylic acid;
6-chloro-7-(1,1-dimethylethyl)-2-trifluoromethyl-2H-1-benzopyran-3-carboxylic acid;
6-chloro-8-(1-methylethyl)-2-trifluoromethyl-2H-1-benzopyran-3-carboxylic acid;
2-trifluoromethyl-3H-naphthopyran-3-carboxylic acid;
7-(1,1-dimethylethyl)-2-trifluoromethyl-2H-1-benzopyran-3-carboxylic acid;
6-bromo-2-trifluoromethyl-2H-1-benzopyran-3-carboxylic acid;
8-chloro-2-trifluoromethyl-2H-1-benzopyran-3-carboxylic acid;
6-trifluoromethoxy-2-trifluoromethyl-2H-1-benzopyran-3-carboxylic acid;
5,7-dichloro-2-trifluoromethyl-2H-1-benzopyran-3-carboxylic acid;
8-phenyl-2-trifluoromethyl-2H-1-benzopyran-3-carboxylic acid;
7,8-dimethyl-2-trifluoromethyl-2H-1-benzopyran-3-carboxylic acid;
6,8-bis(dimethylethyl)-2-trifluoromethyl-2H-1-benzopyran-3-carboxylic acid;
7-(1-methylethyl)-2-trifluoromethyl-2H-1-benzopyran-3-carboxylic acid;
7-phenyl-2-trifluoromethyl-2H-1-benzopyran-3-carboxylic acid;
6-chloro-7-ethyl-2-trifluoromethyl-2H-1-benzopyran-3-carboxylic acid;
6-chloro-8-ethyl-2-trifluoromethyl-2H-1-benzopyran-3-carboxylic acid;
6-chloro-7-phenyl-2-trifluoromethyl-2H-1-benzopyran-3-carboxylic acid;
6,7-dichloro-2-trifluoromethyl-2H-1-benzopyran-3-carboxylic acid;
6,8-dichloro-2-trifluoromethyl-2H-1-benzopyran-3-carboxylic acid;
2-trifluoromethyl-3H-naptho[2,1-b]pyran-3-carboxylic acid;
6-chloro-8-methyl-2-trifluoromethyl-2H-1-benzopyran-3-carboxylic acid;
8-chloro-6-methyl-2-trifluoromethyl-2H-1-benzopyran-3-carboxylic acid;
8-chloro-6-methoxy-2-trifluoromethyl-2H-1-benzopyran-3-carboxylic acid;
6-bromo-8-chloro-2-trifluoromethyl-2H-1-benzopyran-3-carboxylic acid;
8-bromo-6-fluoro-2-trifluoromethyl-2H-1-benzopyran-3-carboxylic acid;
8-bromo-6-methyl-2-trifluoromethyl-2H-1-benzopyran-3-carboxylic acid;
8-bromo-5-fluoro-2-trifluoromethyl-2H-1-benzopyran-3-carboxylic acid;
6-chloro-8-fluoro-2-trifluoromethyl-2H-1-benzopyran-3-carboxylic acid;
6-bromo-8-methoxy-2-trifluoromethyl-2H-1-benzopyran-3-carboxylic acid;
6-[[(phenylmethyl)amino]sulfonyl]-2-trifluoromethyl-2H-1-benzopyran-3-carboxylic acid;
6-[(dimethylamino)sulfonyl]-2-trifluoromethyl-2H-1-benzopyran-3-carboxylic acid;
6-[(methylamino)sulfonyl]-2-trifluoromethyl-2H-1-benzopyran-3-carboxylic acid;
6-[(4-morpholino)sulfonyl]-2-trifluoromethyl-2H-1-benzopyran-3-carboxylic acid;
6-[(1,1-dimethylethyl)aminosulfonyl]-2-trifluoromethyl-2H-1-benzopyran-3-carboxylic acid;
6-[(2-methylpropyl)aminosulfonyl]-2-trifluoromethyl-2H-1-benzopyran-3-carboxylic acid;
6-methylsulfonyl-2-trifluoromethyl-2H-1-benzopyran-3-carboxylic acid;
8-chloro-6-[[(phenylmethyl)amino]sulfonyl]-2-trifluoromethyl-2H-1-benzopyran-3-carboxylic acid;
6-phenylacetyl-2-trifluoromethyl-2H-1-benzopyran-3-carboxylic acid;
6,8-dibromo-2-trifluoromethyl-2H-1-benzopyran-3-carboxylic acid;
8-chloro-5,6-dimethyl-2-trifluoromethyl-2H-1-benzopyran-3-carboxylic acid;
6,8-dichloro-(S)-2-trifluoromethyl-2H-1-benzopyran-3-carboxylic acid;
6-benzylsulfonyl-2-trifluoromethyl-2H-1-benzopyran-3-carboxylic acid;
6-[[N-(2-furylmethyl)amino]sulfonyl]-2-trifluoromethyl-2H-1-benzopyran-3-carboxylic acid;
6-[[N-(2-phenylethyl)amino]sulfonyl]-2-trifluoromethyl-2H-1-benzopyran-3-carboxylic acid;
6-iodo-2-trifluoromethyl-2H-1-benzopyran-3-carboxylic acid;
7-(1,1-dimethylethyl)-2-pentafluoroethyl-2H-1-benzopyran-3-carboxylic acid;
5,5-dimethyl-3-(3-fluorophenyl)-4-(4-methylsulfonyl-2(5H)-furanone;
6-chloro-2-trifluoromethyl-2H-1-benzothiopyran-3-carboxylic acid;
4-[5-(4-chlorophenyl)-3-(trifluoromethyl)-1H-pyrazol-1-yl]benzenesulfonamide;
4-[5-(4-methylphenyl)-3-(trifluoromethyl)-1H-pyrazol-1-yl]benzenesulfonamide;
4-[5-(3-fluoro-4-methoxyphenyl)-3-(difluoromethyl)-1H-pyrazol-1-yl]benzenesulfonamide;
3-[1-[4-(methylsulfonyl)phenyl]-4-trifluoromethyl-1H-imidazol-2-yl]pyridine;
2-methyl-5-[1-[4-(methylsulfonyl)phenyl]-4-trifluoromethyl-1H-imidazol-2-yl]pyridine;
4-[2-(5-methylpyridin-3-yl)-4-(trifluoromethyl)-1H-imidazol-1-yl]benzenesulfonamide;
4-[5-methyl-3-phenylisoxazol-4-yl]benzenesulfonamide;
4-[5-hydroxymethyl-3-phenylisoxazol-4-yl]benzenesulfonamide;
[2-trifluoromethyl-5-(3,4-difluorophenyl)-4-oxazolyl]benzenesulfonamide;
4-[2-methyl-4-phenyl-5-oxazolyl]benzenesulfonamide; 4-[5-(3-fluoro-4-methoxyphenyl-2-trifluoromethyl)-4-oxazolyl]benzenesulfonamide;
   and pharmaceutically acceptable salts and prodrugs thereof.

In a further embodiment of the invention, the Cox-2 selective inhibitor used in the present invention can be selected from the class of phenylacetic acid derivatives represented by the general structure of formula (VIII): 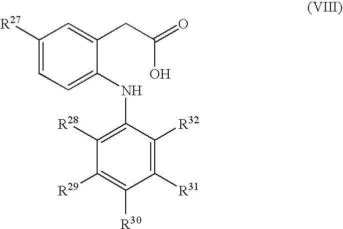
wherein:
R²⁷ is methyl, ethyl or propyl;
R²⁸ is chloro or fluoro;
R²⁹ is hydrogen, fluoro or methyl;
R³⁰ is hydrogen, fluoro, chloro, methyl, ethyl, methoxy, ethoxy or hydroxy;
R³¹ is hydrogen, fluoro or methyl; and
R³² is chloro, fluoro, trifluoromethyl, methyl, or ethyl;
   provided that R²⁸, R²⁹, R³⁰ and R³¹ are not all fluoro when R²⁷ is ethyl and R³⁰ is H; or an isomer, pharmaceutically acceptable salt, ester, or prodrug thereof.

A phenylacetic acid derivative Cox-2 selective inhibitor that is described in International Patent Publication No. WO 99/11605, incorporated by reference herein, is a compound that has the structure shown in formula (VIII), wherein:
R²⁷ is ethyl;
R²⁸ and R³⁰ are chloro;
R²⁹ and R³¹ are hydrogen; and
R³² is methyl.

Another phenylacetic acid derivative Cox-2 selective inhibitor is a compound that has the structure shown in formula (VIII), wherein:
R²⁷ is propyl;
R²⁸ and R³⁰ are chloro;
R²⁹ and R³¹ are methyl; and
R³² is ethyl.

Another phenylacetic acid derivative Cox-2 selective inhibitor, described in International Patent Publication No. WO 02/20090, incorporated by reference herein, is COX-189, also known as lumiracoxib, having the structure shown in formula (VIII), wherein:
R²⁷ is methyl;
R²⁸ is fluoro;
R³² is chloro; and
R²⁹, R³⁰, and R³¹ are hydrogen.

Cox-2 selective inhibitor compounds that have a structure similar to that shown in formula (VIII) are described in the patents individually cited below and incorporated herein by reference.

U.S. Pat. No. 6,310,099.

U.S. Pat. No. 6,291,523.

U.S. Pat. No. 5,958,978.

Other Cox-2 selective inhibitors that can be used in the present invention have the general structure shown in formula (IX), wherein the J group is a carbocycle or a heterocycle. Illustrative embodiments have the structure: 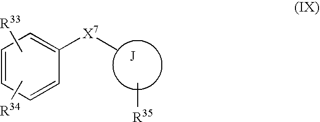
wherein:
X is O; J is 1-phenyl; R³³ is 2-NHSO₂CH₃; R³⁴ is 4-NO₂; and there is no R³⁵ group (nimesulide);
X is O; J is 1-oxo-inden-5-yl; R³³ is 2-F; R³⁴ is 4-F; and R³⁵ is 6-NHSO₂CH₃ (flosulide);
X is O; J is cyclohexyl; R³³ is 2-NHSO₂CH₃; R³⁴ is 5-NO₂; and there is no R³⁵ group (NS-398 or N-(2-cyclohexyloxynitrophenyl)methanesulfonamide);
X is S; J is 1-oxo-inden-5-yl; R³³ is 2-F; R³⁴ is 4-F; and R³⁵ is 6-N⁻SO₂CH₃.Na⁺ (L-745337);
X is S; J is thiophen-2-yl; R³³ is 4-F; there is no R³⁴ group; and R³⁵ is 5-NHSO₂CH₃ (RWJ-63556); or
X is O; J is 2-oxo-5(R)-methyl-5-(2,2,2-trifluoroethyl)furan-(5H)-3-yl; R³³ is 3-F;
R³⁴ is 4-F; and R³⁵ is 4-(p-SO₂CH₃)C₆H₄ (L-784512).

Materials that can serve as the Cox-2 selective inhibitor of the present invention include diarylmethylidenefuran derivatives that are described in U.S. Pat. No. 6,180,651. Such diarylmethylidenefuran derivatives have the general formula shown below in formula (X): 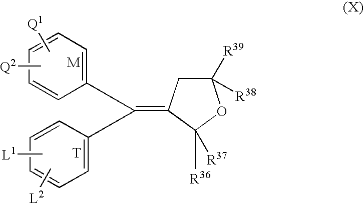
wherein:
rings T and M independently are a phenyl radical, a naphthyl radical, a radical derived from a heterocycle comprising 5 to 6 members and possessing from 1 to 4 heteroatoms, or a radical derived from a saturated hydrocarbon ring having from 3 to 7 carbon atoms;
at least one of the substituents Q¹, Q², L¹ and L² is (a) an —S(O)ₙ—R group, in which n is an integer equal to 0, 1 or 2 and R is a lower alkyl radical having 1 to 6 carbon atoms or a lower haloalkyl radical having 1 to 6 carbon atoms, or (b) an —SO₂NH₂ group, and is located in the para position; the others independently being a hydrogen atom, a halogen atom, a lower alkyl radical having 1 to 6 carbon atoms, a trifluoromethyl radical, or a lower O-alkyl radical having 1 to 6 carbon atoms, or Q¹ and Q² or L¹ and L² form a methylenedioxy group; and
R³⁶, R³⁷, R³⁸ and R³⁹ independently are a hydrogen atom, a halogen atom, a lower alkyl radical having 1 to 6 carbon atoms, a lower haloalkyl radical having 1 to 6 carbon atoms, or an aromatic radical selected from the group consisting of phenyl, naphthyl, thienyl, furyl and pyridyl; or R³⁶ and R³⁷, or R³⁸ and R³⁹ are an oxygen atom, or R³⁶ and R³⁷, or R³⁸ and R³⁹, together with the carbon atom to which they are attached, form a saturated hydrocarbon ring having from 3 to 7 carbon atoms;
   or an isomer or prodrug thereof.

Particular compounds of this family of compounds, which can serve as the Cox-2 selective inhibitor in the present invention, include N-(2-cyclohexyloxynitrophenyl)methanesulfonarmide and (E)-4-[(4-methylphenyl) (tetrahydro-2-oxo-3-furanylidene)methyl]benzenesulfonamide.

Cox-2 selective inhibitors that are useful in the present invention include darbufelone of Pfizer, CS-502 of Sankyo, LAS 34475 and LAS 34555 of Almirall Profesfarma, S-33516 of Servier, SD-8381 of Pharmacia, described in U.S. Pat. No. 6,034,256, BMS-347070 of Bristol Myers Squibb, described in U.S. Pat. No. 6,180,651, MK-966 of Merck, L-783003 and L-748731 of Merck, T-614 of Toyama, D-1367 of Chiroscience, CT3 of Atlantic Pharmaceutical, CGP-28238 of Novartis, BF-389 of Biofor/Scherer, GR-253035 of Glaxo Wellcome, 6-dioxo-9H-purin-8-yl cinnamic acid of Glaxo Wellcome, and S-2474 of Shionogi.

Information about S-33516, mentioned above, can be found in Current Drugs Headline News, at http://www.current-drugs.com/NEWS/Inflam1.htm (2001), where it was reported that S-33516 has IC₅₀ values of 0.1 and 0.001 mM against Cox-1 and Cox-2 respectively.

Compounds that can act as Cox-2 selective inhibitors include multibinding compounds containing from 2 to 10 ligands covalently attached to one or more linkers, as described in U.S. Pat. No. 6,395,724.

Compounds that can act as Cox-2 inhibitors include a conjugated linoleic acid as described in U.S. Pat. No. 6,077,868.

Compounds that can act as Cox-2 selective inhibitors include heterocyclic aromatic oxazole compounds as described in the patents individually cited below and incorporated herein by reference.

U.S. Pat. No. 5,994,381.

U.S. Pat. No. 6,362,209.

Such heterocyclic aromatic oxazole compounds have the formula shown below in formula (XI): 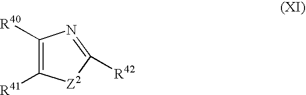
wherein:
Z² is an oxygen atom;
one of R⁴⁰ and R⁴¹ is a group of the formula 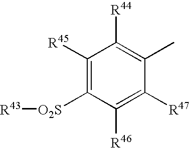
   wherein R⁴³ is lower alkyl, amino or lower alkylamino; and R⁴⁴, R⁴⁵, R⁴⁶ and R⁴⁷ are the same or different and each is hydrogen, halogen, lower alkyl, lower alkoxy, trifluoromethyl, hydroxy or amino, provided that at least one of R⁴⁴, R⁴⁵, R⁴⁶ and R⁴⁷ is not hydrogen;
the other of R⁴⁰ and R⁴¹ is an optionally substituted cycloalkyl, heterocyclyl or aryl; and
R⁴² is a lower alkyl or a halogenated lower alkyl,
   or a pharmaceutically acceptable salt thereof.

Cox-2 selective inhibitors useful herein include compounds described in the patents individually cited below and incorporated herein by reference.

U.S. Pat. No. 6,080,876.

U.S. Pat. No. 6,133,292.

Such compounds are described by formula (XII): 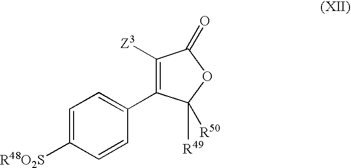
wherein:
Z³ is selected from the group consisting of (a) linear or branched C₁₋₆ alkyl, (b) linear or branched C₁₋₆ alkoxy, (c) unsubstituted, mono-, di- or tri-substituted phenyl or naphthyl wherein the substituents are selected from the group consisting of hydrogen, halo, C₁₋₃ alkoxy, CN, C₁₋₃ fluoroalkyl, C₁₋₃ alkyl and —CO₂H;
R⁴⁸ is selected from the group consisting of NH₂ and CH₃,
R⁴⁹ is selected from the group consisting of C₁₋₆ alkyl unsubstituted or substituted with C₃₋₆ cycloalkyl, and C₃₋₆ cycloalkyl;
R⁵⁰ is selected from the group consisting of C₁₋₆ alkyl unsubstituted or substituted with one, two or three fluoro atoms; and C₃₋₆ cycloalkyl;
with the proviso that R⁴⁹ and R⁵⁰ are not the same.

Compounds that can act as Cox-2 selective inhibitors include pyridines described in the patents individually cited below and incorporated herein by reference.

U.S. Pat. No. 6,369,275.

U.S. Pat. No. 6,127,545.

U.S. Pat. No. 6,130,334.

U.S. Pat. No. 6,204,387.

U.S. Pat. No. 6,071,936.

U.S. Pat. No. 6,001,843.

U.S. Pat. No. 6,040,450.

Such compounds have the general formula described by formula (XIII): 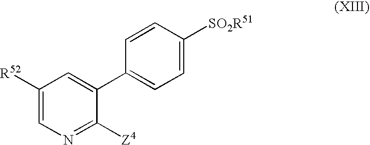
wherein:
R⁵¹ is selected from the group consisting of: CH₃, NH₂, NHC(O)CF₃ and NHCH₃;
Z⁴ is a mono-, di-, or trisubstituted phenyl or pyridinyl (or the N-oxide thereof), having substituents selected from the group consisting of hydrogen, halo, C₁₋₆ alkoxy, C₁₋₆ alkylthio, CN, C₁₋₆ alkyl, C₁₋₆ fluoroalkyl, N₃, —CO₂R⁵³, hydroxy, —C(R⁵⁴)(R⁵⁵)—OH, —C₁₋₆alkyl-CO₂—R⁵⁶ and C₁₋₆ fluoroalkoxy;
R⁵² is selected from the group consisting of halo, C₁₋₆ alkoxy, C₁₋₆ alkylthio, CN, C₁₋₆ alkyl, C₁₋₆ fluoroalkyl, N₃, —CO₂R⁵⁷, hydroxy, —C(R⁵⁸)(R⁵⁹)—OH, —C₁₋₆alkyl-CO₂—R⁶⁰, C₁₋₆ fluoroalkoxy, NO₂, NR⁶¹R⁶² and NHCOR⁶³; and
R⁵³, R⁵⁴, R⁵⁵, R⁵⁶, R⁵⁷, R⁵⁸, R⁵⁹, R⁶⁰, R⁶¹, R⁶² and R⁶³ are each independently selected from the group consisting of hydrogen and C₁₋₆ alkyl; or R⁵⁴ and R⁵⁵, R⁵⁸ and R⁵⁹, or R⁶¹ and R⁶², together with the atom to which they are attached, form a saturated monocyclic ring of 3, 4, 5, 6 or 7 atoms.

Compounds that can act as Cox-2 selective inhibitors include diarylbenzopyran derivatives as described in U.S. Pat. No. 6,340,694, incorporated herein by reference. Such diarylbenzopyran derivatives have the general formula shown below in formula (XIV): 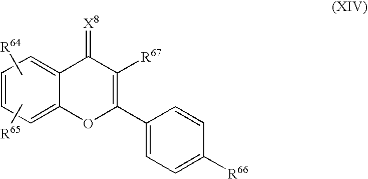
wherein:
X⁸ is an oxygen atom or a sulfur atom;
R⁶⁴ and R⁶⁵, identical to or different from each other, are independently hydrogen, halogen, C₁-C₆ lower alkyl, trifluoromethyl, alkoxy, hydroxy, nitro, nitrile or carboxyl;
R⁶⁶ is a group of a formula S(O)ₙR⁶⁸ where n is an integer of 0 to 2, R⁶⁸ is hydrogen, C₁-C₆ lower alkyl, or a group of formula NR⁶⁹R⁷⁰ wherein R⁶⁹ and R⁷⁰, identical to or different from each other, are independently hydrogen or C₁-C₆ lower alkyl group; and
R⁶⁷ is oxazolyl, benzo[b]thienyl, furanyl, thienyl, naphthyl, thiazolyl, indolyl, pyrrolyl, benzofuranyl, pyrazolyl, pyrazolyl substituted with a C₁-C₆ lower alkyl group, indanyl, pyrazinyl, or a substituted group represented by one of the following structures: 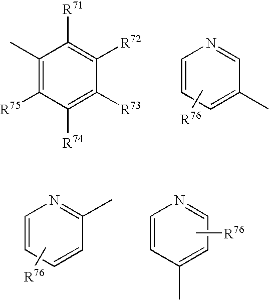
   wherein:
R⁷¹ through R⁷⁵, identical to or different from one another, are independently hydrogen, halogen, C₁-C₆ lower alkyl, trifluoromethyl, alkoxy, hydroxy, hydroxyalkyl, nitro, a group of formula S(O)ₙR⁶⁸, a group of formula NR⁶⁹R⁷⁰, trifluoromethoxy, nitrile, carboxyl, acetyl or formyl, wherein n, R⁶⁸, R⁶⁹ and R⁷⁰ have the same meaning as defined by R⁶⁶ above; and
R⁷⁶ is hydrogen, halogen, C₁-C₆ lower alkyl, trifluoromethyl, alkoxy, hydroxy, trifluoromethoxy, carboxyl or acetyl.

Compounds that can act as Cox-2 selective inhibitors include 1-(4-sulfamylaryl)-3-substituted-5-aryl-2-pyrazolines as described in U.S. Pat. No. 6,376,519, incorporated herein by reference. Such compounds have the formula shown below in formula (XV): 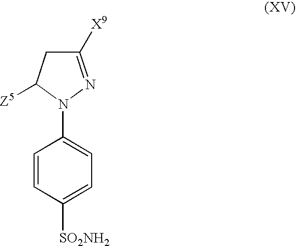
wherein:
X⁹ is selected from the group consisting of C₁-C₆ trihalomethyl, for example trifluoromethyl C₁-C₆ alkyl; and an optionally substituted or di-substituted phenyl group of formula 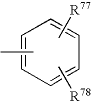
   wherein R⁷⁷ and R⁷⁸ are independently selected from the group consisting of hydrogen, halogen (e.g., chlorine, fluorine or bromine), hydroxyl, nitro, C₁-C₆ (e.g., C₁-C₃) alkyl, C₁-C₆ (e.g., C₁-C₃) alkoxy, carboxy, C₁-C₆ trihaloalkyl (e.g., trihalomethyl such as trifluoromethyl), and cyano; and
Z⁵ is selected from the group consisting of substituted and unsubstituted aryl.

Compounds that can act as Cox-2 selective inhibitors of the present invention include heterocycles as described in U.S. Pat. No. 6,153,787, incorporated herein by reference. Such heterocycles have the general formulas shown below in formulas (XVI) and (XVII): 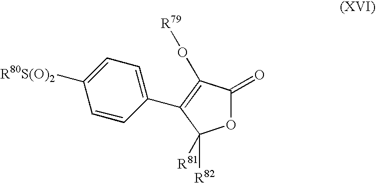
wherein:
R⁷⁹ is mono-, di- or tri-substituted C₁₋₁₂ alkyl, unsubstituted or mono-, di- or tri-substituted linear or branched C₂₋₁₀ alkenyl, unsubstituted or mono-, di- or tri-substituted linear or branched C₂₋₁₀ alkynyl, unsubstituted ormono-, di- or tri-substituted C₃₋₁₂ cycloalkenyl, or unsubstituted or mono-, di- or tri-substituted C₅₋₁₂ cycloalkynyl, wherein the substituents are chosen from the group consisting of halo (selected from F, Cl, Br, and I), OH, CF₃, C₃₋₆ cycloalkyl, ═O, dioxolane and CN;
R⁸⁰ is selected from the group consisting of: CH₃, NH₂, NHC(O)CF₃ and NHCH₃;
R⁸¹ and R⁸² are independently chosen from the group consisting of hydrogen and C₁₋₁₀ alkyl; or R⁸¹ and R⁸² together with the carbon to which they are attached form a saturated monocyclic carbon ring of 3, 4, 5, 6 or 7 atoms.

Formula (XVII) is: 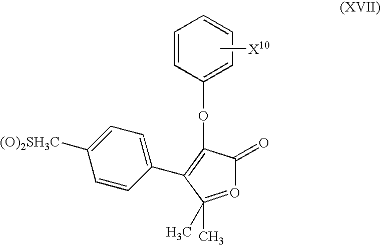
wherein X¹⁰ is fluoro or chloro.

Compounds that can act as Cox-2 selective inhibitors include 2,3,5-trisubstituted pyridines as described in U.S. Pat. No. 6,046,217, incorporated herein by reference. Such compounds have the general formula shown below in formula (XVIII): 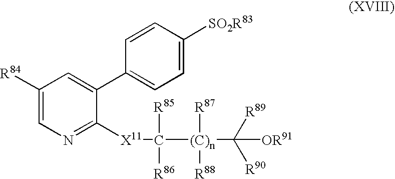
or a pharmaceutically acceptable salt thereof, wherein:
X¹¹ is selected from the group consisting of O, S and bond;
n is 0 or 1;
R⁸³ is selected from the group consisting of CH₃, NH₂ and NHC(O)CF₃;
R⁸⁴ is selected from the group consisting of halo, C₁₋₆ alkoxy, C₁₋₆ alkylthio, CN, C₁₋₆ alkyl, C₁₋₆ fluoroalkyl, N₃, —CO₂R⁹², hydroxy, —C(R⁹³)(R⁹⁴)—OH, C₁₋₆ alkyl-CO₂—R⁹⁵, C₁₋₆ fluoroalkoxy, NO₂, NR⁹⁶R⁹⁷ and NHCOR⁹⁸;
R⁸⁵ to R⁹⁸ are independently chosen from the group consisting of hydrogen and C₁₋₆ alkyl; or R⁸⁵ and R⁸⁹, or R⁸⁹ and R⁹⁰, together with the atoms to which they are attached, form a carbocyclic ring of 3, 4, 5, 6 or 7 atoms; or R⁸⁵ and R⁸⁷ are joined to form a bond.

One exemplary embodiment of the Cox-2 selective inhibitor of formula (XVIII) is that wherein X is a bond.

Another exemplary embodiment of the Cox-2 selective inhibitor of formula (XVIII) is that wherein X is O.

Another exemplary embodiment of the Cox-2 selective inhibitor of formula (XVIII) is that wherein X is S.

Another exemplary embodiment of the Cox-2 selective inhibitor of formula (XVIII) is that wherein R⁸³ is CH₃.

Another exemplary embodiment of the Cox-2 selective inhibitor of formula (XVIII) is that wherein R⁸⁴ is halo or C₁₋₆ fluoroalkyl.

Compounds that can act as Cox-2 selective inhibitors include diaryl bicyclic heterocycles as described in U.S. Pat. No. 6,329,421. Such compounds have the general formula shown below in formula (XIX): 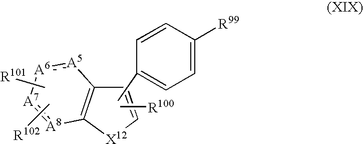
and pharmaceutically acceptable salts thereof, wherein:
-A⁵=A⁶-A⁷=A⁸- is selected from the group consisting of (a) —CH═CH—CH═CH—, (b) —CH₂—CH₂—CH₂—C(O)—, —CH₂—CH₂—C(O)—CH₂—, —CH₂—C(O)—CH₂—CH₂, —C(O)—CH₂—CH₂—CH₂, (c) —CH₂—CH₂—C(O)—, —CH₂—C(O)—CH₂—, —C(O)—CH₂—CH₂—, (d) —CH₂—CH₂—O—C(O)—, —CH₂—O—C(O)—CH₂—, —O—C(O)—CH₂—CH₂—, (e) —CH₂—CH₂—C(O)—O—, —CH₂—C(O)—OCH₂—, —C(O)—O—CH₂—CH₂—, (f) —C(R¹⁰⁵)₂—O—C(O)—, —C(O)—O—C(R¹⁰⁵)₂—, —O—C(O)—C(R¹⁰⁵)₂—, —C(R¹⁰⁵)₂—C(O)—O—, (g) —N═CH—CH═CH—, (h) —CH═N—CH═CH—, (i) —CH═CH—N═CH—, (j) —CH═CH—CH═N—, (k) —N═CH—CH═N—, (l) —N═CH—N═CH—, (m) —CH═N—CH═N—, (n) —S—CH═N—, (o) —S—N═CH—, (p) —N═N—NH—, (q) —CH═N—S— and (r) —N═CH—S—;
R⁹⁹ is selected from the group consisting of S(O)₂CH₃, S(O)₂NH₂, S(O)₂NHCOCF₃, S(O)(NH)CH₃, S(O)(NH)NH₂, S(O)(NH)NHCOCF₃, P(O)(CH₃)OH and P(O)(CH₃)NH₂;
R¹⁰⁰ is selected from the group consisting of (a) C₁₋₆ alkyl, (b) C₃₋₇ cycloalkyl, (c) mono- or di-substituted phenyl or naphthyl where the substituent is selected from the group consisting of hydrogen, halo including F, Cl, Br and I, C₁₋₆ alkoxy, C₁₋₆ alkylthio, CN, CF₃, C₁₋₆ alkyl, N₃, —CO₂H, —CO₂—C₁₋₄ alkyl, —C(R¹⁰³)(R¹⁰⁴)—OH, —C(R¹⁰³)(R¹⁰⁴)—O—C₁₋₄ alkyl and —C₁₋₆ alkyl-CO₂—R¹⁰⁶; (d) mono- or di-substituted heteroaryl where the heteroaryl is a monocyclic aromatic ring of 5 atoms, said ring having one hetero atom which is S, O, or N, and optionally 1, 2 or 3 additional N atoms; or a monocyclic ring of 6 atoms, said ring having one hetero atom which is N, and optionally 1, 2, 3 or 4 additional N atoms; and said substituents are selected from the group consisting of hydrogen, halo including F, Cl, Br and I, C₁₋₆ alkoxy, C₁₋₆ alkylthio, CN, CF₃, C₁₋₆ alkyl, N₃, —CO₂H, —CO₂—C₁₋₄ alkyl, —C(R¹⁰³)(R¹⁰⁴)—OH and —C(R¹⁰³)(R¹⁰⁴)—O—C₁₋₄ alkyl; and (e) benzoheteroaryl which includes the benzo fused analogs of (d);
R¹⁰¹ and R¹⁰² are substituents residing on any position of -A⁵=A⁶-A⁷=A⁸- and are selected independently from the group consisting of hydrogen, CF₃, CN, C₁₋₆ alkyl, Q⁴, CO₂H, C(R¹⁰³)(R¹⁰⁴)OH, —O-Q⁴, —S-Q⁴, and optionally C₁₋₃ alkyl substituted —C₁₋₅ alkyl-Q³, —O—C₁₋₅ alkyl-Q³, —S—C₁₋₅ alkyl-Q³, —C₁₋₃ alkyl-O—C₁₋₃ alkyl-Q³, —C₁₋₃ alkyl-S—C₁₋₃ alkyl-Q³, —C₁₋₅ alkyl-O-Q⁴ and —C₁₋₅ alkyl-S-Q⁴, wherein the substituent resides on the alkyl chain; where Q³ is Q⁴, CO₂H or C(R¹⁰³)(R¹⁰⁴)OH and Q⁴ is CO₂—C₁₋₄ alkyl, tetrazolyl-5-yl, or C(R¹⁰³)(R¹⁰⁴)O—C₁₋₄ alkyl;
R¹⁰³, R¹⁰⁴ and R¹⁰⁵ are each independently selected from the group consisting of hydrogen and C₁₋₆ alkyl; or R¹⁰³ and R¹⁰⁴ together with the carbon to which they are attached form a saturated monocyclic carbon ring of 3, 4, 5, 6 or 7 atoms, or two R¹⁰⁵ groups on the same carbon form a saturated monocyclic carbon ring of 3, 4, 5, 6 or 7 atoms;
R¹⁰⁶ is hydrogen or C₁₋₆ alkyl;
R¹⁰⁷ is hydrogen, C₁₋₆ alkyl or aryl; and
X⁷ is O, S, NR¹⁰⁷, CO, C(R¹⁰⁷)₂, C(R¹⁰⁷)(OH), —C(R¹⁰⁷)═C(R¹⁰⁷)—, —C(R¹⁰⁷)═N— or —N═C(R¹⁰⁷)—.

Compounds that can act as Cox-2 selective inhibitors include salts of a 5-amino- or substituted amino-1,2,3-triazole compound as described in U.S. Pat. No. 6,239,137. These salts are of a class of compounds of formula (XX): 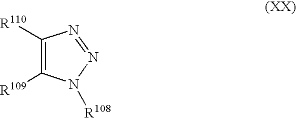
wherein:
R¹⁰⁸ is 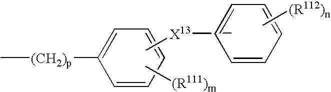
   where p is 0 to 2; m is 0 to 4; n is 0 to 5; X¹³ is O, S, SO, SO₂, CO, CHCN, CH₂ or C═NR¹¹³ where R¹¹³ is hydrogen, lower alkyl, hydroxy, lower alkoxy, amino, lower alkylamino, di(lower alkyl)amino or cyano; and R¹¹¹ and R¹¹² are independently halogen, cyano, trifluoromethyl, lower alkanoyl, nitro, lower alkyl, lower alkoxy, carboxy, lower carbalkoxy, trifluoromethoxy, acetamido, lower alkylthio, lower alkylsulfinyl, lower alkylsulfonyl, trichlorovinyl, trifluoromethylthio, trifluoromethylsulfinyl or trifluoromethylsulfonyl;
R¹⁰⁹ is amino, mono or di(lower alkyl)amino, acetamido, acetimido, ureido, formamido, formamido or guanidino; and
R¹¹⁰ is carbamoyl, cyano, carbazoyl, amidino or N-hydroxycarbamoyl;
wherein the lower alkyl, lower alkyl containing, lower alkoxy and lower alkanoyl groups contain from 1 to 3 carbon atoms.

Compounds that can act as Cox-2 selective inhibitors include pyrazole derivatives as described in U.S. Pat. No. 6,136,831. Such compounds have the formula shown below in formula (XXI): 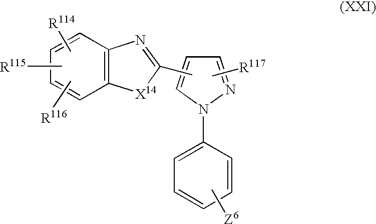
wherein:
R¹¹⁴ is hydrogen or halogen;
R¹¹⁵ and R¹¹⁶ are each independently hydrogen, halogen, lower alkyl, lower alkoxy, hydroxy or lower alkanoyloxy;
R¹¹⁷ is lower haloalkyl or lower alkyl;
X¹⁴ is sulfur, oxygen or NH; and
Z⁶ is lower alkylthio, lower alkylsulfonyl or sulfamoyl;
   or a pharmaceutically acceptable salt thereof.

Compounds that can act as Cox-2 selective inhibitors include substituted derivatives of benzosulfonamides as described in U.S. Pat. No. 6,297,282. Such compounds have the formula shown below in formula (XXII): 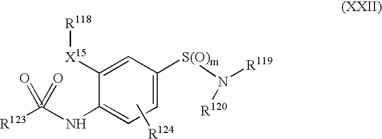
wherein:
X¹⁵ denotes oxygen, sulfur or NH;
R¹¹⁸ is an optionally unsaturated alkyl or alkyloxyalkyl group, optionally mono- or polysubstituted or mixed substituted by halogen, alkoxy, oxo or cyano, a cycloalkyl, aryl or heteroaryl group optionally mono- or polysubstituted or mixed substituted by halogen, alkyl, CF₃, cyano or alkoxy;
R¹¹⁹ and R¹²⁰, independently from one another, denote hydrogen, an optionally polyfluorized alkyl group, an aralkyl, aryl or heteroaryl group or a group (CH₂)ₙ—X¹⁶; or R¹¹⁹ and R¹²⁰, together with the N atom, denote a 3- to 7-membered, saturated, partially or completely unsaturated heterocycle with one or more heteroatoms N, O or S, which can optionally be substituted by oxo, an alkyl, alkylaryl or aryl group, or a group (CH₂)ₙ—X¹⁶;
X¹⁶ denotes halogen, NO₂, —OR¹²¹, —COR¹²¹, —CO₂R¹²¹, —OCO₂R¹²¹, —CN, —CONR¹²¹OR¹²², —CONR¹²¹R¹²², —SR¹²¹, —S(O)R¹²¹, —S(O)₂R¹²¹, —NR¹²¹R¹²², —NHC(O)R¹²¹ or —NHS(O)₂R¹²¹;
n denotes a whole number from 0 to 6;
R¹²³ denotes a straight-chained or branched alkyl group with 1-10 C-atoms, a cycloalkyl group, an alkylcarboxyl group, an aryl group, aralkyl group, a heteroaryl or heteroaralkyl group which can optionally be mono- or polysubstituted or mixed substituted by halogen or alkoxy;
R¹²⁴ denotes halogen, hydroxy, a straight-chained or branched alkyl, alkoxy, acyloxy or alkyloxycarbonyl group with 1-6 C-atoms, which can optionally be mono- or polysubstituted by halogen, NO₂, —OR¹²¹, —COR¹²¹, —CO₂R¹²¹, —OCO₂R¹²¹, —CN, —CONR¹²¹OR¹²², —CONR¹²¹R¹²², —SR¹²¹, —S(O)R¹²¹, —S(O)₂R¹²¹, —NR¹²¹R¹²², —NHC(O)R¹²¹, —NHS(O)₂R¹²¹, or a polyfluoroalkyl group;
R¹²¹ and R¹²², independently from one another, denote hydrogen, alkyl, aralkyl or aryl; and
m denotes a whole number from 0 to 2;
   and pharmaceutically-acceptable salts thereof.

Compounds that can act as Cox-2 selective inhibitors include 3-phenyl-4-(4(methylsulfonyl)phenyl)-2-(5H)-furanones as described in U.S. Pat. No. 6,239,173. Such compounds have the formula shown below in formula (XXIII): 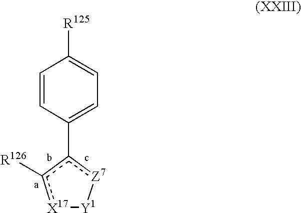
or pharmaceutically acceptable salts thereof, wherein:
—X¹⁷—Y¹-Z⁷-, when side b is a double bond, and sides a and c are single bonds, is selected from the group consisting of —CH₂CH₂CH₂—, —C(O)CH₂CH₂—, —CH₂CH₂C(O)—, —CR¹²⁹ (R^{129′})—O—C(O)—, —C(O)—O—CR¹²⁹(R^{129′})—, —CH₂—NR¹²⁷—CH₂—, —CR¹²⁹(R^{129′})—NR¹²⁷—C(O)—, —CR¹²⁸═CR^{128′}—S—, —S—CR¹²⁸ ═CR^{128′}, —S—N═CH—, —CH═N—S—, —N═CR¹²⁸—O—, —O—CR¹²⁸═N—, —N═CR¹²⁸—NH—, —N═CR¹²⁸—S—, —S—CR¹²⁸═N—, —C(O)—NR¹²⁷—CR¹²⁹(R^{129′})—, —R¹²⁷N—CH═CH— provided R¹²² is not —S(O)₂CH₃, and —CH═CH—NR¹²⁷— provided R¹²⁵ is not —S(O)₂CH₃;
—X¹⁷—Y¹-Z⁷-, when sides a and c are double bonds and side b is a single bond, is selected from the group consisting of ═CH—O—CH═, ═CH—NR¹²⁷—CH═, ═N—S—CH═, ═CH—S—N═, ═N—O—CH═, ═CH—O—N═, ═N—S—N═ and ═N—O—N═;
R¹²⁵ is selected from the group consisting of S(O)₂CH₃, S(O)₂NH₂, S(O)₂NHC(O)CF₃, S(O)(NH)CH₃, S(O)(NH)NH₂, S(O)(NH)NHC(O)CF₃, P(O)(CH₃)OH and P(O)(CH₃)NH₂;
R¹²⁶ is selected from the group consisting of (a) C₁₋₆ alkyl; (b) C₃, C₄, C₅, C₆ or C₇ cycloalkyl; (c) mono-, di- or tri-substituted phenyl or naphthyl, where the substituent is selected from the group consisting of hydrogen, halo, C₁₋₆ alkoxy, C₁₋₆ alkylthio, CN, CF₃, C₁₋₆ alkyl, N₃, —CO₂H, —CO₂—C₁₋₄ alkyl, —C(R¹²⁹)(R¹³⁰)—OH, —C(R¹²⁹)(R¹³⁰)—O—C₁₋₄ alkyl, and —C₁₋₆ alkyl-CO₂—R¹²⁹; (d) mono-, di- or tri-substituted heteroaryl wherein the heteroaryl is a monocyclic aromatic ring of 5 atoms, said ring having one hetero atom which is S, O or N, and optionally 1, 2 or 3 additional N atoms, or the heteroaryl is a monocyclic ring of 6 atoms, said ring having one hetero atom which is N, and optionally 1, 2, 3 or 4 additional N atoms, where the substituents are selected from the group consisting of hydrogen, halo (including fluoro, chloro, bromo and iodo), C₁₋₆ alkyl, C₁₋₆ alkoxy, C₁₋₆ alkylthio, CN, CF₃, N₃, —C(R¹²⁹)(R¹³⁰)—OH, and —C(R¹²⁹)(R¹³⁰)—O—C₁₋₄ alkyl; and (e) benzoheteroaryl including the benzo-fused analogs of (d);
R¹²⁷ is selected from the group consisting of hydrogen, CF₃, CN, C₁₋₆ alkyl, hydroxy-C₁₋₆ alkyl, —C(O)—C₁₋₆ alkyl, optionally C₁₋₃ alkyl-substituted —C₁₋₅ alkyl-Q⁵, —C₁₋₃ alkyl-O—C₁₋₃ alkyl-Q⁵, —C₁₋₃ alkyl-S—C₁₋₃ alkyl-Q⁵, —C₁₋₅ alkyl-O-Q⁵ and —C₁₋₅ alkyl-S-Q⁵ where the substituent resides on the alkyl; and -Q⁵;
R¹²⁸ and R^{128′} are each independently selected from the group consisting of hydrogen, CF₃, CN, C₁₋₆ alkyl, -Q⁵, —O-Q⁵; —S-Q⁵, and optionally C₁₋₃ alkyl-substituted —C₁₋₅ alkyl-Q⁵, —O—C₁₋₅ alkyl-Q⁵, —S—C₁₋₅ alkyl-Q⁵, —C₁₋₃ alkyl-O—C₁₋₃ alkyl-Q⁵, —C₁₋₃ alkyl-S—C₁₋₃ alkyl-Q⁵, —C₁₋₅ alkyl-O-Q⁵, —C₁₋₅ alkyl-S-Q⁵ wherein the substituent resides on the alkyl;
R¹²⁹, R^{129′}, R¹³⁰, R¹³¹ and R¹³² are each independently selected from the group consisting of hydrogen and C₁₋₆ alkyl; or R¹²⁹ and R¹³⁰, or R¹³¹ and R¹³², together with the carbon to which they are attached, form a saturated monocyclic carbon ring of 3, 4, 5, 6 or 7 atoms; and
Q⁵ is CO₂H, CO₂—C₁₋₄ alkyl, tetrazolyl-5-yl, C(R¹³¹)(R¹³²)(OH) or C(R¹³¹)(R¹³²)(O—C₁₋₄ alkyl);
provided that when —X¹⁷—Y¹-Z⁷- is —S—CR¹²⁸═CR^{128′}, then R¹²⁸ and R^{128′} are other than CF₃.

Compounds that can act as Cox-2 selective inhibitors include bicyclic carbonyl indole compounds as described in U.S. Pat. No. 6,303,628. Such compounds have the formula shown below in formula (XXIV): 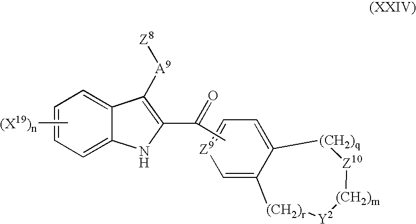
or pharmaceutically acceptable salts thereof, wherein:
A⁹ is C₁₋₆ alkylene or —NR¹³³—;
Z⁸ is C(=L³)R¹³⁴ or SO₂R¹³⁵;
Z⁹ is CH or N;
Z¹⁰ and Y² are independently selected from —CH₂—, O, S and —N—R¹³³;
m is 1, 2 or 3;
q and r are independently 0, 1 or 2;
X¹⁸ is independently selected from halogen, C₁₋₄ alkyl, halo-substituted C₁₋₄ alkyl, hydroxy, C₁₋₄ alkoxy, halo-substituted C₁₋₄ alkoxy, C₁₋₄ alkylthio, nitro, amino, mono- or di(C₁₋₄ alkyl)amino and cyano;
n is 0, 1, 2, 3 or 4;
L³ is oxygen or sulfur;
R¹³³ is hydrogen or C₁₋₄ alkyl;
R¹³⁴ is hydroxy, C₁₋₆ alkyl, halo-substituted C₁₋₆ alkyl, C₁₋₆ alkoxy, halo-substituted C₁₋₆ alkoxy, C₃₋₇ cycloalkoxy, C₁₋₄ alkyl(C₃₋₇ cycloalkoxy), —NR¹³⁶R¹³⁷, C₁₋₄ alkylphenyl-O— or phenyl-O—, said phenyl being optionally substituted with one to five substituents independently selected from halogen, C₁₋₄ alkyl, hydroxy, C₁₋₄ alkoxy and nitro;
R¹³⁵ is C₁₋₆ alkyl or halo-substituted C₁₋₆ alkyl; and
R¹³⁶ and R¹³⁷ are independently selected from hydrogen, C₁₋₆ alkyl and halo-substituted C₁₋₆ alkyl.

Compounds that can act as a Cox-2 selective inhibitors include benzimidazole compounds as described in U.S. Pat. No. 6,310,079. Such compounds have the formula shown below in formula (XXV): 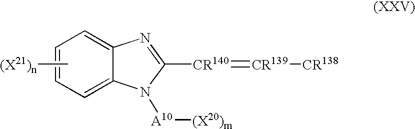
or a pharmaceutically acceptable salt thereof, wherein:
A¹⁰ is heteroaryl selected from (a) a 5-membered monocyclic aromatic ring having one hetero atom selected from O, S and N and optionally containing one to three N atom(s) in addition to said hetero atom, and (b) a 6-membered monocyclic aromatic ring having one N atom and optionally containing one to four N atom(s) in addition to said N atom; said heteroaryl being connected to the nitrogen atom on the benzimidazole through a carbon atom on the heteroaryl ring;
X²⁰ is independently selected from halo, C₁-C₄ alkyl, hydroxy, C₁-C₄ alkoxy, halo-substituted C₁-C₄ alkyl, hydroxy-substituted C₁-C₄ alkyl, (C₁-C₄ alkoxy)C₁-C₄ alkyl, halo-substituted C₁-C₄ alkoxy, amino, N-(C₁-C₄ alkyl)amino, N,N-di(C₁-C₄ alkyl)amino, [N-(C₁-C₄ alkyl)amino]C₁-C₄ alkyl, [N,N-di(C₁-C₄ alkyl)amino]C₁-C₄ alkyl, N-(C₁-C₄ alkanoyl)amino, N-(C₁-C₄ alkyl)(C₁-C₄ alkanoyl)amino, N-[(C₁-C₄ alkyl)sulfonyl]amino, N-[(halo-substituted C₁-C₄ alkyl)sulfonyl]amino, C₁-C₄ alkanoyl, carboxy, (C₁-C₄ alkoxy)carbonyl, carbamoyl, [N-(C₁-C₄ alkyl)amino]carbonyl, [N,N-di(C₁-C₄ alkyl)amino]carbonyl, cyano, nitro, mercapto, (C₁-C₄ alkyl)thio, (C₁-C₄ alkyl)sulfinyl, (C₁-C₄ alkyl)sulfonyl, aminosulfonyl, [N-(C₁-C₄ alkyl)amino]sulfonyl and [N,N-di(C₁-C₄ alkyl)amino]sulfonyl;
X²¹ is independently selected from halo, C₁-C₄ alkyl, hydroxy, C₁-C₄ alkoxy, halo-substituted C₁-C₄ alkyl, hydroxy-substituted C₁-C₄ alkyl, (C₁-C₄ alkoxy)C₁-C₄ alkyl, halo-substituted C₁-C₄ alkoxy, amino, N-(C₁-C₄ alkyl)amino, N,N-di(C₁-C₄ alkyl)amino, [N-(C₁-C₄ alkyl)amino]C₁-C₄ alkyl, [N,N-di(C₁-C₄ alkyl)amino]C₁-C₄ alkyl, N-(C₁-C₄ alkanoyl)amino, N-(C₁-C₄ alkyl)-N-(C₁-C₄ alkanoyl)amino, N-[(C₁-C₄ alkyl)sulfonyl]amino, N-[(halo-substituted C₁-C₄ alkyl)sulfonyl]amino, C₁-C₄ alkanoyl, carboxy, (C₁-C₄ alkoxy)carbonyl, carbamoyl, [N-(C₁-C₄ alkyl)amino]carbonyl, [N,N-di(C₁-C₄ alkyl)amino]carbonyl, N-carbamoylamino, cyano, nitro, mercapto, (C₁-C₄ alkyl)thio, (C₁-C₄ alkyl)sulfinyl, (C₁-C₄ alkyl)sulfonyl, aminosulfonyl, [N-(C₁-C₄ alkyl)amino]sulfonyl and [N,N-di(C₁-C₄ alkyl)amino]sulfonyl;
R¹³⁸ is selected from hydrogen; straight or branched C₁-C₄ alkyl optionally substituted with one to three substituent(s) independently selected from halo, hydroxy, C₁-C₄ alkoxy, amino, N-(C₁-C₄ alkyl)amino and N,N-di(C₁-C₄ alkyl)amino; C₃-C₈ cycloalkyl optionally substituted with one to three substituent(s) independently selected from halo, C₁-C₄ alkyl, hydroxy, C₁-C₄ alkoxy, amino, N-(C₁-C₄ alkyl)amino and N,N-di(C₁-C₄ alkyl)amino; C₄-C₈ cycloalkenyl optionally substituted with one to three substituent(s) independently selected from halo, C₁-C₄ alkyl, hydroxy, C₁-C₄ alkoxy, amino, N-(C₁-C₄ alkyl)amino and N,N-di(C₁-C₄ alkyl)amino; phenyl optionally substituted with one to three substituent(s) independently selected from halo, C₁-C₄ alkyl, hydroxy, C₁-C₄ alkoxy, halo-substituted C₁-C₄ alkyl, hydroxy-substituted C₁-C₄ alkyl, (C₁-C₄ alkoxy)C₁-C₄ alkyl, halo-substituted C₁-C₄ alkoxy, amino, N-(C₁-C₄ alkyl)amino, N,N-di(C₁-C₄ alkyl)amino, [N-(C₁-C₄ alkyl)amino]C₁-C₄ alkyl, [N,N-di(C₁-C₄ alkyl)amino]C₁-C₄ alkyl, N-(C₁-C₄ alkanoyl)amino, N-[C₁-C₄ alkyl)(C₁-C₄ alkanoyl)]amino, N-[(C₁-C₄ alkyl)sulfonyl]amino, N-[(halo-substituted C₁-C₄ alkyl)sulfonyl]amino, C₁-C₄ alkanoyl, carboxy, (C₁-C₄ alkoxy)carbonyl, carbamoyl, [N-(C₁-C₄ alkyl)amino]carbonyl, [N,N-di(C₁-C₄ alkyl)amino]carbonyl, cyano, nitro, mercapto, (C₁-C₄ alkyl)thio, (C₁-C₄ alkyl)sulfinyl, (C₁-C₄ alkyl)sulfonyl, aminosulfonyl, [N-(C₁-C₄ alkyl)amino]sulfonyl and [N,N-di(C₁-C₄ alkyl)amino]sulfonyl; and heteroaryl selected from (a) a 5-membered monocyclic aromatic ring having one hetero atom selected from O, S and N and optionally containing one to three N atom(s) in addition to said hetero atom; and (b) a 6-membered monocyclic aromatic ring having one N atom and optionally containing one to four N atom(s) in addition to said N atom; said heteroaryl being optionally substituted with one to three substituent(s) selected from X²⁰;
R¹³⁹ and R¹⁴⁰ are independently selected from hydrogen; halo; C₁-C₄ alkyl; phenyl optionally substituted with one to three substituent(s) independently selected from halo, C₁-C₄ alkyl, hydroxy, C₁-C₄ alkoxy, amino, N-(C₁-C₄ alkyl)amino and N,N-di(C₁-C₄ alkyl)amino; or R¹³⁸ and R¹³⁹ can form, together with the carbon atom to which they are attached, a C₃-C₇ cycloalkyl ring;
m is 0, 1, 2,3, 4 or 5; and
n is 0, 1, 2, 3 or 4.

Compounds that can act as Cox-2 selective inhibitors include indole compounds that are described in U.S. Pat. No. 6,300,363. Such compounds have the formula shown below in formula (XXVI): 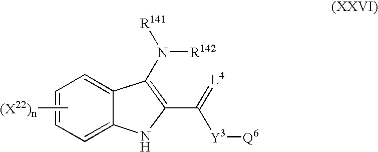
and pharmaceutically acceptable salts thereof, wherein:
L⁴ is oxygen or sulfur;
Y³ is a direct bond or C₁₋₄ alkylidene;
Q⁶ is (a) C₁₋₆ alkyl or halosubstituted C₁₋₆ alkyl, said alkyl being optionally substituted with up to three substituents independently selected from hydroxy, C₁₋₄ alkoxy, amino and mono- or di-(C₁₋₄ alkyl)amino; (b) C₃₋₇ cycloalkyl optionally substituted with up to three substituents independently selected from hydroxy, C₁₋₄ alkyl and C₁₋₄ alkoxy; (c) phenyl or naphthyl, said phenyl or naphthyl being optionally substituted with up to four substituents independently selected from halo, C₁₋₄ alkyl, halo-substituted C₁₋₄ alkyl, hydroxy, C₁₋₄ alkoxy, halo-substituted C₁₋₄ alkoxy, S(O)ₘR¹⁴³, SO₂NH₂, SO₂N(C₁₋₄ alkyl)₂, amino, mono- or di-(C₁₋₄ alkyl)amino, NHSO₂R¹⁴³, NHC(O)R¹⁴³, CN, CO₂H, CO₂(C₁₋₄ alkyl), C₁₋₄ alkyl-OH, C₁₋₄ alkyl-OR¹⁴³, CONH₂, CONH(C₁₋₄ alkyl), CON(C₁₋₄ alkyl)₂ and —O—Y-phenyl, said phenyl being optionally substituted with one or two substituents independently selected from halo, C₁₋₄ alkyl, CF₃, hydroxy, OR¹⁴³, S(O)ₘR¹⁴³, amino, mono- or di-(C₁₋₄ alkyl)amino and CN; (d) a monocyclic aromatic group of 5 atoms, said aromatic group having one heteroatom selected from O, S and N and optionally containing up to three N atoms in addition to said heteroatom, and said aromatic group being substituted with up to three substituents independently selected from halo, C₁₋₄ alkyl, halo-substituted C₁₋₄ alkyl, hydroxy, C₁₋₄ alkoxy, halo-substituted C₁₋₄ alkoxy, C₁₋₄ alkyl-OH, S(O)ₘR¹⁴³, SO₂NH₂, SO₂N(C₁₋₄ alkyl)₂, amino, mono- or di-(C₁₋₄ alkyl)amino, NHSO₂R¹⁴³, NHC(O)R¹⁴³, CN, CO₂H, CO₂(C₁₋₄ alkyl), C₁₋₄ alkyl-OR¹⁴³, CONH₂, CONH(C₁₋₄ alkyl), CON(C₁₋₄ alkyl)₂, phenyl, and mono-, di- or tri-substituted phenyl wherein the substituent is independently selected from halo, CF₃, C₁₋₄ alkyl, hydroxy, C₁₋₄ alkoxy, OCF₃, SR¹⁴³, SO₂CH₃, SO₂NH₂, amino, C₁₋₄ alkylamino and NHSO₂R¹⁴³; or (e) a monocyclic aromatic group of 6 atoms, said aromatic group having one heteroatom which is N and optionally containing up to three atoms in addition to said heteroatom, and said aromatic group being substituted with up to three substituents independently selected from halo, C₁₋₄ alkyl, halo-substituted C₁₋₄ alkyl, hydroxy, C₁₋₄ alkoxy, halo-substituted C₁₋₄ alkoxy, C₁₋₄ alkyl-OH, S(O)ₘR¹⁴³, SO₂NH₂, SO₂N(C₁₋₄ alkyl)₂, amino, mono- or di-(C₁₋₄ alkyl)amino, NHSO₂R¹⁴³, NHC(O)R¹⁴³, CN, CO₂H, CO₂(C₁₋₄ alkyl), C₁₋₄ alkyl-OR¹⁴³, CONH₂, CONH(C₁₋₄ alkyl), CON(C₁₋₄ alkyl)₂, phenyl, and mono-, di- or tri-substituted phenyl wherein the substituent is independently selected from halo, CF₃, C₁₋₄ alkyl, hydroxy, C₁₋₄ alkoxy, OCF₃, SR¹⁴³, SO₂CH₃, SO₂NH₂, amino, C₁₋₄ alkylamino and NHSO₂R¹⁴³;
R¹⁴¹ is hydrogen or C₁₋₆ alkyl optionally substituted with a substituent selected independently from hydroxy, OR¹⁴³, nitro, amino, mono- or di-(C₁₋₄ alkyl)amino, CO₂H, CO₂(C₁₋₄ alkyl), CONH₂, CONH(C₁₋₄ alkyl) and CON(C₁₋₄ alkyl)₂;
R¹⁴² is hydrogen; C₁₋₄ alkyl; C(O)R¹⁴⁵ where R¹⁴⁵ is selected from (a) C₁₋₂₂ alkyl or C₂₋₂₂ alkenyl, said alkyl or alkenyl being optionally substituted with up to four substituents independently selected from halo, hydroxy, OR¹⁴³, S(O)ₘR¹⁴³, nitro, amino, mono- or di-(C₁₋₄ alkyl)amino, NHSO₂R¹⁴³, CO₂H, CO₂(C₁₋₄ alkyl), CONH₂, CONH(C₁₋₄ alkyl), CON(C₁₋₄ alkyl)₂, OC(O)R¹⁴³, thienyl, naphthyl and groups of the following formulae: 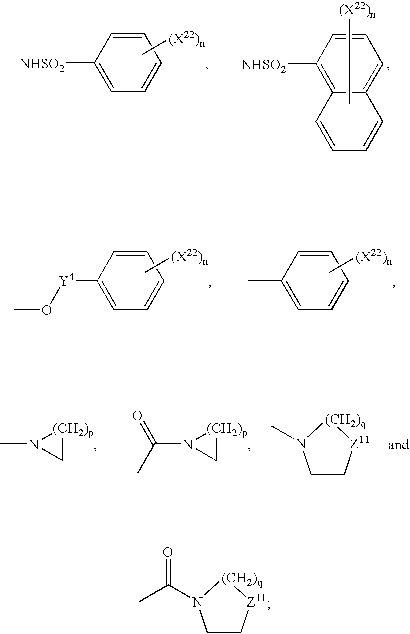
   (b) C₁₋₂₂ alkyl or C₂₋₂₂ alkenyl, said alkyl or alkenyl being optionally substituted with 5 to 45 halogen atoms; (c) —Y⁵—C₃₋₇ cycloalkyl or —Y⁵—C₃₋₇ cycloalkenyl, said cycloalkyl or cycloalkenyl being optionally substituted with up to three substituent independently selected from C₁₋₄ alkyl, hydroxy, OR¹⁴³, S(O)ₘR¹⁴³, amino, mono- or di-(C₁₋₄ alkyl)amino, CONH₂, CONH(C₁₋₄ alkyl) and CON(C₁₋₄ alkyl)₂; (d) phenyl or naphthyl, said phenyl or naphthyl being optionally substituted with up to seven substituents independently selected from halo, C₁₋₈ alkyl, C₁₋₄ alkyl-OH, hydroxy, C₁₋₈ alkoxy, halo-substituted C₁₋₈ alkyl, halo-substituted C₁₋₈ alkoxy, CN, nitro, S(O)ₘR¹⁴³, SO₂NH₂, SO₂NH(C₁₋₄ alkyl), SO₂N(C₁₋₄ alkyl)₂, amino, C₁₋₄ alkylamino, di-(C₁₋₄ alkyl)amino, CONH₂, CONH(C₁₋₄ alkyl), CON(C₁₋₄ alkyl)₂, OC(O)R¹⁴³, and phenyl optionally substituted with up to three substituents independently selected from halo, C₁₋₄ alkyl, hydroxy, OCH₃, CF₃, OCF₃, CN, nitro, amino, mono- or di-(C₁₋₄ alkyl)amino, CO₂H, CO₂(C₁₋₄ alkyl) and CONH₂; (e) a monocyclic aromatic group of 5 atoms, said aromatic group having one heteroatom selected from O, S and N and optionally containing up to three N atoms in addition to said heteroatom, and said aromatic group being substituted with up to three substituents independently selected from halo, C₁₋₄ alkyl, halo-substituted C₁₋₄ alkyl, hydroxy, C₁₋₄ alkoxy, halo-substituted C₁₋₄ alkoxy, C₁₋₄ alkyl-OH, S(O)ₘR¹⁴³, SO₂NH₂, SO₂N(C₁₋₄ alkyl)₂, amino, mono- or di-(C₁₋₄ alkyl)amino, NHSO₂R¹⁴³, NHC(O)R¹⁴³, CN, CO₂H, CO₂(C₁₋₄ alkyl), C₁₋₄ alkyl-OR¹⁴³, CONH₂, CONH(C₁₋₄ alkyl), CON(C₁₋₄ alkyl)₂, phenyl, and mono-, di- or tri-substituted phenyl wherein the substituent is independently selected from halo, CF₃, C₁₋₄ alkyl, hydroxy, C₁₋₄ alkoxy, OCF₃, SR¹⁴³, SO₂CH₃, SO₂NH₂, amino, C₁₋₄ alkylamino and NHSO₂R¹⁴³; (f) a monocyclic aromatic group of 6 atoms, said aromatic group having one heteroatom which is N and optionally containing up to three atoms in addition to said heteroatom, and said aromatic group being substituted with up to three substituents independently selected from halo, C₁₋₄ alkyl, halo-substituted C₁₋₄ alkyl, hydroxy, C₁₋₄ alkoxy, halo-substituted C₁₋₄ alkoxy, C₁₋₄ alkyl-OH, S(O)ₘR¹⁴³, SO₂NH₂, SO₂N(C₁₋₄ alkyl)₂, amino, mono- or di-(C₁₋₄ alkyl)amino, NHSO₂R¹⁴³, NHC(O)R¹⁴³, CN, CO₂H, CO₂(C₁₋₄ alkyl), C₁₋₄ alkyl-OR¹⁴³, CONH₂, CONH(C₁₋₄ alkyl), CON(C₁₋₄ alkyl)₂, phenyl, and mono-, di- or tri-substituted phenyl wherein the substituent is independently selected from halo, CF₃, C₁₋₄ alkyl, hydroxy, C₁₋₄ alkoxy, OCF₃, SR¹⁴³, SO₂CH₃, SO₂NH₂, amino, C₁₋₄ alkylamino and NHSO₂R¹⁴³; or (g) a group of the following formula: 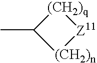
X²² is halo, C₁₋₄ alkyl, hydroxy, C₁₋₄ alkoxy, halo-substituted C₁₋₄ alkoxy, S(O)ₘR¹⁴³, amino, mono- or di-(C₁₋₄ alkyl)amino, NHSO₂R¹⁴³, nitro, halo-substituted C₁₋₄ alkyl, CN, CO₂H, CO₂ (C₁₋₄ alkyl), C₁₋₄ alkyl-OH, C₁₋₄ alkyl-OR¹⁴³, CONH₂, CONH(C₁₋₄ alkyl) or CON(C₁₋₄ alkyl)₂;
R¹⁴³ is C₁₋₄ alkyl or halo-substituted C₁₋₄ alkyl;
m is 0, 1 or 2;
n is 0, 1, 2 or 3;
p is 1, 2, 3, 4 or 5;
q is 2 or 3;
Z¹¹ is oxygen, sulfur or NR¹⁴⁴; and
R¹⁴⁴ is hydrogen, C₁₋₆ alkyl, halo-substituted C₁₋₄ alkyl or —Y⁵-phenyl, said phenyl being optionally substituted with up to two substituents independently selected from halo, C₁₋₄ alkyl, hydroxy, C₁₋₄ alkoxy, S(O)ₘR¹⁴³, amino, mono- or di-(C₁₋₄ alkyl)amino, CF₃, OCF₃, CN and nitro;
with the proviso that a group of formula —Y⁵-Q is not methyl or ethyl when X²² is hydrogen, L⁴ is oxygen, R¹⁴¹ is hydrogen and R¹⁴² is acetyl.

Compounds that can act as Cox-2 selective inhibitors include aryl phenylhydrazides as described in U.S. Pat. No. 6,077,869. Such compounds have the formula shown below in formula (XXVII): 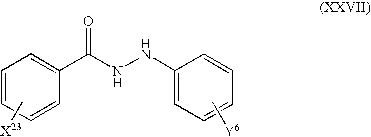
wherein X²³ and Y⁶ are selected from hydrogen, halogen, alkyl, nitro, amino and other oxygen- and sulfur-containing functional groups such as hydroxy, methoxy and methylsulfonyl.

Compounds that can act as Cox-2 selective inhibitors include 2-aryloxy-4-aryl furan-2-ones as described in U.S. Pat. No. 6,140,515. Such compounds have the formula shown below in formula (XXVIII): 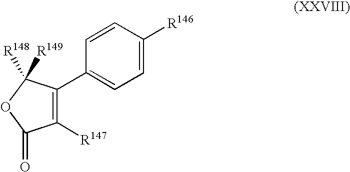
or a pharmaceutically acceptable salt thereof, wherein:
R¹⁴⁶ is selected from the group consisting of SCH₃, —S(O)₂CH₃ and —S(O)₂NH₂;
R¹⁴⁷ is selected from the group consisting of OR¹⁵⁰, mono- or di-substituted phenyl or pyridyl wherein the substituents are selected from the group consisting of methyl, chloro and fluoro;
R¹⁵⁰ is unsubstituted or mono- or di-substituted phenyl or pyridyl wherein the substituents are selected from the group consisting of methyl, chloro and fluoro;
R¹⁴⁸ is H or C₁₋₄ alkyl optionally substituted with 1 to 3 groups of F, Cl or Br; and
R¹⁴⁹ is H and C₁₋₄ alkyl optionally substituted with 1 to 3 groups of F, Cl or Br;
with the proviso that R¹⁴⁸ and R¹⁴⁹ are not the same.

Compounds that can act as Cox-2 selective inhibitors include bisaryl compounds as described in U.S. Pat. No. 5,994,379. Such compounds have the formula shown below in formula (XXIX): 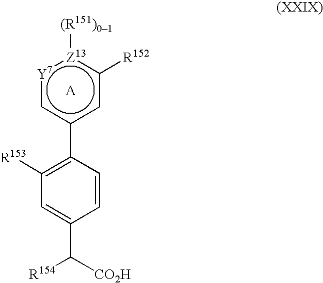
or a pharmaceutically acceptable salt, ester or tautomer thereof, wherein:
Z¹³ is C or N;
when Z¹³ is N, R¹⁵¹ represents H or is absent, or is taken in conjunction with R¹⁵² as described below;
when Z¹³ is C, R¹⁵¹ represents H and R¹⁵² is a moiety which has the following characteristics: (a) it is a linear chain of 3-4 atoms containing 0-2 double bonds, which can adopt an energetically stable transoid configuration and if a double bond is present, the bond is in the trans configuration, (b) it is lipophilic except for the atom bonded directly to ring A, which is either lipophilic or non-lipophilic, and (c) there exists an energetically stable configuration planar with ring A to within about 15 degrees; or R¹⁵¹ and R¹⁵² are taken in combination and represent a 5- or 6-membered aromatic or non-aromatic ring D fused to ring A, said ring D containing 0-3 heteroatoms selected from O, S and N; said ring D being lipophilic except for the atoms attached directly to ring A, which are lipophilic or non-lipophilic, and said ring D having available an energetically stable configuration planar with ring A to within about 15 degrees; said ring D further being substituted with one R^{a} group selected from the group consisting of C₁₋₂ alkyl, —O—C₁₋₂ alkyl, —NHC₁₋₂ alkyl, —N(C₁₋₂ alkyl)₂, —C(O)C₁₋₂ alkyl, —S—C₁₋₂ alkyl and —C(S)C₁₋₂ alkyl;
Y⁷ represents N, CH or C—O—C₁₋₃ alkyl, and when Z¹³ is N, Y⁷ can also represent a carbonyl group;
R¹⁵³ represents H, Br, Cl or F; and
R¹⁵⁴ represents H or CH₃.

Compounds that can act as Cox-2 selective inhibitors include 1,5-diarylpyrazoles as described in U.S. Pat. No. 6,028,202. Such compounds have the formula shown below in formula (XXX): 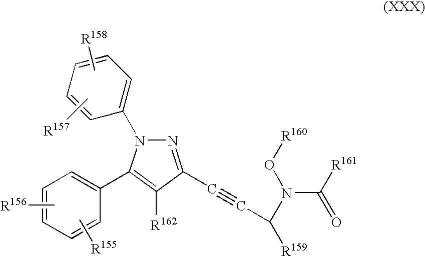
wherein:
R¹⁵⁵, R¹⁵⁶, R¹⁵⁷ and R¹⁵⁸ are independently selected from the group consisting of hydrogen, C₁₋₅ alkyl, C₁₋₅ alkoxy, phenyl, halo, hydroxy, C₁₋₅ alkylsulfonyl, C₁₋₅ alkylthio, trihalo-C₁₋₅ alkyl, amino, nitro and 2-quinolinylmethoxy;
R¹⁵⁹ is hydrogen; C₁₋₅ alkyl; trihalo-C₁₋₅ alkyl; phenyl; substituted phenyl where the phenyl substituents are halogen, C₁₋₅ alkoxy, trihalo-C₁₋₅ alkyl or nitro; or heteroaryl of 5-7 ring members where at least one of the ring members is nitrogen, sulfur or oxygen;
R¹⁶⁰ is hydrogen; C₁₋₅ alkyl; phenyl-C₁₋₅ alkyl; substituted phenyl-C₁₋₅ alkyl where the phenyl substituents are halogen, C₁₋₅ alkoxy, trihalo-C₁₋₅ alkyl or nitro; C₁₋₅ alkoxycarbonyl; phenoxycarbonyl; or substituted phenoxycarbonyl where the phenyl substituents are halogen, C₁₋₅ alkoxy, trihalo-C₁₋₅ alkyl or nitro;
R¹⁶¹ is C₁₋₁₀ alkyl; substituted C₁₋₁₀ alkyl where the substituents are halogen, trihalo-C₁₋₅ alkyl, C₁₋₅ alkoxy, carboxy, C₁₋₅ alkoxycarbonyl, amino, C₁₋₅ alkylamino, di(C₁₋₅ alkyl)amino, di(C₁₋₅ alkyl)amino-C₁₋₅ alkylamino, C₁₋₅ alkylamino-C₁₋₅ alkylamino or a heterocycle containing 4-8 ring atoms where one more of the ring atoms is nitrogen, oxygen or sulfur, said heterocycle being optionally substituted with C₁₋₅ alkyl; phenyl; substituted phenyl where the phenyl substituents are one or more of C₁₋₅ alkyl, halogen, C₁₋₅ alkoxy, trihalo-C₁₋₅ alkyl or nitro; heteroaryl having 5-7 ring atoms where one or more atoms are nitrogen, oxygen or sulfur; fused heteroaryl where one or more 5-7 membered aromatic rings are fused to the heteroaryl; or NR¹⁶³R¹⁶⁴ where R¹⁶³ and R¹⁶⁴ are independently selected from hydrogen and C₁₋₅ alkyl, or R¹⁶³ and R¹⁶⁴ may be taken together with the depicted nitrogen to form a heteroaryl ring of 5-7 ring members where one or more of the ring members is nitrogen, sulfur or oxygen, said heteroaryl ring being optionally substituted with C₁₋₅ alkyl; and
R¹⁶² is hydrogen, C₁₋₅ alkyl, nitro, amino or halogen;
   or pharmaceutically acceptable salts thereof.

Compounds that can act as Cox-2 selective inhibitors include 2-substituted imidazoles as described in U.S. Pat. No. 6,040,320. Such compounds have the formula shown below in formula (XXXI): 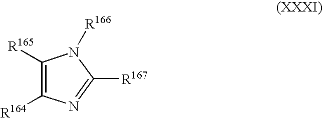
wherein:
R¹⁶⁴ is phenyl; heteroaryl containing 5 to 6 ring atoms; or substituted phenyl wherein the substituents are independently selected from one or members of the group consisting of C₁₋₅ alkyl, halogen, nitro, trifluoromethyl and nitrile;
R¹⁶⁵ is phenyl; heteroaryl containing 5 to 6 ring atoms; substituted heteroaryl wherein the substituents are independently selected from one or more members of the group consisting of C₁₋₅ alkyl and halogen; or substituted phenyl wherein the substituents are independently selected from one or members of the group consisting of C₁₋₅ alkyl, halogen, nitro, trifluoromethyl and nitrile;
R¹⁶⁶ is hydrogen, 2-(trimethylsilyl)ethoxymethyl, C₁₋₅ alkoxycarbonyl, aryloxycarbonyl, aryl-C₁₋₅ alkyloxycarbonyl, aryl-C₁₋₅ alkyl, phthalimido-C₁₋₅ alkyl, amino-C₁₋₅ alkyl, diamino-C₁₋₅ alkyl, succinimido-C₁₋₅ alkyl, C₁₋₅ alkylcarbonyl, arylcarbonyl, C₁₋₅ alkylcarbonyl-C₁₋₅ alkyl, aryloxycarbonyl-C₁₋₅ alkyl, heteroaryl-C₁₋₅ alkyl where the heteroaryl contains 5 to 6 ring atoms, or substituted aryl-C₁₋₅ alkyl wherein the aryl substituents are independently selected from one or more members of the group consisting of C₁₋₅ alkyl, C₁₋₅ alkoxy, halogen, amino, C₁₋₅ alkylamino and di(C₁₋₅ alkyl)amino; and
R¹⁶⁷ is (A¹¹)ₙ—(CH¹⁶⁵)_{q}—X²¹ wherein A¹¹ is sulfur or carbonyl; n is 0 or 1; q is 0-9; and X²⁴ is selected from the group consisting of hydrogen; hydroxy; halogen; vinyl; ethynyl; C₁₋₅ alkyl; C₃₋₇ cycloalkyl; C₁₋₅ alkoxy; phenoxy; phenyl; aryl-C₁₋₅ alkyl; amino; C₁₋₅ alkylamino; nitrile; phthalimido; amido; phenylcarbonyl; C₁₋₅ alkylaminocarbonyl; phenylaminocarbonyl; aryl-C₁₋₅ alkylaminocarbonyl; C₁₋₅ alkylthio; C₁₋₅ alkylsulfonyl; phenylsulfonyl; substituted sulfonamido wherein the sulfonyl substituent is selected from the group consisting of C₁₋₅ alkyl, phenyl, araC₁₋₅ alkyl, thienyl, furanyl and naphthyl; substituted vinyl wherein the substituents are independently selected from one or members of the group consisting of fluorine, bromine, chlorine and iodine; substituted ethynyl wherein the substituents are independently selected from one or more members of the group consisting of fluorine, bromine, chlorine and iodine; substituted C₁₋₅ alkyl wherein the substituents are selected from the group consisting of one or more C₁₋₅ alkoxy, trihaloalkyl, phthalimido and amino; substituted phenyl wherein the phenyl substituents are independently selected from one or more members of the group consisting of C₁₋₅ alkyl, halogen and C₁₋₅ alkoxy; substituted phenoxy wherein the phenyl substituents are independently selected from one or more members of the group consisting of C₁₋₅ alkyl, halogen and C₁₋₅ alkoxy; substituted C₁₋₅ alkoxy wherein the alkyl substituent is selected from the group consisting of phthalimido and amino; substituted aryl-C₁₋₅ alkyl wherein the alkyl substituent is hydroxyl; substituted aryl-C₁₋₅ alkyl wherein the phenyl substituents are independently selected from one or more members of the group consisting of C₁₋₅ alkyl, halogen and C₁₋₅ alkoxy; substituted amido wherein the carbonyl substituent is selected from the group consisting of C₁₋₅ alkyl, phenyl, arylC₁₋₅ alkyl, thienyl, furanyl and naphthyl; substituted phenylcarbonyl wherein the phenyl substituents are independently selected from one or members of the group consisting of C₁₋₅ alkyl, halogen and C₁₋₅ alkoxy; substituted C₁₋₅ alkylthio wherein the alkyl substituent is selected from the group consisting of hydroxy and phthalimido; substituted C₁₋₅ alkylsulfonyl wherein the alkyl substituent is selected from the group consisting of hydroxy and phthalimido; and substituted phenylsulfonyl wherein the phenyl substituents are independently selected from one or members of the group consisting of bromine, fluorine, chlorine, C₁₋₅ alkoxy and trifluoromethyl; with the proviso that (a) if A¹¹ is sulfur and X²⁴ is other than hydrogen, C₁₋₅ alkylaminocarbonyl, phenylaminocarbonyl, aryl-C₁₋₅ alkylaminocarbonyl, C₁₋₅ alkylsulfonyl or phenylsulfonyl, then q must be equal to or greater than 1; (b) if A¹¹ is sulfur and q is 1, then X²⁴ cannot be C₁₋₂ alkyl; (c) if A¹¹ is carbonyl and q is 0, then X²⁴ cannot be vinyl, ethynyl, C₁₋₅ alkylaminocarbonyl, phenylaminocarbonyl, aryl-C₁₋₅ alkylaminocarbonyl, C₁₋₅ alkylsulfonyl or phenylsulfonyl; (d) if A¹¹ is carbonyl, q is 0 and X²⁴ is H, then R¹⁶⁶ is not 2-(trimethylsilyl)ethoxymethyl; (e) if n is 0 and q is 0, then X²⁴ cannot be hydrogen;
   and pharmaceutically acceptable salts thereof.

Compounds that can act as Cox-2 selective inhibitors include 1,3- and 2,3-diarylcycloalkano- and cycloalkenopyrazoles as described in U.S. Pat. No. 6,083,969. Such compounds have the general formulas (XXXII) and (XXXIII) shown below: 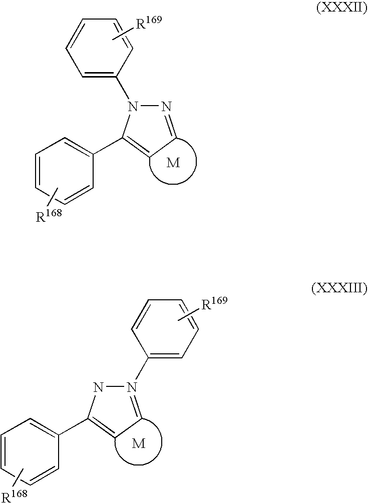
wherein:
R¹⁶⁸ and R¹⁶⁹ are independently selected from the group consisting of hydrogen, halogen, (C₁-C₆)alkyl, (C₁-C₆)alkoxy, nitro, amino, hydroxy, trifluoro, —S(C₁-C₆)alkyl, —SO(C₁-C₆)alkyl and —SO₂(C₁-C₆)alkyl; and
the fused moiety M is selected from the group consisting of an optionally substituted cyclohexyl and cycloheptyl group having the formulae: 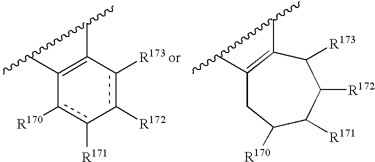
   wherein:
R¹⁷⁰ is selected from the group consisting of hydrogen, halogen, hydroxy and carbonyl;
R¹⁷¹ and R¹⁷² are independently selected from the group consisting of hydrogen, halogen, hydroxy, carbonyl, amino, (C₁-C₆)alkyl, (C₁-C₆)alkoxy, ═NOH, —NR¹⁷⁴R¹⁷⁵, —OCH₃, —OCH₂CH₃, —OSO₂NHCO₂CH₃, ═CHCO₂CH₂CH₃, —CH₂CO₂H, —CH₂CO₂CH₃, —CH₂CO₂CH₂CH₃, —CH₂CON(CH₃)₂, —CH₂CO₂NHCH₃, —CHCHCO₂CH₂CH₃, —OCON(CH₃)OH, —C(COCH₃)₂, di(C₁-C₆)alkyl and di(C₁-C₆)alkoxy; and
R¹⁷³ is selected from the group consisting of hydrogen, halogen, hydroxy, carbonyl, amino, (C₁-C₆)alkyl, (C₁-C₆)alkoxy and optionally substituted carboxyphenyl, wherein substituents on the carboxyphenyl group are selected from the group consisting of halogen, hydroxy, amino, (C₁-C₆)alkyl and (C₁-C₆)alkoxy;
or R¹⁷⁰ and R¹⁷¹ taken together form a moiety selected from the group consisting of —OCOCH₂—, —ONH(CH₃)COCH₂—, —OCOCH.dbd. and —O—;
and/or R¹⁷² and R¹⁷³ taken together form a moiety selected from the group consisting of —O— and 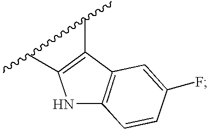
R¹⁷⁴ is selected from the group consisting of hydrogen, OH, —OCOCH₃, —COCH₃ and (C₁-C₆)alkyl; and
R¹⁷⁵ is selected from the group consisting of hydrogen, OH, —OCOCH₃, —COCH₃, (C₁-C₆)alkyl, —CONH₂ and —SO₂CH₃;
with the proviso that if M is a cyclohexyl group, then R¹⁷⁰ through R¹⁷³ may not all be hydrogen;
   and pharmaceutically acceptable salts, esters and pro-drug forms thereof.

Compounds that can serve as Cox-2 selective inhibitors include esters derived from indolealkanols and amides derived from indolealkylamides as described in U.S. Pat. No. 6,306,890. Such compounds have the general formula shown below in formula (XXXIV): 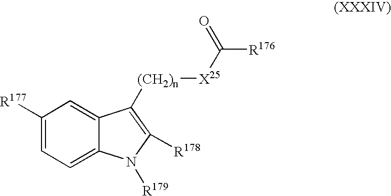
wherein:
R¹⁷⁶ is C₁-C₆ alkyl, C₁-C₆ branched alkyl, C₄-C₈ cycloalkyl, C₁-C₆ hydroxyalkyl, branched C₁-C₆ hydroxyalkyl, hydroxy-substituted C₄-C₈ aryl, primary, secondary or tertiary C₁-C₆ alkylamino, primary, secondary or tertiary branched C₁-C₆ alkylamino, primary, secondary or tertiary C₄-C₈ arylamino, C₁-C₆ alkylcarboxylic acid, branched C₁-C₆ alkylcarboxylic acid, C₁-C₆ alkylester, branched C₁-C₆ alkylester, C₄-C₈ aryl, C₄-C₈ arylcarboxylic acid, C₄-C₈ arylester, C₄-C₈ aryl-substituted C₁-C₆ alkyl, C₄-C₈ heterocyclic alkyl or aryl with O, N or S in the ring, alkyl-substituted or aryl-substituted C₄-C₈ heterocyclic alkyl or aryl with O, N or S in the ring, or halo-substituted versions thereof, where halo is chloro, bromo, fluoro or iodo;
R¹⁷⁷ is halo, C₁-C₆ alkyl, C₁-C₆ branched alkyl, C₄-C₈ cycloalkyl, C₄-C₈ aryl, C₄-C₈ aryl-substituted C₁-C₆ alkyl, C₁-C₆ alkoxy, C₁-C₆ branched alkoxy, C₄-C₈ aryloxy, or halo-substituted versions thereof, where halo is chloro, fluoro, bromo, or iodo;
R¹⁷⁸ is hydrogen, C₁-C₆ alkyl or C₁-C₆ branched alkyl;
R¹⁷⁹ is C₁-C₆ alkyl, C₄-C₈ aroyl, C₄-C₈ aryl, C₄-C₈ heterocyclic alkyl or aryl with O, N or S in the ring, C₄-C₈ aryl-substituted C₁-C₆ alkyl, alkyl-substituted or aryl-substituted C₄-C₈ heterocyclic alkyl or aryl with O, N or S in the ring, alkyl-substituted C₄-C₈ aroyl, or alkyl-substituted C₄-C₈ aryl, or halo-substituted versions thereof where halo is chloro, bromo, or iodo;
n is 1, 2, 3, or 4; and
X²⁵ is O, NH, or N—R¹⁸⁰, where R¹⁸⁰ is C₁-C₆ alkyl or C₁-C₆ branched alkyl.

Compounds that can act as Cox-2 selective inhibitors include pyridazinone compounds as described in U.S. Pat. No. 6,307,047. Such compounds have the formula (XXXV): 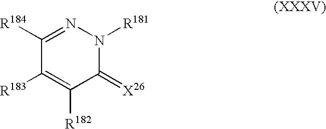
or a pharmaceutically acceptable salt, ester, or prodrug thereof, wherein:
X²⁶ is selected from the group consisting of O, S, —NR¹⁸⁵, —NOR^{a} and —NNR^{b}R^{c};
R¹⁸⁵ is selected from the group consisting of alkenyl, alkyl, aryl, arylalkyl, cycloalkenyl, cycloalkenylalkyl, cycloalkyl, cycloalkylalkyl, heterocyclic, and heterocyclic alkyl;
R^{a}, R^{b} and R^{c} are independently selected from the group consisting of alkyl, aryl, arylalkyl, cycloalkyl, and cycloalkylalkyl;
R¹⁸¹ is selected from the group consisting of alkenyl, alkoxy, alkoxyalkyl, alkoxyiminoalkoxy, alkyl, alkylcarbonylalkyl, alkylsulfonylalkyl, alkynyl, aryl, arylalkenyl, arylalkoxy, arylalkyl, arylalkynyl, arylhaloalkyl, arylhydroxyalkyl, aryloxy, aryloxyhaloalkyl, aryloxyhydroxyalkyl, arylcarbonylalkyl, carboxyalkyl, cyanoalkyl, cycloalkenyl, cycloalkenylalkyl, cycloalkyl, cycloalkylalkyl, cycloalkylidenealkyl, haloalkenyl, haloalkoxyhydroxyalkyl, haloalkyl, haloalkynyl, heterocyclic, heterocyclic alkoxy, heterocyclic alkyl, heterocyclic oxy, hydroxyalkyl, hydroxyiminoalkoxy, —(CH₂)ₙC(O)R¹⁸⁶, —(CH₂)ₙCH(OH)R¹⁸⁶, —(CH₂)ₙC(NOR^{d})R¹⁸⁶, —(CH₂)ₙCH(NOR^{d})R¹⁸⁶, —(CH₂)ₙCH(NR^{d}R^{e})R¹⁸⁶, —R¹⁸⁷R¹⁸⁸, —(CH₂)ₙC≡CR¹⁸⁸, —(CH₂)[CH(CX^{26′}₃)]ₘ(CH₂)ₚR¹⁸⁸, —(CH₂)ₙ(CX^{26′}₂)ₘ(CH₂)ₚR¹⁸⁸, and —(CH₂)ₙ(CHX^{26′})ₘ(CH₂)ₘR¹⁸⁸;
R¹⁸⁶ is selected from the group consisting of hydrogen, alkenyl, alkyl, alkynyl, aryl, arylalkyl, cycloalkenyl, cycloalkyl, haloalkenyl, haloalkyl, haloalkynyl, heterocyclic, and heterocyclic alkyl;
R¹⁸⁷ is selected from the group consisting of alkenylene, alkylene, halo-substituted alkenylene, and halo-substituted alkylene;
R¹⁸⁸ is selected from the group consisting of hydrogen, alkenyl, alkyl, alkynyl, aryl, arylalkyl, cycloalkyl, cycloalkenyl, haloalkyl, heterocyclic, and heterocyclic alkyl;
R^{d} and R^{e} are independently selected from the group consisting of hydrogen, alkenyl, alkyl, alkynyl, aryl, arylalkyl, cycloalkenyl, cycloalkyl, haloalkyl, heterocyclic, and heterocyclic alkyl;
X^{26′} is halogen;
m is an integer from 0 to 5;
n is an integer from 0 to 10;
p is an integer from 0 to 10;
R¹⁸², R¹⁸³ and R¹⁸⁴ are independently selected from the group consisting of hydrogen, alkenyl, alkoxyalkyl, alkoxyiminoalkoxy, alkoxyiminoalkyl, alkyl, alkynyl, alkylcarbonylalkoxy, alkylcarbonylamino, alkylcarbonylaminoalkyl, aminoalkoxy, aminoalkylcarbonyloxyalkoxy aminocarbonylalkyl, aryl, arylalkenyl, arylalkyl, arylalkynyl, carboxyalkylcarbonyloxyalkoxy, cyano, cycloalkenyl, cycloalkyl, cycloalkylidenealkyl, haloalkenyloxy, haloalkoxy, haloalkyl, halogen, heterocyclic, hydroxyalkoxy, hydroxyiminoalkoxy, hydroxyiminoalkyl, mercaptoalkoxy, nitro, phosphonatoalkoxy, Y⁸ and Z¹⁴, provided that one of R¹⁸², R¹⁸³ or R¹⁸⁴ must be Z¹⁴, and further provided that only one of R¹⁸², R¹⁸³ or R¹⁸⁴ is Z¹⁴;
Z¹⁴ is selected from the group consisting of: 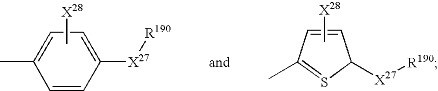
X²⁷ is selected from the group consisting of S(O)₂, S(O)(NR¹⁹¹), S(O), Se(O)₂, P(O)(OR¹⁹²) and P(O)(NR¹⁹³R¹⁹⁴);
X²⁸ is selected from the group consisting of hydrogen, alkenyl, alkyl, alkynyl and halogen;
R¹⁹⁰ is selected from the group consisting of alkenyl, alkoxy, alkyl, alkylamino, alkylcarbonylamino, alkynyl, amino, cycloalkenyl, cycloalkyl, dialkylamino, —NNNH₂ and —NCHN(R¹⁹′)R¹⁹²;
R¹⁹¹, R¹⁹², R¹⁹³ and R¹⁹⁴ are independently selected from the group consisting of hydrogen, alkyl, and cycloalkyl, or R¹⁹³ and R¹⁹⁴ can be taken together, with the nitrogen to which they are attached, to form a 3-6 membered ring containing 1 or 2 heteroatoms selected from the group consisting of O, S, and NR¹⁸⁸;
Y⁸ is selected from the group consisting of —OR¹⁹⁵, —SR¹⁹⁵, —C(R¹⁹⁷)(R¹⁹⁸)R¹⁹⁵, —C(O)R¹⁹⁵, —C(O)OR¹⁹⁵, —N(R¹⁹⁷)C(O)R¹⁹⁵, —NC(R¹⁹⁷)R¹⁹⁵ and —N(R¹⁹⁷)R¹⁹⁵;
R¹⁹⁵ is selected from the group consisting of hydrogen, alkenyl, alkoxyalkyl, alkyl, alkylthioalkyl, alkynyl, cycloalkenyl, cycloalkenylalkyl, cycloalkyl, cycloalkylalkyl, aryl, arylalkyl, heterocyclic, heterocyclic alkyl, hydroxyalkyl and NR¹⁹⁹R²⁰⁰; and
R¹⁹⁷, R¹⁹⁸, R¹⁹⁹ and R²⁰⁰ are independently selected from the group consisting of hydrogen, alkenyl, alkoxy, alkyl, cycloalkenyl, cycloalkyl, aryl, arylalkyl, heterocyclic and heterocyclic alkyl.

Compounds that can act as Cox-2 selective inhibitors include benzosulfonamide derivatives as described in U.S. Pat. No. 6,004,948. Such compounds have the formula (XXXVI): 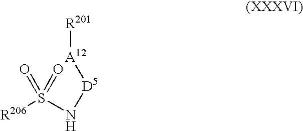
wherein:
A¹² denotes oxygen, sulfur or NH;
R²⁰¹ denotes a cycloalkyl, aryl or heteroaryl group optionally mono- or polysubstituted by halogen, alkyl, CF₃ or alkoxy;
D⁵ denotes a group: 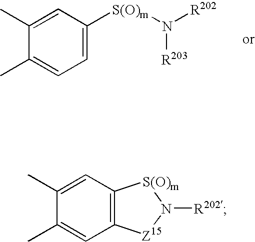
R²⁰² and R²⁰³ independently of each other denote hydrogen, an optionally polyfluorinated alkyl radical, an aralkyl, aryl or heteroaryl radical or a radical (CH₂)ₙ—X²⁹; or R²⁰² and R²⁰³ together with the N-atom denote a 3- to 7-membered, saturated, partially or totally unsaturated heterocycle with one or more heteroatoms N, O, or S, which can optionally be substituted by oxo, an alkyl, alkylaryl or aryl group or a group (CH₂)ₙ—X²⁹;
R²⁰²′ denotes hydrogen, an optionally polyfluorinated alkyl group, an aralkyl, aryl or heteroaryl group or a group (CH₂)ₙ—X²⁹;
X²⁹ denotes halogen, NO₂, —OR²⁰⁴, —COR²⁰⁴, —CO₂R²⁰⁴, —OCO₂R²⁰⁴, —CN, CONR²⁰⁴OR²⁰⁵, CONR²⁰⁴R²⁰⁵, —SR²⁰⁴, —S(O)R²⁰⁴, —S(O)₂R²⁰⁴, —NR²⁰⁴R²⁰⁵, —NHC(O)R²⁰⁴ or —NHS(O)₂R²⁰⁴;
Z¹⁵ denotes —CH₂—, —CH₂—CH₂—, —CH₂—CH₂—CH₂—, —CH₂—CH═CH—, —CH═CH—CH₂—, —CH₂—CO—, —CO—CH₂—, —NHCO—, —CONH—, —NHCH₂—, —CH₂NH—, —N═CH—, —NHCH—, —CH₂—CH₂—NH—, —CH═CH—, >N—R²⁰³, >C═O or >S(O)ₘ;
R²⁰⁴ and R²⁰⁵ independently of each other denote hydrogen, alkyl, aralkyl or aryl;
n is an integer from 0 to 6;
R²⁰⁶ is CF₃ or a straight-chained or branched C₁₋₄ alkyl group optionally mono- or polysubstituted by halogen or alkoxy; and
m denotes an integer from 0 to 2;
with the proviso that A¹² does not represent 0 if R²⁰⁶ denotes CF₃;
   and pharmaceutically acceptable salts thereof.

Compounds that can act as Cox-2 selective inhibitors include methanesulfonyl-biphenyl derivatives as described in U.S. Pat. No. 6,583,321. Such compounds have the formula (XXXVII): 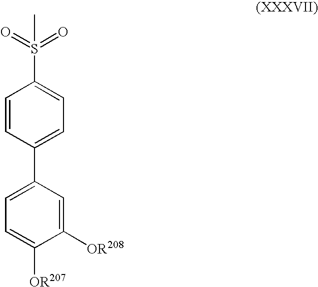
wherein R²⁰⁷ and R²⁰⁸ are individually hydrogen; C₁-C₄ alkyl, substituted or not substituted by halogen atoms; C₃-C₇ cycloalkyl; C₁-C₅ alkyl containing 1-3 ether bonds and/or an aryl substitute; substituted or unsubstituted phenyl; or substituted or unsubstituted 5- or 6-ring-cycled heteroaryl containing more than one hetero atom selected from the group consisting of nitrogen, sulfur and oxygen (wherein phenyl or heteroaryl can be mono- or multi-substituted by a substituent selected from the group consisting of hydrogen, methyl, ethyl and isopropyl).

Compounds that can act as Cox-2 selective inhibitors include 1H-indole derivatives as described in U.S. Pat. No. 6,599,929. Such compounds have the formula (XXXVIII): 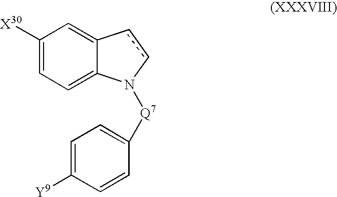
wherein:
X³⁰ is —NHSO₂R²⁰⁹ wherein R²⁰⁹ represents hydrogen or C₁-C₃ alkyl;
Y⁹ is hydrogen, halogen, C₁-C₃ alkyl substituted or not substituted by halogen atoms, NO₂, NH₂, OH, OMe, CO₂H or CN; and
Q⁷ is C═O, C═S or CH₂.

Compounds that can act as Cox-2 selective inhibitors include prodrugs as described in U.S. Pat. No. 6,436,967 and U.S. Pat. No. 6,613,790. Such compounds have the formula (XXXIX): 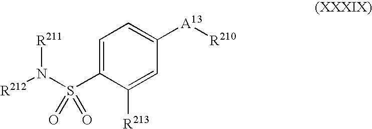
wherein:
A¹³ is a ring substituent selected from partially unsaturated heterocyclic, heteroaryl, cycloalkenyl and aryl, wherein A¹³ is unsubstituted or substituted with one or more radicals selected from alkylcarbonyl, formyl, halo, alkyl, haloalkyl, oxo, cyano, nitro, carboxyl, alkoxy, aminocarbonyl, alkoxycarbonyl, carboxyalkyl, cyanoalkyl, hydroxyalkyl, haloalkylsulfonyloxy, alkoxyalkyloxyalkyl, carboxyalkoxyalkyl, cycloalkylalkyl, alkenyl, alkynyl, heterocycloxy, alkylthio, cycloalkyl, aryl, heterocyclyl, cycloalkenyl, aralkyl, heterocyclylalkyl, alkylthioalkyl, arylcarbonyl, aralkylcarbonyl, aralkenyl, alkoxyalkyl, arylthioalkyl, aryloxyalkyl, aralkylthioalkyl, araalkoxyalkyl, alkoxycarbonylalkyl, aminocarbonylalkyl, alkylaminocarbonyl, N-arylaminocarbonyl, N-alkyl-N-arylaminocarbonyl, alkylaminocarbonylalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl and N-alkyl-N-arylaminosulfonyl;
R²¹⁰ is selected from heterocyclyl, cycloalkyl, cycloalkenyl and aryl, wherein R²¹⁰ is unsubstituted or substituted with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;
R²¹¹ is selected from hydrido and alkoxycarbonylalkyl;
R²¹² is selected from alkyl, carboxyalkyl, acyl, alkoxycarbonyl, heteroarylcarbonyl, alkoxycarbonylalkylcarbonyl, alkoxycarbonylcarbonyl, amino acid residue and alkylcarbonylaminoalkylcarbonyl;
provided A¹³ is not tetrazolium or pyridinium; further provided A¹³ is not indanone when R²¹² is alkyl or carboxyalkyl; and further provided A¹³ is not thienyl when R²¹⁰ is 4-fluorophenyl, R²¹¹ is hydrido and R²¹² is methyl or acyl; and
R²¹³ is hydrido;
   and pharmaceutically acceptable salts thereof.

Specific non-limiting examples of substituted sulfonamide prodrugs of Cox-2 inhibitors disclosed in U.S. Pat. No. 6,436,967 that are useful in the present invention include:
N-[[4-[3-(difluoromethyl)-5-(3-fluoro-4-methoxyphenyl)-1H-pyrazol-1-yl]phenyl]sulfonyl]propanamide;
N-[[4-[3-(difluoromethyl)-5-(3-fluoro-4-methoxyphenyl)-1H-pyrazol-1-yl]phenyl]sulfonyl]butanamide;
N-[[4-[1,5-dimethyl)-3-phenyl-1H-pyrazol-4-yl]phenyl]sulfonyl]acetamide;
N-[[4-(2-(3-pyridinyl)-4-(trifluoromethyl)-1H-imidazol-1-yl)phenyl]sulfonyl]acetamide;
N-[[4-[2-(5-methylpyridin-3-yl)-4-(trifluoromethyl)-1H-imidazol-1-yl]phenyl]sulfonyl]acetamide;
N-[[4-[2-(2-methylpyridin-3-yl)-4-(trifluoromethyl)-1H-imidazol-1-yl]phenyl]sulfonyl]acetamide;
N-[[4-[2-(5-methylpyridin-3-yl)-4-(trifluoromethyl)-1H-imidazol-1-yl]phenyl]sulfonyl]butanamide;
N-[[4-[2-(2-methylpyridin-3-yl)-4-(trifluoromethyl)-1H-imidazol-1-yl]phenyl]sulfonyl]butanamide;
N-[[4-[2-(3-chloro-5-methylphenyl)-4-(trifluoromethyl)-1H-imidazol-1-yl]phenyl]sulfonyl]acetamide;
N-[[4-[3-(3-fluorophenyl)-5-methylisoxazol-4-yl]phenyl]sulfonyl]acetamide;
2-methyl-N-[[4-(5-methyl-3-phenylisoxazol-4-yl)phenyl]sulfonyl]propanamide;
N-[[4-(5-methyl-3-phenylisoxazol-4-yl]phenyl]sulfonyl]propanamide;
N-[[4-(5-methyl-3-phenylisoxazol-4-yl)phenyl]sulfonyl]benzamide;
2,2-dimethyl-N-[[4-(5-methyl-3-phenylisoxazol-4-yl)phenyl]sulfonyl]propanamide;
N-[[4-5-methyl-3-phenylisoxazol-4-yl)phenyl]sulfonyl]butanamide;
N-[[4-(5-methyl-3-phenylisoxazol-4-yl)phenyl]sulfonyl]pentanamide;
N-[[4-(5-methyl-3-phenylisoxazol-4-yl)phenyl]sulfonyl]hexanamide;
3-methoxy-N-[[4-(5-methyl-3-phenylisoxazol-4-yl)phenyl]sulfonyl]propanamide;
2-ethoxy-N-[[4-(5-methyl-3-phenylisoxazol-4-yl)phenyl]sulfonyl]acetamide;
N-[[4-[5-methyl-3-phenylisoxazol-4-yl]phenyl]sulfonyl]acetamide;
N-[[4-[5-(4-chlorophenyl)-3-(trifluoromethyl)-1H pyrazol-1-yl]phenyl]sulfonyl]propanamide;
N-[[4-[5-(4-chlorophenyl)-3-(trifluoromethyl)-1H-pyrazol-1-yl]phenyl]sulfonyl]butanamide;
N-[[4-[5-(4-chlorophenyl)-3-(trifluoromethyl)-1H-pyrazol-1-yl]phenyl]sulfonyl]acetamide;
N-[[4-[3-(difluoromethyl)-6-fluoro-1,5-dihydro-7-methoxy-[2]benzothiopyrano[
4,3-c]pyrazol-1-yl)phenyl]sulfonyl]acetamide;
N-[[4-[6-fluoro-1,5-dihydro-7-methoxy-3-(trifluoromethyl)-[2]benzothiopyrano[
4,3-c]pyrazol-1-yl]phenyl]sulfonyl]acetamide;
N-[[4-[3-(difluoromethyl)-5-(3-fluoro-4-methoxyphenyl)-1H-pyrazol-1-yl]phenyl]sulfonyl]acetamide;
N-[[4-(2-methyl-4-phenyloxazol-5-yl)phenyl]sulfonyl]acetamide;
methyl [[[4-(5-methyl-3-phenylisoxazol-4-yl)phenyl]sulfonyl]amino]oxoacetate;
2-methoxy-N-[[4-(5-methyl-3-phenylisoxazol-4-yl)phenyl]sulfonyl]acetamide;
N-[[4-[5-(difluoromethyl)-3-phenylisoxazol-4-yl]phenyl]sulfonyl]propanamide;
N-[[4-[5-(difluoromethyl)-3-phenylisoxazol-4-yl]phenyl]sulfonyl]butanamide;
N-[[4-(5-methyl-3-phenylisoxazol-4-yl)phenyl]sulfonyl]formamide;
1,1-dimethylethyl-N-[[4-(5-methyl-3-phenylisoxazol-4-yl)phenyl]sulfonyl]carbamate;
N-[[.sup.4-(5-methyl-3-phenylisoxazol-4-yl)phenyl]sulfonyl]glycine;
2-amino-N-[[4-(5-methyl-3-phenylisoxazol-4-yl)phenyl]sulfonyl]acetamide;
2-(acetylamino)-N-[[4-(5-methyl-3-phenylisoxazol-4-yl)phenyl]sulfonyl]acetamide;
methyl 4-[[[4-(5-methyl-3-phenylisoxazol-4-yl)phenyl]sulfonyl]amino]-4-oxobutanoate;
methyl N-[[4-(5-methyl-3-phenylisoxazol-4-yl)phenyl]sulfonyl]carbamate;
N-acetyl-N-[[4-(5-methyl-3-phenylisoxazol-4-yl)phenyl]sulfonyl]glycine, ethyl ester;
N-[[4-(5-(4-methylphenyl)-3-(trifluoromethyl)-1H-pyrazol-1-yl)phenyl]sulfonyl]acetamide;
methyl 3-[[[4-(5-methyl-3-phenylisoxazol-4-yl)phenyl]sulfonyl]amino]-3-oxopropanoate;
4-[5-(3-bromo-5-fluoro-4-methoxyphenyl)-2-(trifluoromethyl)oxazol-4-yl]-N-methylbenezenesulfonamide;
N-(1,1-dimethylethyl)-4-(5-methyl-3-phenylisoxazol-4-yl)benzenesulfonamide;
4-[5-(4-fluorophenyl)-3-(trifluoromethyl)-1H-pyrazol-1-yl]-N-methylbenzene sulfonamide;
N-methyl-4-(5-methyl-3-phenylisoxazol-4-yl)benzenesulfonamide;
N-[[4-[5-(hydroxymethyl)-3-phenylisoxazol-4-yl]phenyl]sulfonyl]acetamide;
N-[[4-[5-(acetoxymethyl)-3-phenylisoxazol-4-yl]phenyl]sulfonyl]acetamide;
N-[[4-[2-(3-chloro-4-fluorophenyl)cyclopenten-1-yl)phenyl]sulfonyl]acetamide;
4-[2-(4-fluorophenyl)-1H-pyrrol-1-yl]-N-methylbenzenesulfonamide;
N-[[4-(3,4-dimethyl-1-phenyl-1H-pyrazol-5-yl]phenyl]sulfonyl]propanamide;
N-[[4-[2-(2-methylpyridin-3-yl)-4-trifluoromethylimidazol-1-yl]phenyl)sulfonyl]propanamide;
4-[2-(4-fluorophenyl)cyclopenten-1-yl]-N-methylbenezenesulfonamide; and
N-[[4-(3-phenyl-2,3-dihydro-2-oxofuran-4-yl)phenyl]sulfonyl]propanamide.

Prodrugs disclosed in U.S. Pat. No. 6,613,790 have formula (XXXIX) wherein:
A¹³ is a pyrazole group optionally substituted at a substitutable position with one or more radicals independently selected at each occurrence from the group consisting of alkylcarbonyl, formyl, halo, alkyl, haloalkyl, oxo, cyano, intro, carboxyl, alkoxy, aminocarbonyl, alkoxycarbonyl, carboxyalkyl, cyanoalkyl, hydroxyalkyl, haloalkylsulonyloxy, alkoxyalkyloxyalkyl, carboxyalkoxyalkyl, alkenyl, alkynyl, alkylthio, alkylthioalkyl, alkoxyalkyl, alkoxycarbonylalkyl, aminocarbonylalkyl, alkylaminocarbonyl, alkylaminocarbonylalkyl, alkylamino, aminoalkyl, alkylaminoalkyl, alkylsutfinyl, alkylsulfonyl, aminosulfonyl and alkylaminosulfonyl;
R²¹⁰ is a phenyl group optionally substituted at a substitutable position with one or more radicals independently selected at each occurrence from the group consisting of alkyl, haloalkyl, cyano, carboxyl, alkoxycarbonyl, hydroxyl, hydroxyalkyl, haloalkoxy, amino, alkylamino, nitro, alkoxyalkyl, alkylsulfinyl, halo, alkoxy, and alkylthio;
R²¹¹ and R²¹² are independently selected from the group consisting of hydroxyalkyl and hydrido but at least one of R²¹¹ and R²¹² is other than hydrido; and
   R²¹³ is selected from the group consisting of hydrido and fluoro.

Specific non-limiting examples of substituted sulfonamide prodrugs of Cox-2 inhibitors disclosed in U.S. Pat. No. 6,613,790 that are useful in the present invention include:
N-(2-hydroxyethyl)-4-[5-(4-methylphenyl)-3-(trifluoromethyl)-1H-pyrazol-1-yl]benzenesulfonamide;
N,N-bis(2-hydroxyethyl)-4-[5-(4-methylphenyl)-3-(trifluoromethyl)-1H-pyrazol-1-yl]benzenesulfonamide;
   and pharmaceutically acceptable salts thereof.

Compounds that can act as Cox-2 selective inhibitors include sulfamoylheteroaryl pyrazole compounds as described in U.S. Pat. No. 6,583,321. Such compounds have the formula (XL): 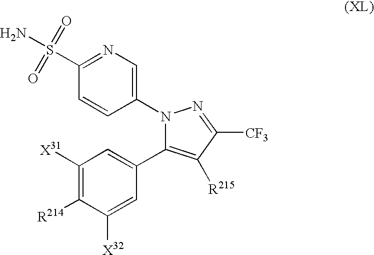
wherein:
R²¹⁴ is furyl, thiazolyl or oxazolyl;
R²¹⁵ is hydrogen, fluoro or ethyl; and
X³¹ and X³² are independently hydrogen or chloro.

Compounds that can act as Cox-2 selective inhibitors include heteroaryl substituted amidinyl and imidazolyl compounds as described in U.S. Pat. No. 6,555,563. Such compounds have the formula (XLI): 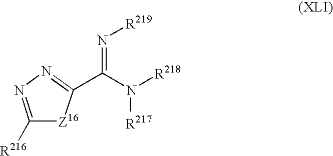
wherein:
Z¹⁶ is O or S;
R²¹⁶ is optionally substituted aryl;
R²¹⁷ is aryl optionally substituted with aminosulfonyl; and
R²¹⁸ and R²¹⁹ cooperate to form an optionally substituted 5-membered ring.

Compounds that can act as Cox-2 selective inhibitors include substituted hydroxamic acid derivatives as described in U.S. Pat. No. 6,432,999, U.S. Pat. No. 6,512,121, U.S. Pat. No. 6,515,014 and U.S. Pat. No. 6,555,563. These compounds also act as inhibitors of the lipoxygenase-5 enzyme. Such compounds have the formulas (XLII) and (XLIII):

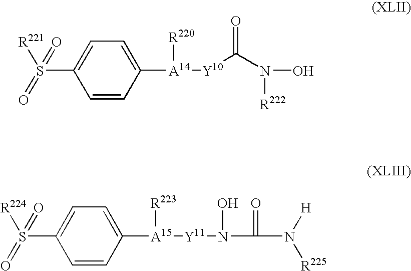

Pyrazole-substituted hydroxamic acid derivatives described in U.S. Pat. No. 6,432,999 can have formula (XLII), wherein:
A¹⁴ is pyrazolyl optionally substituted with a substituent selected from acyl, halo, hydroxyl, lower alkyl, lower haloalkyl, oxo, cyano, nitro, carboxyl, lower alkoxy, aminocarbonyl, lower alkoxycarbonyl, lower carboxyalkyl, lower cyanoalkyl and lower hydroxyalkyl;
Y¹⁰ is selected from lower alkenylene and lower alkynylene;
R²²⁰ is a substituent selected from 5- and 6-membered heterocyclo, lower cycloalkyl, lower cycloalkenyl and aryl selected from phenyl, biphenyl and naphthyl, wherein R²²⁰ is optionally substituted at a substitutable position with one or more substituents selected from lower alkyl, lower haloalkyl, cyano, carboxyl, lower alkoxycarbonyl, hydroxyl, lower hydroxyalkyl, lower haloalkoxy, amino, lower alkylamino, phenylmino, nitro, lower alkoxyalkyl, lower alkylsulfinyl, halo, lower alkoxy and lower alkylthio;
R²²¹ is selected from lower alkyl and amino; and
R²²² is selected from hydrido, lower alkyl, phenyl, 5- and 6-membered heterocyclo and lower cycloalkyl;
   and pharmaceutically acceptable salts thereof.

Pyrazole-substituted hydroxamic acid derivatives described in U.S. Pat. No. 6,432,999 can alternatively have formula (XLIII), wherein:
A¹⁵ is pyrazolyl optionally substituted with a substituent selected from acyl, halo, hydroxyl, lower alkyl, lower haloalkyl, oxo, cyano, nitro, carboxyl, lower alkoxy, aminocarbonyl, lower alkoxycarbonyl, lower carboxyalkyl, lower cyanoalkyl and lower hydroxyalkyl;
Y¹¹ is selected from lower alkylene, lower alkenylene and lower alkynylene;
R²²³ is a substituent selected from 5- and 6-membered heterocyclo, lower cycloalkyl, lower cycloalkenyl and aryl selected from phenyl, biphenyl and naphthyl, wherein R²²³ is optionally substituted at a substitutable position with one or more substituents selected from lower alkyl, lower haloalkyl, cyano, carboxyl, lower alkoxycarbonyl, hydroxyl, lower hydroxyalkyl, lower haloalkoxy, amino, lower alkylamino, phenylmino, nitro, lower alkoxyalkyl, lower alkylsulfinyl, halo, lower alkoxy and lower alkylthio;
R²²⁴ is selected from lower alkyl and amino; and
R²²⁵ is selected from hydrido and lower alkyl;
   and pharmaceutically acceptable salts thereof.

Heterocyclo-substituted hydroxamic acid derivatives described in U.S. Pat. No. 6,512,121 can have formula (XLII), wherein:
A¹⁴ is a ring substituent selected from oxazolyl, furyl, pyrrolyl, thiazolyl, imidazolyl, isothiazolyl, isoxazolyl, cyclopentenyl, phenyl, and pyridyl; wherein A¹⁴ is optionally substituted with a substituent selected from acyl, halo, hydroxy, lower alkyl, lower haloalkyl, oxo, cyano, nitro, carboxyl, lower alkoxy, aminocarbonyl, lower alkoxycarbonyl, lower carboxyalkyl, lower cyanoalkyl and lower hydroxyalkyl;
Y¹⁰ is selected from lower alkylene, lower alkenylene and lower alkynylene;
R²²⁰ is a substituent selected from 5- and 6-membered heterocyclo, lower cycloalkyl, lower cycloalkenyl and aryl selected from phenyl, biphenyl and naphthyl, wherein R²²⁰ is optionally substituted at a substitutable position with one or more substituents selected from lower alkyl, lower haloalkyl, cyano, carboxyl, lower alkoxycarbonyl, hydroxyl, lower hydroxyalkyl, lower haloalkoxy, amino, lower alkylamino, phenylamino, nitro, lower alkoxyalkyl, lower alkylsulfinyl, halo, lower alkoxy and lower alkylthio;
R²²¹ is selected from lower alkyl and amino; and
R²²² is selected from hydrido, lower alkyl, phenyl, 5- and 6-membered heterocyclo and lower cycloalkyl;
   and pharmaceutically acceptable salts thereof.

Heterocyclo-substituted hydroxamic acid derivatives described in U.S. Pat. No. 6,512,121 can alternatively have formula (XLIII), wherein:
A¹⁵ is a ring substituent selected from oxazolyl, furyl, pyrrolyl, thiazolyl, imidazolyl, isothiazolyl, isoxazolyl, cyclopentenyl, phenyl, and pyridyl; wherein A¹⁵ is optionally substituted with a substituent selected from acyl, halo, hydroxy, lower alkyl, lower haloalkyl, oxo, cyano, nitro, carboxyl, lower alkoxy, aminocarbonyl, lower alkoxycarbonyl, lower carboxyalkyl, lower cyanoalkyl and lower hydroxyalkyl;
Y¹¹ is selected from lower alkyl, lower alkenyl and lower alkynyl;
R²²³ is a substituent selected from 5- and 6-membered heterocyclo, lower cycloalkyl, lower cycloalkenyl and aryl selected from phenyl, biphenyl and naphthyl, wherein R²²³ is optionally substituted at a substitutable position with one or more substituents selected from lower alkyl, lower haloalkyl, cyano, carboxyl, lower alkoxycarbonyl, hydroxyl, lower hydroxyalkyl, lower haloalkoxy, amino, lower alkylamino, phenylamino, nitro, lower alkoxyalkyl, lower alkylsulfinyl, halo, lower alkoxy and lower alkylthio;
R²²⁴ is selected from lower alkyl and amino; and
R²²⁵ is selected from hydrido and alkyl;
   or a pharmaceutically-acceptable salt thereof.

Thiophene-substituted hydroxamic acid derivatives described in U.S. Pat. No. 6,515,014 can have formula (XLII), wherein:
A¹⁴ is thienyl optionally substituted with a substituent selected from acyl, halo, hydroxy, lower alkyl, lower haloalkyl, oxo, cyano, nitro, carboxyl, lower alkoxy, aminocarbonyl, lower alkoxycarbonyl, lower carboxyalkyl, lower cyanoalkyl and lower hydroxyalkyl;
Y¹⁰ is selected from ethylene, isopropylene, propylene, butylene, lower alkenylene and lower alkynylene;
R²²⁰ is a substituent selected from 5- and 6-membered heterocyclo, lower cycloalkyl, lower cycloalkenyl and aryl selected from phenyl, biphenyl and naphthyl, wherein R²²⁰ is optionally substituted at a substitutable position with one or more substituents selected from lower alkyl, lower haloalkyl, cyano, carboxyl, lower alkoxycarbonyl, hydroxyl, lower hydroxyalkyl, lower haloalkoxy, amino, lower alkylamino, phenylamino, nitro, lower alkoxyalkyl, lower alkylsulfinyl, halo, lower alkoxy and lower alkylthio;
R²²¹ is selected from lower alkyl and amino; and
R²²² is selected from hydrido, lower alkyl, phenyl, 5- and 6-membered heterocyclo and lower cycloalkyl;
   and pharmaceutically acceptable salts thereof.

Thiophene substituted hydroxamic acid derivatives described in U.S. Pat. No. 6,515,014 can alternatively have formula (XLIII), wherein:
A¹⁵ is thienyl optionally substituted with a substituent selected from acyl, halo, hydroxy, lower alkyl, lower haloalkyl, oxo, cyano, nitro, carboxyl, lower alkoxy, aminocarbonyl, lower alkoxycarbonyl, lower carboxyalkyl, lower cyanoalkyl and lower hydroxyalkyl;
Y¹¹ is selected from lower alkyl, lower alkenyl and lower alkynyl;
R²²³ is a substituent selected from 5- and 6-membered heterocyclo, lower cycloalkyl, lower cycloalkenyl and aryl selected from phenyl, biphenyl and naphthyl, wherein R²²³ is optionally substituted at a substitutable position with one or more substituents selected from lower alkyl, lower haloalkyl, cyano, carboxyl, lower alkoxycarbonyl, hydroxyl, lower hydroxyalkyl, lower haloalkoxy, amino, lower alkylamino, phenylamino, nitro, lower alkoxyalkyl, lower alkylsulfinyl, halo, lower alkoxy and lower alkylthio;
R²²⁴ is selected from lower alkyl and amino; and
R²²⁵ is selected from hydrido and alkyl;
   and pharmaceutically acceptable salts thereof.

Compounds that can act as Cox-2 selective inhibitors include pyrazolopyridine compounds as described in U.S. Pat. No. 6,498,166. Such compounds have the formula (XLIV): 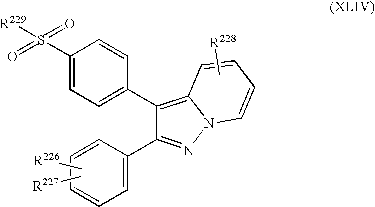
wherein:
R²²⁶ and R²²⁷ are independently selected from the group consisting of H, halogen, C₁-C₆ alkyl, C₁-C₆ alkoxy, and C₁-C₆ alkoxy substituted by one or more fluorine atoms;
R²²⁸ is halogen, CN, CONR²³⁰R²³¹, CO₂H, CO₂(C₁-C₆ alkyl) or NHSO₂R²³⁰;
R²²⁹ is C₁-C₆ alkyl or NH₂; and
R²¹⁰ and R²³¹ are independently selected from the group consisting of H, C₁-C₆ alkyl, phenyl, and phenyl substituted by one or more atoms or groups selected from the group consisting of halogen, C₁-C₆ alkyl, C₁-C₆ alkoxy, and C₁-C₆ alkoxy substituted by one or more fluorine atoms;
   or a pharmaceutically acceptable salt, solvate or ester thereof, or salt or solvate of such ester.

Compounds that can act as Cox-2 selective inhibitors include 4,5-diaryl-3(2H)-furanone derivatives as described in U.S. Pat. No. 6,492,416. Such compounds have the formula (XLV): 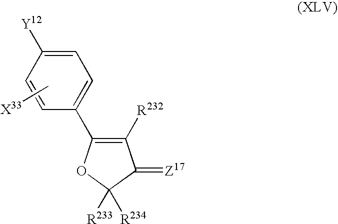
wherein:
X³³ is halo, hydrido or alkyl;
Y¹² is alkylsulfonyl, aminosulfonyl, alkylsulfinyl, (N-acylamino)sulfonyl, (N-alkylamino)sulfonyl or alkylthio;
Z¹⁷ is an oxygen or sulfur atom;
R²³³ and R²³⁴ are selected independently from lower alkyl radicals; and
R²³² represents a substituted or non-substituted aromatic group of 5 to 10 atoms;
   and pharmaceutically acceptable salts thereof.

Compounds that can act as Cox-2 selective inhibitors include 2-phenyl-1,2-benzisoselenazol-3(2H)-one derivatives and 2-phenylcarbamylphenylselenyl derivatives as described in U.S. Pat. No. 6,492,416. Such compounds have the formulas (XLVI) and (XLVII): 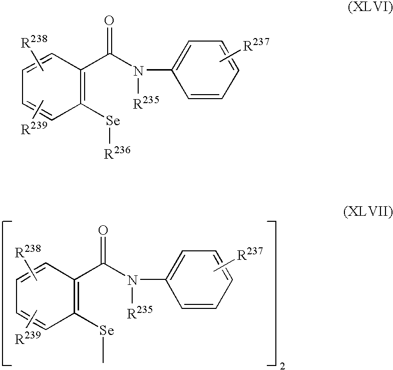
wherein:
R²³⁵ is a hydrogen atom or an alkyl group having 1-3 carbon atoms;
R²³⁶ is a hydrogen atom, a hydroxyl group, an organothiol group that is bound to the selenium atom by its sulfur atom, or R²³⁵ and R²³⁶ are joined to each other by a single bond;
R²³⁷ is a hydrogen atom, a halogen atom, an alkyl group having 1-3 carbon atoms, an alkoxy group, having 1-3 carbon atoms, a trifluoromethyl group, or a nitro group;
R²³⁸ and R²³⁹ are identical to or different from each other, and each is a hydrogen atom, a halogen atom, an alkoxyl group having 1-4 carbon atoms, a trifluoromethyl group, or R²³⁸ and R²³⁹ are joined to each other to form a methylenedioxy group,
   and pharmaceutically acceptable salts thereof, and hydrates thereof.

Compounds that can act as Cox-2 selective inhibitors include pyrones as described in U.S. Pat. No. 6,465,509. Such compounds have the formula (XLVIII): 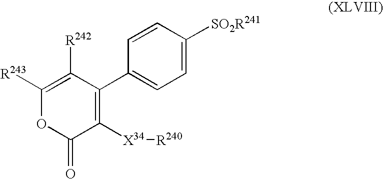
wherein:
X³⁴ is selected from the group consisting of a bond, —(CH₂)ₘ— wherein m is 1 or 2, —C(O)—, —O—, —S— and —N(R²⁴⁴)—;
R²⁴⁰ is selected from the group consisting of (a) C₁-C₁₀ alkyl, optionally substituted with 1-3 substituents independently selected from the group consisting of hydroxy, halo, C₁-C₁₀ alkoxy, C₁-C₁₀ alkylthio and CN, (b) phenyl, (c) naphthyl, and (d) heteroaryl comprising a monocyclic aromatic ring of 5 atoms having one hetero atom which is S, O or N, and optionally 1, 2 or 3 additional N atoms, or a monocyclic ring of 6 atoms having one hetero atom which is N, and optionally 1, 2 or 3 additional N atoms; wherein groups (b), (c) and (d) are optionally substituted with 1-3 substituents independently selected from the group consisting of halo, C₁-C₁₀ alkoxy, C₁-C₁₀ alkylthio, CN, C₁-C₁₀ alkyl optionally substituted to its maximum with halo, and N₃;
R²⁴¹ is selected from the group consisting of (a) C₁-C₆ alkyl optionally substituted to its maximum with halo, (b) NH₂, and (c) NHC(O)(C₁-C₁₀ alkyl) optionally substituted to its maximum with halo;
R²⁴² and R²⁴³ are independently selected from the group consisting of hydrogen, halo and C₁-C₆ alkyl optionally substituted to its maximum with halo; and
R²⁴⁴ is selected from the group consisting of hydrogen and C₁-C₆ alkyl optionally substituted to its maximum with halo.

Examples of pyrone compounds that are useful as Cox-2 selective inhibitors of the present invention include, but are not limited to:
4-(4-methylsulfonyl)phenyl-3-phenyl-pyran-2-one;
3-(4-fluorophenyl)-6-methyl-4-(4-methylsulfonyl)phenyl-pyran-2-one;
3-(3-fluorophenyl)-6-methyl-4-(4-methylsulfonyl)phenyl-pyran-2-one;
6-methyl-4-(4-methylsulfonyl)phenyl-3-phenyl-pyran-2-one;
6-difluoromethyl-4-(4-methylsulfonyl)phenyl-3-phenyl-pyran-2-one;
6-fluoromethyl-4-(4-methylsulfonyl)phenyl-3-phenyl-pyran-2-one;
6-methyl-4-(4-methylsulfonyl)phenyl-3-phenylthio-pyran-2-one;
6-methyl-4-(4-methylsulfonyl)phenyl-3-phenoxy-pyran-2-one;
6-methyl-4-(4-methylsulfonyl)phenyl-3-pyridin-3-yl-pyran-2-one;
3-isopropylthio-6-methyl-4-(4-methylsulfonyl)phenyl-pyran-2-one;
4-(4-methylsulfonyl)phenyl)-3-phenylthio-6-trifluoromethyl-pyran-2-one;
3-isopropylthio-4-(4-methylsulfonyl)phenyl-6-trifluoromethyl-pyran-2-one;
4-(4-methylsulfonyl)phenyl-3-phenyl-6-(2,2,2-trifluoroethyl)-pyran-2-one; and
3-(3-hydroxy-3-methylbutyl)-6-methyl-4-(4-methylsulfonyl)phenyl-pyran-2-one.

Compounds that can act as Cox-2 selective inhibitors include free-B-ring flavonoids as described in U.S. Patent Application Publication No. 2003/0165588. Such compounds, organically synthesized or purified from plant sources, have the formula (XLIX): 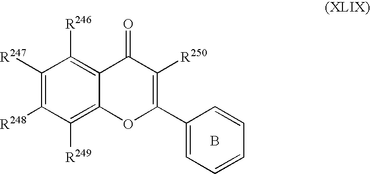
wherein R²⁴⁶, R²⁴⁷, R²⁴⁸, R²⁴⁹ and R²⁵⁰ are independently selected from the group consisting of —H, —OH, —SH, —OR, —SR, —NH₂, —NHR²⁴⁵, —N(R²⁴⁵)₂, —N(R²⁴⁵)₃⁺X³⁵⁻, a carbon, oxygen, nitrogen or sulfur glycoside of a single or a combination of multiple sugars selected from aldopentoses, methyl-aldopentose, aldohexoses, ketohexose and chemical derivatives thereof; where R²⁴⁵ is an alkyl group having 1-10 carbon atoms, and X³⁵ is selected from the group of pharmaceutically acceptable counter-anions consisting of hydroxyl, chloride, iodide, sulfate, phosphate, acetate, fluoride and carbonate.

Compounds that can act as Cox-2 selective inhibitors include heterocycloalkylsulfonyl pyrazoles as described in European Patent Publication No. EP 1 312 367. Such compounds have the formula (L): 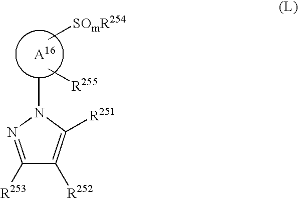
wherein:
ring A¹⁶ is selected from the group consisting of 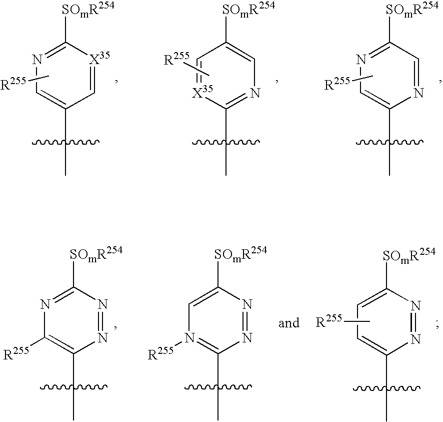
m is 0, 1 or 2;
X³⁵ is >CR²⁵⁵ or >N;
R²⁵¹ is a radical selected from the group consisting of H, NO₂, CN, C₁-C₆ alkyl, (C₁-C₆ alkyl)-SO₂—, (C₆-C₁₀ aryl)-SO₂—, H—(C═O)—, (C₁-C₆ alkyl)-(C═O), (C₁-C₆ alkyl)-(C═O)—, (C₁-C₉ heteroaryl)-(C═O)—, (C₁-C₆ heterocyclyl)-(C═O)—, H₂N—(C═O)—, (C₁-C₆ alkyl)-NH—(C═O)—, (C₁-C₆ alkyl)₂—N—(C═O)—, (C₆-C₁₀ aryl)₂—NH—(C═O)—, (C₁-C₆ alkyl)-[(C₆-C₁₀ aryl)-N]—(C═O)—, HO—NH—(C═O)— and (C₁-C₆ alkyl)-O—NH—(C═O)—;
R²⁵² is a radical selected from the group consisting of H, NO₂, CN, C₂-C₆ alkenyl, C₂-C₆ alkynyl, C₃-C₇ cycloalkyl, C₆-C₁₀ aryl, C—C₉ heteroaryl, C₁₋₉ heterocyclyl, (C₁-C₆ alkyl)-O—, (C₃-C₇ cycloalkyl)-O—, (C₆-C₁₀ aryl)-O—, (C₁-C₉ heteroaryl)-O—, (C₆-C₉ heterocyclyl)-O—, H—(C═O)—, (C₁-C₆ alkyl)-(C═O)—, (C₃-C₇ cycloalkyl)-(C═O)—, (C₆-C₁₀ aryl)-(C═O)—, (C₁-C₉ heteroaryl)(C═O)—, (C₁-C₉ heterocyclyl)-(C═O)—, (C₁-C₆ alkyl)-O—(C═O)—, (C₃-C₇ cycloalkyl)-O—(C═O)—, (C₆-C₁₀ aryl)-O—(C═O)—, (C₁-C₉ heteroaryl)-O—(C═O)—, (C₁-C₉ heterocyclyl)-O—(C═O)—, (C₁-C₆ alkyl)-(C═O)—O—, (C₃-C₇ cycloalkyl)-(C═O)—O—, (C₆-C₁₀ aryl)-(C═O)—O—, (C₁-C₉ heteroaryl)-(C═O)—O—, (C₁-C₉ heterocyclyl)-(C═O)—O—, (C₁-C₆ alkyl)-(C═O)—NH—, (C₃-C₇ cycloalkyl)-(C═O)—NH—, (C₆-C₁₀ aryl)-(C═O)—NH—, (C₁-C₉ heteroaryl)-(C═O)—NH—, (C₁-C₉ heterocyclyl)-(C═O)—NH—, (C₁-C₆ alkyl)-O—(C═O)—NH—, (C₁-C₆ alkyl)-NH, (C₁-C₆ alkyl)₂—N—, (C₃-C₇ cycloalkyl)-NH—, (C₃-C₇ cycloalkyl)₂—N—, (C₆-C₁₀ aryl)-NH—, (C₆-C₁₀ aryl)₂—N—, (C₁-C₆ alkyl)-[(C₆-C₁₀ aryl)-N]—, (C₁-C₉ heteroaryl)-NH—, (C₁-C₉ heteroaryl)₂—N—, (C₁-C₉ heterocyclyl)-NH—, (C₁-C₉ heterocyclyl)₂—N—, H₂N—(C═O)—, HO—NH—(C═O)—, (C₁-C₆ alkyl)-O—NH—(C═O)—, (C₁-C₆ alkyl)-NH—(C═O)—, (C₁-C₆ alkyl)₂—N—(C═O)—, (C₃-C₇ cycloalkyl)-NH—(C═O)—, (C₃-C₇ cycloalkyl)₂—N—(C═O)—, (C₆-C₁₀ aryl)-NH—(C═O)—, (C₆-C₁₀ aryl)₂—N—(C═O)—, (C₁-C₆ alkyl)-[(C₆-C₁₀ aryl)-N]-(C═O)—, (C₁-C₉ heteroaryl)-NH—(C═O)—, (C₁-C₉ heteroaryl)₂—N—(C═O)—, (C₁-C₉ heterocyclyl)-NH—(C═O)—, (C₁-C₆ alkyl)-S— and C₁-C₆ alkyl optionally substituted by one —OH substituent or by one to four fluoro substituents;
R²⁵³ is a saturated 3- to 4-membered heterocyclyl ring radical; or a saturated, partially saturated or aromatic 7- to 9-membered heterocyclyl ring radical; wherein said ring radical (a) optionally contains one to four ring heteroatoms independently selected from the group consisting of —N═, —NH—, —O— and —S—; (b) optionally is substituted on any ring carbon atom by one to three substituents per ring independently selected from the group consisting of halo, OH, CN, NO₂, C₂-C₆ alkenyl, C₂-C₆ alkynyl, C₃-C₇ cycloalkyl, C₆-C₁₀ aryl, C₂-C₉ hetorocyclyl, (C₁-C₆ alkyl)-O—, H—(C═O)—, (C₁-C₆ alkyl)(C═O)—, HO—(C═O)—, (C₁-C₆ alkyl)-O—(C═O)—, —NH₂, (C₁-C₆ alkyl)-NH—, (C₁-C₆ alkyl)₂—N—, (C₃-C₇ cycloalkyl)-NH—, (C₆-C₁₀ aryl)-NH—, (C₁-C₆ alkyl)-[(C₆-C₁₀ aryl)-N]—, (C₁-C₉ heteroaryl)-NH—, H₂N—(C═O)—(C₁-C₆ alkyl)-NH—(C═O)—, (C₁-C₆ alkyl)₂—N—(C═O)—, (C₆-C₁₀ aryl)-NH—(C═O)—, (C₁-C₆ alkyl)-[(C₆-C₁₀ aryl)-N]-(C═O)—, (C₁-C₆ alkyl)-O—NH—(C═O)—, (C₁-C₆ alkyl)-(C═O)—HN—, (C₁-C₆ alkyl)-(C═O)—, (C₁-C₆ alkyl)-N]—, —SH, (C₁-C₆ alkyl)S—, (C₁-C₆ alkyl)-(S═O)—, (C₁-C₆ alkyl)-SO₂—, and C₁-C₆ alkyl optionally substituted with one to four fluoro moieties; and (c) optionally is substituted on any ring nitrogen atom by one to three substituents per ring independently selected from the group consisting of C₃-C₇ cycloalkyl, C₆-C₁₀ aryl, C₂-C₉ heterocyclyl, H—(C═O)—, (C₁-C₆ alkyl)-(C═O)—, (C₁-C₆ alkyl)-O—(C═O)—, H₂N—(C═O)—, (C₁-C₆ alkyl)-NH—(C═O)—, (C₁-C₆ alkyl)₂—N—(C═O)—, (C₆-C₁₀ aryl)-NH—(C═O)—, (C₁-C₆ alkyl)-[(C₆-C₁₀ aryl)-N]-(C═O)—, (C₁-C₆ alkyl)-O—NH—(C═O)—, and C₁-C₆ alkyl optionally substituted with one to four fluoro moieties;
R²⁵⁴ is a C₁-C₆ alkyl radical optionally substituted by one to four fluoro substituents; and
R²⁵⁵ is a radical selected from the group consisting of H, halo, OH, (C₁-C₆ alkyl)-O—, C₂-C₆ alkenyl, C₂-C₆ alkynyl, C₃-C₇ cycloalkyl, CN, H—(C═O)—, (C₁-C₆ alkyl-(C═O)—, (C₁-C₆ alkyl)-(C═O)—O—, HO—(C═O)—, (C₁-C₆ alkyl)-O—(C═O)—, (C₁-C₆ alkyl)-NH—, (C₁-C₆ alkyl)₂—N—, (C₃-C₇ cycloalkyl)-NH—, (C₆-C₁₀ aryl)-NH—, (C₁-C₆ alkyl)-[(C₆-C₁₀ aryl)-N]—, (C, —C heteroaryl)-NH—, H₂N—(C═O)—, (C₁-C₆ alkyl)-NH—(C═O)—, (C₁-C₆ alkyl)₂—N—(C═O)—, (C₆-C₁₀ aryl)-(C═O)—, (C₁-C₆ alkyl)-[(C₆-C₁₀ aryl)-N]-(C═O)—, (C₁-C₆ alkyl-O—NH—(C═O)—, (C₁-C₆ alkyl)-S—, and C₁-C₆ alkyl optionally substituted by one to four fluoro substituents;
   and pharmaceutically acceptable salts thereof.

Compounds that can act as Cox-2 selective inhibitors include 2-phenylpyran-4-one derivatives as described in U.S. Pat. No. 6,518,303. Such compounds have the formula (LI): 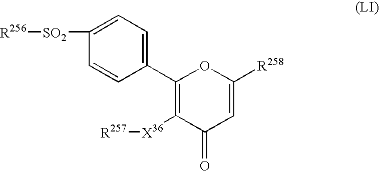
wherein:
R²⁵⁶ is an alkyl or —NR²⁵⁹R²⁶⁰ group, where R²⁵⁹ and R²⁶⁰ are independently selected from a hydrogen atom and an alkyl group;
R²⁵⁷ is an alkyl, C₃-C₇ cycloalkyl, naphthyl, tetrahydronaphthyl or indanyl group, or a phenyl group which is unsubstituted or substituted by one or more halogen atoms or alkyl, trifluoromethyl, hydroxy, alkoxy, methylthio, amino, mono- or dialkylamino, hydroxyalkyl or hydroxycarbonyl groups;
R²⁵⁸ is a methyl, hydroxymethyl, alkoxymethyl, C₃-C₇ cycloalkoxymethyl, benzyloxymethyl, hydroxycarbonyl, nitrile, trifluoromethyl or difluoromethyl group or a CH₂—R²⁶¹ group where R²⁶¹ is an alkyl group; and
X³⁶ is a single bond, an oxygen atom, a sulfur atom or a methylene group;
   and pharmaceutically acceptable salts thereof.

Examples of 2-phenylpyran-4-one derivatives useful in the present invention include, but are not limited to:
3-(4-fluorophenyl)-2-(4-methanesulfonylphenyl)-6-methylpyran-4-one;
3-(2-fluorophenyl)-2-(4-methanesulfonylphenyl)-6-methylpyran-4-one;
3-(4-chlorophenyl)-2-(4-methanesulfonylphenyl)-6-methylpyran-4-one;
3-(4-bromophenyl)-2-(4-methylsulfonylphenyl)-6-methylpyran-4-one;
3-(2,4-difluorophenyl)-2-(4-methanesulfonylphenyl)-6-methylpyran-4-one;
3-(3,4-dichlorophenyl)-2-(4-methanesulfonylphenyl)-6-methylpyran-4-one;
3-(3-chloro-4-methylphenyl)-2-(4-methanesulfonylphenyl)-6-methylpyran-4-one;
2-(4-methanesulfonylphenyl)-6-methyl-3-phenoxypyran-4-one;
3-(4-fluorophenoxy)-2-(4-methanesulfonylphenyl)-6-methylpyran-4-one;
3-(2-fluorophenoxy)-2-(methanesulfonylphenyl)-6-methylpyran-4-one;
3-(4-chlorophenoxy)-2-(methanesulfonylphenyl)-6-methylpyran-4-one;
3-(2-chlorophenoxy)-2-(methanesulfonylphenyl)-6-methylpyran-4-one;
3-(4-bromophenoxy)-2-(4-methanesulfonylphenyl)-6-methylpyran-4-one;
2-(4-methanesulfonylphenyl)-6-methyl-3-(4-methylphenoxy)pyran-4-one;
3-(2,4-difluorophenoxy)-2-(4-methanesulfonylphenyl)-6-methylpyran-4-one;
3-(2,5-difluorophenoxy)-2-(methanesulfonylphenyl)-6-methylpyran-4-one;
3-(4-chlorophenyl)-2-(4-methanesulfonylphenyl)-6-methoxymethylpyran-4-one;
3-(4-chlorophenyl)-6-difluoromethyl-2-(4-methanesulfonylphenyl)pyran-4-one;
   and pharmaceutically acceptable salts thereof.

Cox-2 selective inhibitors useful in the subject methods and compositions can include compounds described in the patents individually cited below and incorporated herein by reference.

U.S. Pat. No. 6,472,416.

U.S. Pat. No. 6,451,794.

U.S. Pat. No. 6,169,188.

U.S. Pat. No. 6,020,343.

U.S. Pat. No. 5,981,576.

U.S. Pat. No. 6,222,048.

U.S. Pat. No. 6,057,319.

U.S. Pat. No. 6,046,236.

U.S. Pat. No. 6,002,014.

U.S. Pat. No. 5,945,539.

U.S. Pat. No. 6,359,182.

U.S. Pat. No. 6,538,116.

Cox-2 selective inhibitors useful in the present invention can be supplied by any source as long as the Cox-2 selective inhibitor is pharmaceutically acceptable. Cox-2 selective inhibitors can be isolated and purified from natural sources or can be synthesized. Cox-2 selective inhibitors should be of a quality and purity that is conventional in the trade for use in pharmaceutical products.

Celecoxib useful in the combinations, method, kits and compositions of the invention can be prepared, for example, as set forth in U.S. Pat. No. 5,466,823.

Valdecoxib useful in the combinations, method, kits and compositions of the invention can be prepared, for example, as set forth in U.S. Pat. No. 5,633,272.

Parecoxib useful in the combinations, method, kits and compositions of the invention can be prepared, for example, as set forth in U.S. Pat. No. 5,932,598.

Rofecoxib useful in the combinations, method, kits and compositions of the invention can be prepared, for example, as set forth in U.S. Pat. No. 5,968,974.

Japan Tobacco JTE-522 useful in the combinations, method, kits and compositions of the invention can be prepared, for example, as set forth in Japanese Patent Publication No. JP 90/52882.

Pyrazoles useful in the combinations, method, kits and compositions of the invention can be prepared, for example, as set forth in International Patent Publication No. WO 95/15316.

Pyrazoles can also be prepared as set forth in International Patent Publication No. WO 95/15315.

Pyrazoles can also be prepared as set forth in International Patent Publication No. WO 96/03385.

Thiophene analogs useful in the combinations, method, kits and compositions of the invention can be prepared, for example, as set forth in International Patent Publication No. WO 95/00501.

Thiophene analogs can also be prepared as set forth in International Patent Publication No. WO 94/15932.

Oxazoles useful in the combinations, method, kits and compositions of the invention can be prepared, for example, as set forth in International Patent Publication No. WO 95/00501.

Oxazoles can also be prepared as set forth in International Patent Publication No. WO 94/27980.

Isoxazoles useful in the combinations, method, kits and compositions of the invention can be prepared, for example, as set forth in International Patent Publication No. WO 96/25405.

Imidazoles useful in the combinations, method, kits and compositions of the invention can be prepared, for example, as set forth in International Patent Publication No. WO 96/03388.

Imidazoles can also be prepared as set forth in International Patent Publication No. WO 96/03387.

Cyclopentene Cox-2 inhibitors useful in the combinations, method, kits and compositions of the invention can be prepared, for example, as set forth in U.S. Pat. No. 5,344,991.

Cyclopentene Cox-2 inhibitors can also be prepared as set forth in International Patent Publication No. WO 95/00501.

Terphenyl compounds useful in the combinations, method, kits and compositions of the invention can be prepared, for example, as set forth in International Patent Publication No. WO 96/16934.

Thiazole compounds useful in the combinations, method, kits and compositions of the invention can be prepared, for example, as set forth in International Patent Publication No. WO 96/03,392.

Pyridine compounds useful in the combinations, method, kits and compositions of the invention can be prepared, for example, as set forth in International Patent Publication No. WO 96/03392.

Pyridine compounds can also be prepared as set forth in International Patent Publication No. WO 96/24585.

Illustratively, a Cox-2 selective inhibitor can be a tricyclic compound, for example a compound of formula (VII), a substituted benzopyran derivative, for example a compound of formulas (I) to (VI), or a phenylacetic acid derivative, for example a compound of formula (VIII).

Illustratively, the Cox-2 selective inhibitor can be selected from the group consisting of celecoxib, parecoxib, deracoxib, valdecoxib, etoricoxib, meloxicam, rofecoxib, lumiracoxib, RS 57067, T-614, BMS-347070, JTE-522, S-2474, SVT-2016, CT-3, ABT-963, SC-58125, nimesulide, flosulide, NS-398, L-745337, RWJ-63556, L-784512, darbufelone, CS-502, LAS-34475, LAS-34555, S-33516, SD-8381, prodrugs of any of them, and mixtures thereof.

More particularly, the Cox-2 selective inhibitor can be selected from the group consisting of celecoxib, valdecoxib, parecoxib, rofecoxib, etoricoxib, lumiracoxib, and pharmaceutically acceptable salts thereof.

In one embodiment the Cox-2 selective inhibitor comprises celecoxib.

In another embodiment the Cox-2 selective inhibitor comprises valdecoxib.

In yet another embodiment the Cox-2 selective inhibitor comprises parecoxib sodium.

In certain embodiments, the Cox-2 selective inhibitor is selected from compounds of formulas (XXXVII) to (LI) hereinabove.

The antineoplastic agent for use according to the invention can illustratively be selected from the agents listed in Tables 3-17 below. Grouping of agents by function or mode of action below does not limit the invention to embodiments wherein the antineoplastic agent operates by the function or mode of action indicated.

The invention encompasses all novel combinations of (a) a Cox-2 inhibitor, more particularly a selective Cox-2 inhibitor such as celecoxib, parecoxib, deracoxib, valdecoxib, lumiracoxib, etoricoxib, rofecoxib, and prodrugs and pharmaceutically acceptable salts thereof including, for example, parecoxib sodium, and (b) an antineoplastic agent selected from those disclosed in Tables 3-17 below.

The invention further encompasses all novel combinations of (a) a Cox-2 selective inhibitor selected from compounds of formulas (XXXVII) to (LI) above, and (b) an antineoplastic agent disclosed in above-cited International Patent Publication No. WO 00/38730 or its priority document U.S. Provisional Patent Application Ser. No. 60/113,786, both of which are incorporated herein in their entirety by reference. For convenience, a non-limiting list of illustrative antineoplastic agents is presented in Table 18 below.

TABLE 3
Antimetabolite agents

| Agent | Trade name | Company | Mode of action | Reference | Dosage | Toxicity | Indication |
|---|---|---|---|---|---|---|---|
| IV hydroxyurea | | National | | | | | |
| | | Cancer | | | | | |
| | | Institute | | | | | |
| ZD 9331; | | AstraZeneca | | CAS: | | | |
| (2S)-[4-[N-(2,7-dimethyl-4- | | | | 153537-73-6 | | | |
| oxo-3,4-dihydroquinazolin-6- | | | | | | | |
| ylmethyl)-N-(2-propynyl) | | | | | | | |
| amino]-2-fluorobenzamido]-4- | | | | | | | |
| (1H-tetrazol-5-yl) butyric acid | | | | | | | |
| arzoxifene; | | Eli Lilly | antiestrogen; | CAS: | | | cancer |
| arzoxifene hydrochloride | | | estrogen | 182133-25-1 | | | |
| | | | agonist | (arzoxifene) | | | |
| | | | | 182133-27-3 | | | |
| | | | | (HCl) | | | |
| ERA 923; WAY 138923; | | Ligand | antiestrogen; | CAS: | | | cancer |
| 2-(4-hydroxyphenyl)-3- | | Pharma- | estrogen | 198480-55-6; | | | |
| methyl-1-[[4-[2-(1-piperidinyl) | | ceuticals | agonist | 245124-69-0 | | | |
| ethoxy]phenyl]methyl]-1H- | | | | (mono-HCl) | | | |
| indol-5-ol | | | | | | | |
| pure antiestrogen | | Schering- | antiestrogen; | | | | |
| | | Plough | estrogen | | | | |
| | | | agonist | | | | |
| GTx 006 | | GTx | antiestrogen | | | | |
| T 904064 | Lometrexol | Tularik | antifolate; | | | | |
| | 64 | | disrupts | | | | |
| | | | DNA synthesis | | | | |
| troxacitabine; BCH 4556; | Troxatyl | BioChem | DNA | CAS: | | | cancer |
| 4-amino-1-[(2S,4S)-2- | | Pharma | polymerase | 145918-75-8 | | | |
| (hydroxymethyl)-1,3-dioxolan- | | | inhibitor | | | | |
| 4-yl]-2(1H)-pyrimidinone | | | | | | | |
| nolatrexed; | Thymitaq | Zarix | thymidylate | CAS: | nolatrexed | | cancer |
| nolatrexed dihydrochloride; | | | synthase | 147149-76-6 | administered as | | |
| AG 337; | | | inhibitor; | (nolatrexed); | a 24 h infusion | | |
| 2-amino-6-methyl-5-(4- | | | antimetabolite | 152946-64-0 | of 75-1350 | | |
| pyridinylthio)-4(1H)- | | | | (mono-HCl); | mg/m² to | | |
| quinazolinone | | | | 152946-68-4 | patients with | | |
| | | | | (di-HCl) | advanced | | |
| | | | | | tumors is well | | |
| | | | | | tolerated. | | |
| motexafin gadolinium | Xcytrin | Pharmacyclics | disrupts | | | | brain |
| | | | cellular | | | | metastases |
| | | | metabolism; | | | | |
| | | | inhibits | | | | |
| | | | cellular | | | | |
| | | | adhesion; | | | | |
| | | | enhances | | | | |
| | | | cellular | | | | |
| | | | response | | | | |
| | | | to radiation | | | | |
| tezacitabine (FMdC); | | Matrix | ribonucleotide | | | generally | lung, colon, |
| nucleoside analogue | | Pharmaceutical | reductase | | | well | breast |
| | | | inhibitor; | | | tolerated | (estrogen |
| | | | DNA | | | in | dependent |
| | | | chain | | | Phase I | and |
| | | | terminator; | | | clinical | independent), |
| | | | inhibits | | | trials; | prostate, |
| | | | DNA | | | most | and |
| | | | synthesis | | | commonly | pancreas; |
| | | | | | | reported | also |
| | | | | | | side | effective |
| | | | | | | effects | against |
| | | | | | | were | multi-drug |
| | | | | | | fevers | resistant |
| | | | | | | and | cell lines |
| | | | | | | clinically | |
| | | | | | | manageable | |
| | | | | | | reductions | |
| | | | | | | in white | |
| | | | | | | blood | |
| | | | | | | cell counts | |
| pemetrexed disodium | Alimta | Eli Lilly | multitargeted | | | | |
| | | | antifolate; | | | | |
| | | | inhibits | | | | |
| | | | thymidylate | | | | |
| | | | synthase | | | | |
| | | | and other folate | | | | |
| | | | dependent | | | | |
| | | | enzymes | | | | |
| 17-AAG | | National | Binds to | | | | |
| | | Cancer | heat | | | | |
| | | Institute | shock | | | | |
| | | | protein | | | | |
| | | | Hsp90, | | | | |
| | | | estrogen | | | | |
| | | | receptor | | | | |
| | | | enhances | | | | |
| | | | effect of | | | | |
| | | | paclitaxel | | | | |
| SB 596168 | | Glaxo | selective | | | | |
| | | SmithKline | RNA | | | | |
| | | | polymerase inhibitor | | | | |

TABLE 4
Alkylating agents

| Agent | Trade name | Company | Mode of action | Reference | Dosage | Toxicity | Indication |
|---|---|---|---|---|---|---|---|
| DTI 015 | | Direct | alkylating agent | | | | |
| | | Therapeutics | | | | | |
| methanesulfonic acid, 1-(2- | | Vion | | CAS: | | | |
| chloroethyl)-2-[(methylamino) | | Pharmaceuticals | | 173424-77-6 | | | |
| carbonyl]-2-(methylsulfonyl) | | | | | | | |
| hydrazide | | | | | | | |
| VNP 40101M | | Vion | | | in clinical trials: | | |
| (sulfonyl hydrazine prodrug) | | Pharmaceuticals | | | 15 minute IV | | |
| | | | | | infusion every 4 | | |
| | | | | | weeks; same | | |
| | | | | | weekly in | | |
| | | | | | second trial | | |

TABLE 5
Retinoids

| Agent | Trade name | Company | Mode of action | Reference | Dosage | Toxicity | Indication |
|---|---|---|---|---|---|---|---|
| LGD 1550; ALRT 1550; | | Ligand | | CAS: | | no weight loss or | |
| LG 100550; AGN 193101; | | Pharmaceuticals | | 178600-20-9 | | mucocutaneous | |
| LG 1550; ALRT 550 | | | | | | toxicity at doses of | |
| | | | | | | 30 μg/kg or less in | |
| | | | | | | mice | |
| MX6 | | Maxia | | | | | |
| | | Pharmaceuticals | | | | | |
| trans-retinoic acid | | National Cancer | | CAS: | | | |
| | | Institute | | 302-79-4 | | | |
| alitretinoin; | Panretin | Ligand | | CAS: | | | Kaposi's |
| 9-cis-retinoic acid | | | | 5300-03-8 | | | sarcoma; |
| | | | | | | | leukemia |

TABLE 6
Angiogenesis inhibitors

| Agent | Trade name | Company | Mode of action | Reference | Dosage | Toxicity | Indication |
|---|---|---|---|---|---|---|---|
| IMC-1C11 | | ImClone Systems | angiogenesis inhibitor | | | | |
| recombinant interferon-beta-1a | Aronex | Serono | angiogenesis | | | | |
| | | | inhibitor; | | | | |
| | | | antiproliferative | | | | |
| AE-941 | Neovastat; | Aeterna | angiogenesis | | | | cancer; |
| | Neoretna; | Laboratories | inhibitor; NSAID | | | | psoriasis; |
| | Psovascar; | | | | | | rheumatoid |
| | Arthrovas | | | | | | arthritis; eye |
| | | | | | | | disease; |
| | | | | | | | retinopathy |
| 2-methoxyestradiol; | Panzem | EntreMed | angiogenesis | CAS: | | | cancer |
| 2-ME | | | inhibitor; | 362-07-2 | | | |
| | | | estrogen inhibitor | | | | |
| cilengitide; | | National Cancer | angiogenesis | | | well tolerated | |
| cyclo(L-arginylglycyl-L-alpha- | | Institute; | inhibitor | | | and safe in | |
| aspartyl-D-phenylalanyl-N- | | Merck | | | | patients with | |
| methyl-L-valyl) | | | | | | advanced tumors | |
| IM 862; | | Alza; Cytran | angiogenesis inhibitor | CAS: | | no hematological | AIDS-related |
| L-alpha-glutamyl-L-tryptophan | | | | 38101-54-6 | | toxicities, decline | Kaposi's |
| | | | | | | in viral load, | sarcoma |
| | | | | | | headache, | |
| | | | | | | fatigue, tingling | |
| | | | | | | and moodiness | |
| bevacizumab | Avastin | Genentech; | angiogenesis inhibitor | | | | |
| | | National Cancer | | | | | |
| | | Institute | | | | | |
| CAI; | | National Cancer | angiogenesis | | | | |
| carboxyamidotriazole | | Institute | inhibitor; | | | | |
| | | | antimetastatic | | | | |
| PKC 412 | | Novartis | angiogenesis inhibitor | | | | advanced |
| | | | | | | | cancers |

TABLE 7
Anticancer antibiotics

| | Trade | | | | | | |
|---|---|---|---|---|---|---|---|
| Agent | name | Company | Mode of action | Reference | Dosage | Toxicity | Indication |
| E 7070; ER 35744; | | Eisai | antibiotic; | CAS: | | | cancer |
| N-(3-chloro-1H-indol-7-yl)- | | | sulfonamide | 165668-41-7 | | | |
| 1,4-benzenedisulfonamide | | | | | | | |
| taurolidine; | Taurolin | Carter-Wallace | | CAS: | in vivo, 25 daily | | bacterial |
| 4,4′-methylenebis[tetrahydro- | | | | 19388-87-5 | injections of | | infection; |
| 2H-1,2,4-thiadiazine]1,1,1′,1′- | | | | | taurolidine at | | cancer |
| tetraoxide | | | | | doses of | | |
| | | | | | 350 mg/kg/d | | |
| | | | | | are | | |
| | | | | | well tolerated | | |
| | Oramed | Biosyn | anti-fungal agent | | 75 mg/d for two | safe and well | active against |
| | | | | | weeks | tolerated | azole-resistant |
| | | | | | | | candida |
| | | | | | | | strains |
| valrubicin | Valstar | Anthra | arrests cell in G2; | | | | papillary bladder |
| | | Pharmaceuticals | inhibits DNA | | | | cancer |
| | | | topoisomerase II | | | | |
| trimetrexate glucuronate | Neutrexin | MedImmune | | | 45 mg/m²once | very serious | treatment of |
| | | Oncology | | | daily by IV | and | moderate to |
| | | | | | infusion over | potentially | severe PCP |
| | | | | | 60-90 minutes. | life- | pneumonia in |
| | | | | | Leucovorin | threatening | people with |
| | | | | | must be given | side- | compromised |
| | | | | | daily during | effects | immune systems |
| | | | | | trimetrexate | | |
| | | | | | treatment and | | |
| | | | | | for 72 hours | | |
| | | | | | afterward. | | |
| | | | | | Leucovorin may | | |
| | | | | | be given IV at | | |
| | | | | | 20 mg/m² over | | |
| | | | | | 5-10 minutes | | |
| | | | | | every 6 hours or | | |
| | | | | | orally as 4 doses | | |
| | | | | | of 20 mg/m² | | |
| | | | | | spaced evenly | | |
| | | | | | throughout the | | |
| | | | | | day | | |
| 5-azacytidine; | | National Cancer | antibiotic; | CAS: | | | acute myelocytic |
| 4-amino-1-beta-D-ribofuranosyl- | | Institute | RNA/DNA | 320-67-2 | | | leukemia and |
| 1,3,5-triazin-2(1H)-one | | | antimetabolite | | | | myelodysplastic |
| | | | | | | | syndrome |
| nystatin (IV) | Nyotran | Aronex | anti-fungal agent | | | | fungal infections |
| | | Pharmaceuticals | | | | | |

TABLE 8
DNA topoisomerase I inhibitors

| Agent | Trade name | Company | Mode of action | Reference | Dosage | Toxicity | Indication |
|---|---|---|---|---|---|---|---|
| irinotecan | Camptosar | Pharmacia | DNA topoisomerase I | | | | metastatic colon |
| | Oral | | inhibitor | | | | cancer |
| camptothecin glycoconjugate | | Bayer | DNA topoisomerase I | | | | refractory solid |
| | | | inhibitor | | | | tumors |

TABLE 9
Hormonal anticancer agents

| Agent | Trade name | Company | Mode of action | Reference | Dosage | Toxicity | Indication |
|---|---|---|---|---|---|---|---|
| leuprolide acetate 7.5 mg in | Leuprogel 1 | Atrix | LHRH antagonist; | | | | |
| the Atrigel drug delivery | Month | Laboratories | hormonal therapy | | | | |
| system for subcutaneous depot | | | | | | | |
| injection | | | | | | | |
| leuprolide acetate 22.5 mg in | Leuprogel 3 | Atrix | LHRH antagonist; | | | | |
| the Atrigel drug delivery | Month | Laboratories | hormonal therapy | | | | |
| system for subcutaneous depot | | | | | | | |
| injection | | | | | | | |
| leuprolide acetate 30 mg in the | Leuprogel 4 | Atrix | LHRH antagonist; | | | | |
| Atrigel drug delivery system | Month | Laboratories | hormonal therapy | | | | |
| for subcutaneous depot | | | | | | | |
| injection | | | | | | | |
| SPD-424 | | Shire | Subcutaneous | | | | prostate cancer |
| | | Pharmaceutical | implant containing | | | | |
| | | | GnRH agonist | | | | |
| Dynepo gene activated | | Aventis; | Anti-anemic; human | | | | anemia associated |
| erythropoietin | | Transkaryotic | erythropoietin; | | | | with |
| | | Therapies | stimulates production | | | | chemotherapy |
| | | | of red blood cells | | | | |

TABLE 10
Immunomodulator agents

| Agent | Trade name | Company | Mode of action | Reference | Dosage | Toxicity | Indication |
|---|---|---|---|---|---|---|---|
| BCI immune | | Intracel | immunostimulant; | | | | |
| stimulator | | | antigenic protein | | | | |
| SMART 1D10 | | Protein Design | immunosuppressant | | in patients | | autoimmune |
| | | Laboratories | | | undergoing renal | | disease; transplant |
| | | | | | transplantation, | | rejection; psoriasis; |
| | | | | | treatment with | | rheumatoid arthritis |
| | | | | | 0.012, 0.06 or | | |
| | | | | | 0.12 mg/kg | | |
| | | | | | MEDI-507, 6 and | | |
| | | | | | 60-72 h after | | |
| | | | | | transplantation, is | | |
| | | | | | well tolerated | | |
| interferon-alpha | | Valentis | gene therapy; | | | | |
| gene therapy | | | immunostimulant | | | | |
| | Xcellerate | Xcyte Therapies | immunostimulant | | | | |
| gene therapy, | | Valentis | gene therapy | | | | |
| interleukin-2 + | | | | | | | |
| staphylococcal | | | | | | | |
| enterotoxin B | | | | | | | |
| NBI-3001; IL-4 PE | | Neurocrine | IL-4 fusion toxin | | | | recurrent |
| (interleukin-4 | | Biosciences | | | | | glioblastomas |
| pseudomonas | | | | | | | |
| exotoxin) | | | | | | | |
| Vaccinia/fowlpox | | Therion | immunostimulant | | | | colorectal, lung |
| CEA/TRICOM | | Biologics | CEA vaccine | | | | cancer |
| interleukin-2 gene | | Valentis | gene therapy | | | | |
| therapy in | | | | | | | |
| conjunction with | | | | | | | |
| chemotherapy | | | | | | | |
| OSI-774 | Tarceva | Genentech; | EGFR inhibitor; | | Phase II dose of | generally well | cancers including |
| | | Hoffmann-La | small molecule | | 150 mg/day | tolerated at the | ovarian, |
| | | Roche; | tyrosine kinase | | | Phase II dose | pancreatic, |
| | | OSI | inhibitor | | | with a generally | nonsmall cell |
| | | Pharmaceuticals | | | | reversible | lung, breast, and |
| | | | | | | acneiform rash | head and neck |
| | | | | | | and occasional | |
| | | | | | | diarrhea that | |
| | | | | | | responds to | |
| | | | | | | therapy | |
| histamine | Ceplene | Maxim | immunostimulant; | | | | |
| dihydrochloride | Injection | Pharmaceuticals | prevents release of | | | | |
| | | | oxygen free radicals; | | | | |
| | | | reduces oxidative | | | | |
| | | | stress | | | | |
| pegylated | Pegasys | Hoffmann-La | immunomodulator; | | once-weekly | fatigue, headache, | chronic hepatitis C |
| interferon | | Roche | protection against | | subcutaneous | myalgia, rigors, | |
| alpha-2a | | | enzymatic degradation; | | dose of 180 μg | pyrexia, nausea, | |
| | | | reduces renal | | for 48 weeks | abdominal pain, | |
| | | | clearance; anti- | | | and depression; | |
| | | | inflammatory activity | | | neutropenia and | |
| | | | | | | thrombocytopenia | |
| | | | | | | reported | |
| beta-alethine | Beta LT | Dovetail | immunomodulator | | | | B-cell lymphoma; |
| | | Technologies | | | | | multiple myeloma |
| APC-8020 | Mylovenge | Dendreon | vaccine; M-protein; | | | | B-cell |
| | | | immunomodulator | | | | malignancies |
| interleukin-2/ | | Valentis | immunotherapeutic | | | | metastatic |
| superantigen B | | | treatment of tumor | | | | malignant |
| gene combination | | | cells by direct | | | | melanoma |
| | | | injection | | | | |
| | Melacine | Corixa; | immunostimulant | | 2 shots once a | | malignant |
| | vaccine | Schering-Plough | consisting of lysed | | week for 5 | | melanoma |
| | | | cells from 2 human | | weeks, 2 week | | |
| | | | melanoma cell lines | | break, weekly | | |
| | | | combined with | | injections for 5 | | |
| | | | DETOX | | weeks | | |
| SD/01 | | Amgen | stimulates growth of | | | | neutropenia |
| | | | white blood cells; | | | | |
| | | | prevents infection | | | | |
| OSI-774 in | | Genentech; | anti-EGFR | | | | |
| combination with | | Hoffmann-La | | | | | |
| Taxotere | | Roche; OSI | | | | | |
| | | Pharmaceuticals | | | | | |

TABLE 11
Miscellaneous antineoplastic agents

| Agent | Trade name | Company | Mode of action | Reference | Dosage | Toxicity | Indication |
|---|---|---|---|---|---|---|---|
| gallium maltolate | | Titan | ribonucleotide | CAS: | once or twice | good safety | cancer; |
| | | Pharmaceuticals | reductase inhibitor | 108560-70-9 | daily dosing | profile | HIV infection |
| | | | | | schedule | | |
| mivobulin; CI-980; | CI 980, NSC 613862 | National Cancer Institute | mitosis inhibitor; | CAS | | | cancer |
| NSC 613862; | | | tubulin polymerase inhibitor | 122332-18-7 | | | |
| (S)-(5-amino-1,2-dihydro-2- | | | | | | | |
| methyl-3-phenylpyrido(3,4- | | | | | | | |
| b)pyrazin-7-yl)carbamic acid | | | | | | | |
| ethyl ester | | | | | | | |
| T138067; T-67; | | Tularik | microtubule assembly inhibitor | CAS | well tolerated | | cancer |
| 2,3,4,5,6-pentafluoro-N-(3- | | | | 195533-53-0 | up to 440 mg/m² | | |
| fluoro-4-methoxyphenyl) | | | | 195533-98-3 | by 3- | | |
| benzenesulfonamide | | | | (Na salt); | hour infusion | | |
| | | | | 195533-97-2 | every 4 weeks | | |
| | | | | (K salt) | | | |
| 5-aza-2-deoxycytidine | | National Cancer Institute | antiproliferative; | | 125 mg/m² 12 h | myelosuppression, | solid tumors |
| | | | activates tumor | | every 6 d + amsacrine | especially neutropenia; | (head and neck, |
| | | | suppressor genes | | 120 mg/m² | mild to | colon, kidney, |
| | | | | | on 6 d & | moderate non- | testis, melanoma, |
| | | | | | 7 d; IV infusion | hematological | ovaries, cervix |
| | | | | | 15 mg/m² 8 h for | toxicity including | and lung; |
| | | | | | 3 d every 8 wks | nausea, vomiting, | leukemia |
| | | | | | | obstipation, | |
| | | | | | | diarrhea, | |
| | | | | | | cerebellar or | |
| | | | | | | cerebral toxicity, | |
| | | | | | | phlebitis, increase | |
| | | | | | | in liver enzyme | |
| | | | | | | levels; rarely | |
| | | | | | | alopecia and | |
| | | | | | | mucositis | |
| N,N-dimethyl-L-valyl-L-valyl- | | ILEX | microtubule assembly | | | | |
| N-methyl-L-valyl-L-prolyl-N- | | | inhibitor; peptide | | | | |
| (1,1-dimethylethyl)-L- | | | | | | | |
| prolinamide | | | | | | | |
| N-[3-[(aminocarbonyl)amino]- | | Tularik | microtubule assembly | | 60-100 mg/kg/wk; | | MDR-expressing |
| 4-methoxyphenyl]-2,3,4,5,6- | | | inhibitor | | lower doses | | subcutaneous |
| pentafluoro-benzenesulfonamide | | | | | in combination | | tumors |
| | | | | | with other drugs | | |
| CCI-779 | | Wyeth-Ayerst | signal transduction | | | | cancer |
| | | | inhibitor | | | | |
| NC-100150 | Clariscan | Nycomed | MRI agent; contrast | | | | diagnosis |
| | | Amersham | medium | | | | |
| lasofoxifene | | Ligand | | | | | |
| | | Pharmaceuticals | | | | | |
| antigens | Necrosis Therapy; TNT | | antibody; | | | | |
| | | | radiotherapeutic | | | | |
| GTI-2040 | | Lorus | antisense | | 185 mg/m²/d as | | regression of |
| | | Therapeutics | oligonucleotide | | a 3 wk | | tumors from |
| | | | | | continuous IV | | several cancers |
| | | | | | infusion | | |
| MGS-rCEA | | Protein Sciences | Recombinant | | | | |
| | | | carcinoma embryonic | | | | |
| | | | antigen | | | | |
| R-115777 | | Janssen Pharmaceutica | farnesyl protein | CAS: | | | cancer |
| 6-[amino-(4-chlorophenyl)(1- | | | transferase inhibitor; | 192185-68-5 | | | |
| methyl-1H-imidazol-5-yl) | | | signal transduction | | | | |
| methyl]-4-(3-chlorophenyl)-1- | | | inhibitor | | | | |
| methyl-2(1H)-quinolinone | | | | | | | |
| MedPulser and disposable | | Genetronics | | | | | |
| sterile electrode applicators | | | | | | | |
| used in combination with | | | | | | | |
| bleomycin | | | | | | | |
| liposome NDDP; L-NDDP | Aroplatin; Platar | Aronex | platinum complex | | | | |
| | | Pharmaceuticals | | | | | |
| CDC-801 | | Celgene | cytokine inhibitor; | | | | Crohn disease; |
| | | | TNF inhibitor; | | | | inflammation |
| | | | phosphodiesterase IV | | | | |
| | | | inhibitor | | | | |
| arsenic trioxide | Trisenox | Cell Therapeutics | apoptosis inducer; | CAS: | | | cancer |
| | | | differentiation | 1327-53-3 | | | |
| | | | inducer | | | | |
| | Filmix | Cav-Con | drug delivery system | | | | |
| NC-100100; DD-723 | New Ultrasound; | Nycomed | echo contrast | | | | diagnosis |
| | NUS; Sonazoid | Amersham | medium | | | | |
| BAM-002 | | Novelos | peptide | | | | cancer |
| | | Therapeutics | | | | | |
| depsipeptide; NSC 630176 | | National Cancer | peptide | CAS: | 24.9 mg/m² | grade 3 fatigue, | cancer |
| N-[(3S,4E)-3-hydroxy-7- | | Institute | | 128517-07-7 | | nausea and | |
| mercapto-1-oxo-4-heptenyl]- | | | | | | vomiting, grade | |
| D-valyl-D-cysteinyl-(2Z)-2- | | | | | | 4 thrombocytopenia | |
| amino-2-butenoyl-L-valine (4- | | | | | | and cardiac | |
| 1)-lactone cyclic (1-2)disulfide | | | | | | arrhythmia | |
| K-ras antisense | | National Cancer | gene therapy; | | | | cancer |
| oligonucleotide | | Institute | antisense | | | | |
| | | | oligonucleotide | | | | |
| chloroquinoxaline | | National Cancer | | CAS: | repetitive 1000 mg/m² | | cancer |
| sulfonamide; | | Institute | | 97919-22-7 | doses | | |
| chlorsulfaquinoxaline; | | | | | | | |
| 4-amino-N-(5-chloro-2-quin- | | | | | | | |
| oxalinyl)benzenesulfonamide | | | | | | | |
| NX-211 | | Gilead Sciences | DNA topoisomerase | | maximum dose | neutropenia and | |
| liposome lurtotecan | | | inhibitor | | 1.8 mg/m²/d in | thrombocytopenia | |
| | | | | | patients with | | |
| | | | | | more than two | | |
| | | | | | prior | | |
| | | | | | chemotherapies; | | |
| | | | | | patients with | | |
| | | | | | one or less prior | | |
| | | | | | chemotherapies | | |
| | | | | | under | | |
| | | | | | evaluation at | | |
| | | | | | 2.1 mg/m²/d | | |
| CDC-501 | | Celgene | TNF modulator | | | | cancer |
| N-acetyl-3-(2-naphthalenyl)- | Abarelix- | Amgen | gonadorelin | CAS: | | | cancer; benign |
| D-alanyl-4-chloro-D-phenyl- | depot | | antagonist | 183552-38-7; | | | prostate |
| alanyl-3-(3-pyridinyl)-D- | | | | 186837-47-8 | | | hypertrophy; |
| alanyl-L-seryl-N-methyl-L- | | | | trifluoro- | | | endometriosis |
| tyrosyl-D-asparaginyl-L-N6- | | | | acetate salt | | | |
| (1-methylethyl)-L-lysyl-L- | | | | | | | |
| prolyl-D-alaninamide | | | | | | | |
| atrasentan; A-127722; | | Abbott | endothelin A | CAS: | 2.5 or 10 mg/d | increase in | cancer; restenosis; |
| (2R,3R,4S)-4-(1,3-benzodioxol- | | Laboratories | antagonist | 173937-91-2; | well tolerated | rhinitis | myocardial |
| 5-yl)-1-[2-(dibutylamino)-2- | | | | 195733-43-8 | with no grade | | infarction |
| oxoethyl]-2-(4-methoxy- | | | | (HCl salt) | III/IV toxicities. | | |
| phenyl)-3-pyrrolidinecarboxylic | | | | | | | |
| acid | | | | | | | |
| CV-787 | | Calydon | gene therapy | | | | cancer |
| L-377202 | | Merck | | | | | |
| MPC-7869; (R)-flurbiprofen; | | Myriad | NSAID | CAS: | well tolerated at | | cancer |
| (alphaR)-2-fluoro-alpha- | | Genetics | | 51543-40-9 | multiple doses | | |
| methyl-[1,1′-biphenyl]-4-acetic | | | | | of 1200 mg qd | | |
| acid | | | | | and 800 mg bid | | |
| (R)-2,3,4,5-tetrahydro-1-(1H- | | Bristol-Myers | farnesyl protein | | IV 36-225 mg/m² | mainly | solid tumors |
| imidazol-4-ylmethyl)-3- | | Squibb | transferase inhibitor; | | and oral | gastrointestinal | |
| (phenylmethyl)-4-(2- | | | apoptosis inducer | | 36-168 mg/m² | | |
| thienylsulfonyl)-1H-1,4- | | | | | | | |
| benzodiazepine-7-carbonitrile | | | | | | | |
| N-[4-[(3-chloro-4-fluoro- | | Pfizer | tyrosine kinase | | well tolerated at | no signs of | suppresses tumor |
| phenyl)amino]-7-[3-(4- | | | inhibitor; EGF | | 50-650 mg/d | toxicity | growth |
| morpholinyl)propoxy]-6- | | | receptor inhibitor | | | | |
| quinazolinyl]-2-propenamide | | | | | | | |
| GW-572016 | | Glaxo- | tyrosine kinase | | | | |
| | | SmithKline | inhibitor; signal | | | | |
| | | | transduction inhibitor | | | | |
| INX-3280; LR-3280; | | Inex | antisense | | | well tolerated for | |
| BW-4003W94; TA-3280 | | | oligonucleotide | | | wide range of | |
| | | | | | | dosages | |
| Egr-1 + TNF-alpha | TNFerade | GenVac | enhances apoptosis | | | | induces tumor |
| | | | by radiation | | | | shrinkage |
| aprepitant; | | Merck | neurokinin 1 | | | | |
| 5-[[(2R,3S)-2-[(1R)-1-[3,5- | | | antagonist | | | | |
| bis(trifluoromethyl)phenyl] | | | | | | | |
| ethoxy]-3-(4-fluorophenyl)-4- | | | | | | | |
| morpholinyl]methyl]-1,2- | | | | | | | |
| dihydro-3H-1,2,4-triazol-3-one | | | | | | | |
| erythropoiesis stimulating | Aranesp | Amgen | | | well tolerated at | hemoglobin | anemia in cancer |
| protein; erythropoietin [30- | | | | | 0.5 to 4.5 μg/kg | response is dose- | patients |
| asparagine, 32-threonine, 87- | | | | | over 12 wks | dependent | |
| valine, 88-asparagine, 90- | | | | | | | |
| threonine] (human) | | | | | | | |
| mucositis therapy; RK 0202 | | Elan; RxKinetix | | | | | |
| SB-251353 | | Glaxo- | growth factor; | | | | |
| | | SmithKline | hematopoietic factor; | | | | |
| | | | immunostimulant; | | | | |
| | | | chemokine | | | | |
| rasburicase; urate oxidase | Fasturtec | Sanofi- | ureate oxidase; | | 0.2 mg/kg IV | 3 of 95 subjects | converts uric acid |
| (Aspergillus flavus clone | | Synthelabo | enzyme | | daily for 1 to 7 d | report vomiting, | into water soluble |
| 9C/9A reduced) | | | | | to subjects | rash, pruritis | alantoin |
| | | | | | receiving | | |
| | | | | | chemotherapy | | |
| CP-609754 | | OSI; Pfizer | RAS inhibitor | | | well tolerated | |
| 1,25(OH)2-16ene-23yne-26,27 | | Ilex | vitamin D3 analog | | | | |
| hexafluorovitamin D3 | | | | | | | |
| (1S,3S,7S,10R,11S,12S,16R)- | | Bristol-Myers | | CAS: | | | shrinks paclitaxel |
| 7,11-dihydroxy-8,8,10,12,16- | | Squibb | | 219989-84-1 | | | resistant caner |
| pentamethyl-3-[(1E)-1-methyl- | | | | | | | tumors |
| 2-(2-methyl-4-thiazolyl) | | | | | | | |
| ethenyl]-17-oxa-4-azabicyclo | | | | | | | |
| [14.1.0] heptadecane-5,9-dione | | | | | | | |
| flavopiridol; | | Aventis | cyclin dependent | | 24 h continuous | dose limiting | |
| cis-2-(2-chlorophenyl)-5,7- | | | kinase inhibitior | | infusion of | toxicities are | |
| dihyroxy-8-(3-hydroxy-1- | | | | | paclitaxel 135 | pulmonary and | |
| methyl-4-piperidinyl)-4H-1- | | | | | or 100 mg/m² | hematologic and | |
| benzopyran-4-one | | | | | followed by | neutropenia | |
| hydrochloride | | | | | escalating doses | | |
| | | | | | of flavopiridol, | | |
| | | | | | 24 h continuous | | |
| | | | | | infusion repeated | | |
| | | | | | every 21 d | | |
| BAY-439006 | | Bayer | Raf protein kinase | | 50-400 mg/day | well tolerated | prevents tumor |
| | | | inhibitor | | | | growth |
| Rebeccamycin analog; | | Bristol-Myers | | | | | |
| BMS-181176 | | Squibb; NCI | | | | | |
| adenoviral p53 | | Introgen | gene therapy | | | | |
| | | Therapeutics; | | | | | |
| | | NCI | | | | | |
| exisulind | Aptosyn | Cell Pathways | selective apoptotic | | | | metastatic breast |
| | | | antineoplastic drug; | | | | cancer |
| | | | cyclic GMP PDE | | | | |
| | | | inhibitor; apoptosis | | | | |
| | | | agonist | | | | |
| phosphorothioate antisense | Genasense | Genta | antisense | | | | cancer |
| oligionucleotide | | | oligonucleotide; | | | | |
| | | | Bcl-2 blocker | | | | |
| MG98 second-generation | | MGI Pharma | antisense; blocks | | | well tolerated in | |
| mRNA inhibitor | | | production of DNA | | | Phase I trials; | |
| | | | methyltransferase | | | transient side | |
| | | | | | | effects including | |
| | | | | | | fatigue, anorexia, | |
| | | | | | | fever and chills, | |
| | | | | | | elevated liver | |
| | | | | | | enzymes | |
| imatinib mesylate; STI-571 | Glivec | Novartis | protein tyrosine | | 400 mg/d for | nausea, fluid | adult patients |
| | | Pharmaceuticals | kinase inhibitor; | | patients in | retention, muscle | with Philadelphia |
| | | | Bcr-Abl inhibitor | | chronic phase | cramps, diarrhea, | chromosome (bcr- |
| | | | | | CML; 600 mg/d | vomiting, | abl) positive |
| | | | | | for patients in | abnormal | chronic myeloid |
| | | | | | accelerated | bleeding, muscle | leukemia (CML) |
| | | | | | phase or in blast | and bone pain, | in chronic phase |
| | | | | | crisis. | skin rash, | leukemia (CML) |
| | | | | | | headache, | in chronic phase |
| | | | | | | fatigue, joint | after failure of |
| | | | | | | pain, indigestion, | interferon-alpha |
| | | | | | | and shortness of | therapy, or in |
| | | | | | | breath; serious | accelerated phase |
| | | | | | | and severe side | or blast crisis |
| | | | | | | effects such as | |
| | | | | | | liver problems, | |
| | | | | | | fluid retention, | |
| | | | | | | and low levels of | |
| | | | | | | certain blood | |
| | | | | | | cells reported in | |
| | | | | | | some patients | |
| MS-275 | | National Cancer | Oral histone | | | | refractory solid |
| | | Institute; | deacetylase inhibitor; | | | | tumors and |
| | | Mitsui | terminal cell | | | | lymphomas |
| | | Pharmaceuticals | differentiation; | | | | |
| | | | apoptosis | | | | |
| phenylacetate | | National Cancer | | | | | |
| | | Institute; | | | | | |
| | | Elan | | | | | |
| | | Pharmaceuticals | | | | | |
| AFP-Scan | | Immunomedics | Tc99m-labeled | | | | diagnosis of AFP- |
| | | | murine antibody | | | | producing tumors; |
| | | | fragment for nuclear | | | | primary liver and |
| | | | imaging | | | | germ cell cancer |
| | | | | | | | staging |
| tirapazamine | Tirazone | Sanofi- | attacks hypoxic cells | | | | |
| | | Synthelabo | | | | | |
| ZD-0473 | | AstraZeneca | platinum agent | | | | solid tumors |
| epratuzumab | LymphoCide | Immunomedics | humanized antibody | | | | non-Hodgkin's |
| | | | targeting CD22 | | | | lymphoma |
| | | | receptor | | | | |
| | LymphoScan | Imunomedics | Tc99m-labeled | | | | diagnosis of |
| | | | murine antibody | | | | CD22-expressing |
| | | | fragment for nuclear | | | | lymphomas |
| | | | imaging | | | | |
| LDP-341 | | Millennium | proteasome inhibitor; | | | | refractory and |
| | | Pharmaceuticals | induces apoptosis; | | | | relapsed multiple |
| | | | inhibits cell growth, | | | | myeloma, solid |
| | | | cell adhesion, | | | | tumors, other |
| | | | angiogenesis | | | | |
| skeletal targeted radiotherapy | | NeoRx | small molecule bone- | | | | bone and bone |
| (STR) | | | targeting agent | | | | marrow related |
| | | | combined with | | | | cancers |
| | | | radionuclide | | | | |
| paclitaxel in Paclimer | | Guilford | biopolymer site- | | | | |
| microspheres | | Pharmaceuticals | specific controlled | | | | |
| | | | drug delivery | | | | |
| peripheral blood lymphocytes | | National Cancer | | | | | |
| transduced with a gene | | Institute | | | | | |
| encoding a chimeric T-cell | | | | | | | |
| receptor | | | | | | | |
| 1-alpha-hydroxy-vitamin D2 | | Bone Care | stimulates osteoblasic | | | | |
| | | International | activity | | | | |
| BUDR | | National Cancer | inhibits mitosis | CAS: | | | |
| | | Institute | | 59-14-3 | | | |
| T4N5 Liposome Lotion; | Dimericine | AGI Dermatics | DNA repair enzyme | | | | photosensitivity |
| T4 endonuclease V | | | | | | | to UV rays in |
| encapsulated in liposomes | | | | | | | patients with |
| | | | | | | | xeroderma |
| | | | | | | | pigmentosa |
| benzoylphenylurea (BPU) | | NCI; Ishihara | beta alethine A | | | | |
| | | Sangyo Kaisha | antitubulin agent | | | | |
| BMS 214662 (farnesyl | | Bristol-Myers | inhibits farnesyl | | | | |
| transferase inhibitor) | | Squibb | transferase | | | | |
| CI-1033 | | Pfizer | ErbB tyrosine kinase | | | | |
| | | | inhibitor; growth | | | | |
| | | | inhibitor | | | | |
| combretastatin | | Bristol-Myers | vascular targeting | | | | |
| | | Squibb; | agent that occludes | | | | |
| | | OXiGENE | blood flow to tumors | | | | |
| cryptophycin | | Eli Lilly | | | | | |
| INGN 241 adenoviral-mda7 | | Introgen | adenoviral vector; | | | | |
| | | Therapeutics | induces apoptosis; | | | | |
| | | | activates PKR | | | | |
| tributyrin | | National Cancer | induces malignant | | | | |
| | | Institute | cells to differentiate; | | | | |
| | | | induces apoptosis | | | | |
| ADL 8-2698 | | Adolor | opioid antagonist | | | | opioid induced |
| | | | | | | | bowel |
| | | | | | | | dysfunction |
| buthionine sulfoximine | | National Cancer | depletes arterial | | | | |
| | | Institute | glutathione; inhibits | | | | |
| | | | glutamylcysteine | | | | |
| | | | synthase | | | | |
| caroxypeptidase G2 | | National Cancer | bacterial enzyme that | | | | methotrexate- |
| | | Institute; | hydrolyzes the C- | | | | induced renal |
| | | Duramed | terminal glutamate | | | | dysfunction; |
| | | Europe | residue from MTX | | | | methotrexate |
| | | | | | | | overdose |
| | | | | | | | following |
| | | | | | | | intrathecal |
| | | | | | | | administration |
| metoclopromide (intranasal | Emitasol | Questcor; | anti-nausea | | | | acute and delayed |
| spray) | | Shire | | | | | emesis in patients |
| | | Pharmaceutical | | | | | undergoing |
| | | | | | | | chemotherapy |
| dalteparin sodium injection | Fragmin | Pharmacia | heparin | | | | clot prevention in |
| | | | | | | | cancer patients |
| MK-869 | | Merck | anti-nausea | | | | |
| multiple drug resistance | | Eli Lilly | | | | | cancer drug |
| inhibitor | | | | | | | resistance |
| oprelvekin | Neumega | Wyeth-Ayerst | | | administered | rather well | chemotherapy- |
| | | | | | under skin | tolerated with | induced |
| | | | | | | few side effects | thrombocytopenia |
| | | | | | | including | |
| | | | | | | swelling of arms | |
| | | | | | | and legs, fatigue, | |
| | | | | | | blurred vision, | |
| | | | | | | cardiac | |
| | | | | | | dysfunction, | |
| | | | | | | fluid retention | |
| N-monomethyl-L-arginine | | National Cancer | nitric oxide synthase | | | | interleukin-2- |
| | | Institute | inhibitor | | | | induced |
| | | | | | | | hypotension |
| repifermin | | Human Genome | human protein | | | | mucositis |
| | | Sciences | | | | | resulting from |
| | | | | | | | chemotherapy |
| rhTPO recombinant human | | Genentech; | mobilization of | | | | prevention of |
| thrombopoietin | | Pharmacia | peripheral blood | | | | chemotherapy- |
| | | | progenitor cells | | | | induced |
| | | | | | | | thrombocytopenia |
| SR29142 urate oxidase | | Sanofi- | uricolytic agent | | | | reduction of uric |
| | | Synthelabo | | | | | acid levels |
| | | | | | | | associated with |
| | | | | | | | chemotherapy |
| ancestim | Stemgen | Amgen | early acting blood | | injection under | | |
| | | | cell growth factor | | skin: 20 μg/kg | | |
| | | | proginitor; reduction | | for 5 days | | |
| | | | of uric acid levels | | | | |
| lutetium-texaphyrin | Lu-Tex | Pharmacyclics | Photosensitizer; | | | | |
| | | | photodynamic cancer | | | | |
| | | | therapy | | | | |
| CP-461 | | Cell Pathways | SAAND; cGMP PDE | | | | |
| | | | inhibitor; activates | | | | |
| | | | PKG | | | | |
| EKB-569 | | Wyeth-Ayerst | EGFR tyrosine kinase | | | | |
| | | | inhibitor | | | | |
| GTI-2501 | | Lorus | antisense | | | | |
| | | Therapeutics | | | | | |
| ILX-651; | | Ilex Oncology | pentapeptide; | | | | |
| dolostatin | | | antitubulin; interrupts | | | | |
| | | | cell mitosis | | | | |
| Perillyl alcohol monoterpenes | | Endorex | inhibits signal transduction | | | | |
| (new formulation) | | | pathways | | | | |
| | | | downstream of | | | | |
| | | | Bcl/Abl kinase; | | | | |
| | | | inhibits cell growth; | | | | |
| | | | induces apoptosis | | | | |
| PTK-787 | | Novartis | inhibits vascular | | | | advanced cancers |
| | | | endothelial GFR, | | | | |
| | | | tyrosine kinases; | | | | |
| | | | impairs vascular | | | | |
| | | | endothelial growth | | | | |
| | | | factor-induced | | | | |
| | | | responses and tumor | | | | |
| | | | growth | | | | |
| T-900607 | | Tularik | binds tubulin | | | | |
| flavopriridol | | Aventis | CDK inhibitor | | | | |

TABLE 12
Matrix metalloproteinase inhibitors

| Agent | Trade name | Company | Mode of action | Reference | Dosage | Toxicity | Indication |
|---|---|---|---|---|---|---|---|
| BMS-275291 | | Bristol-Myers | MMP inhibitor; | | | generally well | metastatic |
| | | Squibb | angiogenesis | | | tolerated | NSCLC |
| | | | inhibitor | | | | |
| prinomastat | | Pfizer | MMP inhibitor; | | | | |
| | | | angiogenesis | | | | |
| | | | inhibitor | | | | |

TABLE 13
Monoclonal antibodies

| | | | | Refer- | | | |
|---|---|---|---|---|---|---|---|
| Agent | Trade name | Company | Mode of action | ence | Dosage | Toxicity | Indication |
| | Rituxan | IDEC | | | | | non-Hodgkin's |
| | | Pharmaceuticals; | | | | | lymphoma, breast |
| | | Genentech; | | | | | and colon cancer, |
| | | Hoffmann-La- | | | | | melanoma |
| | | Roche; | | | | | |
| | | Zenyaku Kogyo | | | | | |
| | Bexxar | Coulter | | | | | non-Hodgkin's |
| | | Pharmaceutical | | | | | lymphoma, breast |
| | | | | | | | and colon cancer, |
| | | | | | | | melanoma |
| HER-2/neu protein | Herceptin | UCLA; | humanized MAb | | | | non-Hodgkin's |
| antibody | | Genetech | against Her-2 growth | | | | lymphoma, breast |
| | | | factor receptor | | | | and colon cancer, |
| | | | | | | | melanoma |
| EGF receptor | | M. D. Anderson | | | | | |
| antibody | | Cancer Center | | | | | |
| | | in Hourston | | | | | |
| | Panorex | Centocor | MAb to 17-1A | | | | late-stage |
| | | | protein | | | | colorectal cancer |
| MAb, interleukin- | | | immunotoxin; | | | | cancer |
| 13-PE38QQR; | | | monoclonal antibody | | | | |
| IL-13-PE38QQR | | | | | | | |
| 2B1 bispecific | | National Cancer | | | | | |
| murine MAb | | Institute | | | | | |
| Mab 3A1 | | National Cancer | | | | | |
| | | Institute | | | | | |
| MAb, BR96 | SGN-10 | Seattle Genetics | immunotoxin | | | | cancer |
| sFv-PE40 | | | | | | | |
| | SGN-15 | Seattle Genetics | monoclonal antibody; | | | | cancer |
| | | | immunotoxin | | | | |
| MAb, SS(dsFv)- | | NeoPharm | immunotoxin; | | | | cancer |
| PE38; SSI-PE38 | | | monoclonal antibody | | | | |
| MAb, iodine-131, | Cotara | Peregrine | monoclonal antibody; | | | | cancer |
| DNA-associated | | Pharmaceuticals | radiotherapeutic | | | | |
| antigens | | | | | | | |
| MAb, C242-DM | | ImmunoGen | monoclonal antibody; | | tumor-bearing | | cancer |
| 1 conjugate; | | | immunotoxin | | mice received | | |
| SB-408075 | | | | | 225-300 μg/kg | | |
| | | | | | over 5 days; | | |
| | | | | | complete | | |
| | | | | | responses seen | | |
| | | | | | in 100% of | | |
| | | | | | animals lasting | | |
| | | | | | 200 days, with | | |
| | | | | | no weight loss. | | |
| Mab CC-49 | | National Cancer | | | | | |
| yttrium-90; | | Insistute | | | | | |
| LU-177 | | | | | | | |
| Mab Hum291 | | National Cancer | | | | | |
| | | Institute | | | | | |
| MEDI 507 | | BioTransplant | | | | | |
| IDEC Y2B8; | Zevalin | IDEC | monoclonal antibody; | | in Hodgkin's | | cancer |
| ibritumomab | | Pharmaceuticals | radiotherapeutic | | lymphoma | | |
| tiuxetan; MAb, | | | | | patients | | |
| pan-B-yttrium; | | | | | receiving 0.2, | | |
| MAb, B- cell | | | | | 0.3 or 0.4 mCi/kg | | |
| radiation therapy | | | | | in | | |
| immuno-globulin | | | | | combination | | |
| G1, anti-(human | | | | | with rituximab, | | |
| CD20 (antigen)) | | | | | overall response | | |
| (mouse monoclonal | | | | | rate is 82% | | |
| IDEC-Y2B8. | | | | | (81% at the | | |
| gamma.1-chain), | | | | | highest dose) | | |
| disulfide with | | | | | | | |
| mouse monoclonal | | | | | | | |
| IDEC-Y2B8. | | | | | | | |
| kappa.-chain, | | | | | | | |
| dimer N-[2-[ | | | | | | | |
| bis(carboxy- | | | | | | | |
| methyl)amino]- | | | | | | | |
| 3-(4-isothio- | | | | | | | |
| cyanatophenyl) | | | | | | | |
| propyl]-N-[2- | | | | | | | |
| [bis(carb | | | | | | | |
| oxymethyl)amino] | | | | | | | |
| propyl]glycine | | | | | | | |
| conjugate | | | | | | | |
| MAb, cancer | HumaRAD- | Intracel | biotechnology; | | Phase I/II | | cancer |
| | HN; | | monoclonal antibody | | studies in head | | |
| | HumaRAD- | | | | and neck cancer | | |
| | OV | | | | show treatment | | |
| | | | | | safe and well | | |
| | | | | | tolerated; can | | |
| | | | | | deliver therapeutic | | |
| | | | | | doses of | | |
| | | | | | radiation | | |
| | | | | | directly to the | | |
| | | | | | tumor site | | |
| INC 225 | | Imclone | | | | | |
| | | Systems | | | | | |
| MAb, humanized | Nuvion | Protein Design | monoclonal antibody; | | single 3 mg/m² | headache, | endstage renal |
| | | | immunosuppressant | | dose or seven | nausea, chills, | disease |
| | | | | | doses of 0.25 mg/m², | and fever during | |
| | | | | | 1.0 mg/m² | first few hours | |
| | | | | | | following dosing | |
| gemtuzumab | Mylotarg | Wyeth-Ayerst | monoclonal antibody; | CAS: | | | cancer |
| ozogamicin; | | | immunotoxin | 220578- | | | |
| gemtuzumab | | | | 59-6 | | | |
| zogamicin; | | | | | | | |
| WAY-CMA 676 | | | | | | | |
| MAb, CTLA-4; | | Medarex | monoclonal antibody; | | | | blockade of |
| | | | immunostimulant | | | | CTLA-4 leads to |
| | | | | | | | immune response |
| | | | | | | | and consequent |
| | | | | | | | rejection of tumor |
| | | | | | | | cells |
| IMC-225 | Erbitux | ImClone | monoclonal antibody | | | | |
| | | Systems | | | | | |
| trastuzumab | Herceptin | NCI; Genentech | HER-2 blocker; | | | | breast, colon, |
| | | | epidermal growth | | | | bladder, lung, |
| | | | factor inhibitor, | | | | pancreatic cancers |
| | | | antibody | | | | |
| bevacizumab; | | Genentech; | monoclonal antibody; | | | | |
| anti-VEGF | | National Cancer | neutralizes vascular | | | | |
| humanized | | Institute | endothelial growth | | | | |
| monoclonal | | | factor (VEGF) | | | | |
| antibody | | | protein; inhibits | | | | |
| | | | tumor growth | | | | |
| 88BV59; | HumAspect | Intracel | human MAb labeled | | | | imaging |
| votumumab | | | with technicium Tc | | | | recurrence of |
| | | | 99 m used as imaging | | | | cancer |
| | | | agent for cancer | | | | |
| | | | diagnosis and | | | | |
| | | | monitoring | | | | |
| BEC2, GD3 | | ImClone | monoclonal antibody | | | | residual tumor |
| anti-idiotype | | Systems | mimics ganglioside | | | | cells; limited |
| vaccine | | | GD3; | | | | disease small cell |
| | | | immunostimulant | | | | lung carcinoma |
| chimeric | | National Cancer | monoclonal antibody | | | | |
| Mab 14.18 | | Institute | | | | | |
| Ova Rex MAb | | AltaRex | targets CA125 in | | 20 minute IV | | |
| | | | circulation; induces | | infusion, low | | |
| | | | broad immune | | dose | | |
| | | | responses | | | | |
| MDX-CTLA4 | | Medarex | inhibits autoimmune | | | | |
| (anti-CTLA4) | | | response; anticytotoxic | | | | |
| | | | T-lymphocyte- | | | | |
| | | | associated antigen-4 | | | | |
| | | | monoclonal antibody | | | | |
| MAb anti- | | National Cancer | monoclonal antibody | | | | |
| transferrin | | Institute | | | | | |
| receptors | | | | | | | |

TABLE 14
Radio/chemo sensitizers and protectors

| Agent | Trade name | Company | Mode of action | Reference | Dosage | Toxicity | Indication |
|---|---|---|---|---|---|---|---|
| biricodar | Incel | Vertex | MDR inhibitor | CAS: | | | cancer |
| | | Pharmaceuticals | | 159997-94-1; | | | |
| | | | | 174254-13-8 | | | |
| | | | | (dicitrate salt) | | | |
| LE AON | | NeoPharm | antisense | | | | |
| | | | oligonucleotide; | | | | |
| | | | radiosensitizer | | | | |
| LE raf AON (liposome- | | NeoPharm | non-viral liposome | | | | |
| encapsulated antisense | | | antisense compound; | | | | |
| oligonucleotide to raf-1 | | | enhances effectiveness | | | | |
| proto oncogene) | | | of radiation and | | | | |
| | | | chemotherapy | | | | |
| gadolinium-texaphyrin | | Pharmacyclics | radiosensitizer for | | | | |
| | | | cancer radiotherapy | | | | |
| mirostipen | | Human Genome | myeloid progenitor | | | | chemoprotection |
| | | Sciences | inhibitory factor; | | | | |
| | | | human protein; | | | | |
| | | | protects blood cells | | | | |
| | | | from effects of cancer | | | | |
| | | | therapies | | | | |
| ILX23-7553 | | Ilex Oncology | chemo/radio sensitizer | | | | |

TABLE 15
Taxane derivatives

| Agent | Trade name | Company | Mode of action | Reference | Dosage | Toxicity | Indication |
|---|---|---|---|---|---|---|---|
| paclitaxel polyglutamic acid | PG-TXL | Cell | taxane; microtubule | | 266 mg/m² | | |
| | | Therapeutics | assembly promoter | | | | |
| BMS-184476 | | Bristol-Myers | taxane | | 20-60 mg/m² | febrile neutropenia, | |
| | | Squibb | | | | mucositis | |
| | | | | | | and diarrhea | |
| taxane (IV) | | Bayer | taxane | | | | brain metastases, |
| | | | | | | | lung, solid tumors |
| BMS-188797 | | Bristol-Myers | injectable taxane | | | | |
| | | Squibb | | | | | |
| epothilone B; BMS-24755 | | NCI; Bristol- | taxane analog | | | | |
| | | Myers Squibb | | | | | |
| epothilone | | Bristol-Myers | taxane analog; | | | | first-line cancers |
| | | Squibb | photoaffinic labeled | | | | |

TABLE 16
Vaccines

| Agent | Trade name | Company | Mode of action | Reference | Dosage | Toxicity | Indication |
|---|---|---|---|---|---|---|---|
| APC-8024 | | Dendreon | vaccine; | | | | cancer |
| | | | immunostimulant | | | | |
| GnRH | | Aphton | vaccine; anti-GnRH | | | | |
| Pharmaccine | | | antibody | | | | |
| therapeutic | | | | | | | |
| vaccine | | | | | | | |
| RV-MUC-1 | | Therion | vaccine | | | | |
| | | Biologics | | | | | |
| HPV-16 E6 | | National Cancer | vaccine | | | | |
| and E7 peptide | | Institute | | | | | |
| vaccine | | | | | | | |
| MEDI-503/51 | | MedImmune | vaccine | | | | cancer |
| HPV-11 vaccine | | | | | | | |
| Allogenic colon | | The Immune | vaccine | | | | |
| | | Response | | | | | |
| | | Corporation | | | | | |
| Allogenic glioma | | The Immune | vaccine | | | | |
| | | Response | | | | | |
| | | Corporation | | | | | |
| Autologous | OncoVAXCL | Intracel | vaccine | | | | |
| vaccine VHL | | National Cancer | vaccine | | | | |
| peptide vaccine | | Institute | | | | | |
| Myeloma-derived | | National Cancer | vaccine | | | | |
| idiotypic anigen | | Institute | | | | | |
| vaccine | | | | | | | |
| CapVax | DCVax | Northwest | vaccine | | | | |
| | Prostate | Biotherapeutics | | | | | |
| APC 8015 | Provenge | Dendreon | vaccine; | | | | cancer |
| | | | immunostimulant | | | | |
| ALVAC-B7.1; | | National Cancer | vaccine; | | | | ovarian |
| AUT-OV-ALVAC- | | Institute | immunostimulant | | | | carcinoma |
| hB7.1 | | | | | | | |
| gp100 vaccine | | National Cancer | gene therapy; vaccine | | | in combination | melanoma tumors |
| | | Institute | | | | with MART-1 | |
| | | | | | | tumor antigen, | |
| | | | | | | safe and well | |
| | | | | | | tolerated | |
| modified gp100 | | NCI; Vical | vaccine; gene therapy | | | | |
| rF-gp100 | | NCI; Therion | vaccine | | | | |
| Vaccine; | | National Cancer | Vaccine | | | | |
| canarypox CEA; | | Institute | vaccine | | | | |
| ALVAC-CEA | | | | | | | |
| Helicobacter | Helivax | Antex | vaccine | | | well tolerated | gastrointestinal |
| pylori vaccine | | | | | | although some | ulcer; gastric |
| | | | | | | reports | cancer |
| | | | | | | of gastric | |
| | | | | | | disturbances | |
| P53 and RAS | | National Cancer | vaccine | | | | colon, lung, |
| vaccine | | Institute | | | | | ovarian cancer |
| vaccinia-CEA | | NCI; Therion | vaccine | | | | breast, lung, |
| vaccine (180KD) | | | | | | | stomach cancer |
| oncophage; | | Antigenics | vaccine; heat shock | | 25 μg intradermal | | cancer |
| HSPPC-96 | | | protein; | | injection | | |
| | | | immunomodulator | | once a week for | | |
| | | | | | 4-8 weeks, then | | |
| | | | | | every other | | |
| | | | | | week | | |
| idiotype KLH | | National Cancer | vaccine | | | | |
| lymphoma vaccine | | Institute | | | | | |
| idiotype vaccine | | Biomira | idiotype vaccine | | | | B-cell lymphoma |
| luteinizing | | United | stimulates antibodies | | | | |
| hormone-releasing | | Biomedical | which neutralize | | | | |
| hormone | | | LHRH | | | | |
| (LHRH) | | | | | | | |
| immunotherapeutic | | | | | | | |
| (synthetic peptide | | | | | | | |
| vaccine) | | | | | | | |
| MAGE-12: | | National Cancer | peptide vaccine | DHAS Ref. | | | |
| 170-178 peptide | | Institute | | E-056-00/0 | | | |
| vaccine | | | | | | | |
| MART-1 | | National Cancer | vaccine | | | | metastatic |
| melanoma | | Institute | | | | | melanoma |
| vaccine | | | | | | | |
| MART-1 with | | Genzyme | vaccine | | | | melanoma |
| gp100 (in vivo) | | | | | | | |
| rF-tyrosine vaccine | | NCI; Therion | vaccine | | | | melanoma |
| rV-gp100 | | Therion | vaccine | | | | melanoma |
| ESO-1: 157-165 | | National Cancer | peptide vaccine | | | | |
| | | Institute | | | | | |
| fowlpox-CEA(6D) | | NCI; Therion | | | | | |
| tricom and | | | | | | | |
| vaccinia-CEA(6D) | | | | | | | |
| tricom vaccine | | | | | | | |
| fowlpox | | NCI; Therion | vaccine | | | | |
| gp100: ES | | | | | | | |
| 209-217 (2m) | | | | | | | |
| vaccine | | | | | | | |
| RAS 5-17 | | National Cancer | vaccine | | | | |
| peptide vaccine | | Institute | | | | | |
| proteinase-3 | | National Cancer | vaccine | | | | advanced cancers |
| peptide vaccine | | Institute | | | | | |

TABLE 17
Vinca alkaloid agents

| Agent | Trade name | Company | Mode of action | Reference | Dosage | Toxicity | Indication |
|---|---|---|---|---|---|---|---|
| tocladesine; NSC 614491 | Adenazole | ICN | cyclic adenosine | CAS: | | | |
| 8-chloroadenosine cyclic 3′,5′- | | Pharmaceuticals | monophosphate | 41941-56-4 | | | |
| (hydrogen phosphate) | | | (cAMP) analog | | | | |
| | | | antagonist | | | | |

TABLE 18
Illustrative antineoplastic agents

| Name | Company | Patent | Oncology Indication | Mode of Action |
|---|---|---|---|---|
| Neu-Sensamide | OXiGENE Inc | | lung tumor, brain tumor, | 5-HT 3 antagonist |
| | | | neoplasm | |
| A-63162 | Abbott Laboratories | | neoplasm | 5-Lipoxygenase inhibitor |
| caracemide | Marion Merrell Dow | DE 3305107 | carcinoma, neoplasm | Acetylcholinesterase inhibitor |
| | Pharmaceuticals Inc | | | |
| Xerecept | Neurobiological Technologies | | brain tumor | ACTH releasing factor |
| | Inc | | | |
| lisofylline | Cell Therapeutics Inc | | myeloid leukemia, neoplasm | Acyltransferase inhibitor |
| IB-MECA | National Institutes of Health | | carcinoma | Adenosine A3 agonist |
| L-249313 | Merck & Co Inc | | neoplasm | Adenosine A3 antagonist |
| adenosine triphosphate, Medco | Medco Research Inc | | lung tumor | Adenosine agonist |
| cladribine | Ortho Biotech Inc | WO 93/23058 | carcinoma, non-Hodgkin's | Adenosine deaminase inhibitor |
| | | | lymphoma, leukemia, solid | |
| | | | tumor | |
| alanosine, Triangle | Triangle Pharmaceuticals Inc | | brain tumor, carcinoma, glioma, | Adenosine modulator |
| | | | lung tumor | |
| MDL-28842 | Hoechst Marion Roussel Inc | EP 0 304 889 | carcinoma | Adenosylhomocysteinase |
| | | | | inhibitor |
| ATP, University of Sydney | University of Sydney | | leukemia | Adenylate cyclase stimulator |
| CD40 ligand, Immunex | Immunex Corp | | neoplasm, non-Hodgkin's | Adjuvant |
| | | | lymphoma | |
| EO-9 | National Institutes of Health | WO 87/06227 | neoplasm | Alkylating agent |
| AP-5070 | ACCESS Pharmaceuticals Inc | | neoplasm | Alkylating agent |
| WIN-33377 | Sterling Winthrop Products Inc | | solid tumor | Alkylating agent |
| piroxantrone | Parke-Davis & Co | EP 0 103 381 | carcinoma, melanoma, neoplasm | Alkylating agent |
| NK-109 | Nippon Kayaku Co Ltd | EP 0 432 630 | neoplasm | Alkylating agent |
| LY-296329 | Eli Lilly & Co | | neoplasm | Alkylating agent |
| LY-297950 | Eli Lilly & Co | | neoplasm | Alkylating agent |
| EO-9 | National Institutes of Health | WO 87/06227 | neoplasm | Alkylating agent |
| BCH-242 | BioChem Pharma Inc | | carcinoma, neoplasm | Alkylating agent |
| PD-115934 | Parke-Davis & Co | EP 0 138 302 | carcinoma | Alkylating agent |
| B.4152 | European Organisation for | | neoplasm | Alkylating agent |
| | Research and Treatment of | | | |
| | Cancer (EORTC) | | | |
| adozelesin | Pharmacia & Upjohn Co | | breast tumor, carcinoma, | Alkylating agent |
| | | | leukemia, neoplasm, solid tumor | |
| ecomustine | Choay SA | WO 85/01050 | carcinoma | Alkylating agent |
| enloplatin | American Cyanamid Co | EP 0 232 784 | carcinoma | Alkylating agent |
| tallimustine | Pharmacia & Upjohn AB | EP 0 246 868 | leukemia, solid tumor | Alkylating agent |
| FCE-26605 | Farmitalia Carlo Erba SpA | WO 91/10649 | carcinoma | Alkylating agent |
| FCE-26752 | Farmitalia Carlo Erba SpA | | carcinoma | Alkylating agent |
| galamustine | Unimed Pharmaceuticals Inc | | carcinoma | Alkylating agent |
| JM-216 | Johnson Matthey plc | EP 0 328 274 | carcinoma, lung tumor, ovary | Alkylating agent |
| | | | tumor, prostate tumor | |
| miboplatin | Chugai Pharmaceutical Co Ltd | EP 0 176 005 | carcinoma, ovary tumor, prostate | Alkylating agent |
| | | | tumor | |
| nedaplatin | Shionogi & Co Ltd | JP 59-222497 | carcinoma | Alkylating agent |
| sebriplatin | Nippon Kayaku Co Ltd | EP 0 219 936 | carcinoma, neoplasm | Alkylating agent |
| ormaplatin | Pharmacia & Upjohn Co | | carcinoma, leukemia, solid | Alkylating agent |
| | | | tumor | |
| temozolomide | The University of Aston In | DE 3231255 | carcinoma, glioma, melanoma, | Alkylating agent |
| | Birmingham | | metastasis | |
| JM-221 | Johnson Matthey plc | | neoplasm | Alkylating agent |
| etopophos | Bristol-Myers Squibb Co | U.S. Pat. No. | carcinoma, kaposis sarcoma, | Alkylating agent |
| | | 5,041,424 | lung tumor, lymphoma, prostate | |
| | | | tumor | |
| FCE-26492 | Farmitalia Carlo Erba SpA | | carcinoma | Alkylating agent |
| losoxantrone | Parke-Davis & Co | EP 0 103 381 | breast tumor, neoplasm | Alkylating agent |
| FCE-27726 | Pharmacia & Upjohn SpA | | neoplasm | Alkylating agent |
| UCT-1072 | Kyowa Hakko Kogyo Co Ltd | WO 97/29099 | neoplasm | Alkylating agent |
| BBR-2778 | Boehringer Mannheim GmbH | | leukemia, lymphoma | Alkylating agent |
| Promycin | Vion Pharmaceuticals Inc | | head & neck tumor, neoplasm | Alkylating agent |
| RSU-1069 | British Technology Group Plc | | neoplasm | Alkylating agent |
| KI-30606 | Il-Yang Pharm Ind Co Ltd | | neoplasm | Alkylating agent |
| cystemustine | INSERM | | neoplasm, melanoma, head & | Alkylating agent |
| | | | neck tumor, renal tumor, | |
| | | | colorectal tumor, glioma, | |
| | | | carcinoma, sarcoma | |
| XP-315 | DuPont Pharmaceuticals Co | | neoplasm | Alkylating agent |
| CB-7646 | Institute of Cancer Research, | | carcinoma | Alkylating agent |
| | UK | | | |
| SKI-2019R | Sunkyong Industries Co Ltd | | neoplasm | Alkylating agent |
| penclomidine | National Cancer Institute | | carcinoma | Alkylating agent |
| OCX-177 | Yale University | | carcinoma | Alkylating agent |
| OCX-247 | Yale University | | carcinoma | Alkylating agent |
| zeniplatin | Lederle Laboratories | | melanoma, ovary tumor | Alkylating agent |
| cycloplatam | Institute of Cancer Research, | | carcinoma | Alkylating agent |
| | UK | | | |
| SK-2053R | Sunkyong Pharmaceutical Ltd. | | carcinoma | Alkylating agent |
| anticancer agents, NIH | National Institutes of Health | | carcinoma | Alkylating agent |
| phosphoramidates, MGI | MGI Pharma Inc | | neoplasm | Alkylating agent |
| electrophilic alkylating agents | Bionumerik Pharmaceuticals Inc | | solid tumor | Alkylating agent |
| DSB-120 | Institute of Cancer Research, | | carcinoma | Alkylating agent |
| | UK | | | |
| drupangtonine | Tokyo University of Pharmacy | | leukemia | Alkylating agent |
| | & Life Sciences | | | |
| tallimustine derivatives, | Pharmacia & Upjohn Inc | | carcinoma | Alkylating agent |
| Pharmacia & Upjohn | | | | |
| alkylating agents, Vion | Vion Pharmaceuticals Inc | | neoplasm | Alkylating agent |
| DT1-015 | Direct Therapeutics Inc | | glioma | Alkylating agent |
| DTI-136 | Direct Therapeutics Inc | | liver tumor | Alkylating agent |
| ADP | US Bioscience Inc | | carcinoma, neoplasm | Alkylating agent |
| ambamustine | Proter | | carcinoma | Alkylating agent |
| BMS-181174 | Bristol-Myers Squibb Co | DE 3413489 | digestive system tumor, lung | Alkylating agent |
| | | | tumor, neoplasm, ovary tumor | |
| calicheamicins | American Cyanamid Co | EP 0 392 376 | breast tumor, female genital tract | Alkylating agent |
| | | | tumor, lung tumor, myeloid | |
| | | | leukemia, ovary tumor | |
| carzelesin | Pharmacia & Upjohn Co | WO 88/04659 | carcinoma, leukemia, neoplasm, | Alkylating agent |
| | | | solid tumor | |
| cisplatin, Takeda | Takeda Chemical Industries Ltd | | carcinoma, prostate tumor, | Alkylating agent |
| | | | uterine cervix tumor | |
| esperamicin-A1 | Bristol-Myers Squibb Co | GB 2 179 649 | carcinoma | Alkylating agent |
| FR-900482 | Fujisawa Pharmaceutical Co Ltd | EP 0 166 389 | colon tumor, leukemia, | Alkylating agent |
| | | | melanoma, solid tumor | |
| hepsulfam | Elf Sanofi | | carcinoma | Alkylating agent |
| kazusamycin | Merck & Co Inc | | carcinoma | Alkylating agent |
| kedarcidin | Bristol-Myers Squibb Co | U.S. Pat. No. | carcinoma | Alkylating agent |
| | | 5,001,112 | | |
| menogaril | Pharmacia & Upjohn Co | U.S. Pat. No. | breast tumor, carcinoma, | Alkylating agent |
| | | 4,183,860 | lymphoma | |
| oxaliplatin | Debiopharm SA | | colorectal tumor, neoplasm, lung | Alkylating agent |
| | | | tumor, ovary tumor | |
| BBR-2378 | Boehringer Mannheim GmbH | | neoplasm | Alkylating agent |
| bisnafide dimesylate | DuPont Pharmaceuticals Co | WO 92/17453 | breast tumor, colorectal tumor, | Alkylating agent |
| | | | neoplasm | |
| bizelesin | Pharmacia & Upjohn Co | EP 0 359 454 | carcinoma, leukemia, neoplasm, | Alkylating agent |
| | | | solid tumor | |
| PCNU | Pharmacia & Upjohn Co | | neoplasm | Alkylating agent |
| U-75559 | Pharmacia & Upjohn Co | | neoplasm | Alkylating agent |
| fotemustine | Servier | FR 2536075 | melanoma, neoplasm | Alkylating agent |
| lobaplatin | ASTA Medica AG | | esophagus tumor, neoplasm, | Alkylating agent |
| | | | ovary tumor | |
| KW-2170 | Kyowa Hakko Kogyo Co Ltd | | carcinoma | Alkylating agent |
| treosulfan | Leo Laboratories Ltd | | neoplasm, ovary tumor | Alkylating agent |
| glufosfamide | ASTA Medica AG | | neoplasm | Alkylating agent |
| BBR-3005 | Boehringer Mannheim GmbH | | neoplasm | Alkylating agent |
| JM-335 | Johnson Matthey plc | | neoplasm | Alkylating agent |
| TER-286 | Telik Inc | | carcinoma, neoplasm | Alkylating agent |
| SKI-2053R | Sunkyong Industries Co Ltd | | neoplasm, stomach tumor, | Alkylating agent |
| | | | uterine cervix tumor, lung | |
| | | | tumor, head & neck tumor | |
| MEN-10718 | Menarini Ltd | | neoplasm | Alkylating agent |
| trimelamol | Institute of Cancer Research, | | neoplasm | Alkylating agent |
| | UK | | | |
| FCE-25450A | Pharmacia & Upjohn AB | | carcinoma, leukemia | Alkylating agent |
| SKI-2034R | Sunkyong Industries Co Ltd | | neoplasm | Alkylating agent |
| FCE-28102 | Pharmacia & Upjohn SpA | | carcinoma | Alkylating agent |
| FCE-28164 | Pharmacia & Upjohn SpA | | carcinoma | Alkylating agent |
| ME6C | Oregon Health Sciences | | neoplasm | Alkylating agent |
| | University | | | |
| tauromustine | Pharmacia & Upjohn AB | EP 0 106 123 | carcinoma | Alkylating agent |
| KW-2189 | Kyowa Hakko Kogyo Co Ltd | | carcinoma, melanoma, neoplasm | Alkylating agent |
| GI-231818 | Glaxo Wellcome plc | | prostate tumor | Alpha 1 adrenoceptor antagonist |
| SNAP-6107 | Synaptic Pharmaceutical Corp | WO 97/17969 | prostatic hypertrophy | Alpha 1 adrenoceptor antagonist |
| alfuzosin | Synthelabo | U.S. Pat. No. | prostate tumor | Alpha 1 adrenoceptor antagonist |
| | | 4,315,007 | | |
| tamsulosin | Yamanouchi Pharmaceutical Co | U.S. Pat. No. | prostate tumor | Alpha 1 adrenoceptor antagonist |
| | Ltd | 4,868,216 | | |
| doxazosin | Pfizer Ltd | DE 2847623 | prostate tumor | Alpha 1 adrenoceptor antagonist |
| SNAP-6201 | Synaptic Pharmaceutical Corp | | prostate tumor | Alpha 2 adrenoceptor antagonist |
| A-76202M | Sankyo KK | J 09-003090 | neoplasm | Alpha glucosidase inhibitor |
| DMNJ, KRIBB | Korea Research Institute of | | metastasis | Alpha glucosidase inhibitor |
| | Bioscience and Biotechnology | | | |
| castanospermine, Fujisawa | Fujisawa Pharmaceutical Co Ltd | JP 61-227566 | carcinoma | Alpha glycosidase inhibitor |
| swainsonine, Fujisawa | Fujisawa Pharmaceutical Co Ltd | JP 61-227566 | carcinoma | Alpha mannosidase inhibitor, |
| | | | | Adjuvant |
| L-751788 | Merck & Co Inc | | prostate tumor | Alpha reductase inhibitor |
| MK-386 | Merck & Co Inc | WO 93/23419 | prostate tumor | Alpha reductase inhibitor |
| GI-198745 | Glaxo Wellcome plc | | prostate tumor | Alpha reductase inhibitor |
| LY-320236 | Eli Lilly & Co | EP 0 703 221 | prostate tumor, neoplasm | Alpha reductase inhibitor |
| MR-387B | Korea Research Institute of | | neoplasm | Aminopeptidase inhibitor |
| | Bioscience and Biotechnology | | | |
| APN inhibitors, Ishihara | Ishihara Sangyo KK | | neoplasm | Aminopeptidase inhibitor |
| Bestatin | Nippon Kayaku Co Ltd | | carcinoma, leukemia, lung | Aminopeptidase inhibitor |
| | | | tumor, Hodgkin's disease, non- | |
| | | | Hodgkin's lymphoma | |
| dehydro-epiandrosterone, | Jenapharm GmbH | | carcinoma | Androgen |
| Jenapharm | | | | |
| MDL-27302 | Hoechst Marion Roussel Inc | EP 0 288 053 | carcinoma | Androgen antagonist |
| LG-2293 | Ligand Pharmaceuticals Inc | | neoplasm, prostate tumor | Androgen antagonist |
| L-245976 | Merck & Co Inc | | prostate tumor | Androgen antagonist |
| bicalutamide | Zeneca Group Plc | EP 0 100 172 | prostate tumor | Androgen antagonist |
| zanoterone | Sanofi Winthrop Inc | EP 0 207 375 | carcinoma, prostate tumor | Androgen antagonist |
| Osaterone acetate | Teikoku Hormone | | prostate tumor | Androgen antagonist |
| | Manufacturing Co Ltd | | | |
| androgen antagonists, Karo Bio | Karo Bio AB | | neoplasm, prostate tumor | Androgen antagonist |
| flutamide | Schering-Plough Corp | | carcinoma, ovary tumor, prostate | Androgen antagonist |
| | | | tumor | |
| androgen blocking agents, RCT | Research Corp Technologies Inc | | prostate tumor | Androgen antagonist |
| RU-59063 | Roussel Uclaf SA | EP 0 580 459 | prostate tumor | Androgen antagonist |
| RU-56187 | Roussel Uclaf SA | EP 0 494 819 | prostate tumor | Androgen antagonist |
| WB-2838 | Fujisawa Pharmaceutical Co Ltd | | carcinoma | Androgen antagonist |
| I-23 | Research Corp Technologies Inc | | prostate tumor | Androgen antagonist |
| nilutamide | Roussel Uclaf Corp | | prostate tumor | Androgen antagonist |
| topical pain therapy, American | American Pharmed Labs Inc | | pain | Anesthetic, local |
| Pharmed Inc | | | | |
| polysulphonic acid derivatives, | Fuji Photo Film Co Ltd | JP 09059163 | neoplasm | Angiogenesis inhibitor |
| Fuji | | | | |
| SELEX | NeXstar Pharmaceuticals Inc | U.S. Pat. No. | neoplasm | Angiogenesis inhibitor |
| | | 5,270,163 | | |
| SB-220025 | SmithKline Beecham | | neoplasm | Angiogenesis inhibitor |
| | Pharmaceuticals | | | |
| CHIR-11509 | Chiron Corp | WO 96/40747 | neoplasm | Angiogenesis inhibitor |
| anti-flk-1, ImClone systems Inc | Imclone Systems Inc | WO 95/21868 | angiogenesis disorder, | Angiogenesis inhibitor |
| | | | carcinoma | |
| NX-278-L | NeXstar Pharmaceuticals Inc | WO 96/27604 | angiogenesis disorder, kaposis | Angiogenesis inhibitor |
| | | | sarcoma | |
| suramin | Warner-Lambert Co | | prostate tumor | Angiogenesis inhibitor |
| thalidomide, Celgene | Celgene Corp | WO 92/14455 | carcinoma, rheumatoid arthritis | Angiogenesis inhibitor |
| squalamine | Magainin Pharmaceuticals Inc | | brain tumor, solid tumor, breast | Angiogenesis inhibitor |
| | | | tumor, lung tumor | |
| CT-2584 | Cell Therapeutics Inc | | breast tumor, carcinoma, | Angiogenesis inhibitor |
| | | | leukemia, lung tumor, | |
| | | | melanoma, ovary tumor, prostate | |
| | | | tumor, renal tumor, sarcoma, | |
| | | | solid tumor | |
| 2-methoxyestradiol | Harvard University | | breast tumor | Angiogenesis inhibitor |
| GM-1603 | Glycomed Inc | | neoplasm, carcinoma | Angiogenesis inhibitor |
| anti VEGF antibody, Toagosei | Toagosei Co Ltd | | neoplasm | Angiogenesis inhibitor |
| combretastatin A-4 prodrug, | Arizona State University | | solid tumor | Angiogenesis inhibitor |
| Arizona State Uni | | | | |
| 2-nitroimidazole derivatives, | Otsuka Pharmaceutical Co Ltd | JP 09025268 | carcinoma, inflammation | Angiogenesis inhibitor |
| Otsuka | | | | |
| gene therapy | Genetix Pharmaceuticals | | neoplasm, solid tumor | Angiogenesis inhibitor |
| (Endostatin/Angiostatin), | | | | |
| Genetix | | | | |
| Dival | Hedral Therapeutics Inc | | carcinoma | Angiogenesis inhibitor |
| TAN-1323D | Takeda Chemical Industries Ltd | | neoplasm | Angiogenesis inhibitor |
| angiogenesis inhibitor, Schering | Schering AG | | carcinoma | Angiogenesis inhibitor |
| AG | | | | |
| angiostatin | Entremed Inc | WO 95/29242 | neoplasm, angiogenesis disorder | Angiogenesis inhibitor |
| GM-1306 | Glycomed Inc | | neoplasm | Angiogenesis inhibitor |
| polymeric delivery sytems, | Angiotech Pharmaceuticals Inc | | neoplasm | Angiogenesis inhibitor |
| Angiotech | | | | |
| RPI-4610 | Ribozyme Pharmaceuticals Inc | | neoplasm | Angiogenesis inhibitor |
| MB-102 | Megabios Corp | | lung tumor | Angiogenesis inhibitor |
| TZ-93 | Tsumura & Co Ltd | | carcinoma | Angiogenesis inhibitor |
| AE-941 | AEterna Laboratories Inc | | breast tumor, lung tumor, | Angiogenesis inhibitor |
| | | | prostate tumor, solid tumor | |
| SR-25989 | Sanofi SA | | carcinoma | Angiogenesis inhibitor |
| SU-302 | Max-Planck-Gesellschaft zur | | carcinoma | Angiogenesis inhibitor |
| | Foederung der Wissenschaten | | | |
| | EV | | | |
| Cartilage-derived Inhibitor, | Boston Life Sciences Inc | | solid tumor | Angiogenesis inhibitor |
| Boston Life Sciences | | | | |
| endostatin | Children's Hospital of Boston | WO 97/15666 | angiogenesis disorder, neoplasm | Angiogenesis inhibitor |
| RG-8803 | RepliGen Corp | | carcinoma | Angiogenesis inhibitor |
| thalidomide, EntreMed | Entremed Inc | | breast tumor, glioma, kaposis | Angiogenesis inhibitor |
| | | | sarcoma, prostate tumor | |
| eponemycin analog, BioChem | BioChem Therapeutic Inc | | angiogenesis disorder, neoplasm | Angiogenesis inhibitor |
| troponin-1, Boston Life Sciences | Boston Life Sciences Inc | | breast tumor, prostate tumor | Angiogenesis inhibitor |
| BST-2001 | BioStratum Inc | | solid tumor | Angiogenesis inhibitor |
| thymidine phosphorylase | Genzyme Molecular Oncology | | neoplasm | Angiogenesis inhibitor |
| inhibitors, Genzyme | | | | |
| angiogenesis inhibitor, | GeneMedicine Inc | | neoplasm | Angiogenesis inhibitor |
| GeneMedicine/UCSF | | | | |
| 4,6-diarylpyrimidine derivatives | Otsuka Pharmaceutical Co Ltd | WO 96/32384 | neoplasm | Angiogenesis inhibitor |
| 2-nitroimidazole, Taiyo | Taiyo Yakuhin Kogyo Co Ltd | JP 07033658 | carcinoma | Angiogenesis inhibitor |
| substituted hydrindanes, Nestle | Nestle SA | WO 94/04143 | carcinoma | Angiogenesis inhibitor |
| 1,3,5-triazines, Nippon Shinyaku | Nippon Shinyaku Co Ltd | WO 96/32945 | neoplasm | Angiogenesis inhibitor |
| pyridazinamine derivatives, | Johnson & Johnson | WO 97/26258 | neoplasm | Angiogenesis inhibitor |
| Johnson & Johnson | | | | |
| angiogenesis inhibitors, Noxxon | Noxxon Pharma AG | | carcinoma | Angiogenesis inhibitor |
| fumagillin analogs, BioChem | BioChem Therapeutic Inc | | neoplasm | Angiogenesis inhibitor |
| Therapeutics | | | | |
| plasminogen kringle 5, Abbott | Abbott Laboratories | | neoplasm | Angiogenesis inhibitor |
| angiogenesis inhibitor, Merck | Merck KGaA | | neoplasm, breast tumor, | Angiogenesis inhibitor |
| | | | colorectal tumor, lung tumor | |
| gene therapy (anti-angiogenesis), | Regeneron Pharmaceuticals Inc | | neoplasm | Angiogenesis inhibitor |
| Regeneron/Duke | | | | |
| TAS-202 | Taiho Pharmaceutical Co Ltd | | neoplasm | Angiogenesis inhibitor |
| angiogenesis inhibitors, Upjohn | Pharmacia & Upjohn Co | | carcinoma, neoplasm | Angiogenesis inhibitor |
| CI-994 | Parke-Davis & Co | DE 3613571 | carcinoma, neoplasm | Angioenesis inhibitor |
| tecogalan sodium | Daiichi Seiyaku Co Ltd | EP 0 391 410 | breast tumor, kaposis sarcoma, | Angiogenesis inhibitor |
| | | | melanoma, prostate tumor, renal | |
| | | | tumor, solid tumor | |
| FR-111142 | Fujisawa Pharmaceutical Co Ltd | JP 02233610 | carcinoma, leukemia, lymphoma | Angiogenesis inhibitor |
| FCE-26950 | Pharmacia & Upjohn SpA | | angiogenesis disorders, | Angiogenesis inhibitor |
| | | | carcinoma | |
| TAN-1120 | Takeda Chemical Industries Ltd | EP 0 376 177 | angiogenesis disorder, | Angiogenesis inhibitor |
| | | | carcinoma | |
| titanocene dichloride | Medac GmbH | | carcinoma, neoplasm | Angiogenesis inhibitor |
| FR-118487 | Fujisawa Pharmaceutical Co Ltd | | solid tumor | Angiogenesis inhibitor |
| L-651582 | Merck & Co Inc | EP 0 151 529 | neoplasm | Angiogenesis inhibitor |
| TAS-102 | Taiho Pharmaceutical Co Ltd | | carcinoma, angiogenesis | Angiogenesis inhibitor |
| | | | disorder, colon tumor | |
| FR-901448 | Fujisawa Pharmaceutical Co Ltd | JP 04224559 | neoplasm | Angiogenesis inhibitor |
| U-42129 | Pharmacia & Upjohn Co | | neoplasm | Angiogenesis inhibitor |
| anti-VEGF antibody, Genentech | Genentech Inc | | neoplasm, solid tumor | Angiogenesis inhibitor |
| RNasin, Promega | Promega Corp | | neoplasm | Angiogenesis inhibitor |
| Vitaxin | Scripps Research Institute | | carcinoma, neoplasm | Angiogenesis inhibitor |
| ovalicin | Harvard University | | carcinoma | Angiogenesis inhibitor |
| CM-101 | CarboMed | | neoplasm | Angiogenesis inhibitor |
| RGDfV | Merck AG | | carcinoma, inflammation | Angiogenesis inhibitor |
| LM-609 | Scripps Research Institute | | neoplasm | Angiogenesis inhibitor |
| Thrombospondin-1 peptides, | National Cancer Institute | | carcinoma | Angiogenesis inhibitor |
| NCI | | | | |
| SP-42 | Sequus Pharmaceuticals Inc | | angiogenesis disorder, | Angiogenesis inhibitor |
| | | | carcinoma | |
| paclitaxel, NaPro | NaPro BioTherapeutics Inc | | carcinoma, kaposis sarcoma, | Angiogenesis inhibitor |
| | | | brain tumor, ovary tumor | |
| angiogenesis inhibitor, Boston | Boston Life Sciences Inc | | neoplasm | Angiogenesis inhibitor |
| Life Sci | | | | |
| EMPA | Meiji Milk Products Co Ltd | | neoplasm | Angiogenesis inhibitor |
| borrelidin, Eisai | Eisai Co Ltd | | neoplasm | Angiogenesis inhibitor |
| del-1 gene, Progenitor | Progenitor Inc | | neoplasm | Angiogenesis modulator |
| angiopoietins, Regeneron | Regeneron Pharmaceuticals Inc | WO 96/11269 | angiogenesis disorder, neoplasm | Angiogenesis modulator |
| MGI-114 | MGI Pharma Inc | | breast tumor, carcinoma, colon | AntAlkylating agent |
| | | | tumor, lung tumor, neoplasm, | |
| | | | ovary tumor, uterine cervix | |
| | | | tumor | |
| CCRL-1033 | University of Bradford | | breast tumor | Antibacterial |
| boanmycin | Chinese Academy of Medical | | neoplasm | Antibacterial |
| | Science | | | |
| antibiotic/anticancer, | Theratechnologies Inc | | neoplasm | Antibacterial |
| Theratechnologies/Ecopia | | | | |
| hydramycin | Bristol-Myers Squibb Co | | carcinoma, leukemia | Antibacterial |
| duocarmycin SA | Kyowa Hakko Kogyo Co Ltd | EP 0 376 300 | neoplasm | Antibacterial |
| hatomamicin | Yamanouchi Pharmaceutical Co | JP 07238018 | carcinoma | Antibacterial |
| | LTD | | | |
| NSC-145669 | National Cancer Institute | | carcinoma | Antibacterial |
| NSC-175635 | National Cancer Institute | | carcinoma | Antibacterial |
| NSC-175636 | National Cancer Institute | | carcinoma | Antibacterial |
| A-83669 | Abbott Laboratories Ltd | | carcinoma | Antibacterial |
| FD-211 | Taisho Pharmaceutical Co Ltd | JP 07215978 | neoplasm | Antibacterial, Anticancer |
| leinamycin | Kyowa Hakko Kogyo Co Ltd | | neoplasm | Antibacterial, Anticancer |
| drug screening, Xenova/Parke- | Xenova Group plc | | neoplasm | Antibacterial, Anticancer |
| Davis | | | | |
| Sch-50673 | Schering-Plough Corp | | neoplasm | Antibacterial, Anticancer |
| GE-3 | Kyowa Hakko Kogyo Co Ltd | | carcinoma, pancreas tumor | Antibacterial, Anticancer |
| NK-130119 | Nippon Kayaku Co Ltd | EP 0 381 124 | carcinoma | Antibacterial, Anticancer, |
| | | | | Antimicrobial |
| placetins | Yamanouchi Pharmaceutical Co | | carcinoma | Antibacterial, Platelet |
| | Ltd | | | aggregation inhibitor |
| indium In 111 satumomab | CYTOGEN Corp | | ovary tumor, colorectal tumor, | Anticancer |
| pendetide | | | breast tumor | |
| hN901-DM1, ImmunoGen | ImmunoGen Inc | | lung tumor | Anticancer |
| 4-iodo-3-nitro-benzamide, | Octamer Inc | WO 94/26730 | carcinoma | Anticancer |
| Octamer | | | | |
| modified thionucelosides, | Yamasa Shoyu Co Ltd | | neoplasm | Anticancer |
| Yamasa | | | | |
| LAC-83 | Shumeido Co | | neoplasm | Anticancer |
| AccuSite | Matrix Pharmaceutical Inc | | skin tumor, squamous cell | Anticancer |
| | | | carcinoma, carcinoma | |
| SPC-104065 | Sphinx Pharmaceuticals Corp | | neoplasm | Anticancer |
| MAb ICR-62 | Institute of Cancer Research, | WO 95/20045 | carcinoma | Anticancer |
| | UK | | | |
| EL-530 | Elan Corp Plc | | prostate tumor | Anticancer |
| ONYX-015 | ONYX Pharmaceuticals Inc | WO 94/18992 | head & neck tumor, pancreas | Anticancer |
| | | | tumor, ovary tumor, digestive | |
| | | | system tumor | |
| perillyl alcohol, Endorex | National Cancer Institute | | breast tumor, prostate tumor, | Anticancer |
| | | | ovary tumor, neoplasm | |
| MDM2/p53 inhibitors, Genzyme | Genzyme Molecular Oncology | | neoplasm, sarcoma | Anticancer |
| Molecular Oncology/Xenova | | | | |
| WMC-26 | National Cancer Institute | | neoplasm, colon tumor, prostate | Anticancer |
| | | | tumor | |
| sesbanimide analogues, NCI/Ash | National Cancer Institute | | leukemia | Anticancer |
| Stevens | | | | |
| CRD-401 | Chong Kun Dang Corp | | neoplasm | Anticancer |
| AT-3510 | Kyorin Pharmaceutical Co Ltd | | carcinoma | Anticancer |
| Gliadel | Scios Inc | | brain tumor, glioma | Anticancer |
| NSC-654891 | University of Auckland | | neoplasm | Anticancer |
| HT-003 | Hanhyo Science & Technology | | neoplasm | Anticancer |
| | Institute | | | |
| electroporation therapy, | Genetronics Inc | | angiogenesis disorder, head & | Anticancer |
| | | | neck tumor, kaposis sarcoma, | |
| Genetronics | | | liver tumor, melanoma, | |
| | | | neoplasm, pain, pancreas tumor, | |
| | | | prostate tumor, squamous cell | |
| | | | carcinoma | |
| valrubicin | Anthra Pharmaceuticals | | bladder tumor, ovary tumor, | Anticancer |
| | | | precancer | |
| FK-973 | Fujisawa Pharmaceutical Co Ltd | | carcinoma | Anticancer |
| TA-077 | Tanabe Seiyaku Co Ltd | | neoplasm | Anticancer |
| OSW-1 | Tokyo University | | carcinoma | Anticancer |
| 3622W94 | Glaxo Wellcome plc | | prostate tumor, lung tumor, | Anticancer |
| | | | stomach tumor | |
| 1209W95 | Glaxo Wellcome plc | | lung tumor, prostate tumor, | Anticancer |
| | | | stomach tumor | |
| SPI-77 | Sequus Pharmaceuticals Inc | | carcinoma, lung tumor, solid | Anticancer |
| | | | tumor | |
| podophyllotoxin derivative, | Pharma Mar SA | | breast tumor, colon tumor | Anticancer |
| PharmaMar | | | | |
| 506U | Duke University | | leukemia, non-Hodgkin's | Anticancer |
| | | | lymphoma | |
| sheep monoclonals, KS | KS Biomedix Ltd | WO 92/15699 | angiogenesis disorder, bladder | Anticancer |
| Biomedix | | | tumor, breast tumor, colon | |
| | | | tumor, crohns disease, lung | |
| | | | tumor, skin tumor | |
| betulinic acid | University of Illinois | | melanoma | Anticancer |
| mitoxantrone hydrochloride | Immunex Corp | | breast tumor, liver tumor, | Anticancer |
| | | | myeloid leukemia, non- | |
| | | | Hodgkin's lymphoma, ovary | |
| | | | tumor, prostate tumor | |
| DOX-LL2, Immunomedics | Immunomedics Inc | | lymphoma | Anticancer |
| vaccine (cancer), Immunomedics | Immunomedics Inc | EP 0 438 803 | breast tumor, carcinoma, | Anticancer |
| | | | colorectal tumor, digestive | |
| | | | system tumor | |
| mitomycin-C analogs, US | University of Georgetown | | breast tumor, stomach tumor | Anticancer |
| Bioscience | | | | |
| anticancer (dinuclear platinum), | Virginia Commonwealth | | carcinoma | Anticancer |
| Boehringer Mannheim | University | | | |
| anticancer (ADEPT), University | University of Auckland | | carcinoma Anticancer | |
| of Auckland | | | | |
| Regressin | Bioniche Inc | | bladder tumor, colon tumor, | Anticancer |
| | | | esophagus tumor, leukemia | |
| oxanosine analogs, Nippon | Nippon Kayaku Co Ltd | | carcinoma | Anticancer |
| Kayaku | | | | |
| RX-465 | Chugai Pharmaceutical Co Ltd | | sarcoma | Anticancer |
| cT84.66 | Abbott Laboratories | | colorectal tumor | Anticancer |
| DTPA-BrE-3 | Coulter Corp | | breast tumor | Anticancer |
| cobalt hematoporphyrin | University of Illinois | | carcinoma | Anticancer |
| ZYN-198 | Zynaxis Inc | | ovary tumor, lung tumor | Anticancer |
| ZYN-191 | Zynaxis Inc | | ovary tumor | Anticancer |
| BCNU analogs, BMS | Bristol-Myers Squibb Co | | carcinoma | Anticancer |
| silaplatin | National Cancer Institute | | carcinoma | Anticancer |
| FCE-28068 | Pharmacia & Upjohn Inc | | neoplasm | Anticancer |
| Bowman Birk Inhibitor | University of Pennsylvania | | carcinoma, neoplasm | Anticancer |
| Concentrate (BBIC) | | | | |
| NOVOMAb-G2 | Novopharm Biotech | | carcinoma, breast tumor, colon | Anticancer |
| | | | tumor, prostate tumor, | |
| | | | melanoma, non-Hodgkin's | |
| | | | lymphoma | |
| MEN-10755 | Menarini Richerche Sud SpA | | lung tumor, uterine cervix | Anticancer |
| | | | tumor, ovary tumor, uterus | |
| | | | tumor, breast tumor | |
| JM-473 | Johnson Matthey plc | | ovary tumor | Anticancer |
| C-1311 | University of Bradford | | colon tumor | Anticancer |
| EL-532 | Elan Corp Plc | | glioma | Anticancer |
| neuregulin inhibitors, Cambridge | Cambridge NeuroScience Inc | | breast tumor, ovary tumor, brain | Anticancer |
| Neuroscience | | | tumor | |
| anticancer, Panax | InKine Pharmaceuticals Co Inc | | neoplasm | Anticancer |
| KP-692 | Deutsches Krebs- | | neoplasm | Anticancer |
| | forschungszentrum | | | |
| SDZ-MKT-077 | Novartis AG | | neoplasm | Anticancer |
| MitoExtra | SuperGen Inc | | breast tumor, colorectal tumor, | Anticancer |
| | | | lung tumor, neoplasm, pancreas | |
| | | | tumor, stomach tumor | |
| p53-inverse agents, NCI | National Cancer Institute | | carcinoma | Anticancer |
| GB-21 | Andrulis | | breast tumor, lung tumor, | Anticancer |
| | | | neoplasm, ovary tumor, renal | |
| | | | tumor | |
| MeDZQ | University of Colorado at | | lung tumor | Anticancer |
| | Boulder | | | |
| LS-4565 | Pharmacia & Upjohn AB | | colorectal tumor, pancreas tumor | Anticancer |
| ALVAC-hIL-2 | Virogenetics Corp | | neoplasm | Anticancer |
| equol | University of Leicester | | breast tumor | Anticancer |
| CD-437 | CIRD Galderma | | neoplasm | Anticancer |
| phenylbutyrate | University of Virginia | | solid tumor | Anticancer |
| CB-30865 | Zeneca Group Plc | | neoplasm | Anticancer |
| BZQ | National Cancer Institute | | neoplasm | Anticancer |
| UFT, Bristol-Myers Squibb | Bristol-Myers Squibb Co | | colorectal tumor | Anticancer |
| C242-May | ImmunoGen Inc | | colorectal tumor | Anticancer |
| G-0069B | Taiho Pharmaceutical Co Ltd | | solid tumor | Anticancer |
| reumycin derivatives, RAMS | Russian Academy Medical | | neoplasm | Anticancer |
| | Science | | | |
| NSC-641536 | National Cancer Institute | | neoplasm | Anticancer |
| NSC-671136 | National Cancer Institute | | neoplasm | Anticancer |
| NSC-674066 | National Cancer Institute | | neoplasm | Anticancer |
| Estrasine | Russian Academy Medical | | prostate tumor | Anticancer |
| | Science | | | |
| D-7991 | Chiroscience Group plc | | neoplasm | Anticancer |
| BBR-3409 | Boehringer Mannheim GmbH | | neoplasm | Anticancer |
| L-NDDP | University of Texas System | | neoplasm | Anticancer |
| illudin M analogs, Sandoz | Novartis AG | | neoplasm | Anticancer |
| calicheamicin MAb conjugate, | American Cyanamid Co | | neoplasm | Anticancer |
| American Home Products | | | | |
| MDX-H210 | Medarex Inc | | neoplasm | Anticancer |
| gene therapy (cancer), Glaxo | Glaxo Wellcome plc | | colorectal tumor, neoplasm | Anticancer |
| Wellcome | | | | |
| gene therapy (cancer), Oxford | Oxford Biomedica Ltd | | neoplasm | Anticancer |
| BioMedica | | | | |
| gene therapy (colon cancer), | GenVec Inc | | colon tumor | Anticancer |
| GenVec | | | | |
| KYN-54 | Kuraray Co Ltd | | mouth tumor, neoplasm | Anticancer |
| FD-549 | Taisho Pharmaceutical Co Ltd | JP 08003097 | neoplasm | Anticancer |
| interferon gamma-activated | IDM Immuno-Designed | WO 94/26875 | lung tumor | Anticancer |
| macrophage, ImmunoDesigned | Molecules | | | |
| Molecules | | | | |
| anticancer therapeutics, | BASF Corp | | carcinoma | Anticancer |
| Mitotix/BASF | | | | |
| gene therapy (cancer), | Vical Inc | | neoplasm | Anticancer |
| Vical/Corixa | | | | |
| MPI-5011 | Matrix Pharmaceutical Inc | | pancreas tumor | Anticancer |
| cdk4 inhibitors, Agouron | Agouron Pharmaceuticals Inc | | neoplasm | Anticancer |
| CGP-75182A | Novartis AG | | carcinoma | Anticancer |
| anti-EGFR 225 MAb, Sloan- | Memorial Sloan-Kettering | | colon tumor | Anticancer |
| Kettering | Cancer Center Institute | | | |
| anti-p185 HER2 mAb, Sloan | Memorial Sloan-Kettering | | carcinoma | Anticancer |
| Kettering | Cancer Center Institute | | | |
| Vasopermeation Enhancement, | Techniclone Corp | | solid tumor | Anticancer |
| Techniclone | | | | |
| ellipravin | Suntory Ltd | | carcinoma | Anticancer |
| MM-590 | Mediter | | carcinoma | Anticancer |
| altretamine | US Bioscience Inc | | ovary tumor | Anticancer |
| OGT-719 | Oxford GlycoSciences plc | | liver tumor | Anticancer |
| epirubicin | Pharmacia & Upjohn Ltd | | breast tumor, carcinoma, uterine | Anticancer |
| | | | cervix tumor | |
| osthol analogs, Tokyo | Tokyo University | | carcinoma | Anticancer |
| University | | | | |
| cancer therapy, Therexsys | Cobra Therapeutics | | head & neck tumor | Anticancer |
| PTL-68001 | Imperial Cancer Research | | colorectal tumor, lung tumor, | Anticancer |
| | Technology Ltd | | pancreas tumor | |
| edelfosine analog, Liposome | The Liposome Company Inc | | breast tumor, leukemia | Anticancer |
| anticancer agent, Indena/Torii | Indena SpA | | neoplasm | Anticancer |
| glutathione diesters | University of Nottingham | | carcinoma | Anticancer |
| cyclin D1 inhibitors, Prolifix | Prolifix Ltd | | breast tumor, carcinoma | Anticancer |
| torilin | Il Dong Pharm Co Ltd | | carcinoma, colon tumor, | Anticancer |
| | | | stomach tumor | |
| RPR-109881 | Rhone-Poulenc Rorer Inc | | solid tumor | Anticancer |
| anticancer agents (2), | University of Illinois | | neoplasm | Anticancer |
| NIH/Illinois | | | | |
| breast cancer therapy, SRI/Taiho | SRI International | | breast tumor | Anticancer |
| Theragyn | Antisoma plc | | ovary tumor | Anticancer |
| anticancer, Cancer Therapeutics | Cancer Therapeutics Ltd | | neoplasm | Anticancer |
| autotaxin, NIH | National Institutes of Health | | melanoma | Anticancer |
| OMT peptides, NIH | National Institutes of Health | | neoplasm | Anticancer |
| TES-23-NCS | Chugai Pharmaceutical Co Ltd | | carcinoma | Anticancer |
| FK-317 | Fujisawa Pharmaceutical Co Ltd | | carcinoma, neoplasm | Anticancer |
| anticancer, SuperGen/Galenica | SuperGen Inc | | neoplasm | Anticancer |
| ISIS-7817 | ISIS Pharmaceuticals Inc | | neoplasm | Anticancer |
| Maspin | Dana Farber Cancer Institute Inc | | breast tumor, carcinoma, | Anticancer |
| | | | prostate tumor | |
| metallo-organic compounds, | SuperGen Inc | | carcinoma | Anticancer |
| SuperGen | | | | |
| CN-716 | Calydon Inc | WO 95/19434 | prostate tumor | Anticancer |
| CN-72X series | Calydon Inc | WO 95/19434 | prostate tumor | Anticancer |
| CN-73X series | Calydon Inc | WO 95/19434 | prostate tumor | Anticancer |
| CN-74X series | Calydon Inc | | liver tumor | Anticancer |
| CN-75X series | Calydon Inc | | breast tumor | Anticancer |
| CN-76X series | Calydon Inc | | ovary tumor | Anticancer |
| cdc25a inhibitor, Ontogen | Ontogen Corp | | carcinoma | Anticancer |
| monoclonal antibody (breast | National Institutes of Health | | breast tumor | Anticancer |
| cancer), NIH | | | | |
| Leuknil | Advanced Plant Pharmaceuticals | | leukemia | Anticancer |
| | Inc | | | |
| senescence gene, Lark | Baylor College of Medicine | | neoplasm | Anticancer |
| anti-TAG-72 cell therapy, Cell | Cell Genesys Inc | | colon tumor, ovary tumor | Anticancer |
| Genesys/NCI | | | | |
| gene therapy (cancer), Cell | Cell Genesys Inc | | breast tumor | Anticancer |
| Genesys/Dana-Farber | | | | |
| cancer therapy, Cell | Cell Genesys Inc | | neoplasm | Anticancer |
| Genesys/University of Arizona | | | | |
| gene therapy (prostate cancer), | Incyte Pharmaceuticals Inc | | prostate tumor, breast tumor | Anticancer |
| Incyte/Affymetrix | | | | |
| ProCon Vector | Bavarian Nordic Research | | pancreas tumor, breast tumor | Anticancer |
| | Institute AS | | | |
| hexamethylenebisacetamide | National Cancer Institute | | neoplasm | Anticancer |
| Halomon | University of Maryland | | brain tumor, renal tumor, colon | Anticancer |
| | | | tumor | |
| Cordycepin, OXiGENE | OXiGENE Inc | | leukemia | Anticancer |
| 506U78 | Glaxo Wellcome plc | | leukemia, non-Hodgkin's | Anticancer |
| | | | lymphoma | |
| gene therapy (prostate tumor), | University of California | | prostate tumor | Anticancer |
| UCLA | | | | |
| EpiCyte | EpiGenesis Pharmaceuticals Inc | | carcinoma, colon tumor, | Anticancer |
| | | | myeloid leukemia | |
| SC-101g | Scotia Holdings plc | | neoplasm, bladder tumor | Anticancer |
| Alkasar-18 | Universitat Tubingen | | leukemia, carcinoma | Anticancer |
| NF-02411A | Nippon Kayaku Co Ltd | | neoplasm | Anticancer |
| vincristine (liposome- | NeoPharm Inc | | colon tumor | Anticancer |
| encapsulated), NeoPharm | | | | |
| paclitaxel (liposome- | NeoPharm Inc | | breast tumor, ovary tumor | Anticancer |
| encapsulated), NeoPharm | | | | |
| vaccine (cancer), University of | University of Alberta | | brain tumor, melanoma | Anticancer |
| Alberta/Briana | | | | |
| MDX-220 | Medarex Inc | | neoplasm | Anticancer |
| A-MYB gene, Temple | Temple University | | breast tumor, testis tumor | Anticancer |
| University | | | | |
| Pretarget | NeoRx Corp | | carcinoma | Anticancer |
| oncologicals, | InflaZyme Pharmaceuticals Ltd | | lung tumor, colon tumor, skin | Anticancer |
| SuperGen/InflaZyme | | | tumor, leukemia, lymphoma | |
| AMD-473 | Institute of Cancer Research, | | neoplasm, ovary tumor | Anticancer |
| | UK | | | |
| J-107088 | Banyu Pharmaceutical Co Ltd | | solid tumor | Anticancer |
| RB-90745 | British Technology Group Plc | | neoplasm | Anticancer |
| Pseurotin A | Nippon Kayaku Co Ltd | | ovary tumor, nervous system | Anticancer |
| | | | tumor | |
| tgDCC-E1A, Targeted | Targeted Genetics Corp | | ovary tumor, solid tumor, breast | Anticancer |
| Genetics/MD Anderson | | | tumor | |
| KM-966 | Kyowa Hakko Kogyo Co Ltd | | neoplasm | Anticancer |
| aplidine | Pharma Mar SA | | neoplasm | Anticancer |
| INXC-gTK | Inex Pharmaceuticals Corp | | neoplasm | Anticancer |
| anticancer agents, Lilly/ILEX | Eli Lilly & Co Ltd | | neoplasm | Anticancer |
| KB-8498 | Kanebo KK | | neoplasm | Anticancer |
| G-207 virus construct, | NeuroVir | WO 96/39841 | glioma | Anticancer |
| Neuro Vir/NCI/Georgetown | | | | |
| Univ | | | | |
| CN-706 | Calydon Inc | | prostate tumor | Anticancer |
| conjugated doxorubicin, UL | University of London | | neoplasm | Anticancer |
| School of Pharmacy | | | | |
| paclitaxel analogs, Xechem | Xechem International Inc | | neoplasm | Anticancer |
| anticancer screening, Genzyme | Genzyme Molecular Oncology | | neoplasm | Anticancer |
| Molecular/NCI | | | | |
| BE-4-4-4-4 | University of Wisconsin, | | prostate tumor | Anticancer |
| | Madison | | | |
| BE-3-7-3 | University of Wisconsin, | | prostate tumor | Anticancer |
| | Madison | | | |
| TALL-104 cell therapy, Wistar | Wistar Institute of Anatomy & | | breast tumor, neoplasm, prostate | Anticancer |
| | Biology | | tumor | |
| anticancers, Biota/BRI | Biota Holdings ltd | | neoplasm | Anticancer |
| cancer genetics, Genzyme | Genzyme Molecular Oncology | | neoplasm | Anticancer |
| Molecular/JHU | | | | |
| AMP-301 | Amplimed Inc | | neoplasm | Anticancer |
| aminopterin, University of Texas | University of Texas System | | leukemia, neoplasm, uterus | Anticancer |
| | | | tumor | |
| SBT-1514 | Stony Brook University | | neoplasm | Anticancer |
| horse raddish extract (cancer), | Kyoto University | | neoplasm | Anticancer |
| Kyoto | | | | |
| peptides (anticancer), | University of British Columbia | | neoplasm | Anticancer |
| Micrologix/British Columbia | | | | |
| MMA-383 | Novartis AG | | colon tumor | Anticancer |
| anticancer therapy, Demeter | Demeter Biotechnologies Ltd | | prostate tumor, neoplasm | Anticancer |
| gene discovery, deCODE/Roche | deCODE genetics | | prostate tumor, breast tumor, | Anticancer |
| | | | colon tumor, neoplasm | |
| Ad-TK, RPR Gencell | RPR Gencell | | brain tumor | Anticancer |
| anticancer therapeutics, | Tularik Inc | | neoplasm | Anticancer |
| Tularik/Amplicon | | | | |
| anticancer therapeutics, | Imclone Systems Inc | | neoplasm | Anticancer |
| ImClone/CombiChem | | | | |
| gene therapy (hepatoma), | National Institutes of Health | | liver tumor | Anticancer |
| NIH/Copernicus | | | | |
| G-009 | Il-Yang Pharm Ind Co Ltd | | neoplasm | Anticancer |
| CPR-1007 | Clarion Pharmaceuticals Inc | | neoplasm | Anticancer |
| FCE-28987 | Pharmacia & Upjohn Inc | | neoplasm | Anticancer |
| S-448 | Searle & Co | | carcinoma, solid tumor | Anticancer |
| CS-682 | Sankyo KK | | carcinoma | Anticancer |
| GERI-BP002-A | Korea Research Institute of | | neoplasm | Anticancer |
| | Chemical Technology | | | |
| VE-cadherin-2 antagonists, | Imclone Systems Inc | | angiogenesis disorder, neoplasm | Anticancer |
| ImClone/Marco Negri | | | | |
| NU-3076 | The University of Newcastle | | neoplasm | Anticancer |
| | Upon Tyne | | | |
| AO-90 | Otsuka Pharmaceutical Co Ltd | | neoplasm, stomach tumor | Anticancer |
| 4-PBA, Johns Hopkins | Johns Hopkins University | | solid tumor | Anticancer |
| bromotaxol, Liposome Company | The Liposome Company Inc | | lung tumor, neoplasm, ovary | Anticancer |
| | | | tumor | |
| gene therapy (cancer), NuGene | NuGene Technologies Inc | | solid tumor | Anticancer |
| camptothecin analogs, St Jude | St Jude Childrens Hospital | | neoplasm | Anticancer |
| Hospital | | | | |
| Heteroarotinoids | Oklahoma State University | | carcinoma, neoplasm | Anticancer |
| CHS-828 | Leo Denmark | | neoplasm | Anticancer |
| anticancers. Tokyo/Taisho | Tokyo University of Pharmacy | | neoplasm | Anticancer |
| | & Life Sciences | | | |
| BMY-45012 | Bristol-Myers Squibb Co | | carcinoma | Anticancer |
| anticancer agents, Targon/Duke | Targon Corp | | neoplasm | Anticancer |
| vitamin 1,25-D3 + | University of Pittsburgh | | neoplasm | Anticancer |
| dexamethasone | | | | |
| gene therapy (cancer), | Hyseq Inc | | neoplasm | Anticancer |
| Hyseg/Chiron | | | | |
| J-104134 | Banyu Pharmaceutical Co Ltd | | neoplasm | Anticancer |
| photodynamic therapy, Steba | Weizmann Institute of Science | | neoplasm | Anticancer |
| Beheer | | | | |
| IM-862 | Cytran Inc | | kaposis sarcoma | Anticancer |
| anticancer agents, Icos/CAT | Icos Corp | | neoplasm | Anticancer |
| cryptophycin 8, Wayne State | Wayne State University | | neoplasm | Anticancer |
| University | | | | |
| EGF-Genistein | Wayne Hughes Institute | | neoplasm, breast tumor | Anticancer |
| KI-60606 | Il-Yang Pharm Ind Co Ltd | | neoplasm | Anticancer |
| arenastatin A analogs, BioChem | BioChem Therapeutic Inc | | neoplasm | Anticancer |
| Therapeutics | | | | |
| SLT-1, Select/OCI/Toronto | Select Therapeutics Inc | | neoplasm | Anticancer |
| University | | | | |
| immunoliposomes (breast | University of California San | | breast tumor | Anticancer |
| cancer), UCSF | Francisco | | | |
| Pep: Trans | Synt: em | | neoplasm | Anticancer |
| varacin analog | University of Missouri | | neoplasm | Anticancer |
| anticancer agents, Cellomics | Cellomics Inc | | neoplasm | Anticancer |
| mda-7 gene, GenQuest/Introgen | GenQuest Inc | | neoplasm | Anticancer |
| INK4a | St Jude Childrens Hospital | | neoplasm | Anticancer |
| NBQ-59 | University of Puerto Rico | | neoplasm | Anticancer |
| TRAIL protein, Immunex | Immunex Corp | | neoplasm | Anticancer |
| 4-1BB ligand, Immunex | Immunex Corp | | neoplasm | Anticancer |
| Isolex 300 Stem Cell Selection | Nexell Therapeutics Inc | | neoplasm | Anticancer |
| System | | | | |
| anticancer agents, Imutec/NCI | Imutec Pharma Inc | | neoplasm | Anticancer |
| autologous lymphocyte therapy, | CYTOGEN Corp | | renal tumor, carcinoma | Anticancer |
| Cytogen | | | | |
| TGF-alpha and EGFR antisense | University of Pittsburgh | | neoplasm | Anticancer |
| therapy, UPCI | | | | |
| vitamin D3, UPCI | University of Pittsburgh | | neoplasm | Anticancer |
| IL-2, UPCI | University of Pittsburgh | | neoplasm | Anticancer |
| dequalinium | New York University | | neoplasm | Anticancer |
| EPH-88 | University of Innsbruck | | neoplasm, breast tumor, colon | Anticancer |
| | | | tumor, carcinoma, melanoma | |
| AM-132 | Kyowa Hakko Kogyo Co Ltd | | neoplasm | Anticancer |
| glyfoline | National Taiwan University | | carcinoma | Anticancer |
| polyamine analogs, Johns | Johns Hopkins University | | solid tumor | Anticancer |
| Hopkins University | | | | |
| deferoxamine | University of Maryland | | leukemia, nervous system tumor | Anticancer |
| tBCEU | CHUQ | | neoplasm | Anticancer |
| Ech-7 | Yonsei University | | neoplasm | Anticancer |
| C2-ceramide | Children's Hospital of Los | | neoplasm, nervous system tumor | Anticancer |
| | Angeles | | | |
| Coriolus versicolor extract | Hong Kong University | | neoplasm | Anticancer |
| CAPE | Strang Cancer Prevention Center | | neoplasm | Anticancer |
| sanguinarium chloride | Memorial University | | neoplasm | Anticancer |
| arsenic trioxide | Mount Sinai School of Medicine | | leukemia, neoplasm | Anticancer |
| VEGF antisense oligonucletide, | Hoechst Marion Roussel Ltd | | angiogenesis disorder, solid | Anticancer |
| HMR | | | tumor | |
| integrin antagonists, Merck | Merck KGaA | | neoplasm | Anticancer |
| vitamin D analog 1- | University of Illinois | | breast tumor, carcinoma | Anticancer |
| alpha(OH)D5 | | | | |
| vitamin 1,25-D3, Georgetown | University of Georgetown | | breast tumor | Anticancer |
| University | | | | |
| suberanilohydroxamic acid | Memorial Sloan-Kettering | | neoplasm | Anticancer |
| | Cancer Center Institute | | | |
| GTE-TP91 | MD Anderson Cancer Center | | neoplasm | Anticancer |
| JM-3286 | Johnson Matthey plc | | carcinoma | Anticancer |
| PARP inhibitors, University of | University of Bath | | neoplasm | Anticancer |
| Bath | | | | |
| pleurotin | University of Arizona | | neoplasm | Anticancer |
| doxorubicin analogs, MD | MD Anderson Cancer Center | | neoplasm | Anticancer |
| Anderson | | | | |
| cisplatin analogs, MD Anderson | MD Anderson Cancer Center | | neoplasm | Anticancer |
| platinum anticancer agents, Peter | Peter Maccallum Cancer | | neoplasm | Anticancer |
| MacCallum | Institute | | | |
| poly-plat | Michigan State University | | neoplasm | Anticancer |
| BBR-3611 | Boehringer Mannheim Italia | | neoplasm | Anticancer |
| | SpA | | | |
| baccatin III | Medical University of South | | neoplasm | Anticancer |
| | Carolina | | | |
| FGF-2 adenovirus, Prizm | Prizm Pharmaceuticals Inc | | neoplasm | Anticancer |
| JBT-3002 | MD Anderson Cancer Center | | neoplasm | Anticancer |
| antisense oligonucleotide, (HER- | ISIS Pharmaceuticals Inc | | neoplasm | Anticancer |
| 2/neu), ISIS | | | | |
| TAS-106 | Taiho Pharmaceutical Co Ltd | | neoplasm | Anticancer |
| P-108 | University of Georgetown | | brain tumor | Anticancer |
| gene therapy (cancer), | DNAX Research Institute of | | neoplasm | Anticancer |
| DNAX/McMaster | Molecular & Cellular Biology | | | |
| | Inc | | | |
| gene therapy (SCLC), University | University of Nottingham | | lung tumor | Anticancer |
| of Nottingham | | | | |
| adenoviral vector (glioma), | GenVec Inc | | glioma, neoplasm | Anticancer |
| GenVec | | | | |
| UCH-9 | Kyowa Hakko Kogyo Co Ltd | | neoplasm | Anticancer |
| EC-708 | Biovation Ltd | | prostate tumor | Anticancer |
| deimmunized Abs (colon | Cancer Research Campaign | | colon tumor | Anticancer |
| cancer), Biovation/CRC | (UK) | | | |
| DW-2282 | Dong-Wha Pharmaceutical | WO 98/07719 | neoplasm | Anticancer |
| | Industry Co Ltd | | | |
| gene therapy (cancer), Schering- | Schering-Plough Corp | | neoplasm | Anticancer |
| Plough/Genzyme | | | | |
| FE-399 | Ajinomoto Co Inc | WO 97/44479 | neoplasm | Anticancer |
| captopril, University of | University of Missouri | | breast tumor, carcinoma | Anticancer |
| Missouri/Northwestern | | | | |
| University | | | | |
| ALVAC-GM-CSF | Pasteur Merieux Connaught | | neoplasm | Anticancer |
| SMT-487 | Novartis Pharma AG | | neoplasm | Anticancer |
| DT388-GM-CSF, Univ South | Medical University of South | | myeloid leukemia | Anticancer |
| Carolina | Carolina | | | |
| BCH-4556 | BioChem Therapeutic Inc | | neoplasm, prostate tumor, renal | Anticancer |
| | | | tumor, leukemia, sarcoma, solid | |
| | | | tumor | |
| NIK-333 | Nikken Chemicals Co Ltd | | liver tumor | Anticancer |
| DW-2143 | Dong-Wha Pharmaceutical | | neoplasm | Anticancer |
| | Industry Co Ltd | | | |
| NSC-364432 | National Cancer Institute | | neoplasm | Anticancer |
| TH: TNF gene therapy, | University of Pittsburgh | | glioma | Anticancer |
| University of Pittsburgh | | | | |
| gene therapy (HSV-TK/GCV), | Rijksuniversiteit Te Leiden | | neoplasm | Anticancer |
| University of Leiden | | | | |
| helper virus-free HSV-1 | Harvard Medical School | | glioma | Anticancer |
| amplicon, Harvard | | | | |
| gene vector (anti-angiogenesis), | University of Alabama in | | neoplasm | Anticancer |
| Univ of Birmingham | Birmingham | | | |
| BE-56384 | Banyu Pharmaceutical Co Ltd | JP 10101676 | neoplasm | Anticancer |
| BCH-2051 | BioChem Therapeutic Inc | | neoplasm | Anticancer |
| AdCMV.CD, HepaVec | HepaVec GmbH | | neoplasm | Anticancer |
| AdCMV.Y28, RPR Gencell | RPR Gencell | | neoplasm | Anticancer |
| gene therapy (p53 analog), RPR | RPR Gencell | | neoplasm | Anticancer |
| Gencell | | | | |
| gene therapy (prostate cancer), | Baylor College of Medicine | | glioma, prostate tumor | Anticancer |
| Baylor College | | | | |
| gene therapy (glioblastoma), | University of Pennsylvania | | glioma | Anticancer |
| University of Pennsylvania | | | | |
| gene vector (IFN-beta), Biogen | Biogen Inc | | solid tumor | Anticancer |
| gene therapy (HSV-tk/cytokine), | RPR Gencell | | neoplasm, metastasis | Anticancer |
| RPR Gencell | | | | |
| anti CD44 monoclonals, | Boehringer Ingelheim Corp | | neoplasm | Anticancer |
| Boehringer/Sloan Kettering | | | | |
| DaunoXome | NeXstar Pharmaceuticals Inc | | carcinoma, kaposis sarcoma, | Anticancer |
| | | | uterine cervix tumor, colon | |
| | | | tumor, breast tumor, lung tumor, | |
| | | | liver tumor, leukemia, brain | |
| | | | tumor, bladder tumor, | |
| | | | lymphoma | |
| LS2D617 | Eli Lilly & Co | | carcinoma | Anticancer |
| CC49-SCA | Enzon Labs Inc | WO 93/11161 | neoplasm | Anticancer |
| BMS-191352 | Bristol-Myers Squibb Co | | neoplasm | Anticancer |
| LY-282242 | Eli Lilly & Co | | neoplasm | Anticancer |
| DMDC | Yoshitomi Pharmaceutical | | neoplasm | Anticancer |
| | Industries Ltd | | | |
| CMB-401 | Celltech Group plc | | ovary tumor, lung tumor, breast | Anticancer |
| | | | tumor | |
| vinorelbine | Pierre Fabre Participations SA | EP 0 010 458 | breast tumor, lung tumor, head | Anticancer |
| | | | & neck tumor, brain tumor, | |
| | | | prostate tumor | |
| D-20133 | Degussa AG | | neoplasm | Anticancer |
| aragusterol A | Taisho Pharmaceutical Co Ltd | EP 0 467 664 | neoplasm | Anticancer |
| KRN-5500 | Kirin Brewery Co Ltd | EP 0 525 479 | carcinoma, colon tumor, | Anticancer |
| | | | digestive system tumor, | |
| | | | neoplasm | |
| ADEPT, Zeneca/CRC | Zeneca Group Plc | | neoplasm, breast tumor, | Anticancer |
| Technology | | | colorectal tumor | |
| anthracyclines, Servier | Servier | | carcinoma | Anticancer |
| OncoRad PR356 | CYTOGEN Corp | | neoplasm, prostate tumor | Anticancer |
| DOX-CEA | Immunomedics Inc | | breast tumor | Anticancer |
| monoclonal antibodies (EGFR), | Imclone Systems Inc | | neoplasm | Anticancer |
| ImClone | | | | |
| gene therapy (lung cancer), NCI | National Cancer Institute | | neoplasm, lung tumor | Anticancer |
| monoclonals (cancer), Scotgen | Scotgen Biopharmaceuticals Inc | | neoplasm, pancreas tumor | Anticancer |
| Allovectin-7 | Vical Inc | | melanoma, renal tumor, | Anticancer |
| | | | colorectal tumor, neoplasm, | |
| | | | breast tumor, non-Hodgkin's | |
| | | | lymphoma, head & neck tumor | |
| DA-125 | Dong-A Pharmaceutical Co Ltd | | neoplasm, breast tumor, lung | Anticancer |
| | | | tumor, stomach tumor | |
| Doxil | Sequus Pharmaceuticals Inc | | kaposis sarcoma, sarcoma, | Anticancer |
| | | | breast tumor, ovary | |
| | | | tumor, liver tumor, | |
| | | | prostate tumor, leukemia, lung | |
| | | | tumor, renal tumor, colorectal | |
| | | | tumor, head & neck tumor | |
| Annamycin LF | University of Texas System | | breast tumor, neoplasm | Anticancer |
| S-16209 | Servier | | neoplasm | Anticancer |
| Sch-58500 | Canji Inc | WO 96/34969 | breast tumor, carcinoma, | Anticancer |
| | | | colorectal tumor, head & neck | |
| | | | tumor, leukemia, liver tumor, | |
| | | | lung tumor, melanoma, | |
| | | | neoplasm, ovary tumor | |
| INGN-101 | Introgen Therapeutics Inc | | lung tumor, head & neck tumor | Anticancer |
| retinoblastoma protein therapy, | Canji Inc | | bladder tumor | Anticancer |
| Canji | | | | |
| AN-1006 | Meiji Seika Kaisha Ltd | | carcinoma, leukemia | Anticancer |
| D-1411 | Chiroscience Group plc | WO 96/00075 | carcinoma | Anticancer |
| anti-B1 antibody, Coulter | Coulter Pharmaceutical Inc | U.S. Pat. No. | non-Hodgkin's lymphoma | Anticancer |
| | | 5,595,721 | | |
| DepoCyt | DepoTech Corp | | brain tumor, lymphoma, | Anticancer |
| | | | leukemia | |
| D-21805 | ASTA Medica AG | EP 0 594 999 | neoplasm | Anticancer |
| oligonucleotides, Yale | Yale University | | neoplasm | Anticancer |
| RGG-0853 | RGene Therapeutics Inc | | breast tumor, lung tumor, ovary | Anticancer |
| | | | tumor | |
| tretinoin, Roche | Roche Holdings Inc | | leukemia | Anticancer |
| Onconase | Alfacell Corp | WO 91/07435 | breast tumor, carcinoma, lung | Anticancer |
| | | | tumor, pancreas tumor, prostate | |
| | | | tumor, renal tumor | |
| BN-52207 | Ipsen-Beaufour | | neoplasm | Anticancer |
| amonafide | Knoll Ltd | | carcinoma, neoplasm | Anticancer |
| KRN-8602 | Kirin Brewery Co Ltd | | brain tumor, breast tumor, | Anticancer |
| | | | carcinoma, leukemia | |
| anthracyclines, Mercian | Mercian Corp | | carcinoma | Anticancer |
| emitefur | Otsuka Pharmaceutical Co Ltd | | carcinoma, lung tumor | Anticancer |
| SPC-103600 | Sphinx Pharmaceuticals Corp | | neoplasm | Anticancer |
| saptomycins, Sapporo | Sapporo Breweries Ltd | | carcinoma | Anticancer |
| dexifosfamide | Chiroscience Group plc | WO 96/00075 | neoplasm | Anticancer |
| TY-10721 | Toa Eiyo KK | | carcinoma | Anticancer |
| anticancer prodrug, Nippon | Nippon Kayaku Co Ltd | | neoplasm | Anticancer |
| geldanamycin | Pfizer Inc | | neoplasm | Anticancer |
| ursodiol, Axcan | Axcan Pharma Inc | | colorectal tumor | Antihypercholesterolemic agent |
| boron oligonucleotides, Duke | Duke University | | neoplasm | Antihyperlipidemic agent |
| University | | | | |
| LY-354899 | Eli Lilly & Co | | neoplasm | Antimetabolite |
| LY-309887 | Eli Lilly & Co | | carcinoma, neoplasm | Antimetabolite |
| LY-231514 | Eli Lilly & Co | EP 0 432 677 | breast tumor, carcinoma, | Antimetabolite |
| | | | colorectal tumor, lung tumor, | |
| | | | pancreas tumor | |
| lometrexol | Eli Lilly & Co | EP 0 248 573 | carcinoma, neoplasm | Antimetabolite |
| LY-223592 | Eli Lilly & Co | | carcinoma, neoplasm | Antimetabolite |
| LY-207702 | Eli Lilly & Co | | carcinoma | Antimetabolite |
| antitumor nucleosides, Hokkaido | Hokkaido University | | neoplasm | Antimetabolite |
| University | | | | |
| S-1 | Taiho Pharmaceutical Co Ltd | | breast tumor, lung tumor, head | Antimetabolite |
| | | | & neck tumor, neoplasm, | |
| | | | digestive system tumor | |
| gemcitabine | Eli Lilly & Co | GB 2 136 425 | lung tumor, pancreas tumor, | Antimetabolite |
| | | | carcinoma, uterine cervix tumor, | |
| | | | bladder tumor, urinary tract | |
| | | | tumor, breast tumor, renal | |
| | | | tumor, neoplasm, head & | |
| | | | neck tumor | |
| LY-254155 | Eli Lilly & Co | | carcinoma, neoplasm | Antimetabolite |
| MDL-101731 | Hoechst Marion Roussel Inc | EP 0 372 268 | breast tumor, colon tumor, | Antimetabolite |
| | | | leukemia, lung tumor, prostate | |
| | | | tumor, solid tumor | |
| raltitrexed | Zeneca Group Plc | EP 0 239 362 | colorectal tumor, neoplasm, | Antimetabolite |
| | | | ovary tumor, pancreas tumor | |
| LY-298207 | Eli Lilly & Co | WO 95/09845 | neoplasm | Antimetabolite |
| LY-316373 | Eli Lilly & Co | WO 95/09845 | neoplasm | Antimetabolite |
| LY-335518 | Eli Lilly & Co | | leukemia, neoplasm | Antimetabolite |
| LY-335738 | Eli Lilly & Co | | neoplasm, leukemia | Antimetabolite |
| LY-288784 | Eli Lilly & Co | | neoplasm | Antimetabolite |
| LY-295248 | Eli Lilly & Co | | neoplasm | Antimetabolite |
| fludarabine | Southern Research Inst | | leukemia, lymphoma, non- | Antimetabolite |
| | | | Hodgkin's lymphoma | |
| gene therapy (cancer), Southern | UAB Research Foundation | | neoplasm | Antimetabolite |
| Research/UAB | | | | |
| S-1286 | Sankyo KK | | carcinoma | Antimetabolite |
| canavanine analogues | Louisiana University | | pancreas tumor | Antimetabolite |
| capecitabine | Hoffmann-La Roche Inc | | breast tumor, colorectal tumor, | Antimetabolite |
| | | | solid tumor, stomach tumor | |
| cell signaling modulators, | Paracelsian Inc | | kaposis sarcoma, neoplasm | Antimetabolite |
| Paracelsian/NCI | | | | |
| lonidamine | Angelini Ricerche SpA | DE 2310031 | neoplasm, carcinoma | Antimetabolite |
| marine therapeutics, Pfizer | Pfizer Inc | | carcinoma | Antimicrobial |
| mycalamide analogs, Kaken | Kaken Pharmaceutical Co Ltd | | neoplasm | Antimicrobial |
| SOD, Oxis | OXIS International Inc | | head & neck tumor | Antioxidant agent |
| TEMPOL | US Department of Health & | WO 96/40127 | neoplasm | Antioxidant agent |
| | Human Services | | | |
| agaro-oligosaccharide, Takara | Takara Shuzo Co Ltd | | neoplasm | Antioxidant agent |
| agaro-oligosaccharide, Takara | Takara Shuzo Co Ltd | | neoplasm | Antioxidant agent |
| CR-6 | Lipotec SA | | neoplasm | Antioxidant agent |
| J-1025 | Jenapharm GmbH | | carcinoma, neoplasm | Antioxidant agent |
| CV-3611 | Takeda Chemical Industries Ltd | EP 0 146 121 | neoplasm | Antioxidant agent |
| masoprocol | Chemex Pharmaceuticals Inc | | neoplasm | Antioxidant agent |
| ODN-2009 | University Hospital Zurich | | lung tumor | Apoptosis inhibitor |
| Bcl-2 antagonists, IDUN | IDUN Pharmaceuticals Inc | | lung tumor, breast tumor, colon | Apoptosis inhibitor |
| | | | tumor, prostate tumor | |
| apoptosis inhibitors, TLC | The Liposome Company Inc | | neoplasm | Apoptosis inhibitor |
| anticancers, BioChem | BioChem Pharma Inc | | neoplasm | Apoptosis modulator |
| Pharma/Apoptosis Tech | | | | |
| FGN-1 | Cell Pathways Inc | | breast disease, uterine cervix | Apoptosis modulator |
| | | | tumor, precancer | |
| apoptosis regulators, | Zeneca Pharmaceuticals | | neoplasm, pain | Apoptosis modulator |
| Zeneca/Rutgers | | | | |
| ADAT technology, GEMMA | GEMMA Biotechnology | | neoplasm | Apoptosis modulator |
| SR-45023A | Symphar SA | | neoplasm | Apoptosis modulator |
| apoptosis modulators, | Apoptogen Inc | | neoplasm | Apoptosis modulator |
| Apoptogen | | | | |
| gene therapy (cancer), | LXR Biotechnology Inc | | neoplasm | Apoptosis modulator |
| LXR/Copernicus | | | | |
| cyclin dependent kinase | Mitotix Inc | | neoplasm | Apoptosis modulator |
| inhibitors, Mitotix/DuPont | | | | |
| Merck | | | | |
| cancer therapeutics, | Tripos Inc | | neoplasm | Apoptosis modulator |
| Tripos/Panlabs/Cell Pathways | | | | |
| MGI-114 | MGI Pharma Inc | | breast tumor, carcinoma, colon | Apoptosis stimulator |
| | | | tumor, lung tumor, neoplasm, | |
| | | | ovary tumor, uterine cervix | |
| | | | tumor | |
| AN-9 | Ansan Pharmaceuticals Inc | | neoplasm | Apoptosis stimulator |
| ALRT-620 | Allergan Ligand Retinoid | | lymphoma, solid tumor, | Apoptosis stimulator |
| | Therapeutics Inc | | squamous cell carcinoma | |
| UCN-01 | Kyowa Hakko Kogyo Co Ltd | | neoplasm | Apoptosis stimulator |
| CEP-2563 | Cephalon Inc | WO 96/31515 | prostate tumor | Apoptosis stimulator |
| agaro-oligosaccharide, Takara | Takara Shuzo Co Ltd | | neoplasm | Apoptosis stimulator |
| verteporfin | QLT PhotoTherapeutics Inc | | radiation sickness, carcinoma | Apoptosis stimulator |
| apoptin | Rijksuniversiteit Te Leiden | | neoplasm | Apoptosis stimulator |
| casiopeina II | University of Surrey | | neoplasm | Apoptosis stimulator |
| LDI-200 | Milkhaus Laboratory Inc | | leukemia, kaposis sarcoma, pain, | Apoptosis stimulator |
| | | | malignant neoplastic disease, | |
| | | | prostate tumor | |
| apoptosis inducer, Temple | Temple University | | breast tumor | Apoptosis stimulator |
| University | | | | |
| Oncodon | Vyrex Corp | | carcinoma | Apoptosis stimulator |
| LAN-7 | University of California | | carcinoma | Apoptosis stimulator |
| anticancer, Biota/Hitachi/Nippon | Hitachi Kasei Kogyo KK | | neoplasm, prostate tumor | Apoptosis stimulator |
| MX-3350-1 | Maxia Pharmaceuticals Inc | | neoplasm | Apoptosis stimulator |
| IDN-5109 | Stony Brook University | | carcinoma | Apoptosis stimulator |
| alpha-Anordrin | Shanghai Institute of Materia | | carcinoma, neoplasm, uterine | Apoptosis stimulator |
| | Medica | | cervix tumor | |
| MKK4 tumor suppressor gene, | Myriad Genetics Inc | | neoplasm | Apoptosis stimulator |
| Myriad | | | | |
| BRCA1 modulator, Allegheny | Allegheny University of the | | breast tumor, ovary tumor | Apoptosis stimulator |
| | Health Sciences | | | |
| SR-11262 | F Hoffmann-La Roche Ltd | | neoplasm | Apoptosis stimulator |
| BMD-188 | Biomide Investment Ltd | | neoplasm, prostate tumor | Apoptosis stimulator |
| | Partnership | | | |
| EGF-P-154 | Wayne Hughes Institute | | neoplasm | Apoptosis stimulator |
| NU-2058 | University of Newcastle | | neoplasm | Apoptosis stimulator |
| merocil | Baylor College of Medicine | | carcinoma | Apoptosis stimulator |
| merodantoin | Baylor College of Medicine | | carcinoma | Apoptosis stimulator |
| BBR-3464 | Boehringer Mannheim Italia | | neoplasm | Apoptosis stimulator |
| | SpA | | | |
| vinflunine | Pierre Fabre Participations SA | | neoplasm | Apoptosis stimulator |
| CNI-1493 | Picower Institute for Medical | | neoplasm | Arginine modulator |
| | Research | | | |
| CNI-1493 | Picower Institute for Medical | | neoplasm | Arginine modulator |
| | Research | | | |
| anastrozole | Zeneca Group Plc | EP 0 296 749 | breast tumor | Aromatase inhibitor |
| minamestane | Pharmacia & Upjohn AB | DE 3604179 | carcinoma | Aromatase inhibitor |
| atamestane | Schering AG | DE 3322285 | carcinoma, neoplasm, breast | Aromatase inhibitor |
| | | | tumor | |
| exemestane | Pharmacia & Upjohn AB | DE 3622841 | breast tumor | Aromatase inhibitor |
| fadrozole hydrochloride | Novartis AG | U.S. Pat. No. | breast tumor, carcinoma | Aromatase inhibitor |
| | | 4,588,732 | | |
| liarozole | Janssen Pharmaceutica NV | EP 0 260 744 | carcinoma, head & neck tumor, | Aromatase inhibitor |
| | | | leukemia, lung tumor, prostate | |
| | | | tumor | |
| letrozole | Novartis AG | EP 0 236 940 | breast tumor | Aromatase inhibitor |
| vorozole | Janssen Pharmaceutica NV | | carcinoma, breast tumor | Aromatase inhibitor |
| formestane | Novartis AG | U.S. Pat. No. | breast tumor, carcinoma | Aromatase inhibitor |
| | | 4,235,893 | | |
| TAN-931 | Takeda Chemical Industries Ltd | U.S. Pat. No. | neoplasm | Aromatase inhibitor |
| | | 5,013,757 | | |
| MFT-279 | Hoechst Marion Roussel Inc | | breast tumor | Aromatase inhibitor |
| pentrozole | Schering AG | | neoplasm | Aromatase inhibitor |
| CGP-45688 | Novartis AG | EP 0 408 509 | carcinoma, neoplasm | Aromatase inhibitor |
| rogletimide | British Technology Group Plc | | breast tumor, carcinoma, | Aromatase inhibitor |
| | | | neoplasm | |
| RU-54115 | Roussel Uclaf SA | EP 0 434 570 | breast tumor | Aromatase inhibitor |
| YM-511 | Yamanouchi Pharmaceutical Co | | neoplasm, breast tumor, uterus | Aromatase inhibitor |
| | Ltd | | tumor | |
| NKS-01 | Snow Brand Milk Products Co | | breast tumor | Aromatase inhibitor |
| | Ltd | | | |
| RU-56152 | Roussel Uclaf SA | | neoplasm | Aromatase inhibitor |
| CGS-47645 | Novartis AG | | breast tumor | Aromatase inhibitor |
| Org-33201 | Organon NV | | breast tumor | Aromatase inhibitor |
| YM-553 | Yamanouchi Pharmaceutical Co | | neoplasm | Aromatase inhibitor |
| | Ltd | | | |
| FCE-27993 | Pharmacia & Upjohn SpA | | breast tumor, prostate tumor | Aromatase inhibitor |
| GW-114 | Universitat des Saarlandes | | prostate tumor | Aromatase inhibitor |
| GW-124 | Universitat des Saarlandes | | prostate tumor | Aromatase inhibitor |
| Oncaspar | Enzon Inc | | carcinoma, leukemia, neoplasm | Asparaginase stimulator |
| sparfosic acid | Warner-Lambert Co | U.S. Pat. No. | carcinoma, colorectal tumor, | Aspartate carbamoyltransferase |
| | | 4,215,070 | neoplasm | inhibitor |
| U-0126 | DuPont Pharmaceuticals Co | | neoplasm | ATPase inhibitor |
| interleukin-6, American Home | American Home Products Corp | | neoplasm, carcinoma | B cell differentiating factor |
| Products | | | | |
| ursodiol, Axcan | Axcan Pharma Inc | | colorectal tumor | Bile acid modulator |
| EO-9 | National Institutes of Health | WO 87/06227 | neoplasm | Bioreducible cytotoxin |
| RB-6145 | British Technology Group Plc | EP 0 319 329 | carcinoma, neoplasm | Bioreducible cytotoxin |
| AQ4N | De Montfort University | | neoplasm | Bioreducible cytotoxin |
| imidocaptate | Louisiana State University | | neoplasm | Bioreducible cytotoxin |
| Promycin | Vion Pharmaceuticals Inc | | head & neck tumor, neoplasm | Bioreducible cytotoxin |
| tirapazamine | SRI International | | solid tumor, lung tumor, breast | Bioreducible cytotoxin |
| | | | tumor, ovary tumor, head & | |
| | | | neck tumor | |
| NSC-646394 | University of Auckland | | neoplasm | Bioreducible cytotoxin |
| RB-90740 | British Technology Group Plc | | neoplasm | Bioreducible cytotoxin |
| SN-23862 | University of Auckland | | neoplasm | Bioreducible cytotoxin |
| NSC-672819 | University of Auckland | | neoplasm | Bioreducible cytotoxin |
| ZM-81853 | Zeneca Group Plc | | neoplasm | Bioreducible cytotoxin |
| SR-4941 | SRI International | | neoplasm | Bioreducible cytotoxin |
| bioreductive cytotoxin, St John's | St John's Univeristy | | solid tumor | Bioreducible cytotoxin |
| CKD-608 | Chong Kun Dang Corp | WO 97/13748 | solid tumor | Bioreducible cytotoxin |
| SN-24771 | Warner-Lambert Co | | neoplasm | Bioreducible cytotoxin |
| RMP-7 | Alkermes Inc | WO 92/18529 | brain tumor, glioma | BK agonist |
| RC-3940-II | Pharmacia & Upjohn Inc | | breast tumor, neoplasm | Bombesin antagonist |
| RC-3095 | Pharmacia & Upjohn AB | WO 92/09626 | neoplasm, prostate tumor | Bombesin antagonist |
| PD-168368 | Parke-Davis & Co | | carcinoma | Bombesin antagonist |
| BW-2258-U89 | Burroughs Wellcome Inc | | lung tumor, neoplasm | Bombesin antagonist |
| D-22213 | ASTA Medica Arzneimittel Ges | | colon tumor, lung tumor | Bombesin antagonist |
| | mbH | | | |
| PTC-821 | Peptech Ltd | | carcinoma | Bombesin antagonist |
| olpadronate | Gador SA | WO 96/19998 | carcinoma, Paget's disease | Bone metabolism modulator |
| risedronic acid | Norwich-Eaton Pharmaceuticals | EP 0 186 405 | Paget's disease | Bone resorption inhibitor |
| | Inc | | | |
| raloxifene | Eli Lilly & Co | | colon tumor, neoplasm | Bone resorption inhibitor |
| TNCA | Colgate-Palmolive Co | | carcinoma | Bone resorption inhibitor |
| B-9858 | Cortech Inc | WO 97/09346 | neoplasm | Bradykinin BK-1 antagonist |
| quazepam | Schering-Plough Corp | DE 2138773 | brain tumor, melanoma | BZD agonist |
| salmon calcitonin, Cortecs | Cortecs Ltd | | osteoporosis, Paget's disease | Calcitonin agonist |
| microspheres (calcitonin), | Emisphere Technologies Inc | | Paget's disease | Calcitonin agonist |
| Emisphere | | | | |
| Fortical | Unigene Laboratories Inc | | hypercalcemia, Paget's disease | Calcitonin agonist |
| SRI-62-834 | Novartis AG | | carcinoma | Calcium absorption promotor |
| CMA-676 | Celltech Group plc | | myeloid leukemia | Calcium channel activator |
| anticancer, Johns Hopkins | Johns Hopkins University | U.S. Pat. No. | carcinoma, precancer | Calcium channel activator |
| | | 5,274,142 | | |
| verapamil isomers, | Chiroscience Group plc | WO 95/09150 | colorectal tumor, renal tumor, | Calcium channel blocker |
| Chiroscience/Knoll | | | non-Hodgkin's lymphoma | |
| FCE-28718 | Pharmacia & Upjohn SpA | EP 0 755 931 | breast tumor, ovary tumor, | Calcium channel blocker |
| | | | prostate tumor | |
| Ro-11-2933 | Roche Holding AG | EP 0 523 493 | female genital tract tumor | Calcium channel blocker |
| ibandronic acid | Boehringer Mannheim GmbH | U.S. Pat. No. | hypercalcemia, bone tumor | Calcium metabolic inhibitor |
| | | 4,942,157 | | |
| alendronate sodium | Istituto Gentili SpA | | hypercalcemia, Paget's disease | Calcium metabolic inhibitor |
| pamidronate disodium | Henkel KGaA | DE 2405254 | bone disease, bone tumor, breast | Calcium metabolic inhibitor |
| | | | tumor, hypercalcemia, | |
| | | | myeloproliferative disorder, | |
| | | | Paget's disease, prostate tumor | |
| etidronate disodium | The Procter & Gamble Co | | Paget's disease | Calcium metabolic inhibitor |
| tiludronate | Elf Sanofi | EP 0 100 718 | Paget's disease | Calcium metabolic inhibitor |
| DADS, Pennsylvania Univ | University of Pennsylvania | | neoplasm | Calcium metabolic modulator |
| neridronate | Istituto Gentili SpA | | Paget's disease | Calcium metabolic modulator |
| cathepsin inhibitors, Arris | Arris Pharmaceutical Corp | | carcinoma | Cathepsin B inhibitor |
| K-11002 | Khepri Pharmaceuticals | | neoplasm | Cathepsin inhibitor |
| cathepsin inhibitors, Arris | Arris Pharmaceutical Corp | | carcinoma | Cathepsin L inhibitor |
| YM-57409 | Yamanouchi Pharmaceutical Co | JP 09087265 | Paget's disease | Cathepsin L inhibitor |
| | Ltd | | | |
| cathepsin inhibitors, Arris | Arris Pharmaceutical Corp | | carcinoma | Cathepsin S inhibitor |
| lintitript | Sanofi Recherche SA | EP 0 432 040 | pancreas tumor | CCK A antagonist |
| loxiglumide | Rotta Research Lab SpA | WO 87/03869 | carcinoma | CCK antagonist |
| JB-93182 | James Black Fdn Ltd | WO 95/04720 | neoplasm | CCK B antagonist |
| Ro-09-1540 | Roche Holding AG | | stomach tumor | CCK B antagonist |
| CH-271 | Takara Shuzo Co Ltd | | neoplasm | Cell adhesion inhibitor |
| IC-101 | Microbial Chemistry Research | | carcinoma | Cell adhesion inhibitor |
| | Foundation | | | |
| cytostatin | Microbial Chemistry Research | | carcinoma | Cell adhesion inhibitor |
| | Foundation | | | |
| anti-inflammatories, Genetics | Genetics Institute Inc | | carcinoma | Cell adhesion inhibitor |
| Institute | | | | |
| Contortrostatin | University of Southern | | breast tumor | Cell adhesion inhibitor |
| | California | | | |
| ELAM-1 antagonists, ISIS | ISIS Pharmaceuticals Inc | | melanoma, colon tumor | Cell adhesion inhibitor |
| Pharmaceuticals | | | | |
| INGN-231 | Introgen Therapeutics Inc | | prostate tumor | Cell adhesion modulator |
| cell adhesion regulator, ICRT | Imperial Cancer Research | | neoplasm | Cell adhesion modulator |
| | Technology Ltd | | | |
| alpha-beta integrin peptides, | Integra LifeSciences Corp | | angiogenesis disorder, | Cell adhesion molecule |
| Integra | | | carcinoma, neoplasm | antagonist |
| anchor-linked angiostatic agents, | RedCell Inc | | metastasis, angiogenesis disorder | Cell adhesion molecule |
| RedCell | | | | antagonist |
| SB-265123 | SmithKline Beecham plc | | neoplasm | Cell adhesion molecule |
| | | | | antagonist |
| V-0005 | Hoechst Marion Roussel Inc | | bone tumor, angiogenesis | Cell adhesion molecule |
| | | | disorder | antagonist |
| V-0245 | Hoechst Marion Roussel Inc | | angiogenesis disorder, bone | Cell adhesion molecule |
| | | | tumor, metastasis | antagonist |
| V-0519 | Hoechst Marion Roussel Inc | | angiogenesis disorder, bone | Cell adhesion molecule |
| | | | tumor | antagonist |
| SC-68448 | Monsanto Co | | neoplasm | Cell adhesion molecule |
| | | | | antagonist |
| V-0223 | Hoechst AG | | bone tumor, metastasis, | Cell adhesion molecule |
| | | | angiogenesis disorder | antagonist |
| CAM inhibitors, Tanabe | Tanabe Seiyaku Co Ltd | | carcinoma | Cell adhesion molecule |
| | | | | antagonist |
| DNAM-1 | DNAX Research Institute of | | carcinoma | Cell adhesion molecule ligand |
| | Molecular & Cellular Biology | | | |
| | Inc | | | |
| ICAM modulators, ICOS/Abbott | Icos Corp | | neoplasm | Cell adhesion molecule |
| | | | | modulator |
| CPR-1006 | Clarion Pharmaceuticals Inc | | neoplasm | Cell control agent |
| gene therapy (neoplasm), ICRT | Imperial Cancer Research | | breast tumor, neoplasm | Cell control agent |
| | Technology Ltd | | | |
| cyclin E | Fred Hutchinson Cancer | | carcinoma | Cell control agent |
| | Research Center | | | |
| ps20 gene therapy, Baylor | Baylor College of Medicine | | prostate tumor | Cell control agent |
| College | | | | |
| melanoma susceptibility genes, | Myriad Genetics Inc | | neoplasm | Cell control agent |
| Myriad | | | | |
| SW-064652 | Sanofi Winthrop Inc | | carcinoma | Cell control agent |
| PNU-156692 | Pharmacia & Upjohn Inc | | neoplasm | Cell cycle inhibitor |
| docetaxel analogs, Daiichi | Daiichi Seiyaku Co Ltd | | neoplasm | Cell cycle inhibitor |
| rhizoxin | Fujisawa Pharmaceutical Co Ltd | EP 0 132 772 | carcinoma, solid tumor, breast | Cell cycle inhibitor |
| | | | tumor, lung tumor, head & neck | |
| | | | tumor, melanoma, ovary tumor, | |
| | | | colorectal tumor, renal tumor | |
| BG-anti-TGF-beta, Hebrew | Hebrew University of Jerusalem | | neoplasm | Cell cycle inhibitor |
| University | | | | |
| LY-354899 | Eli Lilly & Co | | neoplasm | Cell cycle inhibitor |
| PNU-166087 | Pharmacia & Upjohn Inc | | neoplasm | Cell cycle inhibitor |
| PNU-156691 | Pharmacia & Upjohn Inc | | neoplasm | Cell cycle inhibitor |
| anticancers, BioChem | BioChem Pharma Inc | | neoplasm | Cell cycle inhibitor |
| Pharma/Apoptosis Tech | | | | |
| PNU-157548 | Pharmacia & Upjohn Inc | | neoplasm | Cell cycle inhibitor |
| LY-355703 | Eli Lilly & Co | | neoplasm | Cell cycle inhibitor |
| ALRT-1500 | Allergan Ligand Retinoid | | neoplasm | Cell cycle inhibitor |
| | Therapeutics Inc | | | |
| CC49-BAMME-CH-DOX | Eli Lilly & Co | | neoplasm | Cell cycle inhibitor |
| ER-35744 | Eisai Co Ltd | | colon tumor, lung tumor | Cell cycle inhibitor |
| LS-4477 | Pharmacia & Upjohn AB | | carcinoma | Cell cycle inhibitor |
| homoharringtonine | Chinese Academy of Medical | | leukemia, myeloid leukemia | Cell cycle inhibitor |
| | Science | | | |
| curacin A | University of Pittsburgh | | neoplasm | Cell cycle inhibitor |
| gene therapy (brain tumor), | IntroGene BV | | brain tumor | Cell cycle inhibitor |
| IntroGene | | | | |
| IL4(38-37)-PE38KDL | National Cancer Institute | | neoplasm | Cell cycle inhibitor |
| antitumor agent, Clarion | Clarion Pharmaceuticals Inc | | breast tumor, colon tumor, | Cell cycle inhibitor |
| | | | leukemia | |
| telomere modulator, Geron | Geron Corp | | neoplasm | Cell cycle inhibitor |
| LS-4559 | Pharmacia & Upjohn AB | | carcinoma | Cell cycle inhibitor |
| anticancer, Prolifix | Prolifix Ltd | | neoplasm | Cell cycle inhibitor |
| docetaxel/paclitaxel analogs, | New York State University | | neoplasm | Cell cycle inhibitor |
| NYSU | | | | |
| vitamin D3 analogs, Hoffmann- | Hoffmann-La Roche | | breast tumor, neoplasm, prostate | Cell cycle inhibitor |
| La Roche | | | tumor | |
| Src inhibitors, Parke-Davis | Parke-Davis & Co | | neoplasm | Cell cycle inhibitor |
| cdc25c inhibitors, Howard | Howard Hughes Medical | | neoplasm | Cell cycle inhibitor |
| Hughes | Institute | | | |
| RKS-1778 | Riken Chemical Industry Co Ltd | | carcinoma | Cell cycle inhibitor |
| INGN-221 | Introgen Therapeutics Inc | | neoplasm | Cell cycle inhibitor |
| HMN-214 | Nippon Shinyaku Co Ltd | | neoplasm | Cell cycle inhibitor |
| UC-162 | University of California | | carcinoma | Cell cycle inhibitor |
| anhydrovinblastine | IGT Pharma Inc | | carcinoma | Cell cycle inhibitor |
| AC-7739 | Ajinomoto Co Inc | | neoplasm | Cell cycle inhibitor |
| Atr gene | Icos Corp | | neoplasm | Cell cycle inhibitor |
| butyrolactone I | National Cancer Institute | | neoplasm | Cell cycle inhibitor |
| discodermolide | Harbor Branch Oceanographic | | neoplasm | Cell cycle inhibitor |
| | Institute Inc | | | |
| vinxaltine | Servier | EP 0 318 392 | carcinoma | Cell cycle inhibitor |
| didemnin-B | Pharma Mar SA | EP 0 048 149 | breast tumor, carcinoma, central | Cell cycle inhibitor |
| | | | nervous system tumor, colorectal | |
| | | | tumor, non-Hodgkin's | |
| | | | lymphoma | |
| giracodazole | Rhone-Poulenc SA | | neoplasm | Cell cycle inhibitor |
| SDZ-GLI-328 | Genetic Therapy Inc | EP 0 476 953 | brain tumor, head & neck tumor, | Cell cycle inhibitor |
| | | | myeloproliferative disorder | |
| SDI-1 | Sennes Drugs Innovations | WO 95/06415 | neoplasm | Cell cycle inhibitor |
| SDZ-281-722 | Novartis AG | | neoplasm | Cell cycle inhibitor |
| cell cycle inhibitor, Cortex | Cortex Pharm Inc | | carcinoma | Cell cycle inhibitor |
| dehydrodidemnin B | Pharma Mar SA | | carcinoma, central nervous | Cell cycle inhibitor |
| | | | system tumor, colorectal tumor, | |
| | | | lung tumor, non-Hodgkin's | |
| | | | lymphoma, prostate tumor | |
| AC-9301 | Anticancer Inc | | carcinoma, stomach tumor, | Cell cycle inhibitor |
| | | | pancreas tumor, lung tumor | |
| CPR-1006 | Clarion Pharmaceuticals Inc | | neoplasm | Cell surface receptor inhibitor |
| Ro-23-7777 | Roche Holding AG | | carcinoma | Cell wall synthesis inhibitor |
| S-9788 | Servier | EP 0 466 586 | carcinoma | Cell wall synthesis inhibitor |
| oxeclosporin | Novartis AG | EP 0 414 632 | neoplasm | Cell wall synthesis inhibitor |
| oxeclosporin | Novartis AG | EP 0 414 632 | neoplasm | Cell wall synthesis inhibitor |
| CP-358 | Kings College London | | carcinoma | Chelating agent |
| BB-10010 | British Biotech plc | | neoplasm, breast tumor, lung | Chemokine |
| | | | tumor | |
| APC-8015 | Dendreon Corp | | prostate tumor | Chemokine |
| RANTES antibody fusion | University of Rochester | | neoplasm | Chemokine |
| protein, UCLA | | | | |
| oltipraz | Rhone-Poulenc SA | WO 94/16563 | neoplasm, prostate tumor | Chemoprotectant |
| NAcSDKP analogs, CNRS | Centre National de la Recherche | | neoplasm | Chemoprotectant |
| | Scientifigue (CNRS) | | | |
| Betafectin | Alpha-Beta Technology Inc | | carcinoma | Chemoprotectant |
| gene therapy (MDR), IntroGene | IntroGene BV | | bladder tumor, brain tumor, | Chemoprotectant |
| | | | breast tumor, carcinoma, | |
| | | | lymphoma | |
| MDR gene therapy, Ingenex | Ingenex | | breast tumor, ovary tumor | Chemoprotectant |
| dexrazoxane | Imperial Cancer Research | DE 1910283 | breast tumor | Chemoprotectant |
| | Technology Ltd | | | |
| mrp vector, Univ de Louvain | Universite Catholique De | | neoplasm | Chemoprotectant |
| | Louvain | | | |
| gene therapy (mdr1 gene), City | City of Hope | | neoplasm | Chemoprotectant |
| of Hope | | | | |
| CD34+ mdr1 gene therapy, | University of Michigan | | neoplasm | Chemoprotectant |
| University of Michigan | | | | |
| seraspenide | Ipsen-Beaufour | | neoplasm | Chemoprotectant |
| CRL-1605 | CytRx Corp | | carcinoma | Chemosensitizer |
| imidazoles, Ontogen | Ontogen Corp | | neoplasm | Chemosensitizer |
| HS-026 | Yonsei University | | neoplasm | Chemosensitizer |
| NLCQ-1 | Evanston Hospital Corp | U.S. Pat. No. | neoplasm | Chemosensitizer |
| | | 5,602,142 | | |
| OXi-104 | OXiGENE Inc | | colon tumor, neoplasm | Chemosensitizer |
| Sensamide | OXiGENE Inc | | lung tumor | Chemosensitizer |
| Indimacis 125 | Cis bio international | | ovary tumor | Chemosensitizer |
| CL-329753 | Wyeth-Ayerst Pharmaceuticals | | carcinoma | Chemosensitizer |
| | Inc | | | |
| B-9309-068 | Byk Gulden | | neoplasm | Chemosensitizer |
| O6-benzylguanine | National Cancer Institute | | brain tumor, colon tumor, | Chemosensitizer |
| | | | neoplasm, rectal tumor | |
| NCLPQ-1 | Evanston Hospital Corp | | neoplasm | Chemosensitizer |
| MCP-1 inhibitor, Teijin | Teijin Ltd | | neoplasm | Chemotactic factor |
| chemokines, Dompe | Dompe Farm Spa | | neoplasm | Chemotactic factor |
| LY-295501 | Eli Lilly & Co | EP 0 555 036 | neoplasm | Chloride channel blocker |
| clotrimazole and analogs, | Sheffield Pharmaceuticals Inc | | carcinoma, neoplasm | Chloride channel blocker |
| Sheffield/Imutec | | | | |
| human chorionic gonadotropin, | National Institutes of Health | | kaposis sarcoma, breast tumor, | Chorionic gonadotropin |
| NIH | | | prostate tumor, ovary tumor, | |
| | | | stomach tumor, nervous system | |
| | | | tumor | |
| chorionic gonadotropin, | Milkhaus Laboratory Inc | U.S. Pat. No. | neoplasm, leukemia | Chorionic gonadotropin |
| Milkhaus | | 5,610,136 | | |
| RheothRx | CytRx Corp | U.S. Pat. No. | ovary tumor | Coagulation inhibitor |
| | | 4,873,083 | | |
| Metastat | CollaGenex Pharmaceutical Inc | | neoplasm | Collagenase inhibitor |
| TIMP-2, Oncologix | Oncologix Inc | | neoplasm | Collagenase inhibitor |
| AG-3340 | Agouron Pharmaceuticals Inc | | lung tumor, neoplasm, prostate | Collagenase inhibitor |
| | | | tumor | |
| batimastat | British Biotech plc | WO 90/05719 | digestive system tumor, lung | Collagenase inhibitor |
| | | | tumor, ocular disease, ocular | |
| | | | tumor, pulmonary disease | |
| collagenase inhibitors, Research | Research Corp Technologies Inc | | neoplasm | Collagenase inhibitor |
| Corp Tech | | | | |
| SCA-proteins, Enzon | Enzon Inc | | neoplasm | Complement cascade modulator |
| lysonin | Imutran Ltd | | neoplasm | Complement cascade stimulator |
| cytokine promoter, Immunex | Immunex Corp | | neoplasm | CSF 1 agonist |
| filgrastim | Amgen Inc | EP 0 396 158 | breast tumor, carcinoma, | CSF 1 agonist |
| | | | leukemia, ovary tumor | |
| sargramostim | Immunex Corp | | melanoma | CSF 1 agonist |
| M-CSF, Genetics | Genetics Institute Inc | | carcinoma, neoplasm | CSF 1 agonist |
| Institute/SciGenics | | | | |
| stem cell factor, Amgen | Amgen Inc | | breast tumor, lymphoma, | CSF 1 agonist |
| | | | myeloproliferative disorder, non- | |
| | | | Hodgkin's lymphoma | |
| TP-72 | Dartmouth Medical School | | neoplasm | Cyclooxygenase 2 inhibitor |
| PD-136005 | Parke-Davis & Co | | carcinoma, leukemia | Cyclooxygenase inhibitor |
| F-18 labelled steroids, Univ of | University of Illinois | | breast tumor, prostate tumor | Cyclooxygenase inhibitor |
| Illinois | | | | |
| cathepsin inhibitors, Arris | Arris Pharmaceutical Corp | | carcinoma | Cysteine protease inhibitor |
| K-11002 | Khepri Pharmaceuticals | | neoplasm | Cysteine protease inhibitor |
| CPIs, British Biotech/SynPhar | Synphar Laboratories Inc | | neoplasm | Cysteine protease inhibitor |
| growth factor modulators, | Regeneron Pharmaceuticals Inc | | neoplasm | Cytokine agonist |
| Regeneron/Pharmacopeia | | | | |
| Multikine | CEL-SCI Corp | EP 0 049 611 | head & neck tumor, prostate | Cytokine agonist |
| | | | tumor, neoplasm | |
| recombinant prolactin, Genzyme | Genzyme Corp | | carcinoma, vaccination | Cytokine agonist |
| promegapoietin | Searle & Co | | neoplasm, thrombocytopenia | Cytokine agonist |
| daniplestim | Searle & Co | | neoplasm | Cytokine agonist |
| SRL-172 | Stanford Rook Holdings plc | | carcinoma, lung tumor | Cytokine agonist |
| | | | melanoma, ovary tumor, prostate | |
| | | | tumor, uterine cervix tumor | |
| growth factor modulators, | Regeneron Pharmaceuticals Inc | | neoplasm | Cytokine antagonist |
| Regeneron/Pharmacopeia | | | | |
| Multikine | CEL-SCI Corp | EP 0 049 611 | head & neck tumor, prostate | Cytokine ligand |
| | | | tumor, neoplasm | |
| 7-thia-8-oxoguanosine | ICN Pharmaceuticals Inc | | carcinoma | Cytokine modulator |
| CNI-1493 | Picower Institute for Medical | | neoplasm | Cytokine release inhibitor |
| | Research | | | |
| CNI-1493 | Picower Institute for Medical | | neoplasm | Cytokine release inhibitor |
| | Research | | | |
| SB-220025 | SmithKline Beecham | | neoplasm | Cytokine synthesis inhibitor |
| | Pharmaceuticals | | | |
| gene therapy (cancer), | GeneMedicine Inc | | head & neck tumor, melanoma | Cytokine synthesis modulator |
| GeneMedicine/Boehringer | | | | |
| KF-20444 | Kyowa Hakko Kogyo Co Ltd | | carcinoma | Dehydrogenase inhibitor |
| brequinar | DuPont Pharmaceuticals Co | EP 0 133 244 | carcinoma, neoplasm | Dehydrogenase inhibitor |
| Onco-TCS (vincristine), Inex | Inex Pharmaceuticals Corp | | pancreas tumor, colorectal | Delivery system |
| | | | tumor, lymphoma | |
| dendrimer gene delivery, UL | University of London | | neoplasm | Delivery system |
| School of Pharmacy | | | | |
| LY-295248 | Eli Lilly & Co | | neoplasm | DHFR inhibitor |
| LY-231514 | Eli Lilly & Co | EP 0 432 677 | breast tumor, carcinoma, | DHFR inhibitor |
| | | | colorectal tumor, lung tumor, | |
| | | | pancreas tumor | |
| edatrexate | SRI International | FR 2 464 956 | carcinoma, lung tumor, | DHFR inhibitor |
| | | | neoplasm | |
| LY-335738 | Eli Lilly & Co | | leukemia, neoplasm | DHFR inhibitor |
| trimetrexate | Warner-Lambert Co | U.S. Pat. No. | carcinoma, colorectal tumor, | DHFR inhibitor |
| | | 4,391,809 | neoplasm, stomach tumor | |
| LY-335518 | Eli Lilly & Co | | leukemia, neoplasm | DHFR inhibitor |
| LY-298207 | Eli Lilly & Co | WO 95/09845 | neoplasm | DHFR inhibitor |
| LY-316373 | Eli Lilly & Co | WO 95/09845 | neoplasm | DHFR inhibitor |
| LY-288784 | Eli Lilly & Co | | neoplasm | DHFR inhibitor |
| TNP-351 | Takeda Chemical Industries Ltd | U.S. Pat. No. | carcinoma | DHFR inhibitor |
| | | 4,997,838 | | |
| piritrexim | Burroughs Wellcome Inc | EP 0 021 292 | carcinoma, bladder tumor, head | DHFR inhibitor |
| | | | & neck tumor, kaposis sarcoma | |
| MDAM, BioNumerik/Johns | Bionumerik Pharmaceuticals Inc | | carcinoma | DHFR inhibitor |
| Hopkins | | | | |
| antifolates, University of | University of Newcastle | | neoplasm | DHFR inhibitor |
| Newcastle | | | | |
| LY-335580 | Eli Lilly & Co | | leukemia, neoplasm | DHFR inhibitor |
| 1954U89 | Glaxo Wellcome plc | | solid tumor | DHFR inhibitor |
| AG-350 | Agouron Pharmaceuticals Inc | | neoplasm | DHFR inhibitor |
| AG-384 | Agouron Pharmaceuticals Inc | | neoplasm | DHFR inhibitor |
| AG-394 | Agouron Pharmaceuticals Inc | | neoplasm | DHFR inhibitor |
| E-7010 | Eisai Co Ltd | EP 0 472 053 | carcinoma | Dihydropteroate |
| | | | | pyrophosphorylase inhibitor |
| S-1 | Taiho Pharmaceutical Co Ltd | | breast tumor, lung tumor, head | Dihydropyrimidine |
| | | | & neck tumor, neoplasm, | dehydrogenase inhibitor |
| | | | digestive system tumor | |
| 776C85 | Glaxo Wellcome plc | | neoplasm, colon tumor, breast | Dihydropyrimidine |
| | | | tumor, prostate tumor, pancreas | dehydrogenase inhibitor |
| | | | tumor | |
| 7U85 | Burroughs Wellcome Inc | WO 91/14688 | carcinoma | DNA gyrase inhibitor |
| 773U82 | Burroughs Wellcome Inc | EP 0 125 702 | carcinoma, pancreas tumor | DNA gyrase inhibitor |
| TLC-D-99 | The Liposome Company Inc | | breast tumor, carcinoma, kaposis | DNA gyrase inhibitor |
| | | | sarcoma | |
| iododoxorubicin | Pharmacia & Upjohn AB | BE 0 892 943 | breast tumor, carcinoma, lung | DNA gyrase inhibitor |
| | | | tumor | |
| teloxantrone | Parke-Davis & Co | | carcinoma, neoplasm | DNA gyrase inhibitor |
| intoplicine | Rhone-Poulenc Rorer Inc | EP 0 402 232 | solid tumor | DNA gyrase inhibitor |
| Ro-23-7777 | Roche Holding AG | | carcinoma | DNA gyrase inhibitor |
| A-65281 | Abbott Laboratories | | neoplasm | DNA gyrase inhibitor |
| DNA gyrase inhibitors, R W | R W Johnson Pharmaceutical | | neoplasm | DNA gyrase inhibitor |
| Johnson | Research Institute | | | |
| WIN-33377 | Sterling Winthrop Products Inc | | solid tumor | DNA intercalator |
| doxorubicin (liposome- | NeoPharm Inc | | breast tumor, kaposis sarcoma, | DNA intercalator |
| encapsulated), NeoPharm | | | ovary tumor, prostate tumor, | |
| | | | solid tumor | |
| NSC-655649 | University of Wisconsin, | | neoplasm | DNA intercalator |
| | Madison | | | |
| antineoplaston A10 | Burzynski Research Institute | | brain tumor, breast tumor, | DNA intercalator |
| | | | carcinoma | |
| BBR-2828 | Boehringer Mannheim GmbH | | neoplasm | DNA intercalator |
| datelliptium chloride | Elf Sanofi | EP 0 209 511 | neoplasm, breast tumor | DNA intercalator |
| idoxuridine, NeoPharm | National Cancer Institute | | sarcoma, renal tumor, pancreas | DNA intercalator |
| | | | tumor | |
| FCE-27726 | Pharmacia & Upjohn SpA | | neoplasm | DNA intercalator |
| UCT-1072 | Kyowa Hakko Kogyo Co Ltd | WO 97/29099 | neoplasm | DNA intercalator |
| BBR-2778 | Boehringer Mannheim GmbH | | leukemia, lymphoma | DNA intercalator |
| TMTA | University of Padova | | neoplasm | DNA intercalator |
| CI-958 | Parke-Davis & Co | EP 0 172 632 | carcinoma, prostate tumor, solid | DNA intercalator |
| | | | tumor | |
| IT-62-B | Taiho Pharmaceutical Co Ltd | | neoplasm | DNA intercalator |
| WP-631 | University of Mississippi | | neoplasm | DNA intercalator |
| MEN-10746 | A Menarini Ind Farm Riunite | WO 95/09173 | neoplasm | DNA intercalator |
| | SrL | | | |
| antitumor agents, University of | University of Arizona | | neoplasm | DNA intercalator |
| Arizona | | | | |
| cisplatin analog, Granada/Sevilla | Universidad de Granada | | breast tumor, neoplasm, ovary | DNA intercalator |
| | | | tumor | |
| crisnatol | Glaxo Wellcome plc | EP 0 125 702 | brain tumor, carcinoma, | DNA intercalator |
| | | | neoplasm | |
| C-1027 | Taiho Pharmaceutical Co Ltd | | neoplasm | DNA intercalator |
| gold(III) complexes, Johnson | Johnson Matthey plc | | carcinoma, ovary tumor | DNA intercalator |
| Matthey | | | | |
| DNA intercalators, Chungbuk | Chungbuk National University | | neoplasm | DNA intercalator |
| Universit | | | | |
| doxorubicin analogs, Menarini | A Menarini Ind Farm Riunite | | neoplasm | DNA intercalator |
| Ricerche | SrL | | | |
| resveratrol, University of Illinois | University of Illinois | | breast tumor, neoplasm | DNA modulator |
| MEN-10710 | Menarini Ltd | | neoplasm | DNA modulator |
| ET-722 | University of Illinois | | leukemia, non-Hodgkin's | DNA modulator |
| | | | lymphoma | |
| leinamycin analogs, Kyowa | Kyowa Hakko Kogyo Co Ltd | | neoplasm | DNA modulator |
| ET-743 | University of Illinois | | breast tumor, carcinoma, lung | DNA modulator |
| | | | tumor, melanoma | |
| antitumor agents, Boehringer | Boehringer Mannheim Italia | | carcinoma | DNA modulator |
| Mannheim | SPA | | | |
| antineoplaston-A5 | Burzynski Research Institute | | carcinoma | DNA modulator |
| antineoplaston-A3 | Burzynski Research Institute | | carcinoma | DNA modulator |
| antitumor agents, National | National Cancer Institute | | carcinoma | DNA modulator |
| Cancer Institute | | | | |
| ET-729 | University of Illinois | | breast tumor, carcinoma, lung | DNA modulator |
| | | | tumor, melanoma | |
| colon cancer therapy, NCI | National Cancer Institute | | colon tumor | DNA modulator |
| 7U85 | Burroughs Wellcome Inc | WO 91/14688 | carcinoma | DNA polymerase inhibitor |
| 773U82 | Burroughs Wellcome Inc | EP 0 125 702 | carcinoma, pancreas tumor | DNA polymerase inhibitor |
| lamivudine | BioChem Pharma Inc | EP 0 382 526 | | DNA polymerase inhibitor |
| retelliptine | Elf Sanofi | EP 0 010 029 | carcinoma | DNA polymerase inhibitor |
| KM-043 | Toyo Pharmaceutical Co Ltd | | neoplasm | DNA polymerase inhibitor |
| epelmycin A | Fujisawa Pharmaceutical Co Ltd | | carcinoma | DNA polymerase inhibitor |
| aphidicolin glycinate | Zeneca Group Plc | JP 59-088438 | carcinoma | DNA polymerase inhibitor |
| Super-LEU-DOX | Coulter Pharmaceutical Inc | | breast tumor, neoplasm, ovary | DNA polymerase inhibitor |
| | | | tumor, prostate tumor | |
| KN-208 | Nagoya University | | digestive system tumor | DNA polymerase inhibitor |
| G-3139 | Genta Inc | | breast tumor, colon tumor, | DNA RNA polymerase inhibitor |
| | | | leukemia, lymphoma, | |
| | | | melanoma, neoplasm, non- | |
| | | | Hodgkin's lymphoma, prostate | |
| | | | tumor, solid tumor | |
| BE-14348B | Banyu Pharmaceutical Co Ltd | | carcinoma, neoplasm | DNA RNA polymerase inhibitor |
| MGI-114 | MGI Pharma Inc | | breast tumor, carcinoma, colon | DNA synthesis inhibitor |
| | | | tumor, lung tumor, neoplasm, | |
| | | | ovary tumor, uterine cervix | |
| | | | tumor | |
| doxorubicin (liposome- | NeoPharm Inc | | breast tumor, kaposis sarcoma, | DNA synthesis inhibitor |
| encapsulated), NeoPharm | | | ovary tumor, prostate tumor, | |
| | | | solid tumor | |
| LY-296329 | Eli Lilly & Co | | neoplasm | DNA synthesis inhibitor |
| fludarabine | Southern Research Inst | | leukemia, lymphoma, non- | DNA synthesis inhibitor |
| | | | Hodgkin's lymphoma | |
| antitumor nucleosides, Hokkaido | Hokkaido University | | neoplasm | DNA synthesis inhibitor |
| University | | | | |
| LY-309887 | Eli Lilly & Co | | carcinoma, neoplasm | DNA synthesis inhibitor |
| lometrexol | Eli Lilly & Co | EP 0 248 573 | carcinoma, neoplasm | DNA synthesis inhibitor |
| aclacinomycin | Il Dong Pharm Co Ltd | | carcinoma | DNA synthesis inhibitor |
| LY-223592 | Eli Lilly & Co | | carcinoma, neoplasm | DNA synthesis inhibitor |
| gemcitabine | Eli Lilly & Co | GB 2 136 425 | lung tumor, pancreas tumor, | DNA synthesis inhibitor |
| | | | carcinoma, uterine cervix tumor, | |
| | | | bladder tumor, urinary tract | |
| | | | tumor, breast tumor, renal | |
| | | | tumor, neoplasm, head & | |
| | | | neck tumor | |
| LY-254155 | Eli Lilly & Co | | carcinoma, neoplasm | DNA synthesis inhibitor |
| LY-297950 | Eli Lilly & Co | | neoplasm | DNA synthesis inhibitor |
| tiricibine analogs, Univ | University of Michigan | | neoplasm | DNA synthesis inhibitor |
| Michigan | | | | |
| decitabine | Eli Lilly GmbH | | leukemia, lung tumor, myeloid | DNA synthesis inhibitor |
| | | | leukemia, prostate tumor | |
| anticancer, Biota/La Trobe | La Trobe University | | colon tumor, lung tumor, | DNA synthesis inhibitor |
| | | | stomach tumor | |
| aphidicolin glycinate | Zeneca Group Plc | JP 59-088438 | carcinoma | DNA synthesis inhibitor |
| mitonafide | BASF AG | | carcinoma | DNA synthesis inhibitor |
| diaziquone | National Institutes of Health | | brain tumor, carcinoma, glioma, | DNA synthesis inhibitor |
| | | | leukemia | |
| Adenazole | ICN Pharmaceuticals Inc | | leukemia, neoplasm | DNA synthesis inhibitor |
| epelmycin A | Fujisawa Pharmaceutical Co Ltd | | carcinoma | DNA synthesis inhibitor |
| adozelesin | Pharmacia & Upjohn Co | | breast tumor, carcinoma, | DNA synthesis inhibitor |
| | | | leukemia, neoplasm, solid tumor | |
| ecomustine | Choay SA | WO 85/01050 | carcinoma | DNA synthesis inhibitor |
| enloplatin | American Cyanamid Co | EP 0 232 784 | carcinoma | DNA synthesis inhibitor |
| tallimustine | Pharmacia & Upjohn AB | EP 0 246 868 | leukemia, solid tumor | DNA synthesis inhibitor |
| FCE-26605 | Farmitalia Carlo Erba SpA | WO 91/10649 | carcinoma | DNA synthesis inhibitor |
| FCE-26752 | Farmitalia Carlo Erba SpA | | carcinoma | DNA synthesis inhibitor |
| galamustine | Unimed Pharmaceuticals Inc | | carcinoma | DNA synthesis inhibitor |
| iproplatin | Johnson Matthey plc | | carcinoma | DNA synthesis inhibitor |
| JM-216 | Johnson Matthey plc | EP 0 328 274 | carcinoma, lung tumor, ovary | DNA synthesis inhibitor |
| | | | tumor, prostate tumor | |
| miboplatin | Chugai Pharmaceutical Co Ltd | EP 0 176 005 | carcinoma, ovary tumor, prostate | DNA synthesis inhibitor |
| | | | tumor | |
| nedaplatin | Shionogi & Co Ltd | JP 59-222497 | carcinoma | DNA synthesis inhibitor |
| sebriplatin | Nippon Kayaku Co Ltd | EP 0 219 936 | carcinoma, neoplasm | DNA synthesis inhibitor |
| ormaplatin | Pharmacia & Upjohn Co | | carcinoma, leukemia, solid | DNA synthesis inhibitor |
| | | | tumor | |
| temozolomide | The University of Aston In | DE 3231255 | carcinoma, glioma, melanoma, | DNA synthesis inhibitor |
| | Birmingham | | metastasis | |
| JM-221 | Johnson Matthey plc | | neoplasm | DNA synthesis inhibitor |
| etopophos | Bristol-Myers Squibb Co | U.S. Pat. No. | carcinoma, kaposis sarcoma, | DNA synthesis inhibitor |
| | | 5,041,424 | lung tumor, lymphoma, prostate | |
| | | | tumor | |
| FCE-26492 | Farmitalia Carlo Erba SpA | | carcinoma | DNA synthesis inhibitor |
| losoxantrone | Parke-Davis & Co | EP 0 103 381 | breast tumor, neoplasm | DNA synthesis inhibitor |
| antineoplaston AS2-5 | Burzynski Research Institute | | carcinoma | DNA synthesis inhibitor |
| azamitosenes, Vital | National Cancer Institute | | neoplasm | DNA synthesis inhibitor |
| Pharmaceutical Del | | | | |
| herboxidiene | Takeda Chemical Industries Ltd | | neoplasm | DNA synthesis inhibitor |
| antineoplaston AS2-1 | Burzynski Research Institute | | carcinoma | DNA synthesis inhibitor |
| Ro-24-5531 | Roche Holding AG | EP 0 580 968 | neoplasm | DNA synthesis inhibitor |
| NSC-361456 | National Institutes of Health | EP 0 182 277 | neoplasm | DNA synthesis inhibitor |
| KW-2149 | Kyowa Hakko Kogyo Co Ltd | | neoplasm | DNA synthesis inhibitor |
| XB-596 | DuPont Pharmaceuticals Co | | neoplasm | DNA synthesis inhibitor |
| nucleoside (anticancer), Yale | Yale University | | neoplasm | DNA synthesis modulator |
| University | | | | |
| vaccine (DNA), Pasteur Merieux | Pasteur Merieux Connaught | | carcinoma | DNA vaccine |
| Connaught | | | | |
| MEN-10710 | Menarini Ltd | | neoplasm | DNase modulator |
| bromocriptine, Novartis | Novartis AG | | breast tumor, colorectal tumor, | Dopamine D2 agonist |
| | | | glioma, head & neck tumor, | |
| | | | lung tumor, non-Hodgkin's | |
| | | | lymphoma, pancreas tumor | |
| P-glycoprotein inhibitors, | Dartmouth Medical School | | neoplasm | Drug metabolism modulator |
| Dartmouth College | | | | |
| HYB-241 | Hybritech Cancer Research Inc | | carcinoma | Drug metabolism modulator |
| VEGF inhibitor, Agouron | Agouron Pharmaceuticals Inc | | angiogenesis disorders, | EGF antagonist |
| | | | carcinoma | |
| GEM-220 | Hybridon Inc | WO 96/27006 | neoplasm | EGF antagonist |
| AR-639 | Aronex Pharmaceuticals Inc | | liver tumor, neoplasm, renal | EGF antagonist |
| | | | tumor | |
| MDX-447 | Merck KGaA | | carcinoma, head & neck tumor, | EGF antagonist |
| | | | prostate tumor | |
| MDX-260 | Medarex Inc | | glioma, melanoma, nervous | EGF antagonist |
| | | | system tumor | |
| DAB-720 | Mitsubishi Chemical Corp | | neoplasm | EGF binding agent |
| HER-2 antagonist, Sugen/Asta | Sugen Inc | | breast tumor, lung tumor, ovary | EGF binding agent |
| | | | tumor, prostate tumor, stomach | |
| | | | tumor | |
| VRCTC-310 | Ventech Research | | neoplasm | EGF binding agent |
| MR1scFvPE38KDEL, NCI | National Cancer Institute | | neoplasm | EGF binding agent |
| ABX-EGF | Abgenix Inc | | neoplasm | EGF binding agent |
| EMD-55900 | Merck KGaA | | carcinoma, glioma | EGF binding agent |
| EMD-72000 | Merck KGaA | | carcinoma | EGF binding agent |
| EGF fusion toxin, Seragen | Seragen Inc | | solid tumor, psoriasis, | EGF binding agent |
| | | | restenosis, carcinoma, | |
| | | | lung tumor | |
| OLX-103 | Merck & Co Inc | | bladder tumor | EGF binding agent |
| SELEX | NeXstar Pharmaceuticals Inc | U.S. Pat. No. | neoplasm | Elastase inhibitor |
| | | 5,270,163 | | |
| acetogenins, Purdue | Purdue University | | neoplasm | Electron transport inhibitor |
| rollimembrin | University of Valencia | | neoplasm | Electron transport inhibitor |
| polyalthidin, Valencia | University of Valencia | | neoplasm | Electron transport inhibitor |
| CGP-62706 | Novartis AG | | neoplasm | Endothelial growth factor |
| | | | | antagonist |
| SU-5271 | Zeneca Group Plc | | psoriasis, neoplasm | Endothelial growth factor |
| | | | | antagonist |
| NX-278-L | NeXstar Pharmaceuticals Inc | WO 96/27604 | angiogenesis disorder, kaposis | Endothelial growth factor |
| | | | sarcoma | antagonist |
| metalloprotease inhibitor, | Glycomed Inc | | neoplasm | Endothelin converting enzyme |
| Glycomed | | | | inhibitor |
| RILON | VimRx Pharmaceuticals Inc | | carcinoma | Enzyme |
| MG-341 | ProScript Inc | | carcinoma | Enzyme inhibitor |
| furin inhibitors, Tsukuba | University of Tsukuba | | carcinoma | Enzyme inhibitor |
| University | | | | |
| kinase inhibitors, Kinetek | Kinetek Pharmaceuticals Inc | | neoplasm | Enzyme inhibitor |
| therapeutics, Arris/Abbott | Arris Pharmaceutical Corp | | neoplasm | Enzyme inhibitor |
| CDK inhibitors, Institut Curie | Institut Curie | | neoplasm | Enzyme inhibitor |
| SF4 | Meiji Seika Kaisha Ltd | | carcinoma | Enzyme inhibitor |
| EGF fusion protein, Seragen | Seragen Inc | | solid tumor | Epidermal growth factor |
| Amphiregulin | Bristol-Myers Squibb Co | | carcinoma | Epidermal growth factor |
| SU-5271 | Zeneca Group Plc | | neoplasm | Epidermal growth factor |
| | | | | antagonist |
| CGP-52411 | Novartis AG | EP 0 516 588 | neoplasm | Epidermal growth factor |
| | | | | antagonist |
| AG-1478 | University of California-San | | neoplasm | Epidermal growth factor |
| | Diego Medical Center | | | antagonist |
| RC-3940-II | Pharmacia & Upjohn Inc | | breast tumor, neoplasm | Epidermal growth factor |
| | | | | antagonist |
| argos | Medical Research Council | | carcinoma | Epidermal growth factor |
| | (MRC) | | | antagonist |
| CP-358774 | OSI Pharmaceuticals Inc | | carcinoma, angiogenesis | Epidermal growth factor |
| | | | disorder, non-Hodgkin's | antagonist |
| | | | lymphoma, head & neck tumor, | |
| | | | breast tumor, bladder tumor | |
| C225 | Imclone Systems Inc | | breast tumor, head & neck | Epidermal growth factor |
| | | | tumor, lung tumor, prostate | antagonist |
| | | | tumor, renal tumor | |
| hbEGF-toxin, Prizm | Prizm Pharmaceuticals Inc | | bladder tumor, carcinoma, ovary | Epidermal growth factor |
| | | | tumor | antagonist |
| MAb 4D5 | Genentech Inc | | breast tumor | Epidermal growth factor |
| | | | | antagonist |
| BBR-1611 | Boehringer Mannheim GmbH | | carcinoma | Epidermal growth factor |
| | | | | antagonist |
| PD-169450 | Parke-Davis & Co | | neoplasm | Epidermal growth factor |
| | | | | antagonist |
| reveromycin-A | Snow Brand Milk Products Co | | carcinoma, neoplasm | Epidermal growth factor |
| | Ltd | | | antagonist |
| RWJ-61718 | Johnson & Johnson | WO 96/40772 | neoplasm | Erythropoietin and modulators |
| TSH-01 | Teijin Ltd | | menopausal disorder, | Estradiol |
| | | | osteoporosis | |
| mifepristone | Roussel Uclaf SA | FR 2 497 807 | breast tumor | Estradiol 17 beta dehydrogenase |
| | | | | stimulator |
| estrogen agonists, Karo Bio | Karo Bio AB | | breast tumor, neoplasm | Estradiol agonist |
| 2-methoxyestradiol | Harvard University | | breast tumor | Estradiol agonist |
| TSH-01 | Teijin Ltd | | menopausal disorder, | Estrogen |
| | | | osteoporosis | |
| estrogen agonists, Karo Bio | Karo Bio AB | | breast tumor, neoplasm | Estrogen agonist |
| sex hormone agonist (tissue | Ligand Pharmaceuticals Inc | | carcinoma, hormone | Estrogen agonist |
| selective), Ligand | | | replacement therapy | |
| anti-estrogen, Schering-Plough | Schering-Plough Corp | | breast tumor | Estrogen agonist |
| panomifene | Egis Gyogyszergyar RT | | carcinoma | Estrogen agonist |
| ZK-119010 | Schering AG | DE 3821148 | carcinoma | Estrogen antagonist |
| LY-326315 | Eli Lilly & Co | | carcinoma, uterus tumor | Estrogen antagonist |
| LY-353381 | Eli Lilly & Co | EP 0 248 573 | breast tumor | Estrogen antagonist |
| BE-14348B | Banyu Pharmaceutical Co Ltd | | carcinoma, neoplasm | Estrogen antagonist |
| ICI-164384 | Zeneca Group Plc | | breast tumor | Estrogen antagonist |
| anastrozole | Zeneca Group Plc | EP 0 296 749 | breast tumor | Estrogen antagonist |
| tamoxifen methiodide, Pharmos | Pharmos Corp | | breast tumor | Estrogen antagonist |
| idoxifene analog, BTG | British Technology Group Plc | | breast tumor | Estrogen antagonist |
| ZK-164015 | Schering AG | | breast tumor | Estrogen antagonist |
| RU-58668 | Roussel Uclaf Corp | | breast tumor | Estrogen antagonist |
| RU-51625 | Roussel Uclaf Corp | | breast tumor | Estrogen antagonist |
| EM-139 | Universite Laval | | breast tumor | Estrogen antagonist |
| anti-estrogens, AVAX | Avax Technologies Inc | | neoplasm | Estrogen antagonist |
| EM-800 | Universite Laval | | breast tumor | Estrogen antagonist |
| droloxifene | Klinge Pharma GmbH | EP 0 054 168 | breast tumor | Estrogen antagonist |
| ZM-182780 | Zeneca Group Plc | EP 0 138 504 | breast tumor, uterus tumor | Estrogen antagonist |
| WS-7528 | Fujisawa Pharmaceutical Co Ltd | JP 02218676 | carcinoma | Estrogen antagonist |
| RU-39411 | Roussel Uclaf SA | | breast tumor | Estrogen antagonist |
| toremifene | Orion Corp Ltd | EP 0 095 875 | breast tumor | Estrogen antagonist |
| RU-45144 | Roussel Uclaf SA | EP 0 280 618 | neoplasm | Estrogen antagonist |
| R-1128B | Fujisawa Pharmaceutical Co Ltd | JP 03007244 | neoplasm | Estrogen antagonist |
| zindoxifene | Degussa AG | | breast tumor | Estrogen antagonist |
| MDL-103323 | Hoechst Marion Roussel Inc | | neoplasm | Estrogen antagonist |
| MDL-104890 | Hoechst Marion Roussel Inc | | neoplasm | Estrogen antagonist |
| MDL-104931 | Hoechst Marion Roussel Inc | | neoplasm | Estrogen antagonist |
| MDL-101906 | Marion Merrell Dow | | carcinoma | Estrogen antagonist |
| | Pharmaceuticals Inc | | | |
| idoxifene | British Technology Group Plc | | breast tumor | Estrogen antagonist |
| miproxifene phosphate | Taiho Pharmaceutical Co Ltd | | breast tumor | Estrogen antagonist |
| LY-353381 | Eli Lilly & Co | EP 0 248 573 | breast tumor | Estrogen modulator |
| LY-329146 | Eli Lilly & Co | | carcinoma | Estrogen modulator |
| estrogen agonists, Karo Bio | Karo Bio AB | | breast tumor, neoplasm | Estrogen modulator |
| raloxifene | Eli Lilly & Co | | colon tumor, neoplasm | Estrogen modulator |
| LY-326315 | Eli Lilly & Co | | carcinoma, uterus tumor | Estrogen modulator |
| GW-5638 | Glaxo Wellcome plc | | neoplasm | Estrogen modulator |
| LY-357489 | Eli Lilly & Co | EP 0 761 669 | breast tumor | Estrogen modulator |
| LY-355124 | Eli Lilly & Co | | carcinoma | Estrogen modulator |
| A-007 | Dekk-Tec Inc | | breast tumor, carcinoma, kaposis | Estrogen modulator |
| | | | sarcoma | |
| bFGF inhibitors, Genzyme Mol | Genzyme Molecular Oncology | | neoplasm | FGF |
| Oncology | | | | |
| aFGF-PE40 | Bristol-Myers Squibb Co | | carcinoma, neoplasm | FGF agonist |
| heparin-binding peptides, NIH | National Institutes of Health | WO 93/11156 | kaposis sarcoma, breast tumor, | FGF antagonist |
| | | | melanoma | |
| SELEX | NeXstar Pharmaceuticals Inc | U.S. Pat. No. | neoplasm | FGF antagonist |
| | | 5,270,163 | | |
| FCE-26644 | Pharmacia & Upjohn SpA | | neoplasm | FGF antagonist |
| FCE-27164 | Pharmacia & Upjohn SpA | | neoplasm | FGF antagonist |
| Pantarin | Prizm Pharmaceuticals Inc | WO 90/12597 | kaposis sarcoma, neoplasm | FGF antagonist |
| TBC-256 | Texas Biotechnology Corp | | neoplasm | FGF antagonist |
| GM-1474 | Glycomed Inc | | carcinoma, neoplasm | FGF antagonist |
| GMI-306 | Glycomed Inc | | neoplasm | FGF antagonist |
| 11A8-SAP | Chiron Corp | | neoplasm, melanoma, | FGF antagonist |
| | | | carcinoma, nervous system | |
| | | | tumor | |
| oligonucleotides (AIDS), NIH | National Institutes of Health | | kaposis sarcoma | FGF antagonist |
| LY-309887 | Eli Lilly & Co | | carcinoma, neoplasm | Folate antagonist |
| lometrexol | Eli Lilly & Co | EP 0 248 573 | carcinoma, neoplasm | Folate antagonist |
| anticancer therapy, Sloan- | Memorial Sloan-Kettering | WO 98/02163 | solid tumor, neoplasm | Folate antagonist |
| Kettering | Cancer Center Institute | | | |
| E-34335 | Eisai Co Ltd | WO 95/07276 | neoplasm | Folate antagonist |
| antifolates, Agouron | Agouron Pharmaceuticals Inc | | neoplasm | Folate antagonist |
| PT-523 | Dana Farber Cancer Institute Inc | | breast tumor, carcinoma, head & | Folate antagonist |
| | | | neck tumor, lung tumor | |
| LY-354899 | Eli Lilly & Co | | neoplasm | Folate modulator |
| brodimoprim | Helsinn | DE 2452889 | carcinoma, neoplasm | Folate synthesis inhibitor |
| fucosyltransferase inhibitors, | Ciba-Geigy Corp | | carcinoma | Fucosidase alpha modulator |
| Novartis | | | | |
| sulfircin analogs, Mitotix | Mitotix Inc | | carcinoma | Fungicide |
| lung cancer therapy, Cadus | Cadus Pharmaceutical Corp | | lung tumor | G Protein modulator |
| LY-309887 | Eli Lilly & Co | | carcinoma, neoplasm | GAR transformylase inhibitor |
| lometrexol | Eli Lilly & Co | EP 0 248 573 | carcinoma, neoplasm | GAR transformylase inhibitor |
| AG-2032 | Agouron Pharmaceuticals Inc | | carcinoma | GAR transformylase inhibitor |
| GAR transformylase inhibitor, | Scripps Research Institute | | neoplasm | GAR transformylase inhibitor |
| Scripps | | | | |
| AG-2034 | Agouron Pharmaceuticals Inc | | neoplasm | GAR transformylase inhibitor |
| GAR transformylase inhibitors, | Agouron Pharmaceuticals Inc | | neoplasm | GAR transformylase inhibitor |
| Agouron | | | | |
| GAR-Tfase, Wellcome | Glaxo Wellcome plc | | neoplasm | GAR transformylase inhibitor |
| JB-93182 | James Black Fdn Ltd | WO 95/04720 | neoplasm | Gastrin antagonist |
| CBS-5 | National Institutes of Health | | colon tumor | Gastrin antagonist |
| CR-2093 | Rotta Research Lab SpA | | intestine tumor, stomach tumor | Gastrin antagonist |
| cytokine promoter, Immunex | Immunex Corp | | neoplasm | GCSF |
| lenograstim | Chugai Pharmaceutical Co Ltd | | bladder tumor, breast tumor, | GCSF |
| | | | carcinoma, head & neck tumor, | |
| | | | leukemia | |
| filgrastim | Amgen Inc | EP 0 396 158 | breast tumor, carcinoma, | GCSF |
| | | | leukemia, ovary tumor | |
| GM-CSF tumor vaccine, | PowderJect Pharmaceuticals | | melanoma | GCSF |
| PowderJect | | | | |
| G-CSF agonist, Arris/Amgen | Arris Pharmaceutical Corp | | neoplasm | GCSF |
| ZD-6003 | Zeneca Group Plc | | carcinoma | GCSF |
| CDP-845 | Celltech Group plc | | breast tumor | Gelatinase inhibitor |
| Bay-12-9566 | Bayer AG | | breast tumor, colorectal tumor, | Gelatinase inhibitor |
| | | | metastasis | |
| gelatinase inhibitors, | Celltech Group plc | | carcinoma, neoplasm | Gelatinase inhibitor |
| Celltech/Zeneca | | | | |
| gelastatin AB, KRIBB | Korea Research Institute of | | metastasis, neoplasm | Gelatinase inhibitor |
| | Bioscience and Biotechnology | | | |
| AG-3340 | Agouron Pharmaceuticals Inc | | lung tumor, neoplasm, prostate | Gelatinase inhibitor |
| | | | tumor | |
| CT-1746 | Celltech Therapeutics Ltd | | colorectal tumor | Gelatinase inhibitor |
| remacemide | Astra Charnwood | EP 0 279 937 | cerebrovascular ischemia, | Glutamate antagonist |
| | | | epilepsy, huntingtons chorea, | |
| | | | alzheimers disease, parkinsons | |
| | | | disease | |
| etacrynic acid | Oncotech Inc | | carcinoma | Glutathione transferase inhibitor |
| oltipraz | Rhone-Poulenc SA | WO 94/16563 | neoplasm, prostate tumor | Glutathione transferase |
| | | | | stimulator |
| glycosidase inhibitors (HIV), | Cornell Research Foundation Inc | WO 93/02091 | neoplasm | Glycosidase inhibitor |
| Cornell | | | | |
| cancer vaccine, Geniva | PowderJect Vaccines | | melanoma, sarcoma, carcinoma, | GM-CSF |
| | | | breast tumor | |
| melanoma vaccine, Immunex | Immunex Corp | | melanoma | GM-CSF |
| sargramostim | Immunex Corp | | melanoma | GM-CSF |
| GM-CSF vaccine, Johns | Johns Hopkins University | | renal tumor | GM-CSF |
| Hopkins | | | | |
| GM-CSF, NPO Vector | NPO Vector | | neoplasm | GM-CSF |
| gene therapy (GM-CSF), Dana | Dana Farber Cancer Institute Inc | | neoplasm | GM-CSF |
| Farber | | | | |
| SDZ-62-406 | Novartis AG | | carcinoma, neoplasm | GM-CSF |
| SDZ-62-826 | Novartis AG | EP 0 213 082 | carcinoma, neoplasm | GM-CSF |
| Macrolin | Cetus Oncology Corp | | neoplasm | GM-CSF |
| Leucotropin | Paladin Labs Inc | EP 0 352 707 | breast tumor | GM-CSF |
| SC-68420 | G D Searle & Co Ltd | | carcinoma | GM-CSF agonist |
| GM-CSF vaccine, University of | University of Wisconsin, | | melanoma | GM-CSF agonist |
| Wisconsin | Madison | | | |
| E21R | Bresatec | | myeloid leukemia | GM-CSF antagonist |
| tryptorelin | Tulane University | | breast tumor, prostate tumor | GNRH agonist |
| nafarelin | Roche Bioscience | U.S. Pat. No. | breast tumor, prostate tumor | GNRH agonist |
| | | 4,234,571 | | |
| deslorelin | The Salk Institute | | prostate tumor | GNRH agonist |
| avorelin | Mediolanum Farmaceutici SpA | | neoplasm | GNRH agonist |
| ganirelix | Roche Bioscience | EP 0 312 052 | breast tumor, carcinoma, | GNRH antagonist |
| | | | prostate tumor | |
| cetrorelix | ASTA Medica AG | EP 0 299 402 | breast tumor, prostate tumor, | GNRH antagonist |
| | | | endometriosis, ovary tumor, | |
| | | | uterus tumor, carcinoma | |
| Gonadimmune | Aphton Corp | | breast tumor, prostate tumor, | GNRH antagonist |
| | | | uterus tumor | |
| D-21775 | ASTA Medica Arzneimittel Ges | | neoplasm | GNRH antagonist |
| | mbH | | | |
| growth factor modulators, | Regeneron Pharmaceuticals Inc | | neoplasm | Growth factor agonist |
| Regeneron/Pharmacopeia | | | | |
| IGF-1, Genentech | Genentech Inc | | neoplasm | Growth factor agonist |
| sonermin | Dainippon Pharmaceutical Co | | breast tumor, squamous cell | Growth factor agonist |
| | Ltd | | carcinoma, neoplasm | |
| lenograstim | Chugai Pharmaceutical Co Ltd | | bladder tumor, breast tumor, | Growth factor agonist |
| | | | carcinoma, head & neck tumor, | |
| | | | leukemia | |
| growth factor modulators, | Regeneron Pharmaceuticals Inc | | neoplasm | Growth factor antagonist |
| Regeneron/Pharmacopeia | | | | |
| octreotide | Novartis AG | EP 0 029 579 | breast tumor, carcinoma, | Growth factor antagonist |
| | | | endocrine tumor, pancreas tumor | |
| RC-3095 | Pharmacia & Upjohn AB | WO 92/09626 | neoplasm, prostate tumor | Growth factor antagonist |
| Neuropeptide receptor blocker, | Peptech Ltd | | lung tumor | Growth factor antagonist |
| Peptide Tech/ICRF | | | | |
| erbB-2 antisense, Duke/INEX | Duke University | | neoplasm | Growth factor antagonist |
| growth factor inhibitors, | RepliGen Corp | | angiogenesis disorder, neoplasm | Growth factor antagonist |
| Repligen/Pfizer | | | | |
| Angiozyme | Inex Pharmaceuticals Corp | | neoplasm | Growth factor antagonist |
| trastuzumab | Genentech Inc | | breast tumor, female genital tract | Growth factor antagonist |
| | | | tumor, ovary tumor | |
| blood growth factor, Oxford | Oxford Molecular Group plc | | neoplasm | Growth factors |
| Molecular/PolyMASC | | | | |
| hGH, OSI Pharmaceuticals | OSI Pharmaceuticals Inc | | cachexia | Growth hormone |
| ProLease delivery system | Alkermes Inc | | carcinoma, neoplasm | Growth hormone |
| IGF-1, Genentech | Genentech Inc | | neoplasm | Growth hormone agonist |
| growth hormone antagonist, | Sensus Drug Development Corp | | breast tumor | Growth hormone antagonist |
| Sensus | | | | |
| seglitide | Merck & Co Inc | | stomach tumor | Growth hormone releasing factor |
| | | | | antagonist |
| BIT | Glaxo Wellcome plc | | neoplasm | Guanylate cyclase inhibitor |
| Maxamine | Maxim Pharmaceuticals Inc | WO 91/04037 | leukemia, melanoma, myeloid | H2 agonist |
| | | | leukemia, myeloproliferative | |
| | | | disorder, neoplasm, renal tumor | |
| NAcSDKP analogs, CNRS | Centre National de la Recherche | | neoplasm | Hematopoietic inhibitor |
| | Scientifigue (CNRS) | | | |
| FLT-3 ligand, DNAX | DNAX Research Institute of | | carcinoma | Hematopoietic modulator |
| | Molecular & Cellular Biology | | | |
| | Inc | | | |
| Flt-3 ligand, Immunex | Immunex Corp | WO 94/28391 | carcinoma | Hematopoietic modulator |
| interleukin-6, Genetics | Genetics Institute Inc | | carcinoma | Hematopoietic stimulant |
| Institute/Novartis | | | | |
| interleukin-3, Genetics | Genetics Institute Inc | | bone marrow transplantation, | Hematopoietic stimulant |
| Institute/Sandoz | | | leukopenia, neoplasm, ovary | |
| | | | tumor, thrombocytopenia | |
| activin, Ajinomoto | Ajinomoto Co Inc | EP 0 210 461 | carcinoma | Hematopoietic stimulant |
| tucaresol | Glaxo Wellcome plc | EP 0 054 924 | melanoma | Hemoglobin modulator |
| PEG-hemoglobin, Enzon | Enzon Inc | WO 94/09027 | carcinoma | Hemoglobin modulator |
| heparin-binding peptides, NIH | National Institutes of Health | WO 93/11156 | Kaposi's sarcoma, breast tumor, | Heparin binding agent |
| | | | melanoma | |
| CH-271 | Takara Shuzo Co Ltd | | neoplasm | Heparin binding agent |
| XMP-300 | XOMA Corp | | angiogenesis disorder, neoplasm | Heparin modulator |
| GM-1603 | Glycomed Inc | | neoplasm, carcinoma | Heparin modulator |
| platelet factor 4, RepliGen | RepliGen Corp | WO 93/13794 | neoplasm, melanoma, colon | Heparin modulator |
| | | | tumor, sarcoma, kaposis | |
| | | | sarcoma, renal tumor, glioma | |
| PI-88 | Progen Industries Ltd | | neoplasm | Heparinase |
| A-72363C | Sankyo KK | | carcinoma | Heparinase inhibitor |
| FCE-26644 | Pharmacia & Upjohn SpA | | neoplasm | Hepatocyte growth factor |
| | | | | antagonist |
| FCE-27164 | Pharmacia & Upjohn SpA | | neoplasm | Hepatocyte growth factor |
| | | | | antagonist |
| FCE-27357A | Pharmacia & Upjohn SpA | | neoplasm | Hepatocyte growth factor |
| | | | | antagonist |
| DPPE, BMS | University of Manitoba | | prostate tumor, breast tumor | Histamine modulator |
| indinavir sulphate | Merck & Co Inc | EP 0 541 168 | | HIV protease inhibitor |
| lovastatin | Merck & Co Inc | | carcinoma, glioma, neoplasm | HMG CoA reductase inhibitor |
| TSH-01 | Teijin Ltd | | menopausal disorder, | Hormone |
| | | | osteoporosis | |
| Dival | Hedral Therapeutics Inc | | carcinoma | Hormone |
| salmon calcitonin, Cortecs | Cortecs Ltd | | osteoporosis, Paget's disease | Hormone |
| human chorionic gonadotropin, | Ares-Serono International SA | | kaposis sarcoma | Hormone |
| recombinant, Serono | | | | |
| D-3967 | Chiroscience Group plc | | breast tumor | Hormone |
| Solarase | Hyal Pharmaceutical Corp | WO 91/04058 | carcinoma | Hyaluronic acid ligand |
| HA oligosaccharides, Anika | Anika Therapeutics Inc | | neoplasm | Hyaluronic acid modulator |
| CB-7661 | Institute of Cancer Research, | | prostate tumor | Hydroxylase inhibitor |
| | UK | | | |
| P450-17-alpha inhibitor, ICR | Institute of Cancer Research, | | prostate tumor | Hydroxylase inhibitor |
| | UK | | | |
| P450 17 alpha inhibitor, | University of Maryland | | prostate tumor | Hydroxylase inhibitor |
| University of Maryland | | | | |
| abiraterone | British Technology Group Plc | GB 2 265 624 | prostate tumor | Hydroxylase inhibitor |
| VX-740 | Vertex Pharmaceuticals Inc | WO 95/35308 | metastasis | ICE inhibitor |
| interferon agonists, | Ligand Pharmaceuticals Inc | | carcinoma | IFN agonist |
| Ligand/Abbott | | | | |
| interferon, Tanox Biosystems | Tanox Biosystems Inc | | neoplasm | IFN agonist |
| interferon (liposomal), NPO | NPO Vector | | neoplasm | IFN agonist |
| Vector | | | | |
| Alferon N Gel | Interferon Sciences Inc | | precancer | IFN alpha |
| CGP-35269 | Novartis AG | | carcinoma | IFN alpha |
| Intron A | Enzon Inc | | bladder tumor, carcinoma, | IFN alpha |
| | | | melanoma, myeloid leukemia, | |
| | | | myeloproliferative disorder, | |
| | | | neoplasm, non-Hodgkin's | |
| | | | lymphoma | |
| interferon alpha-n1, Glaxo | Glaxo Wellcome plc | | neoplasm, leukemia, myeloid | IFN alpha |
| Wellcome | | | leukemia, renal tumor | |
| Alfaferone | Alfa Wassermann SpA | | kaposis sarcoma, leukemia | IFN alpha |
| interferon, Green Cross (alpha) | The Green Cross Corp | | neoplasm | IFN alpha |
| interferon, BioNative (alpha) | BioNative AB | | neoplasm | IFN alpha |
| SM-10500 | Sumitomo Pharmaceuticals Co | | carcinoma | IFN alpha |
| | Ltd | | | |
| Alferon N Injection | Interferon Sciences Inc | | kaposis sarcoma, lung tumor | IFN alpha |
| interferon, Cheil | Cheil Foods & Chem Inc | | neoplasm, sarcoma, leukemia | IFN alpha 2 |
| interferon, Roche (alpha-2a- | Roche Holding AG | | kaposis sarcoma, leukemia, liver | IFN alpha 2 |
| PEG) | | | tumor, lymphoma, myeloid | |
| | | | leukemia, neoplasm, non- | |
| | | | Hodgkin's lymphoma, renal | |
| | | | tumor | |
| Ro-22-8181 | Roche Holding AG | | neoplasm | IFN alpha 2 |
| Ro-25-3925 | Roche Holding AG | | neoplasm | IFN alpha 2 |
| Betaseron | Chiron Corp | EP 0 218 825 | carcinoma, sarcoma | IFN beta |
| interferon, Biogen (beta) | Biogen Inc | | glioma | IFN beta |
| interferon, Sclavo (beta) | Sclavo SpA | | neoplasm | IFN beta |
| recombinant interferon beta-1a, | Ares-Serono International SA | | neoplasm, glioma, colorectal | IFN beta |
| Serono | | | tumor, lung tumor | |
| interleukin-6 mutein, ImClone | Imclone Systems Inc | | carcinoma | IFN beta agonist |
| interferon, Ciba-Geigy (gamma) | Novartis AG | | carcinoma | IFN gamma |
| interferon, Suntory (gamma-1a) | Suntory Ltd | | neoplasm | IFN gamma |
| interferon gamma, Hayashibara | Hayashibara Co Ltd | | skin tumor | IFN gamma |
| interferon (gamma), Lucky | Lucky Ltd | | leukemia | IFN gamma |
| immunostimulants, Cephalon | Cephalon Inc | | neoplasm | IFN gamma agonist |
| HuIGIF | Hayashibara Co Ltd | | neoplasm | IFN gamma agonist |
| interferon, Boehringer Ingelheim | Boehringer Ingelheim Corp | | neoplasm, carcinoma | IFN omega |
| (omega) | | | | |
| interleukin-1, Cistron | Cistron Biotechnology | | neoplasm | IL-1 |
| gludapcin | Fujisawa Pharmaceutical Co Ltd | EP 0 025 842 | carcinoma | IL-1 agonist |
| PEG-Interleukin-1, Enzon | Enzon Inc | | carcinoma | IL-1 agonist, IL-1 alpha |
| interleukin-1 alpha, Immunex | Immunex Corp | | melanoma, thrombocytopenia | IL-1 alpha |
| SDZ-MRL-953 | Novartis AG | EP 0 309 411 | carcinoma | IL-1 antagonist |
| interleukin-1 receptor, Immunex | Immunex Corp | | neoplasm | IL-1 antagonist |
| TAN-2178 | Takeda Shokuhin Kogyo KK | JP 09012595 | carcinoma | IL-1 antagonist |
| Oct-43 | Otsuka Pharmaceutical Co Ltd | | mycosis fungoides | IL-1 beta |
| flezelastine | ASTA Medica AG | | neoplasm | IL-1 release inhibitor |
| flezelastine | ASTA Medica AG | | neoplasm | IL-1 synthesis inhibitor |
| Sch-52000 | Schering-Plough Corp | WO 93/02693 | crohns disease, solid tumor | IL-10 |
| interleukin-10 gene therapy | University of Pittsburgh | | neoplasm | IL-10 |
| Sch-52000 | Schering-Plough Corp | WO 93/02693 | crohns disease, solid tumor | IL-10 agonist |
| interleukin-12, Genetics Institute | Genetics Institute Inc | EP 0 433 827 | neoplasmrenal tumor | IL-12 |
| teceleukin | Biogen Inc | | leukemia, neoplasm | IL-12 |
| interleukin-12 gene therapy | University of Pittsburgh | | neoplasm | IL-12 |
| hIL13-PE38QQR, | National Institutes of Health | | neoplasm, renal tumor | IL-13 |
| NIH/NeoPharm | | | | |
| interleukin- 13, Elf Sanofi | Elf Sanofi | | neoplasm | IL-13 |
| interleukin-2, Immunex | Immunex Corp | | carcinoma, melanoma | IL-2 |
| interleukin-2, Roussel Uclaf | Roussel Uclaf SA | | carcinoma | IL-2 |
| celmoleukin | Takeda Chemical Industries Ltd | | carcinoma | IL-2 |
| interleukin-2, Amgen | Amgen Inc | WO 85/00817 | neoplasm | IL-2 |
| IL-2 fusion protein, Abbott | Abbott Laboratories | | neoplasm | IL-2 |
| aldesleukin | Chiron Therapeutics | EP 0 109 748 | renal tumor, melanoma, ovary | IL-2 |
| | | | tumor, lung tumor | |
| gene therapy (cancer), | Transgene SA | | melanoma, renal tumor, breast | IL-2 |
| Transgene | | | tumor, metastasis, digestive | |
| | | | system tumor, lung tumor, | |
| | | | colorectal tumor, neoplasm | |
| Avectin | Applied Immune Sciences Inc | | breast tumor, neoplasm | IL-2 |
| interleukin-2 gene therapy, | University of California | | colon tumor, melanoma, | IL-2 |
| UCLA | | | neoplasm | |
| interleukin-2 gene therapy, NIH | National Institutes of Health | | colon tumor, melanoma, renal | IL-2 |
| | | | tumor | |
| OncoLIPIN | OncoTherapeutics Inc | | carcinoma, renal tumor | IL-2 |
| PEG-interleukin-2, Chiron | Chiron Technologies | | head & neck tumor | IL-2 |
| IL-2, NPO Vector | NPO Vector | | neoplasm | IL-2 |
| DAB-486-IL-2 | Seragen Inc | | neoplasm | IL-2 |
| INGN-301 | University of Texas System | | neoplasm | IL-2 |
| gene therapy (IL-2), | McMaster University | | neoplasm, breast tumor, | IL-2 |
| McMaster/Baxter | | | melanoma | |
| BIWB-2 | Boehringer Ingelheim Corp | | neoplasm | IL-2 |
| interleukin-2 gene therapy, | Chiron Viagene Inc | | neoplasm | IL-2 |
| Chiron Viagene/Ajinomoto | | | | |
| denileukin diftitox | Seragen Inc | | head & neck tumor, lung tumor, | IL-2 |
| | | | lymphoma, non-Hodgkin's | |
| | | | lymphoma | |
| interleukin-2 gene therapy, | Transkaryotic Therapies Inc | EP 0 750 044 | renal tumor | IL-2 |
| Transkaryotic Therapies | | | | |
| Leuvectin | Vical Inc | | lymphoma, melanoma, | IL-2 |
| | | | neoplasm, prostate tumor, renal | |
| | | | tumor, sarcoma | |
| Multikine | CEL-SCI Corp | EP 0 049 611 | head & neck tumor, prostate | IL-2 agonist |
| | | | tumor, neoplasm | |
| interleukin-2, Amgen | Amgen Inc | WO 85/00817 | neoplasm | IL-2 agonist |
| gludapcin | Fujisawa Pharmaceutical Co Ltd | EP 0 025 842 | carcinoma | IL-2 agonist |
| aldesleukin | Chiron Therapeutics | EP 0 109 748 | renal tumor, melanoma, ovary | IL-2 agonist |
| | | | tumor, lung tumor | |
| interleukin-2 vaccine, ICR | Institute of Cancer Research, | | carcinoma | IL-2 agonist |
| | UK | | | |
| interleukin-2 vaccine, ICR | Institute of Cancer Research, | | carcinoma | IL-2 agonist |
| | UK | | | |
| IL-2 fusion protein, Abbott | Abbott Laboratories | | neoplasm | IL-2 agonist |
| interleukin-2, Immunex | Immunex Corp | | carcinoma, melanoma | IL-2 agonist |
| interleukin-2, Roussel Uclaf | Roussel Uclaf SA | | carcinoma | IL-2 agonist |
| celmoleukin | Takeda Chemical Industries Ltd | | carcinoma | IL-2 agonist |
| interleukin-2 gene therapy, St | St Jude Childrens Hospital | | nervous system tumor | IL-2 agonist |
| Jude | | | | |
| daclizumab | Protein Design Labs Inc | EP 0 451 216 | leukemia | IL-2 antagonist |
| IL-2 gene therapy (cancer), | Sidney Kimmel Cancer Center | | brain tumor, colon tumor | IL-2 synthesis modulator |
| Immune Response/SDRCC | | | | |
| roquinimex | Pharmacia & Upjohn AB | EP 0 059 698 | leukemia | IL-2 synthesis stimulant |
| interleukin-3, Genetics | Genetics Institute Inc | | bone marrow transplantation, | IL-3 |
| Institute/Sandoz | | | leukopenia, neoplasm, ovary | |
| | | | tumor, thrombocytopenia | |
| gene therapy (IL-3), IntroGene | IntroGene BV | | neoplasm | IL-3 |
| promegapoietin | Searle & Co | | neoplasm, thrombocytopenia | IL-3 agonist |
| daniplestim | Searle & Co | | neoplasm | IL-3 agonist |
| interleukin-3, Genetics | Genetics Institute Inc | | bone marrow transplantation, | IL-3 agonist |
| Institute/Sandoz | | | leukopenia, neoplasm, ovary | |
| | | | tumor, thrombocytopenia | |
| SC-68420 | G D Searle & Co Ltd | | carcinoma | IL-3 agonist |
| interleukin-3 synthokine | Searle & Co | | carcinoma | IL-3 agonist |
| interleukin-3 synthokine | Searle & Co | | carcinoma | IL-3 agonist |
| Allevorin | Paladin Labs Inc | | breast tumor | IL-3, IL-3 agonist |
| interleukin-3, Gist-Brocades | Royal Gist-Brocades NV | | carcinoma | IL-3, IL-3 agonist |
| anticancer therapy, | Eli Lilly & Co | | neoplasm | IL-4 |
| Lilly/Millennium | | | | |
| IL-4 gene therapy, Genetic | University of Pittsburgh | | breast tumor, colon tumor, | IL-4 |
| Therapy/Univ Pittsburgh | | | melanoma, renal tumor | |
| interleukin-4 fusion toxin, | Seragen Inc | | leukemia, lymphoma, neoplasm | IL-4 |
| Seragen | | | | |
| interleukin-4, Schering-Plough | Schering-Plough Corp | | digestive system tumor, | IL-4 |
| | | | leukemia, lung tumor, | |
| | | | lymphoma | |
| interleukin-4, Southwest | Texas Technical University | | renal tumor | IL-4 |
| Oncology | | | | |
| interleukin-4, Immunex | Immunex Corp | | carcinoma | IL-4 |
| IL-4 gene therapy, Genetic | University of Pittsburgh | | breast tumor, colon tumor, | IL-4 agonist |
| Therapy/Univ Pittsburgh | | | melanoma, renal tumor | |
| interleukin-4, Schering-Plough | Schering-Plough Corp | | digestive system tumor, | IL-4 agonist |
| | | | leukemia, lung tumor, | |
| | | | lymphoma | |
| interleukin-4, Southwest | Texas Technical University | | renal tumor | IL-4 agonist |
| Oncology | | | | |
| interleukin-4, Immunex | Immunex Corp | | carcinoma | IL-4 agonist |
| interleukin-6, American Home | American Home Products Corp | | neoplasm, carcinoma | IL-6 |
| Products | | | | |
| Betatropin | Paladin Labs Inc | | neoplasm | IL-6 |
| gludapcin | Fujisawa Pharmaceutical Co Ltd | EP- 0 025 842 | carcinoma | IL-6 agonist |
| interleukin-6, Genetics | Genetics Institute Inc | | carcinoma | IL-6 agonist |
| Institute/Novartis | | | | |
| cytokine promoter, Immunex | Immunex Corp | | neoplasm | IL-6 agonist |
| interleukin-6 mutein, ImClone | Imclone Systems Inc | | carcinoma | IL-6 agonist |
| Betatropin | Paladin Labs Inc | | neoplasm | IL-6 agonist |
| interleukin-6, Serono | Ares-Serono International SA | | leukemia, neoplasm, | IL-6 agonist |
| | | | thrombocytopenia | |
| madindoline, Kitasato Institute | Kitasato Institute | EP 0 787 733 | myeloproliferative disorder, | IL-6 antagonist |
| | | | neoplasm | |
| IL-6 antagonist, Regeneron | Regeneron Pharmaceuticals Inc | WO 95/11303 | myeloproliferative disorder, | IL-6 antagonist |
| | | | neoplasm | |
| antileukinate | University of Texas System | | neoplasm | IL-8 antagonist |
| interleukin-9, Genentech | Genentech Inc | | neoplasm | IL-9 |
| SDZ-MRL-953 | Novartis AG | EP 0 309 411 | carcinoma | IL antagonist |
| anticancer therapy, | Eli Lilly & Co | | neoplasm | IL synthesis modulator |
| Lilly/Millennium | | | | |
| leishmanial eukaryotic initiation | Corixa Corp | | neoplasm | IL synthesis modulator |
| factor, Corixa | | | | |
| roquinimex | Pharmacia & Upjohn AB | EP 0 059 698 | leukemia | Immunomodulator |
| Provax, IDEC | IDEC Pharmaceuticals Corp | | carcinoma, vaccination | Immunomodulator |
| anticancer therapy, | Eli Lilly & Co | | neoplasm | Immunomodulator |
| Lilly/Millennium | | | | |
| fucosyl-GM1-KLH, Sloan- | Memorial Sloan-Kettering | | lung tumor | Immunomodulator |
| Kettering | Cancer Center Institute | | | |
| DC-Cholesterol cationic lipid | RGene Therapeutics Inc | | vaccination, neoplasm | Immunomodulator |
| third generaion photosensitizers, | QLT PhotoTherapeutics Inc | | neoplasm | Immunomodulator |
| QLT | | | | |
| Ukrain | Ukranian Anti-Cancer Institute | | neoplasm | Immunomodulator |
| imexon | Amplimed Inc | | neoplasm, myeloproliferative | Immunomodulator |
| | | | disorder, lymphoma | |
| TRP-1/TRP-2, NIH | National Institutes of Health | | melanoma, neoplasm | Immunomodulator |
| Betaseron | Chiron Corp | EP 0 218 825 | carcinoma, sarcoma | Immunomodulator |
| Globo-H-KLH, Memorial Sloan- | Memorial Sloan-Kettering | | prostate tumor | Immunomodulator |
| Kettering | Cancer Center Institute | | | |
| T-cell modulators, ArQule/T | ArQule Inc | | neoplasm | Immunomodulator |
| Cell Sciences | | | | |
| immunomodulators, | Mycosearch Inc | | neoplasm | Immunomodulator |
| MYCOsearch/T Cell Sciences | | | | |
| LK-440 | Lek Pharmaceuticals | | neoplasm | Immunomodulator |
| VP22 technology, Marie | Marie Curie Cancer Care | | neoplasm | Immunomodulator |
| Curie/Phogen/Cantab | | | | |
| immunomodulators, | Massachusetts General Hospital | | neoplasm | Immunomodulator |
| Ergo/Massachusetts General | | | | |
| Hospital | | | | |
| mim 16.1, CDR Therapeutics | Xcyte Therapeutics Inc | | solid tumor, breast tumor, ovary | Immunomodulator |
| | | | tumor, pancreas tumor, prostate | |
| | | | tumor, lung tumor, bladder | |
| | | | tumor | |
| mim 4D5.1, CDR Therapeutics | Xcyte Therapeutics Inc | | solid tumor, prostate tumor, | Immunomodulator |
| | | | pancreas tumor, lung tumor, | |
| | | | ovary tumor, bladder tumor, | |
| | | | breast tumor | |
| immunomodulators, Antigen | Antigen Express Inc | U.S. Pat. No. | neoplasm | Immunomodulator |
| Express | | 5,559,028 | | |
| LEAPS technology (cancer), | CEL-SCI Corp | | prostate tumor, breast tumor | Immunomodulator |
| CEL-SCI | | | | |
| pentostatin | Warner-Lambert Co | | leukemia | Immunomodulator |
| immune modulator (HIV), | PharmaPrint Inc | | neoplasm | Immunomodulator |
| PharmaPrint | | | | |
| dendritic cell vaccine, | GeneMedicine Inc | | neoplasm | Immunomodulator |
| GeneMedicine/UT | | | | |
| TP3-PAP | Wayne Hughes Institute | | bone tumor | Immunomodulator |
| rituximab | IDEC Pharmaceuticals Corp | WO 94/11026 | lymphoma, non-Hodgkin's | Immunomodulator |
| | | | lymphoma | |
| daniplestim | Searle & Co | | neoplasm | Immunostimulant |
| Provax, IDEC | IDEC Pharmaceuticals Corp | | carcinoma, vaccination | Immunostimulant |
| gene therapy (IL-3), IntroGene | IntroGene BV | | neoplasm | Immunostimulant |
| roquinimex | Pharmacia & Upjohn AB | EP 0 059 698 | leukemia | Immunostimulant |
| gene therapy (melanoma), | Bender & Co Ges mbH | | melanoma | Immunostimulant |
| Bender | | | | |
| PDIT, Pacific | Pacific Pharmaceuticals Inc | | neoplasm, breast tumor, | Immunostimulant |
| | | | metastasis | |
| bropirimine | Pharmacia & Upjohn Inc | DE 3008693 | bladder tumor | Immunostimulant |
| HS-026 | Yonsei University | | neoplasm | Immunostimulant |
| promegapoietin | Searle & Co | | neoplasm, thrombocytopenia | Immunostimulant |
| KRN-7000 | Kirin Brewery Co Ltd | | neoplasm | Immunostimulant |
| BG-anti-TGF-beta, Hebrew | Hebrew University of Jerusalem | | neoplasm | Immunostimulant |
| University | | | | |
| metalloproteinase inhibitors, | Polifarma SpA | | carcinoma | Immunostimulant |
| Polifarma | | | | |
| MIF, Genetics Institute | Genetics Institute Inc | | neoplasm | Immunostimulant |
| DISC (cancer therapy), Cantab | Cantab Pharmaceuticals plc | WO 92/05263 | leukemia, neoplasm, colorectal | Immunostimulant |
| | | | tumor, stomach tumor, ovary | |
| | | | tumor, renal tumor, nervous | |
| | | | system tumor, parkinsons | |
| | | | disease | |
| GMK | Memorial Sloan-Kettering | | melanoma | Immunostimulant |
| | Cancer Center Institute | | | |
| TA-HPV | Cancer Research Campaign | | uterine cervix tumor | Immunostimulant |
| | Technology Ltd | | | |
| LP-2307 | Medical Biology Institute | WO 90/11085 | melanoma, neoplasm | Immunostimulant |
| gludapcin | Fujisawa Pharmaceutical Co Ltd | EP 0 025 842 | carcinoma | Immunostimulant |
| Multikine | CEL-SCI Corp | EP 0 049 611 | head & neck tumor, prostate | Immunostimulant |
| | | | tumor, neoplasm | |
| recombinant prolactin, Genzyme | Genzyme Corp | | carcinoma, vaccination | Immunostimulant |
| interleukin-12, Genetics Institute | Genetics Institute Inc | EP 0 433 827 | neoplasmrenal tumor | Immunostimulant |
| Org-6632 | Organon NV | | neoplasm | Immunostimulant |
| monoclonal antibodies (cancer), | A Menarini Ind Farm Riunite | | neoplasm | Immunostimulant |
| Menarini | SrL | | | |
| immunostimulants, Cephalon | Cephalon Inc | | neoplasm | Immunostimulant |
| Primuvant | Dovetail Technologies Inc | | carcinoma | Immunostimulant |
| CGP-19835 | Novartis AG | EP 0 056 560 | neoplasm, sarcoma | Immunostimulant |
| muramyl tripeptide, Ciba | Novartis AG | | carcinoma | Immunostimulant |
| QS-21 | Aquila Biopharmaceuticals Inc | WO 88/09336 | carcinoma, melanoma | Immunostimulant |
| Detox-B | Ribi ImmunoChem Research Inc | | breast tumor, carcinoma | Immunostimulant |
| ImmTher | Endorex Corp | | neoplasm, sarcoma, breast | Immunostimulant |
| | | | tumor, bone tumor | |
| MAK-BAb anticancer agents, | IDM Immuno-Designed | | ovary tumor, breast tumor, | Immunostimulant |
| IDM | Molecules | | prostate tumor, bladder tumor | |
| MMS-1 | SafeScience Inc | U.S. Pat. No. | neoplasm | Immunostimulant |
| | | 5,527,770 | | |
| fomitellan A | Korea Research Institute of | | neoplasm | Immunostimulant |
| | Bioscience and Biotechnology | | | |
| Theradigm-Melanoma | Cytel Corp | | melanoma, neoplasm, uterine | Immunostimulant |
| | | | cervix tumor | |
| DISC (cancer therapy), Cantab | Cantab Pharmaceuticals plc | WO 92/05263 | leukemia, neoplasm, colorectal | Immunostimulant |
| | | | tumor, stomach tumor, ovary | |
| | | | tumor, renal tumor, nervous | |
| | | | system tumor, parkinsons | |
| | | | disease | |
| SDZ-MRL-953 | Novartis AG | EP 0 309 411 | carcinoma | Immunostimulant |
| DNAM-1 | DNAX Research Institute of | | carcinoma | Immunostimulant |
| | Molecular & Cellular Biology | | | |
| | Inc | | | |
| SRI-62-834 | Novartis AG | | carcinoma | Immunostimulant |
| FLT-3 ligand, DNAX | DNAX Research Institute of | | carcinoma | Immunostimulant |
| | Molecular & Cellular Biology | | | |
| | Inc | | | |
| tucaresol | Glaxo Wellcome plc | EP 0 054 924 | melanoma | Immunostimulant |
| interleukin-2, Amgen | Amgen Inc | WO 8 500 817 | neoplasm | Immunostimulant |
| teceleukin | Biogen Inc | | leukemia, neoplasm | Immunostimulant |
| Betafectin | Alpha-Beta Technology Inc | | carcinoma | Immunostimulant |
| nitrullyn | Russian Academy Medical | | lung tumor | Immunostimulant |
| | Science | | | |
| SBAS2 | SmithKline Beecham plc | | carcinoma | Immunostimulant |
| HSPPC-96 | Mount Sinai School of Medicine | | carcinoma, colorectal tumor, | Immunostimulant |
| | | | imelanoma, neoplasm, pancreas | |
| | | | tumor, stomach tumor | |
| CERES-Vax vaccine delivery | Ceres Pharmaceuticals | | carcinoma | Immunostimulant |
| system | | | | |
| cancer vaccine, | Polymasc Pharmaceuticals plc | EP 0 727 438 | carcinoma | Immunostimulant |
| PolyMASC/Hydro Med | | | | |
| cancer vaccine, Cytel/Searle | Cytel Corp | | neoplasm | Immunostimulant |
| GVAX | Cell Genesys Inc | WO 92/05262 | colorectal tumor, lung tumor, | Immunostimulant |
| | | | melanoma, neoplasm, prostate | |
| | | | tumor, renal tumor | |
| neuroendocrine resetting | Ergo Science Corp | | neoplasm | Immunostimulant |
| therapy, Ergo | | | | |
| tumor-antigen-specific | Cellpro Inc | | carcinoma | Immunostimulant |
| lymphocytes, Corixa | | | | |
| BCH-1393 | BioChem Therapeutic Inc | | neoplasm | Immunostimulant |
| HPV E7 peptides, Cytel | Cytel Corp | | uterine cervix tumor | Immunostimulant |
| GM-1/P | Glaxo Wellcome plc | | neoplasm | Immunostimulant |
| KLH-Immune Activator | PerImmune Inc | | bladder tumor | Immunostimulant |
| BCG vaccine, Organon | Organon NV | | bladder tumor | Immunostimulant |
| Xenoject | SangStat Medical Corp | EP 0 510 949 | carcinoma, neoplasm | Immunostimulant |
| G-29 | Norvet Research Pty Ltd | | skin tumor | Immunostimulant |
| immunostimulant, | RepliGen Corp | | neoplasm | Immunostimulant |
| Repligen/Pfizer | | | | |
| MAK therapy, IDM | IDM Immuno-Designed | | melanoma, ovary tumor, lung | Immunostimulant |
| | Molecules | | tumor, colorectal tumor | |
| Theramide | Endorex Corp | | carcinoma | Immunostimulant |
| icadamine B | Cornell Research Foundation Inc | | neoplasm | Immunostimulant |
| MAK anticancer agents, IDM | IDM Immuno-Designed | | neoplasm, bladder tumor, ovary | Immunostimulant |
| | Molecules | | tumor, lung tumor, colorectal | |
| | | | tumor | |
| MAC-DC anticancer agents, | IDM Immuno-Designed | | ovary tumor, lung tumor, | Immunostimulant |
| IDM | Molecules | | bladder tumor, melanoma | |
| cell therapy (glioma), Neurotech | Neurotech SA | | glioma | Immunostimulant |
| gene therapy (non-viral), | Megabios Corp | | melanoma, solid tumor | Immunostimulant |
| Megabios | | | | |
| immunostimulants, CpG | CpG ImmunoPharmaceuticals | | neoplasm | Immunostimulant |
| ImmunoPharmaceuticals | Inc | | | |
| CD26 inhibitors, Point | Point Therapeutics Inc | | neoplasm | Immunostimulant |
| Therapeutics | | | | |
| CTLA-4 blockers, NeXstar | University of California | | neoplasm | Immunostimulant |
| antibody 1A7, University of | University of Kentucky | | melanoma | Immunostimulant |
| Kentucky | | | | |
| anti-GD2 antibody, Fuji | Fuji ImmunoPharmaceuticals Co | | neoplasm, nervous system | Immunostimulant |
| | Ltd | | tumor, melanoma | |
| ChL-6 | Bristol-Myers Squibb Co | | carcinoma, neoplasm | Immunostimulant |
| gp75 antigen, ImClone | Imclone Systems Inc | | carcinoma | Immunostimulant |
| humanized N901/CC-1065 | ImmunoGen Inc | | carcinoma | Immunostimulant |
| conjugate | | | | |
| ING-1 | XOMA Corp | | carcinoma | Immunostimulant |
| Lemonal | Yakult Honsha KK | | carcinoma | Immunostimulant |
| loxoribine | R W Johnson Pharmaceutical | | carcinoma | Immunostimulant |
| | Research Institute | | | |
| Specifid | IDEC Pharmaceuticals Corp | | carcinoma, lymphoma, non- | Immunostimulant |
| | | | Hodgkin's lymphoma | |
| NR-CO-02 | NeoRx Corp | | carcinoma | Immunostimulant |
| Rhenex | NeoRx Corp | | carcinoma | Immunostimulant |
| NR-LU-13 | NeoRx Corp | | colon tumor | Immunostimulant |
| TAb-250 | Berlex Laboratories Inc | | carcinoma | Immunostimulant |
| edrecolomab | Centocor Inc | | carcinoma, colon tumor, | Immunostimulant |
| | | | colorectal tumor, pancreas tumor | |
| RM-06 | Hoechst AG | WO 89/05818 | carcinoma | Immunostimulant |
| rubratin | Fujisawa Pharmaceutical Co Ltd | | carcinoma, neoplasm | Immunostimulant |
| SM3 | Cancer Therapeutics Ltd | | carcinoma | Immunostimulant |
| ST-789 | Sigma-Tau Ind Farm Riunite | EP 0 260 588 | carcinoma | Immunostimulant |
| | SpA | | | |
| TAN-999 | Takeda Chemical Industries Ltd | JP 01149791 | carcinoma | Immunostimulant |
| picibanil | Chugai Pharmaceutical Co Ltd | | carcinoma, benign tumor | Immunostimulant |
| CL-259763 | Lederle Laboratories | | neoplasm | Immunostimulant |
| levamisole | Janssen Pharmaceutica NV | | neoplasm, colon tumor, rectal | Immunostimulant |
| | | | tumor | |
| romurtide | Daiichi Seiyaku Co Ltd | EP 0 021 367 | neoplasm | Immunostimulant |
| tiprotimod | Hoechst AG | DE 3508665 | breast tumor, lung tumor, | Immunostimulant |
| | | | melanoma | |
| SU-201 | Sugen Inc | | neoplasm | Immunostimulant |
| SPR-901 | Sapporo Breweries Ltd | | neoplasm | Immunostimulant |
| LK-409 | Lek Pharmaceuticals | | neoplasm | Immunostimulant |
| MELIMMUNE | IDEC Pharmaceuticals Corp | | melanoma, neoplasm | Immunostimulant |
| gp53, Imclone | Imclone Systems Inc | | neoplasm | Immunostimulant |
| Oncopurge | NeoRx Corp | | neoplasm | Immunostimulant |
| SMART M195 | Protein Design Labs Inc | | leukemia, myeloid leukemia | Immunostimulant |
| MAb32, Cambridge Antibody | Cambridge Antibody | | neoplasm | Immunostimulant |
| Technology | Technology Ltd | | | |
| T-cell therapy (cancer), Cell | Cell Genesys Inc | WO 92/10591 | breast tumor, carcinoma, colon | Immunostimulant |
| Genesys | | | tumor, lung tumor, prostate | |
| | | | tumor | |
| MDX-210 | Medarex Inc | | breast tumor, carcinoma, colon | Immunostimulant |
| | | | tumor, colorectal tumor, lung | |
| | | | tumor, ovary tumor, pancreas | |
| | | | tumor, prostate tumor, renal | |
| | | | tumor | |
| S-27609 | Minnesota Mining & | | neoplasm | Immunostimulant |
| | Manufacturing Co | | | |
| thymosin alpha 1 | Alpha 1 Biomedicals Inc | | angiogenesis disorder, | Immunostimulant |
| | | | carcinoma, lung tumor, | |
| | | | melanoma, neoplasm | |
| Theradigm-HPV | Cytel Corp | | carcinoma, uterine cervix tumor | Immunostimulant |
| Theradigm-prostate | Cytel Corp | | prostate tumor | Immunostimulant |
| Virulizin | Imutec Pharma Inc | WO 95/07089 | kaposis sarcoma, lung tumor, | Immunostimulant |
| | | | melanoma, pancreas tumor, | |
| | | | sarcoma | |
| acemannan | Carrington Laboratories Inc | | pancreas tumor | Immunostimulant |
| interleukin-15, Immunex | Immunex Corp | WO 95/27722 | carcinoma, colorectal tumor, | Immunostimulant |
| | | | gastritis | |
| cytokine releasing agent, Stega | Stega Pharmazeutische Produkte | | carcinoma | Immunostimulant |
| | AG | | | |
| sizofiran | Fidia Farmaceutici Italiani | | carcinoma, lung tumor | Immunostimulant |
| | Deriviate Industriali e Affini | | | |
| LK-410 | Lek Pharmaceuticals | | carcinoma, neoplasm | Immunostimulant |
| Avipro | Avigen Inc | | prostate tumor | Immunostimulant |
| thymocartin | Richter Gedeon VG | | Hodgkin's disease | Immunostimulant |
| GnRH (LHRH) | Proteus Biotechnology Ltd | | breast tumor, prostate tumor | Immunostimulant |
| immunotherapeutic, Proteus | | | | |
| RG-003 | Ribogene Inc | | carcinoma | Immunostimulant |
| mizoribine | Asahi Chemical Industry Co Ltd | JP 48-056894 | carcinoma | Immunosuppressant |
| roquinimex | Pharmacia & Upjohn AB | EP 0 059 698 | leukemia | Immunosuppressant |
| celastrol | Schering AG | | neoplasm | Immunosuppressant |
| sirolimus | Wyeth-Ayerst Pharmaceuticals | DE 2347682 | neoplasm, carcinoma | Immunosuppressant |
| | Inc | | | |
| IC-101 | Microbial Chemistry Research | | carcinoma | Immunosuppressant |
| | Foundation | | | |
| daclizumab | Protein Design Labs Inc | EP 0 451 216 | leukemia | Immunosuppressant |
| peldesine | BioCryst Pharmaceuticals Inc | U.S. Pat. No. | non-Hodgkin's lymphoma | Immunosuppressant |
| | | 4,985,433 | | |
| Oncolysin S | Dana Farber Cancer Institute Inc | | lung tumor, nervous system | Immunosuppressant |
| | | | tumor | |
| Oncolysin CD6 | Dana Farber Cancer Institute Inc | | carcinoma, leukemia, lymphoma | Immunosuppressant |
| TAN-2178 | Takeda Shokuhin Kogyo KK | JP 09012595 | carcinoma | Immunosuppressant |
| MDL-28842 | Hoechst Marion Roussel Inc | EP 0 304 889 | carcinoma | Immunosuppressant |
| KF-20444 | Kyowa Hakko Kogyo Co Ltd | | carcinoma | Immunosuppressant |
| MC-1288 | Leo Denmark | | carcinoma | Immunosuppressant |
| lexacalcitol | Leo Pharmaceutical Products Inc | | skin tumor, breast tumor | Immunosuppressant |
| interleukin-1 receptor, Immunex | Immunex Corp | | neoplasm | Immunosuppressant |
| Org-6632 | Organon NV | | neoplasm | Immunosuppressant |
| immunosuppressant, Shionogi | Shionogi & Co Ltd | | neoplasm | Immunosuppressant |
| MAbs, B-cells, Tanox | Tanox Biosystems Inc | | carcinoma, leukemia, neoplasm, | Immunosuppressant |
| Biosystems | | | non-Hodgkin's lymphoma | |
| immunosuppressant (CD95), | Ceres Pharmaceuticals | | carcinoma | Immunosuppressant |
| CERES | | | | |
| CAMPATH-1H | Cambridge University | | leukemia, non-Hodgkin's | Immunosuppressant |
| | | | lymphoma | |
| etarotene | Roche Holding AG | | neoplasm | Immunosuppressant |
| L-6 | Bristol-Myers Squibb Co | | carcinoma | Immunosuppressant |
| mycophenolate mofetil | Roche Holding AG | EP 0 281 713 | transplant rejection | Immunosuppressant |
| apoptosin, ImmunoGen | ImmunoGen Inc | | neoplasm | Immunosuppressant |
| CT-2576 | Cell Therapeutics Inc | | neoplasm | Immunosuppressant |
| mycophenolic acid derivatives, | Abbott Laboratories | | neoplasm | Immunosuppressant |
| Abbott | | | | |
| HR-325 | Hoechst-Roussel | | neoplasm | Immunosuppressant |
| | Pharmaceuticals Inc | | | |
| AR-209 | Aronex Pharmaceuticals Inc | | bladder tumor, brain tumor, | Immunotoxin |
| | | | breast tumor, lung tumor, | |
| | | | neoplasm | |
| CC49-BAMME-CH-DOX | Eli Lilly & Co | | neoplasm | Immunotoxin |
| Oncolysin M | Dana Farber Cancer Institute Inc | | leukemia | Immunotoxin |
| LMB-1, NIH | National Institutes of Health | | carcinoma, colon tumor, breast | Immunotoxin |
| | | | tumor | |
| LMB-2, NIH | National Cancer Institute | | neoplasm | Immunotoxin |
| CMA-676 | Celltech Group plc | | myeloid leukemia | Immunotoxin |
| MR1scFvPE38KDEL, NCI | National Cancer Institute | | neoplasm | Immunotoxin |
| Oncolysin S | Dana Farber Cancer Institute Inc | | lung tumor, nervous system | Immunotoxin |
| | | | tumor | |
| Oncolysin CD6 | Dana Farber Cancer Institute Inc | | carcinoma, leukemia, lymphoma | Immunotoxin |
| monoclonal antibodies (cancer), | A Menarini Ind Farm Riunite | | neoplasm | Immunotoxin |
| Menarini | SrL | | | |
| BU12-Saporin | The University of Birmingham | | non-Hodgkin's lymphoma | Immunotoxin |
| ZD-2767-P | Zeneca Group Plc | | solid tumor | Immunotoxin |
| IgG-RFB4-SMPT-dgA | National Cancer Institute | | non-Hodgkin's lymphoma | Immunotoxin |
| G-28-5 sFv-PE40 | Bristol-Myers Squibb Co | | neoplasm | Immunotoxin |
| M195 monoclonal antibody, | Memorial Sloan-Kettering | | leukemia | Immunotoxin |
| Sloan Kettering | Cancer Center Institute | | | |
| ZD-9063P | Zeneca Group Plc | | colorectal tumor | Immunotoxin |
| ZD-0490 | Zeneca Group Plc | | carcinoma | Immunotoxin |
| monoclonals, Quest | Quest Biotechnology Inc | | carcinoma, cardiovascular tumor | Immunotoxin |
| monoclonal antibodies (cancer), | Roussel Uclaf SA | | carcinoma | Immunotoxin |
| Roussel-Uclaf | | | | |
| Oncolysin B | Dana Farber Cancer Institute Inc | | carcinoma, lung tumor, | Immunotoxin |
| | | | lymphoma | |
| XomaZyme-Mel | XOMA Corp | | carcinoma, melanoma | Immunotoxin |
| BMS-182248 | Bristol-Myers Squibb Co | | carcinoma, colon tumor, lung | Immunotoxin |
| | | | tumor, breast tumor | |
| EGFR conjugate, ImmunoGen | ImmunoGen Inc | EP 0 425 235 | squamous cell carcinoma, breast | Immunotoxin |
| | | | tumor, head & neck tumor | |
| Immutox (humanized form), | Immunomedics Inc | | neoplasm | Immunotoxin |
| Immunomedics | | | | |
| Avicidin | NeoRx Corp | | breast tumor, colon tumor, lung | Immunotoxin |
| | | | tumor, neoplasm, prostate tumor | |
| LMB-7, NIH | National Institutes of Health | | carcinoma | Immunotoxin |
| MRK16-PE | National Institutes of Health | | carcinoma, urinary tract disease, | Immunotoxin |
| | | | urinary tract tumor | |
| immunotoxins, NIH | National Institutes of Health | | neoplasm | Immunotoxin |
| diphtheria toxin, RCT | Research Corp Technologies Inc | | neoplasm | Immunotoxin |
| ZD-2767 | Zeneca Group Plc | WO 94/02450 | neoplasm, colorectal tumor | Immunotoxin |
| huN901-DC1 | ImmunoGen Inc | | lung tumor | Immunotoxin |
| C242-DM1 | ImmunoGen Inc | EP 0 425 235 | colon tumor | Immunotoxin |
| breast cancer | ImmunoGen Inc | | breast tumor | Immunotoxin |
| immunoconjugates, ImmunoGen | | | | |
| Anti-B4-DC1 | ImmunoGen Inc | U.S. Pat. No. | lymphoma | Immunotoxin |
| | | 5,475,092 | | |
| CI-935 | Parke-Davis & Co | | neoplasm | IMP dehydrogenase inhibitor |
| mizoribine | Asahi Chemical Industry Co Ltd | JP 48-056894 | carcinoma | IMP dehydrogenase inhibitor |
| IMPDH inhibitor, Sloan | Codon Pharmaceuticals Inc | | carcinoma, neoplasm | IMP dehydrogenase inhibitor |
| Kettering | | | | |
| TFAD, Camerino University | Camerino University | | neoplasm | IMP dehydrogenase inhibitor |
| tiazofurin | ICN Pharmaceuticals Inc | EP 0 054 432 | carcinoma, leukemia, lung | IMP dehydrogenase inhibitor |
| | | | tumor, myeloid leukemia | |
| inhibin, Biotech Australia | Biotech Australia | | neoplasm | Inhibin |
| CI-980 | Parke-Davis & Co | EP 0 336 345 | carcinoma, colorectal tumor, | Inotropic agent |
| | | | glioma, neoplasm, ovary tumor, | |
| | | | solid tumor | |
| vesnarinone | Otsuka Pharmaceutical Co Ltd | BE 0 890 942 | neoplasm | Inotropic agent |
| IGF-1, Genentech | Genentech Inc | | neoplasm | Insulin-like growth factor-1 |
| oligonucleotide (glioma), NCI | National Cancer Institute | | glioma | Insulin-like growth factor |
| | | | | antagonist |
| Humalog | Eli Lilly & Co | | diabetes mellitus | Insulin agonist |
| interferon-gamma analogs, NPO | NPO Vector | | neoplasm | Interferon modulator |
| Vector | | | | |
| D-0490 | Yissum Research Development | | carcinoma | Interferon modulator |
| | Co of the Hebrew University of | | | |
| | Jerusalem | | | |
| imiquimod | 3M Pharmaceuticals | EP 0 145 340 | carcinoma | Interferon modulator |
| SCHAL-3 | Sheffield Pharmaceuticals Inc | | kaposis sarcoma | Ion channel modulator |
| iron chelators, James Cook | James Cook University of North | | neoplasm | Iron modulator |
| | Queensland | | | |
| elsamitrucin | Bristol-Myers Squibb Co | BE 0 900 735 | carcinoma, neoplasm | Isomerase inhibitor |
| intoplicine | Rhone-Poulenc Rorer Inc | EP 0 402 232 | solid tumor | Isomerase inhibitor |
| iododoxorubicin | Pharmacia & Upjohn AB | BE 0 892 943 | breast tumor, carcinoma, lung | Isomerase inhibitor |
| | | | tumor | |
| nemorubicin | Pharmacia & Upjohn AB | BE 0 904 431 | carcinoma | Isomerase inhibitor |
| ED-110 | Banyu Pharmaceutical Co Ltd | WO 91/18003 | carcinoma | Isomerase inhibitor |
| CB-38416 | Centre Europeen de | WO 97/26237 | neoplasm | Keratolytic |
| | Bioprospective (CEB) | | | |
| Win-65936 | Sterling-Winthrop Inc | | lung tumor | Leukocyte elastase inhibitor |
| anticancer implant, Peptech | Peptech Ltd | | prostate tumor | LHRH |
| tryptorelin | Tulane University | | breast tumor, prostate tumor | LHRH agonist |
| nafarelin | Roche Bioscience | U.S. Pat. No. | breast tumor, prostate tumor | LHRH agonist |
| | | 4,234,571 | | |
| deslorelin | The Salk Institute | | prostate tumor | LHRH agonist |
| surfagon | Russian Academy Medical | | carcinoma | LHRH agonist |
| | Science | | | |
| MIDAS [cancer therapy] | Elan Corp Plc | | neoplasm | LHRH agonist |
| histrelin | Ortho Pharmaceutical Corp | | prostate tumor, breast tumor | LHRH agonist |
| leuprorelin | Takeda Chemical Industries Ltd | | neoplasm, breast tumor, uterus | LHRH agonist |
| | | | tumor | |
| goserelin | Zeneca Group Plc | | breast tumor, prostate tumor, | LHRH agonist |
| | | | uterus tumor | |
| Antide | Ares-Serono International SA | | carcinoma, neoplasm | LHRH antagonist |
| ganirelix | Roche Bioscience | EP 0 312 052 | breast tumor, carcinoma, | LHRH antagonist |
| | | | prostate tumor | |
| cetrorelix | ASTA Medica AG | EP 0 299 402 | breast tumor, prostate tumor, | LHRH antagonist |
| | | | endometriosis, ovary tumor, | |
| | | | uterus tumor, carcinoma | |
| peptide (prostate cancer), UBI | United Biomedical Inc | | prostate tumor | LHRH antagonist |
| D-23487 | ASTA Medica AG | | carcinoma | LHRH antagonist |
| PPI-149 | Praecis Pharmaceuticals Inc | | breast tumor, prostate tumor | LHRH antagonist |
| A-84861 | Abbott Laboratories | U.S. Pat. No. | prostate tumor | LHRH antagonist |
| | | 5,698,522 | | |
| ramorelix | Hoechst AG | EP 0 451 791 | breast tumor, esophagus tumor, | LHRH antagonist |
| | | | prostate tumor | |
| detirelix | Roche Bioscience | | breast tumor | LHRH antagonist |
| A-76154 | Abbott Laboratories | | prostate tumor | LHRH antagonist |
| Antarelix | Laboratoires Pharmascience | | neoplasm | LHRH antagonist |
| docosanol | Lidak Pharmaceuticals | WO 90/13216 | kaposis sarcoma | Lipase inhibitor |
| CV-6504 | Takeda Chemical Industries Ltd | U.S. Pat. No. | carcinoma | Lipoxygenase inhibitor |
| | | 4,851,413 | | |
| A-63162 | Abbott Laboratories | | neoplasm | Lipoxygenase inhibitor |
| PD-136005 | Parke-Davis & Co | | carcinoma, leukemia | Lipoxygenase inhibitor |
| SC-41661A | Searle & Co | EP 0 190 683 | carcinoma + d2350 | Lipoxygenase inhibitor |
| abiraterone | British Technology Group Plc | GB 2 265 624 | prostate tumor | Lyase inhibitor |
| YM-116 | Yamanouchi Pharmaceutical Co | | prostate tumor | Lyase inhibitor |
| | Ltd | | | |
| P450-17-alpha inhibitor, ICR | Institute of Cancer Research, | | prostate tumor | Lyase inhibitor |
| | UK | | | |
| CB-7661 | Institute of Cancer Research, | | prostate tumor | Lyase inhibitor |
| | UK | | | |
| GI-111924 | Glaxo Wellcome plc | WO 94/27989 | prostate tumor | Lyase inhibitor |
| P450 17 alpha inhibitor, | University of Maryland | | prostate tumor | Lysase inhibitor |
| University of Maryland | | | | |
| MDL-27302 | Hoechst Marion Roussel Inc | EP 0 288 053 | carcinoma | Lysase inhibitor |
| YM-55208 | Yamanouchi Pharmaceutical Co | | prostate tumor | Lysase inhibitor |
| | Ltd | | | |
| cytotoxic macrolides, Ryukyus | University of the Ryukyus | | carcinoma | Macrolide antibiotic |
| MIF, Genetics Institute | Genetics Institute Inc | | neoplasm | Macrophage migration |
| | | | | inhibitory factor |
| MIF, Genetics Institute | Genetics Institute Inc | | neoplasm | Macrophage migration |
| | | | | inhibitory factor |
| GPX-325 | BioResearch Ireland | | neoplasm | MAO inhibitor |
| CI-959 | Parke-Davis & Co | EP 0 187 487 | neoplasm | Mast cell degranulation |
| | | | | inhibitor, Cell control agent |
| DWP-404 | Daewoong Pharmaceutical Co | | neoplasm, thrombocytopenia | Megakaryocyte growth & |
| | Ltd | | | development factor |
| microsponge (melanin) | Advanced Polymer Systems | EP 0 313 380 | skin tumor | Melanin |
| LY-121887 | Eli Lilly & Co | EP 0 748 627 | neoplasm + d2358 | Melatonin ligand |
| marimastat analogs, Zeneca | Zeneca Group Plc | | carcinoma | Metalloproteinase inhibitor |
| batimastat analogs, SB | SmithKline Beecham plc | | carcinoma | Metalloproteinase inhibitor |
| TIMP-2, Oncologix | Oncologix Inc | | neoplasm | Metalloproteinase inhibitor |
| KT5-12 | Kotobuki Seiyaku Co Ltd | | neoplasm | Metalloproteinase inhibitor |
| SoRI-8790 | Southern Research Inst | | neoplasm | Metalloproteinase inhibitor |
| MMP inhibitors, Yissum | Yissum Research Development | | neoplasm, metastasis | Metalloproteinase inhibitor |
| | Co of the Hebrew University of | | | |
| | Jerusalem | | | |
| Metastat | CollaGenex Pharmaceutical Inc | | neoplasm | Metalloproteinase inhibitor |
| metalloprotease inhibitor, | Glycomed Inc | | neoplasm | Metalloproteinase inhibitor |
| Glycomed | | | | |
| MMP8 inhibitors, Boehringer | Boehringer Mannheim GmbH | | neoplasm | Metalloproteinase inhibitor |
| Mannheim | | | | |
| MMP inhibitors, Creative | Creative Biomolecules Inc | | carcinoma | Metalloproteinase inhibitor |
| marimastat | British Biotech plc | W/O 94/02447 | ovary tumor, colorectal tumor, | Metalloproteinase inhibitor |
| | | | pancreas tumor, lung tumor, | |
| | | | prostate tumor, stomach tumor, | |
| | | | head & neck tumor, bone tumor, | |
| | | | melanoma, neoplasm, brain | |
| | | | tumor, esophagus tumor, breast | |
| | | | tumor | |
| PNU-99533 | Pharmacia & Upjohn Inc | | carcinoma | Metalloproteinase inhibitor |
| metalloproteinase inhibitors, | Polifarma SpA | | carcinoma | Metalloproteinase inhibitor |
| Polifarma | | | | |
| MMP inhibitors, Chiroscience | Chiroscience Ltd | | neoplasm | Metalloproteinase inhibitor |
| AG-3287 | Agouron Pharmaceuticals Inc | | neoplasm | Metalloproteinase inhibitor |
| AG-3293 | Agouron Pharmaceuticals Inc | | neoplasm | Metalloproteinase inhibitor |
| AG-3294 | Agouron Pharmaceuticals Inc | | neoplasm | Metalloproteinase inhibitor |
| AG-3296 | Agouron Pharmaceuticals Inc | | neoplasm | Metalloproteinase inhibitor |
| antigenic MMP peptides, NIH | National Institutes of Health | | arthritis, neoplasm, angiogenesis | Metalloproteinase inhibitor |
| | | | disorder | |
| MMP inhibitor, | CollaGenex Pharmaceutical Inc | | neoplasm | Metalloproteinase inhibitor |
| CollaGenex/Boehringer | | | | |
| MMP inhibitors, Proscript | ProScript Inc | | carcinoma | Metalloproteinase inhibitor |
| AG-3365 | Agouron Pharmaceuticals Inc | | neoplasm | Metalloproteinase inhibitor |
| D-1927 | Chiroscience Ltd | | neoplasm | Metalloproteinase inhibitor |
| D-2163 | Chiroscience Ltd | WO 97/19075 | neoplasm | Metalloproteinase inhibitor |
| NSC-683551 | National Cancer Institute | | neoplasm | Metalloproteinase inhibitor |
| AG-3067 | Agouron Pharmaceuticals Inc | | neoplasm | Metalloproteinase inhibitor |
| MMP inhibitors, Shionogi | Shionogi & Co Ltd | | neoplasm | Metalloproteinase inhibitor |
| oligonucleotide (c-jun), Isis | ISIS Pharmaceuticals Inc | | neoplasm | Metalloproteinase inhibitor |
| Pharmaceuticals | | | | |
| OPB-3206 | Otsuka Pharmaceutical Co Ltd | | angiogenesis disorder, | Metalloproteinase inhibitor |
| | | | metastasis, carcinoma | |
| matrix metalloproteinase | DuPont Pharmaceuticals Co | | neoplasm | Metalloproteinase inhibitor |
| inhibitors, Du Pont Merck | | | | |
| marimastat analog, British | British Biotech plc | | carcinoma, neoplasm | Metalloproteinase inhibitor |
| Biotech | | | | |
| matrix metalloproteinase | The Procter & Gamble Co | WO 96/20918 | metastasis | Metalloproteinase inhibitor |
| inhibitors, Procter & Gamble | | | | |
| ilomastat | Glycomed Inc | U.S. Pat. No. | neoplasm | Metalloproteinase inhibitor |
| | | 5,114,953 | | |
| CH-104 | Chiroscience Group plc | WO 95/13289 | carcinoma | Metalloproteinase inhibitor |
| Metastat | CollaGenex Pharmaceutical Inc | | neoplasm | Metastasis inhibitor |
| BG-anti-TGF-beta, Hebrew | Hebrew University of Jerusalem | | neoplasm | Metastasis inhibitor |
| University | | | | |
| cicaprost | Schering AG | DE 3306123 | carcinoma, neoplasm | Metastasis inhibitor |
| | Celltech Group plc | | breast tumor | Metastasis inhibitor |
| macrosphelides, Kitasato | Kitasato Institute | | melanoma | Metastasis inhibitor |
| Institute | | | | |
| polysulphonic acid derivatives, | Fuji Photo Film Co Ltd | JP 09059163 | neoplasm | Metastasis inhibitor |
| Fuji | | | | |
| alpha-beta integrin peptides, | Integra LifeSciences Corp | | angiogenesis disorder, | Metastasis inhibitor |
| Integra | | | carcinoma, neoplasm | |
| AGM-1470 | Takeda Chemical Industries Ltd | EP 0 359 036 | brain tumor, breast tumor, | Metastasis inhibitor |
| | | | carcinoma, kaposis sarcoma, | |
| | | | neoplasm, prostate tumor, | |
| | | | psoriasis, renal tumor, sarcoma, | |
| | | | uterine cervix tumor | |
| madindoline, Kitasato Institute | Kitasato Institute | EP 0 787 733 | myeloproliferative disorder, | Metastasis inhibitor |
| | | | neoplasm | |
| KRN-7000 | Kirin Brewery Co Ltd | | neoplasm | Metastasis inhibitor |
| conophylline | Terumo Corp | | carcinoma, neoplasm | Metastasis inhibitor |
| ATF-HI-8 | Nissin Food Products Co | | carcinoma | Metastasis inhibitor |
| GBC-590 | SafeScience Inc | | metastasis | Metastasis inhibitor |
| thrombospondin, Cornell | Cornell University | WO 92/17499 | neoplasm, metastasis | Metastasis inhibitor |
| Collamers | BioStratum Inc | | carcinoma, metastasis, prostate | Metastasis inhibitor |
| | | | tumor | |
| melanoma gene, Millennium | Millennium Pharmaceuticals Inc | | melanoma | Metastasis inhibitor |
| KiSS-1 gene therapy, Penn State | Pennsylvania State University | | melanoma, breast tumor | Metastasis inhibitor |
| BBR-2550 | Boehringer Mannheim GmbH | | neoplasm | Metastasis inhibitor |
| FCE-27266 | Farmitalia Carlo Erba SpA | | neoplasm | Metastasis inhibitor |
| Sch-49209 | Schering-Plough Corp | | neoplasm | Metastasis inhibitor |
| Sch-50672 | Schering-Plough Corp | | neoplasm | Metastasis inhibitor |
| Sch-49210 | Schering-Plough Corp | | neoplasm | Metastasis inhibitor |
| GW-278884 | Glaxo Wellcome plc | | colorectal tumor, liver tumor | Metastasis inhibitor, Enzyme |
| growth blockers, Receptagen | Receptagen Ltd | | lymphoma, carcinoma | Methionine synthase inhibitor |
| RPR-112378 | Rhone-Poulenc SA | | neoplasm | Microtubule inhibitor |
| erbulozole | Janssen Pharmaceutica NV | | neoplasm | Microtubule inhibitor |
| LY-355703 | Eli Lilly & Co | | neoplasm | Microtubule inhibitor |
| docetaxel analogs, Daiichi | Daiichi Seiyaku Co Ltd | | neoplasm | Microtubule inhibitor |
| SJ-3249 | Sam Jin Pharmaceutical Co | | neoplasm | Microtubule inhibitor |
| dolastatin 15 mimetics, ASTA | ASTA Medica AG | | neoplasm | Microtubule inhibitor |
| paclitaxel analogs, Hauser | Hauser Inc | WO 94/11366 | neoplasm | Microtubule inhibitor |
| TZT-1027 | Teikoku Hormone | | neoplasm | Microtubule inhibitor |
| | Manufacturing Co Ltd | | | |
| dolaphenine androstane | National Cancer Institute | | neoplasm | Microtubule inhibitor |
| Protax-3 | Inex Pharmaceuticals Corp | | neoplasm | Microtubule inhibitor |
| BP-179 | Biophysica Foundation | | carcinoma | Microtubule inhibitor |
| PEG-pacitaxel, Enzon | Enzon Inc | | carcinoma | Microtubule inhibitor |
| GS-164 | Takeda Chemical Industries Ltd | JP 08325147 | carcinoma | Microtubule inhibitor |
| ONCHOLAB paclitaxel, Cortecs | Cortecs International Ltd | | carcinoma | Microtubule inhibitor |
| anticancer agents, BMS/GBF | Bristol-Myers Squibb Co | | neoplasm | Microtubule inhibitor |
| BMS-185660 | Bristol-Myers Squibb Co | | carcinoma | Microtubule inhibitor |
| SB-T-104221 | New York State University | | neoplasm | Microtubule inhibitor |
| epothilones, University of | University of Kansas | | neoplasm | Microtubule inhibitor |
| Kansas | | | | |
| eleutherobin, BMS | Bristol-Myers Squibb Co | | neoplasm | Microtubule inhibitor |
| PNU-166945 | Pharmacia & Upjohn Inc | | solid tumor | Microtubule inhibitor |
| docetaxel | Rhone-Poulenc Rorer Inc | EP 0 253 738 | brain tumor, breast tumor, | Microtubule inhibitor |
| | | | esophagus tumor, head & neck | |
| | | | tumor, lung tumor, melanoma, | |
| | | | ovary tumor, pancreas tumor, | |
| | | | stomach tumor, uterus tumor | |
| 1069C85 | Burroughs Wellcome Inc | EP 0 305 093 | lymphoma, neoplasm, non- | Microtubule inhibitor |
| | | | Hodgkin's lymphoma | |
| dolastatin 10 | National Cancer Institute | | neoplasm | Microtubule inhibitor |
| SB-T-1101 | New York State University | | carcinoma | Microtubule inhibitor |
| SB-T-1211 | New York State University | | carcinoma | Microtubule inhibitor |
| ZYN-176 | Zynaxis Inc | | carcinoma | Microtubule inhibitor |
| aplyronine-A | Yamada Seiyaku Co Ltd | | neoplasm | Microtubule inhibitor |
| paclitaxel-coated stents, UBC | University of British Columbia | | esophagus tumor | Microtubule inhibitor |
| halamide, BMS | Bristol-Myers Squibb Co | | neoplasm | Microtubule inhibitor |
| paclitaxel analogs, BMS | Bristol-Myers Squibb Co | | neoplasm | Microtubule inhibitor |
| CI-980 | Parke-Davis & Co | EP 0 336 345 | carcinoma, colorectal tumor, | Microtubule inhibitor |
| | | | glioma, neoplasm, ovary tumor, | |
| | | | solid tumor | |
| LY-329146 | Eli Lilly & Co | | carcinoma | Multidrug resistance inhibitor |
| MGI-114 | MGI Pharma Inc | | breast tumor, carcinoma, colon | Multidrug resistance inhibitor |
| | | | tumor, lung tumor, neoplasm, | |
| | | | ovary tumor, uterine cervix | |
| | | | tumor | |
| CRL-1605 | CytRx Corp | | carcinoma | Multidrug resistance inhibitor |
| S-9788 | Servier | EP 0 466 586 | carcinoma | Multidrug resistance inhibitor |
| XR-5000 | Cancer Research Campaign | | carcinoma, breast tumor, lung | Multidrug resistance inhibitor |
| | Technology Ltd | | tumor, colon tumor, skin tumor, | |
| | | | brain tumor, melanoma | |
| SDZ-280-446 | Novartis AG | | neoplasm | Multidrug resistance inhibitor |
| KT-5720 | Kyowa Hakko Kogyo Co Ltd | | lymphoma, carcinoma | Multidrug resistance inhibitor |
| cancer therapeutic (antisense), | NeoPharm Inc | | lung tumor, breast tumor, colon | Multidrug resistance inhibitor |
| NeoPharm | | | tumor, digestive system tumor | |
| JSKIV-47 | Rutgers University | U.S. Pat. No. | neoplasm | Multidrug resistance inhibitor |
| | | 5,767,142 | | |
| verapamil isomers, | Chiroscience Group plc | WO 95/09150 | colorectal tumor, renal tumor, | Multidrug resistance inhibitor |
| Chiroscience/Knoll | | | non-Hodgkin's lymphoma | |
| OC-5186 | Philadelphia Biomedical | | carcinoma | Multidrug resistance inhibitor |
| | Research Institute | | | |
| PSC-833 | Novartis AG | EP 0 296 122 | neoplasm, leukemia, non- | Multidrug resistance inhibitor |
| | | | Hodgkin's lymphoma, | |
| | | | lymphoma, ovary tumor | |
| gene therapy (MDR), IntroGene | IntroGene BV | | bladder tumor, brain tumor, | Multidrug resistance inhibitor |
| | | | breast tumor, carcinoma, | |
| | | | lymphoma | |
| MDR gene therapy, Ingenex | Ingenex | | breast tumor, ovary tumor | Multidrug resistance inhibitor |
| VA-033 | Taisho Pharmaceutical Co Ltd | | neoplasm | Multidrug resistance inhibitor |
| MDR gene transfer, Genetix | Genetix Pharmaceuticals | | brain tumor, breast tumor, ovary | Multidrug resistance inhibitor |
| | | | tumor | |
| GR-66234A | Glaxo Wellcome plc | | neoplasm | Multidrug resistance inhibitor |
| oligonucleotides (mdr-1), | Hybridon Inc | WO 96/02556 | neoplasm | Multidrug resistance inhibitor |
| Hybridon | | | | |
| OC104-26 | Ontogen Corp | | carcinoma | Multidrug resistance inhibitor |
| OC42-92 | Ontogen Corp | | carcinoma | Multidrug resistance inhibitor |
| VX-853 | Vertex Pharmaceuticals Inc | WO 96/15101 | carcinoma | Multidrug resistance inhibitor |
| CP-117227 | Pfizer Inc | | carcinoma | Multidrug resistance inhibitor |
| MDR inhibitor, Tsumura | Tsumura & Co Ltd | | multidrug resistant infection, | Multidrug resistance inhibitor |
| | | | carcinoma | |
| CP-114416 | Pfizer Central Research | | neoplasm | Multidrug resistance inhibitor |
| XR-9051 | Xenova Ltd | | carcinoma | Multidrug resistance inhibitor |
| BRI MAb MDR-1, BioResearch | BioResearch Ireland | | carcinoma | Multidrug resistance inhibitor |
| Ireland | | | | |
| XR-9576 | Xenova Group plc | | neoplasm, multidrug resistant | Multidrug resistance inhibitor |
| | | | infection | |
| OC62-805 | Ontogen Corp | | carcinoma | Multidrug resistance inhibitor |
| SB-RA-31012 | Stony Brook University | | neoplasm | Multidrug resistance inhibitor |
| KT-5822 | Kyowa Hakko Kogyo Co Ltd | | neoplasm | Multidrug resistance inhibitor |
| ISIS-7597 analogs | ISIS Pharmaceuticals Inc | | neoplasm | Multidrug resistance inhibitor |
| CR-10-11 | Institute of Organic Chemistry | | neoplasm | Multidrug resistance inhibitor |
| | Moscow | | | |
| N-276-12 | Nikken Chemicals Co Ltd | | neoplasm | Multidrug resistance inhibitor |
| MDR inhibitors, Yissum | Yissum Research Development | | neoplasm | Multidrug resistance inhibitor |
| | Co of the Hebrew University of | | | |
| | Jerusalem | | | |
| cinchonine | Debiopharm SA | | neoplasm | Multidrug resistance inhibitor |
| GF-120918 | Glaxo Wellcome plc | EP 0 494 623 | neoplasm | Multidrug resistance inhibitor |
| S-16317 | Servier | | carcinoma | Multidrug resistance inhibitor |
| S-16324 | Servier | | neoplasm | Multidrug resistance inhibitor |
| MS-209 | Mitsui Pharmaceuticals Inc | | neoplasm | Multidrug resistance inhibitor |
| dexniguldipine | Byk Gulden | | neoplasm | Multidrug resistance inhibitor |
| VX-710 | Vertex Pharmaceuticals Inc | | breast tumor, liver tumor, | Multidrug resistance inhibitor |
| | | | neoplasm, ovary tumor, sarcoma | |
| RS-33295-198 | Roche Bioscience | | neoplasm | Multidrug resistance inhibitor |
| MRK-16 | Hoechst Japan Ltd | | neoplasm | Multidrug resistance inhibitor |
| MRK-17 | Hoechst Japan Ltd | | neoplasm | Multidrug resistance inhibitor |
| XR-1500 | Xenova Ltd | WO 94/04513 | carcinoma, neoplasm | Multidrug resistance inhibitor |
| PAK-200 | Nissan Chemical Industries Ltd | | carcinoma, neoplasm | Multidrug resistance inhibitor |
| FD-895 | Taisho Pharmaceutical Co Ltd | JP 04352783 | neoplasm | Multidrug resistance inhibitor |
| 10-deacetylbaccatin III | Roswell Park Cancer Institute | | neoplasm | Multidrug resistance inhibitor |
| derivatives | | | | |
| imidazoles, Ontogen | Ontogen Corp | | neoplasm | Multidrug resistance inhibitor |
| prostaglandin agonists, | Allergan Inc | | anesthesia, pain | Neuroprotectant |
| Allergan/Acadia | | | | |
| GPI-5000 | Guilford Pharmaceuticals Inc | | prostate tumor | Neuroprotectant |
| BG-anti-TGF-beta, Hebrew | Hebrew University of Jerusalem | | neoplasm | Neuroprotectant |
| University | | | | |
| NGF inhibitors, Parke-Davis | Parke-Davis & Co | | nervous system tumor | NGF antagonist |
| PD-90780 | Parke-Davis & Co | | nervous system tumor | NGF antagonist |
| CEP-751 | Cephalon Inc | | prostate tumor | NGF antagonist |
| NK-1 antagonists (2), Merck & | Merck & Co Inc | | inflammation, pain | NK1 antagonist |
| Co | | | | |
| remacemide | Astra Charnwood | EP 0 279 937 | cerebrovascular ischemia, | NMDA antagonist |
| | | | epilepsy, huntingtons chorea, | |
| | | | alzheimers disease, parkinsons | |
| | | | disease | |
| TP-72 | Dartmouth Medical School | | neoplasm | NO synthesis inhibitor |
| CNI-1493 | Picower Institute for Medical | | neoplasm | NO synthesis inhibitor |
| | Research | | | |
| small molecule NOS | Apex Bioscience Inc | | neoplasm | NO synthesis modulator |
| modulators, Apex | | | | |
| OM-174 | Max-Delbrueck-Centrum fuer | | neoplasm | NO synthesis stimulator |
| | Molekulare Medizin | | | |
| ONO-4007 | Ono Pharmaceutical Co Ltd | EP 0 226 381 | carcinoma, neoplasm | NO synthesis stimulator |
| ABS-200 series | American Biogenetic Sciences | | neoplasm | Nootropic agent |
| | Inc | | | |
| cinnamamide | Chinese Academy of Medical | | neoplasm | Nucleic acid metabolism |
| | Science | | | modulator |
| anticancer therapy, | Eli Lilly & Co | | neoplasm | Oncogene inhibitor |
| Lilly/Millennium | | | | |
| Sch-52901 | Schering-Plough Corp | | neoplasm | Oncogene inhibitor |
| L-779450 | Merck & Co Inc | | neoplasm | Oncogene inhibitor |
| INGN-201 | Introgen Therapeutics Inc | | head & neck tumor, liver tumor, | Oncogene inhibitor |
| | | | lung tumor, neoplasm, prostate | |
| | | | tumor | |
| (-)-epigallocatechin gallate | National Institutes of Health | | carcinoma, neoplasm | Oncogene inhibitor |
| | Japan | | | |
| Sch-52900 | Schering-Plough Overseas Ltd | | carcinoma | Oncogene inhibitor |
| BRCA1 gene, Myriad | Myriad Genetics Inc | | breast tumor, ovary tumor | Oncogene inhibitor |
| INGN-111 | Introgen Therapeutics Inc | | neoplasm | Oncogene inhibitor |
| RAF antagonists, Sugen/Asta | Sugen Inc | | bladder tumor, pancreas tumor | Oncogene inhibitor |
| anti-bcl-2 oligonucleotides, Univ | MD Anderson Cancer Center | | lymphoma | Oncogene inhibitor |
| Texas | | | | |
| HER-2/neu inhibitor, Targeted | Targeted Genetics Corp | | breast tumor, ovary tumor | Oncogene inhibitor |
| Genetics | | | | |
| anticancer agent, XCyte | Xcyte Therapeutics Inc | | carcinoma | Oncogene inhibitor |
| BRCA1 inhibitors, Onyx | ONYX Pharmaceuticals Inc | | breast tumor | Oncogene inhibitor |
| BRCA1 gene, Oncormed | University of California | | breast tumor, ovary tumor | Oncogene inhibitor |
| cancer therapeutics, GenQuest | GenQuest Inc | | breast tumor, melanoma, | Oncogene inhibitor |
| | | | prostate tumor | |
| oligonucleotide (Burkitts), | National Institutes of Health | | burkitts lymphoma | Oncogene inhibitor |
| Orange County Childrens | | | | |
| Hospital | | | | |
| Sch-56396 | Schering-Plough Corp | | neoplasm | Oncogene inhibitor |
| triplex-forming oligonucleotides, | Emory University | | prostate tumor | Oncogene inhibitor |
| Emory/Georgia | | | | |
| CP-147129 | Pfizer Inc | | carcinoma | Oncogene inhibitor |
| CP-149043 | Pfizer Inc | | carcinoma | Oncogene inhibitor |
| CP-202567 | Pfizer Inc | | carcinoma | Oncogene inhibitor |
| FR-901228 | Fujisawa Pharmaceutical Co Ltd | EP 0 352 646 | neoplasm | Oncogene inhibitor |
| CP-202509 | Pfizer Inc | | neoplasm | Oncogene inhibitor |
| antisense (leukemia) oligomer, | La Jolla Institute of Allergy & | | myeloid leukemia | Oncogene inhibitor |
| La Jolla/University College Cork | Immunology | | | |
| CP-358774 | OSI Pharmaceuticals Inc | | carcinoma, angiogenesis | Oncogene inhibitor |
| | | | disorder, non-Hodgkin's | |
| | | | lymphoma, head & neck tumor, | |
| | | | breast tumor, bladder tumor | |
| CGP-52622A | Novartis AG | | neoplasm | Ornithine decarboxylase |
| | | | | inhibitor |
| CGP-54169A | Novartis AG | | neoplasm | Ornithine decarboxylase |
| | | | | inhibitor |
| eflornithine | Hoechst Marion Roussel Inc | | bladder tumor, breast tumor, | Ornithine decarboxylase |
| | | | colon tumor, glioma, kaposis | inhibitor |
| | | | sarcoma, neoplasm, prostate | |
| | | | tumor, skin tumor, uterine | |
| | | | cervix tumor | |
| dihydroxycholecalciferol | Chugai Pharmaceutical Co Ltd | | neoplasm | Ornithine decarboxylase |
| | | | | inhibitor |
| ODC inhibitors, Ciba | Novartis AG | | neoplasm | Ornithine decarboxylase |
| | | | | inhibitor |
| CGP-51905A | Novartis AG | | neoplasm | Ornithine decarboxylase |
| | | | | inhibitor |
| CGP-45300A | Novartis AG | | neoplasm | Ornithine decarboxylase |
| | | | | inhibitor |
| Humalog | Eli Lilly & Co | | diabetes mellitus | Ornithine decarboxylase |
| | | | | stimulator |
| clodronate disodium, Leiras | Leiras Oy | | carcinoma, hypercalcemia, | Osteogenesis inhibitor |
| | | | neoplasm | |
| risedronic acid | Norwich-Eaton Pharmaceuticals | EP 0 186 405 | Paget's disease | Osteogenesis stimulator |
| | Inc | | | |
| minamestane | Pharmacia & Upjohn AB | DE 3604179 | carcinoma | Oxidoreductase inhibitor |
| edatrexate | SRI International | FR 2 464 956 | carcinoma, lung tumor, | Oxidoreductase inhibitor |
| | | | neoplasm | |
| mifepristone | Roussel Uclaf SA | FR 2 497 807 | breast tumor | Oxidoreductase inhibitor |
| liarozole | Janssen Pharmaceutica NV | EP 0 260 744 | carcinoma, head & neck tumor, | Oxidoreductase inhibitor |
| | | | leukemia, lung tumor, prostate | |
| | | | tumor | |
| fadrozole hydrochloride | Novartis AG | U.S. Pat. No. | breast tumor, carcinoma | P450 reductase inhibitor |
| | | 4,588,732 | | |
| minamestane | Pharmacia & Upjohn AB | DE 3604179 | carcinoma | P450 reductase inhibitor |
| liarozole | Janssen Pharmaceutica NV | EP 0 260 744 | carcinoma, head & neck tumor, | P450 reductase inhibitor |
| | | | leukemia, lung tumor, prostate | |
| | | | tumor | |
| exemestane | Pharmacia & Upjohn AB | DE 3622841 | breast tumor | P450 reductase inhibitor |
| MDL-27302 | Hoechst Marion Roussel Inc | EP 0 288 053 | carcinoma | P450 reductase inhibitor |
| YM-116 | Yamanouchi Pharmaceutical Co | | prostate tumor | P450 reductase inhibitor |
| | Ltd | | | |
| E-7010 | Eisai Co Ltd | EP 0 472 053 | carcinoma | PABA antagonist |
| PAF antagonists, Roche | Roche Holding AG | | carcinoma, digestive system | PAF antagonist |
| | | | tumor | |
| SDZ-62-434 | Novartis AG | U.S. Pat. No. | carcinoma, leukemia | PAF antagonist |
| | | 4,910,206 | | |
| XR-5118 | Xenova Ltd | | metastasis | PAI inhibitor |
| XR-334 | Xenova Ltd | GB 2 286 393 | metastasis | PAI inhibitor |
| BIBW-022 | Boehringer Ingelheim Corp | EP 0 362 645 | neoplasm | PDE I inhibitor |
| mopidamol | Boehringer Ingelheim Corp | | carcinoma, lung tumor | PDE inhibitor |
| SU-101 | Sugen Inc | WO 96/33745 | neoplasm, solid tumor, ovary | PDGF antagonist |
| | | | tumor, glioma, kaposis sarcoma, | |
| | | | prostate tumor, lung tumor | |
| PDGF TK antagonists, Sugen | Sugen Inc | | brain tumor, carcinoma, ovary | PDGF antagonist |
| | | | tumor, prostate tumor, solid | |
| | | | tumor | |
| retro-inverso peptidomimetics, | National Cancer Institute | | carcinoma, breast tumor, kaposis | Peptide agonist |
| NCI | | | sarcoma | |
| NSC-645306 | National Cancer Institute | | melanoma, breast tumor | Permeability enhancer |
| cecropin B | Proteus Molecular Design Ltd | | neoplasm | Permeability enhancer |
| AmBisome | NeXstar Pharmaceuticals Inc | | carcinoma | Permeability enhancer |
| amphotericin B lipid complex, | The Liposome Company Inc | U.S. Pat. No. | fungal infection, aspergillus | Permeability enhancer |
| | | 4,897,384 | infection, cryptococcus | |
| | | | infection, leishmania tropica | |
| | | | infection, candida albicans | |
| | | | infection, cryptococcus | |
| | | | neoformans infection | |
| N-1379 | American Cyanamid Co | JP 61-200913 | carcinoma | Permeability enhancer |
| prostaglandin agonists, | Allergan Inc | | anesthesia, pain | PG agonist |
| Allergan/Acadia | | | | |
| MCP-1 inhibitor, Teijin | Teijin Ltd | | neoplasm | PGEI agonist |
| LY-294002 | Eli Lilly & Co | | neoplasm | Phosphoinositide 3-kinase |
| | | | | inhibitor |
| MAP kinase inhibitors, Cortecs | Cortecs International Ltd | | neoplasm | Phosphokinase inhibitor |
| PKC modulators, | University of Georgetown | | neoplasm | Phosphokinase modulator |
| Georgetown/Naval Res/NIH | | | | |
| CRM-51005 | Korea Research Institute of | | neoplasm | Phospholipase C inhibitor |
| | Bioscience and Biotechnology | | | |
| phospholipase C inhibitors, Du | DuPont Pharmaceuticals Co | | neoplasm | Phospholipase C inhibitor |
| Pont Merck | | | | |
| Phosphonate, Inflazyme | InflaZyme Pharmaceuticals Ltd | U.S. Pat. No. | colon tumor, leukemia, | Phospholipase C inhibitor |
| | | 5,369,097 | lymphoma, melanoma | |
| hispidospermidin | Nippon Roche KK | | carcinoma | Phospholipase C inhibitor |
| CT-2584 | Cell Therapeutics Inc | | breast tumor, carcinoma, | Phospholipase inhibitor |
| | | | leukemia, lung tumor, | |
| | | | melanoma, ovary tumor, prostate | |
| | | | tumor, renal tumor, sarcoma, | |
| | | | solid tumor | |
| Sch-53827 | Schering-Plough Research | | carcinoma | Phospholipase inhibitor |
| | Institute | | | |
| VRCTC-310 | Ventech Research | | neoplasm | Phosphorylase modulator |
| temoporfin | Efamol | U.S. Pat. No. | head & neck tumor, neoplasm, | Photosensitizer |
| | | 4,992,257 | pharynx tumor | |
| porfimer sodium | QLT PhotoTherapeutics Inc | | bladder tumor, carcinoma, | Photosensitizer |
| | | | esophagus tumor, head & neck | |
| | | | tumor, kaposis sarcoma, lung | |
| | | | tumor, neoplasm, d2688stomach | |
| | | | tumor, uterine cervix tumor | |
| B43.13, Biomira | Biomira Inc | | ovary tumor | Photosensitizer |
| B43.13, Biomira | Biomira Inc | | ovary tumor | Photosensitizer |
| SC-102 | Scotia Holdings plc | | head & neck tumor, neoplasm | Photosensitizer |
| PDT, Roswell Park Cancer | Roswell Park Cancer Institute | | carcinoma | Photosensitizer |
| Insitute | | | | |
| photodynamic therapy, Ergo | Rowland Institute for Scientific | | neoplasm | Photosensitizer |
| | Research | | | |
| SnET2 | Miravant Medical Technologies | | bladder tumor, breast tumor, | Photosensitizer |
| | | | cardiovascular disease, kaposis | |
| | | | sarcoma, lung tumor, neoplasm, | |
| | | | skin tumor | |
| TH-94-01 | Theratechnologies Inc | | breast tumor, leukemia, lung | Photosensitizer |
| | | | tumor | |
| hypocrellins, AltaRex | AltaRex Corp | | ovary tumor | Photosensitizer |
| SQN-400 | Scotia Holdings plc | | carcinoma | Photosensitizer |
| P-0954 | Yissum Research Development | | carcinoma | Photosensitizer |
| | Co of the Hebrew University of | | | |
| | Jerusalem | | | |
| FP-846 | FMC Corp | | carcinoma | Photosensitizer |
| Levulan | Queen′s University at Kingston | | squamous cell carcinoma, skin | Photosensitizer |
| | | | tumor, bladder tumor | |
| PCI-0123 | Pharmacyclics Inc | | breast tumor, carcinoma, | Photosensitizer |
| | | | melanoma, neoplasm | |
| NPE-6 | Nippon Petrochem Co Ltd | | neoplasm | Photosensitizer |
| BOPP, Pacific | Pacific Pharmaceuticals Inc | | neoplasm, brain tumor | Photosensitizer |
| hypericin | VimRx Pharmaceuticals Inc | | glioma, neoplasm | Photosensitizer |
| third generaion photosensitizers, | QLT PhotoTherapeutics Inc | | neoplasm | Photosensitizer |
| QLT | | | | |
| anti-inflammatories, Genetics | Genetics Institute Inc | | carcinoma | PLA2 inhibitor |
| Institute | | | | |
| IP-3196 | ISIS Pharmaceuticals Inc | | neoplasm | PLA2 inhibitor |
| gene therapy (PAI-1), UT | UT Southwestern Medical | | ocular neoplasm | Plasminogen activaator inhibitor |
| Southwestern | Center | | | |
| NK-109 | Nippon Kayaku Co Ltd | EP 0432 630 | neoplasm | Platelet aggregation inhibitor |
| PN-271 | Paracelsian Inc | | breast tumor, neoplasm, prostate | Platelet aggregation inhibitor |
| | | | tumor | |
| Dauricine | Wuhan Medical College | | neoplasm | Platelet aggregation inhibitor |
| MDL-28314 | Hoechst Marion Roussel Inc | EP 0 399 519 | carcinoma, leukemia, neoplasm, | Polyamine oxidase inhibitor |
| | | | solid tumor | |
| diethylnorspermine | University of Florida | | colon tumor, lung tumor, | Polyamine synthesis inhibitor |
| | | | melanoma, neoplasm, ovary | |
| | | | tumor, pancreas tumor, renal | |
| | | | tumor | |
| polyamine analogs, NIH | National Institutes of Health | | neoplasm | Polyamine synthesis inhibitor |
| mitoguazone | Ilex Oncology | | lymphoma, non-Hodgkin′s | Polyamine synthesis inhibitor |
| | | | lymphoma, prostate tumor, lung | |
| | | | tumor, Hodgkin′s disease, head | |
| | | | & neck tumor | |
| diethylhomospermine | University of Florida | | carcinoma, diarrhea, melanoma, | Polyamine synthesis inhibitor |
| | | | ulcerative colitis | |
| RWJ-25333 | R W Johnson Pharmaceutical | | neoplasm | Progesterone ligand |
| | Research Institute | | | |
| sex hormone agonist (tissue | Ligand Pharmaceuticals Inc | | carcinoma, hormone | Progestogen agonist |
| selective), Ligand | | | replacement therapy | |
| LG-2527 | Ligand Pharmaceuticals Inc | | hormone replacement therapy, | Progestogen agonist |
| | | | neoplasm | |
| LG-2716 | Ligand Pharmaceuticals Inc | | breast tumor, hormone | Progestogen agonist |
| | | | replacement therapy, neoplasm | |
| LG-120794 | Ligand Pharmaceuticals Inc | | hormone replacement therapy, | Progestogen agonist |
| | | | breast tumor | |
| progesterone agonists, Ligand | Ligand Pharmaceuticals Inc | | hormone replacement therapy, | Progestogen agonist |
| | | | breast tumor | |
| Antide | Ares-Serono International SA | | carcinoma, neoplasm | Progestogen antagonist |
| RU-49295 | Roussel Uclaf SA | | neoplasm | Progestogen antagonist |
| Org-31806 | Organon NV | | carcinoma | Progestogen antagonist |
| progesterone antagonists, Ligand | Ligand Pharmaceuticals Inc | | carcinoma | Progestogen antagonist |
| onapristone | Schering AG | DE 3321826 | breast tumor, carcinoma | Progestogen antagonist |
| Org-31710 | Organon NV | EP 0 289 073 | carcinoma | Progestogen antagonist |
| RU-46556 | Roussel Uclaf SA | FR 2 596 395 | neoplasm | Progestogen antagonist |
| ZK-136796 | Schering AG | | carcinoma | Progestogen antagonist |
| Org-33245 | Organon NV | | carcinoma | Progestogen antagonist |
| Org-33628 | Organon NV | | carcinoma | Progestogen antagonist |
| Org-33832 | Organon NV | | carcinoma | Progestogen antagonist |
| ZK-136798 | Schering AG | | carcinoma | Progestogen antagonist |
| ZK-114043 | Schering AG | | carcinoma | Progestogen antagonist |
| LG-1127 | Ligand Pharmaceuticals Inc | | contraception, neoplasm | Progestogen antagonist |
| LG-1447 | Ligand Pharmaceuticals Inc | | contraception, neoplasm | Progestogen antagonist |
| cicaprost | Schering AG | DE 3306123 | carcinoma, neoplasm | Prostacyclin agonist |
| TIMP-2, Oncologix | Oncologix Inc | | neoplasm | Protease inhibitor |
| AR-209 | Aronex Pharmaceuticals Inc | | bladder tumor, brain tumor, | Protease inhibitor |
| | | | breast tumor, lung tumor, | |
| | | | neoplasm | |
| CA-074 | Henry Ford Health System | | glioma | Protease inhibitor |
| protease inhibitors, UCSF | University of California | | neoplasm, metastasis | Protease inhibitor |
| proteosome inhibitors, CV | CV Therapeutics Inc | | neoplasm, inflammation | Protease inhibitor |
| Therapeutics | | | | |
| PS-341 | ProScript Inc | | carcinoma | Protease modulator |
| drug screening, Cytovia | Cytovia Inc | | neoplasm | Protease modulator |
| lisofylline | Cell Therapeutics Inc | | myeloid leukemia, neoplasm | Protectant |
| HGO-0300 | Human Genome Sciences Inc | | leukemia, neoplasm, radiation | Protectant |
| | | | sickness | |
| TEMPOL | US Department of Health & | WO 96/40127 | neoplasm | Protectant |
| | Human Services | | | |
| BB-10010 | British Biotech plc | | neoplasm, breast tumor, lung | Protectant |
| | | | tumor | |
| ICRF 187 analogs, BTG | Imperial Cancer Research | | carcinoma | Protectant |
| | Technology Ltd | | | |
| MAb-81C6 | Duke University | WO 94/21293 | brain tumor | Protein binding inhibitor |
| zaragozic acid C | Merck & Co Inc | | carcinoma | Protein farnesyl transferase |
| | | | | inhibitor |
| zaragozic acid C | Merck & Co Inc | | carcinoma | Protein farnesyl transferase |
| | | | | inhibitor |
| L-745631 | Merck & Co Inc | | carcinoma | Protein farnesyl transferase |
| | | | | inhibitor |
| Sch-44342 | Schering-Plough Research | | carcinoma | Protein farnesyl transferase |
| | Institute | | | inhibitor |
| ras farnesyl transferase | Yissum Research Development | | neoplasm | Protein farnesyl transferase |
| inhibitors, Yissum | Co of the Hebrew University of | | | inhibitor |
| | Jerusalem | | | |
| L-731735 | Merck & Co Inc | | neoplasm | Protein farnesyl transferase |
| | | | | inhibitor |
| L-739749 | Merck & Co Inc | | neoplasm | Protein farnesyl transferase |
| | | | | inhibitor |
| protein farnesyl transferase | Merck & Co Inc | | neoplasm | Protein farnesyl transferase |
| inhibitors, Merck & Co | | | | inhibitor |
| RPR-113829 | Rhone-Poulenc SA | | carcinoma | Protein farnesyl transferase |
| | | | | inhibitor |
| RPR-115135 | Rhone-Poulenc SA | | neoplasm | Protein farnesyl transferase |
| | | | | inhibitor |
| oreganic acid, Merck | Merck & Co Inc | | neoplasm | Protein farnesyl transferase |
| | | | | inhibitor |
| TAN-1831 | Takeda Chemical Industries Ltd | | neoplasm | Protein farnesyl transferase |
| | | | | inhibitor |
| Sch-66336 | Schering-Plough Research | | neoplasm | Protein farnesyl transferase |
| | Institute | | | inhibitor |
| ras farnesyl transferase | Ferring Research Institute | | neoplasm | Protein farnesyl transferase |
| inhibitors, Ferring | | | | inhibitor |
| BMS-185857 | Bristol-Myers Squibb AG | | neoplasm | Protein farnesyl transferase |
| | | | | inhibitor |
| Sch-56580 | Schering-Plough Research | | neoplasm | Protein farnesyl transferase |
| | Institute | | | inhibitor |
| GEM-230 | Hybridon Inc | | colon tumor, breast tumor, ovary | Protein kinase A inhibitor |
| | | | tumor, lung tumor | |
| GEM-231 | Hybridon Inc | WO 95/15378 | lung tumor, colon tumor, breast | Protein kinase A inhibitor |
| | | | tumor, solid tumor | |
| PKC inhibitors, Roche | Roche Holding AG | | neoplasm | Protein kinase C inhibitor |
| perifosine | ASTA Medica AG | | neoplasm, lung tumor, head & | Protein kinase C inhibitor |
| | | | neck tumor, colorectal tumor | |
| UCN-1028 | Kyowa Hakko Kogyo Co Ltd | EP 0 390 181 | neoplasm | Protein kinase C inhibitor |
| ISIS-3521 | ISIS Pharmaceuticals Inc | | neoplasm, solid tumor | Protein kinase C inhibitor |
| staurosporine | Kitasato Institute | | neoplasm | Protein kinase C inhibitor |
| thymidine analogs, Georgia | University of Georgia | | carcinoma | Protein kinase C inhibitor |
| University | | | | |
| ISIS-3521 analogs | ISIS Pharmaceuticals Inc | | neoplasm | Protein kinase C inhibitor |
| HMR-15509 | Hoechst Marion Roussel | WO 97/45397 | neoplasm | Protein kinase C inhibitor |
| | Deutschland GmbH | | | |
| CGP-41251 | Novartis AG | EP 0 296 110 | colorectal tumor, breast tumor, | Protein kinase C inhibitor |
| | | | solid tumor | |
| Ro-31-7549 | Roche Holding AG | EP 0 328 026 | carcinoma | Protein kinase C inhibitor |
| safingol | Sphinx Pharmaceuticals Corp | | neoplasm + d2637 | Protein kinase C inhibitor |
| bryostatin-1, BMS/NCI | Arizona State University | | carcinoma, neoplasm | Protein kinase C inhibitor |
| ilmofosine | Boehringer Mannheim GmbH | EP 0 050 327 | neoplasm | Protein kinase C inhibitor |
| ISIS-4189 | ISIS Pharmaceuticals Inc | | neoplasm | Protein kinase C inhibitor |
| Ro-31-8220 | Roche Holding AG | | inflammation, neoplasm | Protein kinase C inhibitor |
| Goe-7874 | Goedecke AG | | neoplasm | Protein kinase C inhibitor |
| balanol analogs, Sphinx | Sphinx Pharmaceuticals Corp | WO 93/03730 | neoplasm | Protein kinase C inhibitor |
| NSC-639365 | Sphinx Pharmaceuticals Corp | | neoplasm | Protein kinase C inhibitor |
| NSC-639366 | Sphinx Pharmaceuticals Corp | | neoplasm | Protein kinase C inhibitor |
| NSC-646958 | Sphinx Pharmaceuticals Corp | | neoplasm | Protein kinase C inhibitor |
| UCN-01 | Kyowa Hakko Kogyo Co Ltd | | neoplasm | Protein kinase C inhibitor |
| NA-382 | Hokuriku University | | neoplasm | Protein kinase C modulator |
| diacyglycerol analogs, NIH | National Institutes of Health | | neoplasm | Protein kinase C stimulator |
| KT-5720 | Kyowa Hakko Kogyo Co Ltd | | lymphoma, carcinoma | Protein kinase inhibitor |
| MAP kinase, | University of Texas System | | carcinoma, breast tumor | Protein kinase inhibitor |
| Regeneron/University of Texas | | | | |
| CGP-60474 | Novartis AG | | neoplasm | Protein kinase inhibitor |
| protein kinase inhibitors, | Molecumetics Ltd | | neoplasm | Protein kinase inhibitor |
| Molecumetics/Univ of | | | | |
| Washington | | | | |
| phenylamino-pyrimidines, Ciba- | Novartis AG | | neoplasm | Protein kinase inhibitor |
| Geigy | | | | |
| PD-098059 | Parke-Davis & Co | | neoplasm | Protein kinase inhibitor |
| echiguanine derivatives, Keio | Keio University | | carcinoma, neoplasm | Protein kinase inhibitor |
| PD-089828 | Parke-Davis & Co | | neoplasm | Protein kinase inhibitor |
| PD-090560 | Parke-Davis & Co | | neoplasm | Protein kinase inhibitor |
| CDK2 inhibitors, UCSF | University of California San | | neoplasm | Protein kinase inhibitor |
| | Francisco | | | |
| oligonucleotide (PKA-1), NIH | National Institutes of Health | | neoplasm | Protein kinase inhibitor |
| OK-1035 | Banyu Pharmaceutical Co Ltd | | neoplasm | Protein kinase inhibitor |
| Y-27632 | Yoshitomi Pharmaceutical | | metastasis | Protein kinase inhibitor |
| | Industries Ltd | | | |
| NSC-636851 | Sphinx Pharmaceuticals Corp | | neoplasm | Protein kinase inhibitor |
| cyclocreatine | RepliGen Corp | WO 92/08456 | neoplasm | Protein kinase inhibitor |
| ISIS-5132 | ISIS Pharmaceuticals Inc | U.S. Pat. No. | breast tumor, colon tumor, lung | Protein kinase inhibitor |
| | | 5,563,255 | tumor, neoplasm, ovary tumor, | |
| | | | pancreas tumor, prostate tumor | |
| U-98017 | Pharmacia & Upjohn Co | | neoplasm | Protein kinase inhibitor |
| P58, NIH | National Institutes of Health | | carcinoma | Protein kinase inhibitor |
| KT-5823 | Kyowa Hakko Kogyo Co Ltd | | neoplasm | Protein kinase inhibitor |
| Dnacin A1 | Takeda Chemical Industries Ltd | | carcinoma, neoplasm | Protein kinase inhibitor |
| Dnacin B1 | Takeda Chemical Industries Ltd | | carcinoma | Protein kinase inhibitor |
| SPC-103751 | Sphinx Pharmaceuticals Corp | | carcinoma, melanoma | Protein kinase inhibitor |
| flavopiridol | Hoechst AG | | breast tumor, lung tumor, | Protein kinase inhibitor |
| | | | digestive system tumor, | |
| | | | neoplasma, lymphoma | |
| oligonucleotide (cAMP | University of Alabama in | | colon tumor | Protein kinase modulator |
| dependent protein kinase), | Birmingham | | | |
| University of Alabama | | | | |
| bropirimine | Pharmacia & Upjohn Inc | DE 3008693 | bladder tumor | Protein synthesis inhibitor |
| palmitoylrhizoxin | Sankyo KK | | carcinoma | Protein synthesis inhibitor |
| tiricibine analogs, Univ | University of Michigan | | neoplasm | Protein synthesis inhibitor |
| Michigan | | | | |
| sirolimus | Wyeth-Ayerst Pharmaceuticals | DE 2347682 | neoplasm, carcinoma | Protein synthesis inhibitor |
| | Inc | | | |
| BCH-242 | BioChem Pharma Inc | | carcinoma, neoplasm | Protein synthesis inhibitor |
| TNP-351 | Takeda Chemical Industries Ltd | U.S. Pat. No. | carcinoma | Protein synthesis inhibitor |
| | | 4,997,838 | | |
| trimetrexate | Warner-Lambert Co | U.S. Pat. No. | carcinoma, colorectal tumor, | Protein synthesis inhibitor |
| | | 4,391,809 | neoplasm, stomach tumor | |
| Oncolysin M | Dana Farber Cancer Institute Inc | | leukemia | Protein synthesis inhibitor |
| E2 transcription factor regulator, | Signal Pharmaceuticals Inc | | carcinoma | Protein synthesis inhibitor |
| Signal | | | | |
| MSI-130 | Magainin Pharmaceuticals Inc | | carcinoma | Protein synthesis inhibitor |
| MSI-99 | Magainin Pharmaceuticals Inc | | carcinoma | Protein synthesis inhibitor |
| oligonucleotides (antisense), | Gilead Sciences Inc | | neoplasm | Protein synthesis inhibitor |
| Gilead | | | | |
| zilascorb (2H) | Pronova A/S | U.S. Pat. No. | melanoma, neoplasm, ovary | Protein synthesis inhibitor |
| | | 5,032,610 | tumor, pancreas tumor | |
| TP-40 | Merck & Co Inc | | bladder tumor | Protein synthesis inhibitor |
| eudistomins, Solvay | Solvay Duphar BV | | carcinoma | Protein synthesis inhibitor |
| PTH antagonist, Sandoz | Novartis AG | WO 96/03437 | neoplasm | PTH antagonist |
| BIM-44002 | Ipsen-Beaufour | | carcinoma | PTH antagonist |
| peldesine | BioCryst Pharmaceuticals Inc | U.S. Pat. No. | non-Hodgkin's lymphoma | Purine nucleoside phosphorylase |
| | | 4,985,433 | | inhibitor |
| purine nucleoside phosphorylase | Merrell Dow Pharmaceuticals | | lymphoma, leukemia | Purine nucleoside phosphorylase |
| inhibitors, Merrell Dow | Inc | | | inhibitor |
| purine nucleoside phosphorylase | Novartis AG | | neoplasm | Purine nucleoside phosphorylase |
| inhibitors, Ciba | | | | inhibitor |
| PNP inhibitors, Chiroscience | Chiroscience Group plc | WO 96/11200 | carcinoma, neoplasm | Purine nucleoside phosphorylase |
| | | | | inhibitor |
| AG-337 | Agouron Pharmaceuticals Inc | | colon tumor, head & neck | Radiochemosensitizer |
| | | | tumor, liver tumor, lung tumor, | |
| | | | pancreas tumor, prostate tumor, | |
| | | | solid tumor | |
| S-9788 | Servier | EP 0 466 586 | carcinoma | Radiochemosensitizer |
| 776C85 | Glaxo Wellcome plc | | neoplasm, colon tumor, breast | Radiochemosensitizer |
| | | | tumor, prostate tumor, pancreas | |
| | | | tumor | |
| PSC-833 | Novartis AG | EP 0 296 122 | neoplasm, leukemia, non- | Radiochemosensitizer |
| | | | Hodgkin's lymphoma, | |
| | | | lymphoma, ovary tumor, | |
| DPPE, BMS | University of Manitoba | | prostate tumor, breast tumor | Radiochemosensitizer |
| SDZ-280-446 | Novartis AG | | neoplasm | Radiochemosensitizer |
| Ro-11-2933 | Roche Holding AG | EP 0 523 493 | female genital tract tumor | Radiochemosensitizer |
| RB-6145 | British Technology Group Plc | EP 0 319 329 | carcinoma, neoplasm | Radiochemosensitizer |
| erbulozole | Janssen Pharmaceutica NV | | neoplasm | Radiochemosensitizer |
| AK-2123 | Alkermes Inc | | neoplasm | Radiochemosensitizer |
| PR-350 | Pola Chemical Ind Inc | | neoplasm | Radiochemosensitizer |
| PD-130908 | Parke-Davis & Co | U.S. Pat. No. | carcinoma | Radiochemosensitizer |
| | | 4,954,515 | | |
| velaresol | Glaxo Wellcome plc | EP 0 022 229 | carcinoma | Radiochemosensitizer |
| JM-2929 | Johnson Matthey plc | | neoplasm | Radiochemosensitizer |
| 153Sm-EDTMP | The Dow Chemical Co | | prostate tumor, breast tumor, | Radiochemosensitizer |
| | | | pain, neoplasm | |
| CP-100356 | Pfizer Inc | WO 92/07844 | neoplasm | Radiochemosensitizer |
| Gd-Tex | Pharmacyclics Inc | | brain tumor, carcinoma, | Radiochemosensitizer |
| | | | metastasis, neoplasm | |
| RP-170 | Kayaku Co Ltd | | carcinoma | Radiochemosensitizer |
| IPdR | Sparta Pharmaceuticals Inc | | liver tumor, neoplasm | Radiochemosensitizer |
| RSU-1069 | British Technology Group Plc | | neoplasm | Radiochemosensitizer |
| Oncolym | Techniclone Corp | | non-Hodgkin's lymphoma | Radioimmuno-therapeutic |
| Yttrium-conjugated HMFG1 | Imperial Cancer Research | | ovary tumor | Radioimmuno-therapeutic |
| antibody, ICRF | Technology Ltd | | | |
| 90Y-CC49 mAb, University of | University of Alabama in | | colorectal tumor, neoplasm | Radioimmuno-therapeutic |
| Alabama | Birmingham | | | |
| IDEC-Y2B8 | IDEC Pharmaceuticals Corp | WO 94/11026 | non-Hodgkin's lymphoma | Radioimmuno-therapeutic |
| ImmuRAIT-HCG(I-131), | Immunomedics Inc | | neoplasm | Radioimmuno-therapeutic |
| Immunomedics | | | | |
| ImmuRAIT-AFP(I-131), | Immunomedics Inc | | liver tumor, ovary tumor, testis | Radioimmuno-therapeutic |
| Immunomedics | | | tumor | |
| ImmuRAIT-LL2 | Immunomedics Inc | | non-Hodgkin's lymphoma | Radioimmuno-therapeutic |
| CEA-Cide | Immunomedics Inc | | colorectal tumor, liver tumor, | Radioimmuno-therapeutic |
| | | | neoplasm by site, non-Hodgkin's | |
| | | | lymphoma, ovary tumor | |
| ImmuRAID-HCG-Tc-99m, | Immunomedics Inc | EP 0 336 678 | ovary tumor, testis tumor, uterus | Radioimmuno-therapeutic |
| Immunomedics | | | tumor, neoplasm by site | |
| ImmuRAIT-CEA-rhenium-188, | Immunomedics Inc | EP 0 336 678 | colon tumor, colorectal tumor, | Radioimmuno-therapeutic |
| Immunomedics | | | neoplasm | |
| ImmuRAID-AFP-Tc-99m, | Immunomedics Inc | EP 0 336 678 | liver tumor, ovary tumor, testis | Radioimmuno-therapeutic |
| Immunomedics | | | tumor | |
| rhenium-188-LL2, | Immunomedics Inc | EP 0 336 678 | non-Hodgkin's lymphoma | Radioimmuno-therapeutic |
| Immunomedics | | | | |
| CEA-Scan | Immunomedics Inc | EP 0 336 678 | breast tumor, colorectal tumor, | Radioimmuno-therapeutic |
| | | | heart disease, infection, lung | |
| | | | tumor, neoplasm | |
| radiolabeled fusion toxins | UAB Research Foundation | WO 97/42217 | neoplasm, myeloid leukemia, | Radiopharmaceutical |
| (cancer), UAB | | | melanoma, lung tumor, breast | |
| | | | tumor, colon tumor | |
| 88BV59-LiLo.Y-90 | Akzo Nobel NV | | neoplasm | Radiopharmaceutical |
| imaging agents, | Anormed | | neoplasm | Radiopharmaceutical |
| Anormed/DuPont Merck | | | | |
| imaging agents, Resolution | Resolution Pharmaceuticals | | inflammation, carcinoma | Radiopharmaceutical |
| DW-166HC | Dong-Wha Pharmaceutical | | liver tumor, solid tumor | Radiopharmaceutical |
| | Industry Co Ltd | | | |
| BPA, Boron Biologicals | Boron Biologicals Inc | | carcinoma | Radiopharmaceutical |
| Hoe-33342 | Hoechst AG | | neoplasm | Radioprotectant |
| galactosylceramides, Kirin | Kirin Brewery Co Ltd | | neoplasm | Radioprotectant |
| Brewery | | | | |
| cancer therapeutic (antisense), | NeoPharm Inc | | lung tumor, breast tumor, colon | Radiosensitizer |
| NeoPharm | | | tumor, digestive system tumor | |
| temoporfin | Efamol | U.S. Pat. | head & neck tumor, neoplasm, | Radiosensitizer |
| | | 4,992,257 | pharynx tumor | |
| imidocaptate | Louisiana State University | | neoplasm | Radiosensitizer |
| Neu-Sensamide | OXiGENE Inc | | lung tumor, brain tumor, | Radiosensitizer |
| | | | neoplasm | |
| KU-2285 | Kyoto University | | neoplasm | Radiosensitizer |
| CI-1010 | Parke-Davis & Co | | neoplasm | Radiosensitizer |
| delivery system (boron), Ohio | Ohio State University | | brain tumor | Radiosensitizer |
| State University | | | | |
| KIH-802 | University Tokushima | | neoplasm | Radiosensitizer |
| KIN-806 | University Tokushima | | neoplasm | Radiosensitizer |
| SPI-40 | Sequus Pharmaceuticals Inc | | carcinoma | Radiosensitizer |
| RSR-13 | Allos Therapeutics Inc | | carcinoma | Radiosensitizer |
| MPI-5020 | Matrix Pharmaceutical Inc | | breast tumor, carcinoma | Radiosensitizer |
| CT-2412 | Cell Therapeutics Inc | | neoplasm | Radiosensitizer |
| mitolactol | Chinoin Gyogyszer Es | | brain tumor, carcinoma, uterine | Radiosensitizer |
| | Vegyeszeti | | cervix tumor | |
| Boron-anticancers, Univ of | University of Tennessee, | | brain tumor, neoplasm | Radiosensitizer |
| Tennessee | Knoxville | | | |
| broxuridine | National Cancer Institute | | brain tumor, breast tumor, | Radiosensitizer |
| | | | glioma | |
| idoxuridine, NeoPharm | National Cancer Institute | | sarcoma, renal tumor, pancreas | Radiosensitizer |
| | | | tumor | |
| L-739750 | Merck & Co Inc | | carcinoma | RAS protein inhibitor |
| Sch-48755 | Schering-Plough Corp | | neoplasm | RAS protein inhibitor |
| farnesyl transferase inhibitors, | Pierre Fabre Participations SA | | neoplasm | RAS protein inhibitor |
| Pierre Fabre | | | | |
| XR-3005 | Xenova Ltd | | colon tumor, pancreas tumor, | RAS Protein inhibitor |
| | | | solid tumor | |
| L-744832 | Merck & Co Inc | | neoplasm | RAS Protein inhibitor |
| PD-169451 | Parke-Davis & Co | | neoplasm | RAS protein inhibitor |
| Sch-56580 | Schering-Plough Research | | neoplasm | RAS protein inhibitor |
| | Institute | | | |
| farnesyltransferase inhibitors, | Genentech Inc | | colon tumor, pancreas tumor | RAS Protein inhibitor |
| Genentech | | | | |
| FTase inhibitor, Kyowa Hakko | Kyowa Hakko Kogyo Co Ltd | | neoplasm | RAS Protein inhibitor |
| ras transformation inhibitor, | Shionogi & Co Ltd | | carcinoma | RAS protein inhibitor |
| Shionogi | | | | |
| farnesyl protein transferase | University of Iowa | | neoplasm | RAS protein inhibitor |
| inhibitor, Iowa | | | | |
| ISIS-6957 | ISIS Pharmaceuticals Inc | | neoplasm | RAS protein inhibitor |
| ISIS-2503 | ISIS Pharmaceuticals Inc | WO 92/22651 | neoplasm, solid tumor | RAS protein inhibitor |
| ras processing inhibitor, | Harvard University | | neoplasm | RAS protein inhibitor |
| Harvard/ProS | | | | |
| Ras inhibitor, Acacia | Acacia BioSciences Inc | | neoplasm | RAS protein inhibitor |
| farnesyl transferase inhibitors, | Ilex Oncology | | solid tumor | RAS protein inhibitor |
| ILEX | | | | |
| Sch-54429 | Schering-Plough Corp | | neoplasm | RAS protein inhibitor |
| INGN-212 | Introgen Therapeutics Inc | | neoplasm | RAS protein inhibitor |
| FTI-298 | University of Pittsburgh | | glioma, neoplasm | RAS protein inhibitor |
| ISIS-2570 | ISIS Pharmaceuticals Inc | | neoplasm | RAS protein inhibitor |
| KT-7595 | Kyowa Hakko Kogyo Co Ltd | | carcinoma | RAS protein inhibitor |
| CP-225917 | Pfizer Inc | | carcinoma | RAS protein inhibitor |
| ras inhibitors, Agouron | Agouron Pharmaceuticals Inc | | carcinoma | RAS protein inhibitor |
| B-581 | Eisai Co Ltd | | carcinoma | RAS Protein inhibitor |
| BZA-2B | Roche Holding AG | | digestive system tumor, lung | RAS Protein inhibitor |
| | | | tumor, pancreas tumor | |
| PD-83176 | Parke-Davis & Co | | carcinoma | RAS Protein inhibitor |
| BMS-193269 | Bristol-Myers Squibb Co | | carcinoma | RAS protein inhibitor |
| ras inhibitors, Onyx | ONYX Pharmaceuticals Inc | | carcinoma | RAS Protein inhibitor |
| FTI-276 | University of Pittsburgh | | neoplasm | RAS Protein inhibitor |
| FTI-277 | University of Pittsburgh | | neoplasm | RAS Protein inhibitor |
| B-956 | Eisai Co Ltd | | neoplasm | RAS Protein inhibitor |
| PD-83176 derivative | Parke-Davis & Co | | carcinoma | RAS Protein inhibitor |
| anti-ras ribozyme, American | American Cyanamid Co | | neoplasm | RAS protein inhibitor |
| Cyanamid | | | | |
| Ras CAAX mimetics, Univ. | University of Pittsburgh | | neoplasm | RAS Protein inhibitor |
| Pittsburgh | | | | |
| L-745631 | Merck & Co Inc | | carcinoma | RAS Protein inhibitor |
| Sch-44342 | Schering-Plough Research | | carcinoma | RAS Protein inhibitor |
| | Institute | | | |
| ras farnesyl transferase | Yissum Research Development | | neoplasm | RAS Protein inhibitor |
| inhibitors, Yissum | Co of the Hebrew University of | | | |
| | Jerusalem | | | |
| L-731735 | Merck & Co Inc | | neoplasm | RAS Protein inhibitor |
| L-739749 | Merck & Co Inc | | neoplasm | RAS Protein inhibitor |
| R-115777 | Janssen Pharmaceutica BV | | neoplasm | RAS protein modulator |
| tazarotene | Allergan Inc | WO 96/11686 | acne, carcinoma, head & neck | Retinoid modulator |
| | | | tumor, leukemia, squamous cell | |
| | | | carcinoma, uterine cervix tumor | |
| retinoid prophylactic therapy, M | MD Anderson Cancer Center | | prophylaxis, lung tumor | Retinoid modulator |
| D Anderson | | | | |
| retinoid receptors, Chinese | Chinese Academy of Sciences | | neoplasm, kaposis sarcoma, | Retinoid modulator |
| Academy of Sciences | | | lymphoma | |
| MX-895 | Maxia Pharmaceuticals Inc | | neoplasm, breast tumor | Retinoid modulator |
| fenretinide | McNeil Pharmaceuticals Inc | | bladder tumor, breast tumor, | Retinoid modulator |
| | | | carcinoma, prostate tumor | |
| Ro-13-7410 | Roche Holding AG | DE 2854354 | squamous cell carcinoma | Retinoid modulator |
| Targretin | Ligand Pharmaceuticals Inc | | breast tumor, head & neck | Retinoid modulator |
| | | | tumor, kaposis sarcoma, lung | |
| | | | tumor, lymphoma, neoplasm, | |
| | | | ovary tumor, prostate tumor, | |
| | | | renal tumor, squamous cell | |
| | | | carcinoma | |
| mofarotene | Roche Holding AG | EP 0 331 983 | neoplasm | Retinoid modulator |
| CB-38416 | Centre Europeen de | WO 97/26237 | neoplasm | Retinoid modulator |
| | Bioprospective (CEB) | | | |
| ALRT-268 | Allergan Ligand Retinoid | | neoplasm | Retinoid receptor agonist |
| | Therapeutics Inc | | | |
| AGN-193174 | Allergan Inc | | neoplasm | Retinoid receptor agonist |
| ALRT-620 | Allergan Ligand Retinoid | | lymphoma, solid tumor, | Retinoid receptor agonist |
| | Therapeutics Inc | | squamous cell carcinoma | |
| ALRT-1500 | Allergan Ligand Retinoid | | neoplasm | Retinoid receptor agonist |
| | Therapeutics Inc | | | |
| tazarotene | Allergan Inc | WO 96/11686 | acne, carcinoma, head & neck | Retinoid receptor agonist |
| | | | tumor, leukemia, squamous cell | |
| | | | carcinoma, uterine cervix tumor | |
| 13-cis-retinoic acid, UCLA | University of California San | | head & neck tumor | Retinoid receptor agonist |
| | Diego | | | |
| LG-100754 | Ligand Pharmaceuticals Inc | | carcinoma | Retinoid receptor agonist |
| ALRT-550 | Allergan Ligand Retinoid | | carcinoma, leukemia, psoriasis | Retinoid receptor agonist |
| | Therapeutics Inc | | | |
| RAR alpha agonists, ALRT | Allergan Ligand Retinoid | | carcinoma, leukemia, psoriasis | Retinoid receptor agonist |
| | Therapeutics Inc | | | |
| ALRT-792 | Allergan Ligand Retinoid | | lymphoma, solid tumor, | Retinoid receptor agonist |
| | Therapeutics Inc | | squamous cell carcinoma | |
| AM-580 | Hoffmann-La Roche AG | | carcinoma, leukemia | Retinoid receptor agonist |
| AGN-191701 | Allergan Inc | WO 94/17796 | neoplasm | Retinoid receptor agonist |
| retinoid receptor agonists, BMS | Bristol-Myers Squibb Co | | neoplasm | Retinoid receptor agonist |
| SR-11237 | Sanofi Recherche SA | | carcinoma | Retinoid receptor agonist |
| Ro-40-0655 | Roche Holding AG | | colon tumor | Retinoid receptor agonist |
| ALRT-1455 | Allergan Ligand Retinoid | | breast tumor, leukemia, | Retinoid receptor agonist |
| | Therapeutics Inc | | lymphoma | |
| RAR agonists, CIRD Galderma | CIRD Galderma | | neoplasm, lung tumor | Retinoid receptor agonist |
| retinoic acid agonist, Eisai | Eisai Co Ltd | WO 97/34869 | neoplasm | Retinoid receptor agonist |
| UAB-8 | University of Alabama in | | myeloid leukemia | Retinoid receptor agonist |
| | Birmingham | | | |
| UAB-30 | The Burnham Institute. | | myeloid leukemia | Retinoid receptor agonist |
| BMS-181163 | Bristol-Myers Squibb Co | | neoplasm | Retinoid receptor agonist |
| adapalene | CIRD Galderma | | acne, neoplasm | Retinoid receptor agonist |
| Am555S | Taiho Pharmaceutical Co Ltd | | digestive system tumor, liver | Retinoid receptor antagonist |
| | | | tumor, neoplasm | |
| AGN-193174 | Allergan Inc | | neoplasm | Retinoid receptor antagonist |
| AGN-193109 | Allergan Inc | | carcinoma | Retinoid receptor antagonist |
| AHPN, CIRD Galderma | CIRD Galderma | WO 97/03682 | breast tumor, leukemia | Retinoid receptor ligand |
| AHPN, CIRD Galderma | CIRD Galderma | WO 97/03682 | breast tumor, leukemia | Retinoid receptor ligand |
| ALRT-1550 | Ligand Pharmaceuticals Inc | | neoplasm | Retinoid receptor ligand |
| AGN-194204 | Allergan Inc | | carcinoma | Retinoid receptor ligand |
| RAR selective retinoids, | Allergan Inc | | neoplasm | Retinoid receptor ligand |
| Allergan | | | | |
| ALRT-1057 | Allergan Ligand Retinoid | | leukemia, neoplasm, kaposis | Retinoid receptor ligand |
| | Therapeutics Inc | | sarcoma, squamous cell | |
| | | | carcinoma, head & neck tumor, | |
| | | | ovary tumor, non-Hodgkin's | |
| | | | lymphoma, carcinoma, renal | |
| | | | tumor, prostate tumor, breast | |
| | | | tumor | |
| zidovudine/zalcitabine, | Glaxo Wellcome plc | | kaposis sarcoma | Reverse transcriptase inhibitor |
| Glaxo/Roche | | | | |
| quinoxalines, HMR/Bayer/Glaxo | Hoechst Marion Roussel Inc | | carcinoma | Reverse transcriptase inhibitor, |
| Wellcome | | | | non-nucleotide |
| LY-207702 | Eli Lilly & Co | | carcinoma | Ribonucleotide reductase |
| | | | | inhibitor |
| MDL-101731 | Hoechst Marion Roussel Inc | EP 0 372 268 | breast tumor, colon tumor, | Ribonucleotide reductase |
| | | | leukemia, lung tumor, prostate | inhibitor |
| | | | tumor, solid tumor | |
| hydroxyurea, NIH | National Institutes of Health | | uterine cervix tumor | Ribonucleotide reductase |
| | | | | inhibitor |
| LY-186641 derivatives, National | National Taiwan University | | neoplasm | Ribonucleotide reductase |
| Taiwan University | | | | inhibitor |
| 3-AP, Vion | Vion Pharmaceuticals Inc | | solid tumor, lung tumor, breast | Ribonucleotide reductase |
| | | | tumor, colorectal tumor, | inhibitor |
| | | | melanoma | |
| OCX-191 | Vion Pharmaceuticals Inc | | neoplasm | Ribonucleotide reductase |
| | | | | inhibitor |
| trimidox | Molecules for Health | | carcinoma | Ribonucleotide reductase |
| | | | | inhibitor |
| didox | Molecules for Health | | neoplasm | Ribonucleotide reductase |
| | | | | inhibitor |
| ribonucleotide reductase | Yale University | | carcinoma | Ribonucleotide reductase |
| inhibitor, Yale | | | | inhibitor |
| sulofenur | Eli Lilly & Co | EP 0 222 475 | carcinoma, neoplasm | Ribonucleotide reductase |
| | | | | inhibitor |
| eudistomins, Solvay | Solvay Duphar BV | | carcinoma | Ribosomal binding inhibitor |
| PC-766B | Sumitomo Pharmaceuticals Co | JP 62-005990 | carcinoma, leukemia | Ribosomal binding inhibitor |
| | Ltd | | | |
| TAP-29 | National Institutes of Health | | carcinoma | Ribosomal metabolic modulator |
| Gelonin-MAb | XOMA Corp | WO 93/09130 | malignant neoplastic disease, | Ribosome binding agent |
| | | | glioma | |
| antisense molecules, Atlantic | Atlantic Pharmaceuticals Inc | U.S. Pat. No. | myeloid leukemia, neoplasm | RNA modulator |
| | | 5,583,032 | | |
| DHAC | National Institutes of Health | | precancer | RNA modulator |
| minamestane | Pharmacia & Upjohn AB | DE 3604179 | carcinoma | RNA polymerase inhibitor |
| edatrexate | SRI International | FR 2 464 956 | carcinoma, lung tumor, | RNA polymerase inhibitor |
| | | | neoplasm | |
| trimetrexate | Warner-Lambert Co | U.S. Pat. No. | carcinoma, colorectal tumor, | RNA polymerase inhibitor |
| | | 4,391,809 | neoplasm, stomach tumor | |
| vorozole | Janssen Pharmaceutica NV | | carcinoma, breast tumor | RNA polymerase inhibitor |
| antitumor nucleosides, Hokkaido | Hokkaido University | | neoplasm | RNA synthesis inhibitor |
| University | | | | |
| doxorubicin (liposome- | NeoPharm Inc | | breast tumor, kaposis sarcoma, | RNA synthesis inhibitor |
| encapsulated), NeoPharm | | | ovary tumor, prostate tumor, | |
| | | | solid tumor | |
| teloxantrone | Parke-Davis & Co | | carcinoma, neoplasm | RNA synthesis inhibitor |
| LY-223592 | Eli Lilly & Co | | carcinoma, neoplasm | RNA synthesis inhibitor |
| aclacinomycin | Il Dong Pharm Co Ltd | | carcinoma | RNA synthesis inhibitor |
| iododoxorubicin | Pharmacia & Upjohn AB | BE 0 892 943 | breast tumor, carcinoma, lung | RNA synthesis inhibitor |
| | | | tumor | |
| nemorubicin | Pharmacia & Upjohn AB | BE 0 904 431 | carcinoma | RNA synthesis inhibitor |
| G-3139 | Genta Inc | | breast tumor, colon tumor, | RNA synthesis inhibitor |
| | | | leukemia, lymphoma, | |
| | | | melanoma, neoplasm, non- | |
| | | | Hodgkin's lymphoma, prostate | |
| | | | tumor, solid tumor | |
| TLC-D-99 | The Liposome Company Inc | | breast tumor, carcinoma, kaposis | RNA synthesis inhibitor |
| | | | sarcoma | |
| bropirimine | Pharmacia & Upjohn Inc | DE 3008693 | bladder tumor | RNA synthesis inhibitor |
| interferon (gamma 1b), | Genentech Inc | | carcinoma, lung tumor, | RNA synthesis inhibitor |
| Genentech | | | melanoma, neoplasm, renal | |
| | | | tumor, urinary tract tumor | |
| diaziquone | National Institutes of Health | | brain tumor, carcinoma, glioma, | RNA synthesis inhibitor |
| | | | leukemia | |
| Adenazole | ICN Pharmaceuticals Inc | | leukemia, neoplasm | RNA synthesis inhibitor |
| Ampligen | Hemispherx Biopharma Inc | CA 1101849 | melanoma, renal tumor, lung | RNA synthesis inhibitor |
| | | | tumor | |
| fazarabine | National Institutes of Health | | carcinoma | RNA synthesis inhibitor |
| G-1128 | Genta Inc | WO 92/02641 | leukemia, neoplasm | RNA synthesis inhibitor |
| oligomers (PNA), ISIS | ISIS Pharmaceuticals Inc | | bacterial infection, neoplasm | RNA synthesis inhibitor |
| pirarubicin | Microbial Chemistry Research | | breast tumor, carcinoma, female | RNA synthesis inhibitor |
| | Foundation | | genital tract tumor, head & neck | |
| | | | tumor, liver tumor, neoplasm, | |
| | | | pancreas tumor | |
| MDL-73811 | Hoechst Marion Roussel Inc | | neoplasm | S adenosylmethionine |
| | | | | decarboxylase inhibitor |
| CGP-48664 | Novartis AG | | neoplasm | S adenosylmethionine |
| | | | | decarboxylase inhibitor |
| lectin inhibitors, Chiroscience | Chiroscience Group plc | | neoplasm | Selectin antagonist |
| serine protease inhibitor, NIH | National Institutes of Health | | liver tumor | Serine protease inhibitor |
| seine protease inhibitors, Tokyo | Tokyo Institute of Technology | | neoplasm | Serine protease inhibitor |
| Institute | | | | |
| NNC-26-9100 | Novo Nordisk A/S | | neoplasm | Somatostatin agonist |
| seglitide | Merck & Co Inc | | stomach tumor | Somatostatin agonist |
| vapreotide | Debiopharm SA | | prostate tumor | Somatostatin analog |
| somatostatin analogs, Neoprobe | Neoprobe Corp | | neoplasm, neuroendocrine | Somatostatin analog |
| | | | tumor, endocrine tumor, breast | |
| | | | tumor | |
| BIM-23190 | Ipsen-Beaufour | | neoplasm | Somatostatin analog |
| BIM-23034 | Ipsen-Beaufour | | neoplasm | Somatostatin analogue |
| lanreotide | Ipsen-Beaufour | | breast tumor, lung tumor, | Somatostatin analogue |
| | | | pancreas tumor, prostate tumor, | |
| | | | renal tumor | |
| WE-14 | University of Lund | | neoplasm | Somatostatin modulator |
| L-054264 | Merck & Co Inc | | neoplasm | Somatostatin modulator |
| L-363377 | Merck & Co Inc | | neoplasm | Somatostatin modulator |
| zaragozic acid C | Merck & Co Inc | | carcinoma | Squalene synthetase inhibitor |
| zaragozic acid C | Merck & Co Inc | | carcinoma | Squalene synthetase inhibitor |
| farnesyl transferase inhibitors, | Pierre Fabre Participations SA | | neoplasm | Squalene synthetase inhibitor |
| Pierre Fabre | | | | |
| PD-169451 | Parke-Davis & Co | | neoplasm | Squalene synthetase inhibitor |
| zaragozic acid D, Merck | Merck & Co Inc | | neoplasm | Squalene synthetase inhibitor |
| J-104126 | Merck & Co Inc | | neoplasm | Squalene synthetase inhibitor |
| Sch-59228 | Schering-Plough Corp | WO 95/10514 | carcinoma, colon tumor, | Squalene synthetase inhibitor |
| | | | pancreas tumor, solid tumor | |
| CB-7741 | Institute of Cancer Research, | | neoplasm | Squalene synthetase inhibitor |
| | UK | | | |
| Sch-207278 | Schering-Plough Corp | | neoplasm | Squalene synthetase inhibitor |
| L-739750 | Merck & Co Inc | | carcinoma | Squalene synthetase inhibitor |
| Sch-48755 | Schering-Plough Corp | | neoplasm | Squalene synthetase inhibitor |
| fluasterone | Aeson Therapeutics Inc | | neoplasm | Steroid agonist |
| fluasterone | Aeson Therapeutics Inc | | neoplasm | Steroid hormone |
| FCE-28718 | Pharmacia & Upjohn SpA | EP 0 755 931 | breast tumor, ovary tumor, | Steroid reductase inhibitor |
| | | | prostate tumor | |
| etanidazole | National Cancer Institute | | neoplasm | Sterol demethylase inhibitor |
| PNU-99533 | Pharmacia & Upjohn Inc | | carcinoma | Stromelysin inhibitor |
| Bay-12-9566 | Bayer AG | | breast tumor, colorectal tumor, | Stromelysin inhibitor |
| | | | metastasis | |
| stromelysin inhibitors, | Hoffmann-La Roche | | inflammation, neoplasm | Stromelysin inhibitor |
| Hoffmann La-Roche | | | | |
| CGS-27023A | Novartis AG | EP 0 606 046 | colorectal tumor, melanoma, | Stromelysin inhibitor |
| | | | neoplasm | |
| antagonist D, ICRF | Imperial Cancer Research | | carcinoma | Substance P antagonist |
| | Technology Ltd | | | |
| substance P antagonists, | The UK Imperial Cancer | | lung tumor | Substance P antagonist |
| ICRF/CRC | Research Fund | | | |
| VML-275 | Vanguard Medica | | melanoma, skin tumor | Sunscreen |
| CRL-1605 | CytRx Corp | | carcinoma | Surfactant |
| BSU-1051 | University of Texas System | | carcinoma | Telomerase inhibitor |
| antisense molecules, Atlantic | Atlantic Pharmaceuticals Inc | U.S. Pat. No. | myeloid leukemia, neoplasm | Telomerase inhibitor |
| | | 5,583,032 | | |
| GRN-56715 | Geron Corp | | neoplasm | Telomerase inhibitor |
| telomerase inhibitors, | Geron Corp | | carcinoma | Telomerase inhibitor |
| Geron/Pharmacia & Upjohn | | | | |
| telomerase antagonist, Amgen | Amgen Inc | | neoplasm | Telomerase inhibitor |
| telomerase inhibitors, University | University of Texas System | | neoplasm | Telomerase inhibitor |
| of Texas System | | | | |
| GRN-56793 | Geron Corp | | neoplasm | Telomerase inhibitor |
| BSU-1021 | Institute of Cancer Research, | | neoplasm | Telomerase inhibitor |
| | UK | | | |
| telomere modulators, Iowa State | Iowa State University | EP 0 666 313 | neoplasm | Telomerase modulator |
| University | | | | |
| FCE-28260 | Pharmacia & Upjohn Inc | | prostate tumor, breast | Testosterone 5 alpha reductase |
| | | | tumor + d2898 | inhibitor |
| MK-0963 | Merck & Co Inc | EP 0 414 490 | neoplasm | Testosterone 5 alpha reductase |
| | | | | inhibitor |
| abiraterone | British Technology Group Plc | GB 2 265 624 | prostate tumor | Testosterone modulator |
| TAN-1518A | Takeda Chemical Industries Ltd | JP 05306278 | carcinoma | Tetracycline |
| TGF-alpha, Berlex | Berlex Laboratories Inc | | carcinoma | TGF alpha |
| BetaKine | Celtrix Pharmaceuticals Inc | | carcinoma | TGF beta-2 |
| heparin-binding peptides, NIH | National Institutes of Health | WO 93/11156 | kaposis sarcoma, breast tumor, | TGF beta antagonist |
| | | | melanoma | |
| vascular MADs, | Eli Lilly & Co | | neoplasm | TGF beta antagonist |
| Lilly/Millennium | | | | |
| SELEX | NeXstar Pharmaceuticals Inc | U.S. Pat. No. | neoplasm | Thrombin inhibitor |
| | | 5,270,163 | | |
| MDR reversal agent, Immunex | National Institutes of Health | | neoplasm | Thymidine kinase inhibitor |
| gene therapy (brain tumor, HSV- | Avigen Inc | | brain tumor, glioma | Thymidine kinase modulator |
| TK), Avigen | | | | |
| HS-TK gene therapy, Canji | Canji Inc | | liver tumor | Thymidine kinase modulator |
| LY-231514 | Eli Lilly & Co | EP 0 432 677 | breast tumor, carcinoma, | Thymidylate synthase inhibitor |
| | | | colorectal tumor, lung tumor, | |
| | | | pancreas tumor | |
| LY-225693 | Eli Lilly & Co | | carcinoma | Thymidylate synthase inhibitor |
| galocitabine | Roche Holding AG | EP 0 316 704 | breast tumor, carcinoma, | Thymidylate synthase inhibitor |
| | | | digestive system tumor, | |
| | | | neoplasm, stomach tumor, | |
| | | | urinary tract tumor | |
| galocitabine | Roche Holding AG | EP 0 316 704 | breast tumor, carcinoma, | Thymidylate synthase inhibitor |
| | | | digestive system tumor, | |
| | | | neoplasm, stomach tumor, | |
| | | | urinary tract tumor | |
| AG-337 | Agouron Pharmaceuticals Inc | | colon tumor, head & neck | |
| | | | tumor, liver tumor, lung tumor, | |
| | | | pancreas tumor, prostate tumor, | |
| | | | solid tumor | |
| thymidylate synthase inhibitor, | Roswell Park Cancer Institute | | carcinoma | Thymidylate synthase inhibitor |
| Roswell Park | | | | |
| FO-152 | Fuji Chemical Industries Co Ltd | FR 2 470 774 | carcinoma | Thymidylate synthase inhibitor |
| quinazolone antifolate TS | Zeneca Group Plc | | neoplasm | Thymidylate synthase inhibitor |
| inhibitors, Zeneca | | | | |
| thymidylate synthase inhibitor, | Agouron Pharmaceuticals Inc | | carcinoma | Thymidylate synthase inhibitor |
| Agouron | | | | |
| pyrimidine deoxynucleoside | Polish Academy of Sciences | | neoplasm | Thymidylate synthase inhibitor |
| analogs, Polish Academy of | | | | |
| Sciences | | | | |
| ZM-246315 | Zeneca Group Plc | | neoplasm | Thymidylate synthase inhibitor |
| CB-300638 | Zeneca Group Plc | | carcinoma | Thymidylate synthase inhibitor |
| TS inhibitor, University of | University of Ontario | | neoplasm | Thymidylate synthase inhibitor |
| Ontario | | | | |
| metesind glucuronate | Agouron Pharmaceuticals Inc | | neoplasm | Thymidylate synthase inhibitor |
| GW-1843 | Glaxo Wellcome plc | WO 91/19700 | carcinoma | Thymidylate synthase inhibitor |
| DMPDDF | Glaxo Wellcome plc | | neoplasm | Thymidylate synthase inhibitor |
| doxifluridine | Nippon Roche KK | | bladder tumor, breast tumor, | Thymidylate synthase inhibitor |
| | | | digestive system tumor, | |
| | | | neoplasm, uterine cervix tumor | |
| ZD-9331 | Zeneca Group Plc | GB 2 264 946 | neoplasm, solid tumor | Thymidylate synthase inhibitor |
| CB-30900 | Institute of Cancer Research, | | carcinoma | Thymidylate synthase inhibitor |
| | UK | | | |
| ICI-198583 | Zeneca Group Plc | | neoplasm | Thymidylate synthase inhibitor |
| raltitrexed | Zeneca Group Plc | EP 0 239 362 | colorectal tumor, neoplasm, | Thymidylate synthase inhibitor |
| | | | ovary tumor, pancreas tumor | |
| Thyrogen | Genzyme Corp | | thyroid tumor | Thyrotropin |
| TNF-alpha, Innogenetics | Innogenetics NV | | neoplasm | TNF-alpha |
| F-4614 | Ishihara Sangyo KK | | neoplasm | TNF-alpha |
| sonermin | Dainippon Pharmaceutical Co | | breast tumor, squamous cell | TNF agonist |
| | Ltd | | carcinoma, neoplasm | |
| OM-174 | Max-Delbrueck-Centrum fuer | | neoplasm | TNF agonist |
| | Molekulare Medizin | | | |
| tumor necrosis factor, | Mochida Pharmaceutical Co Ltd | | skin tumor | TNF agonist |
| Mochida/Hayashibara | | | | |
| TNF gene therapy, NIH | National Institutes of Health | U.S. Pat. No. | carcinoma, melanoma, neoplasm | TNF agonist |
| | | 5,126,132 | | |
| tumor necrosis factor, | Biogen Inc | | carcinoma | TNF agonist |
| Biogen/Knoll | | | | |
| FK-516 | Genentech Inc | | carcinoma, melanoma, sarcoma | TNE agonist |
| tumor necrosis factor, Asahi | Asahi Chemical Industry Co Ltd | | carcinoma, neoplasm | TNF agonist |
| (-)-epigallocatechin gallate | National Institutes of Health | | carcinoma, neoplasm | TNF alpha antagonist |
| | Japan | | | |
| F4CC-1104 | Massachusetts Institute of | | neoplasm | TNF alpha antagonist |
| | Technology | | | |
| thalidomide, Celgene | Celgene Corp | WO 92/14455 | carcinoma, rheumatoid arthritis | TNF alpha synthesis inhibitor |
| marimastat analogs, Zeneca | Zeneca Group Plc | | carcinoma | TNF alpha synthesis inhibitor |
| batimastat analogs, SB | SmithKline Beecham plc | | carcinoma | TNF alpha synthesis inhibitor |
| thalidomide, | Entremed Inc | | brain tumor, breast tumor, | TNF alpha synthesis inhibitor |
| Entremed/BMS/NCI | | | diabetic retinopathy, kaposis | |
| | | | sarcoma, neoplasm, ocular | |
| | | | disease, prostate tumor | |
| PCM-4 | Omega Pharm Inc | | neoplasm | TNF antagonist |
| BB-2275 | British Biotech plc | | neoplasm | TNF antagonist |
| lymphotoxin, Genentech | Genentech Inc | | neoplasm, leukemia | TNF beta |
| sonermin | Dainippon Pharmaceutical Co | | breast tumor, squamous cell | TNF modulator |
| | Ltd | | carcinoma, neoplasm | |
| alnorin | NPO Vector | | neoplasm | TNF modulator |
| TNF-beta analogs, NPO Vector | NPO Vector | | neoplasm | TNF modulator |
| cytokines, Enzon | Enzon Inc | | neoplasm | TNF modulator |
| AR-324 | Aronex Pharmaceuticals Inc | | neoplasm | TNF modulator |
| OH-1 | Hayashibara Co Ltd | | neoplasm, breast tumor | TNF modulator, IFN agonist |
| NSC-649488 | University of Auckland | | solid tumor | TNF synthesis stimulator |
| DT-5461 | Daiichi Seiyaku Co Ltd | ZA 88/01430 | neoplasm | TNF synthesis stimulator |
| ONO-4007 | Ono Pharmaceutical Co Ltd | EP 0 226 381 | carcinoma, neoplasm | TNF synthesis stimulator |
| tumor necrosis factor, | Biogen Inc | | carcinoma | TNFmodulator |
| Biogen/Knoll | | | | |
| FK-516 | Genentech Inc | | carcinoma, melanoma, sarcoma | TNFmodulator |
| tumor necrosis factor, Asahi | Asahi Chemical Industry Co Ltd | | carcinoma, neoplasm | TNFmodulator |
| tumor necrosis factor, | Mochida Pharmaceutical Co Ltd | | skin tumor | TNFmodulator |
| Mochida/Hayashibara | | | | |
| TNF-alpha, Innogenetics | Innogenetics NV | | neoplasm | TNFr modulator |
| celastrol | Schering AG | | neoplasm | Topoisomerase I inhibitor |
| intoplicine | Rhone-Poulenc Rorer Inc | EP 0 402 232 | solid tumor | Topoisomerase I inhibitor |
| elsamitrucin | Bristol-Myers Squibb Co | BE 0 900 735 | carcinoma, neoplasm | Topoisomerase I inhibitor |
| NSC-665517 | National Cancer Institute | | carcinoma | Topoisomerase I inhibitor |
| topoisomerase inhibitor, Daiichi | Daiichi Seiyaku Co Ltd | | carcinoma | Topoisomerase I inhibitor |
| anhydrous delivery system, | Matrix Pharmaceutical Inc | | carcinoma | Topoisomerase I inhibitor |
| Matrix | | | | |
| XR-5942 | Xenova Group plc | | neoplasm | Topoisomerase I inhibitor |
| BE-13793C | Banyu Pharmaceutical Co Ltd | EP 0 388 956 | carcinoma, neoplasm | Topoisomerase I inhibitor |
| TRK-710 | Toray Industries Inc | | neoplasm | Topoisomerase I inhibitor |
| XR-5000 | Cancer Research Campaign | | carcinoma, breast tumor, lung | Topoisomerase I inhibitor |
| | Technology Ltd | | tumor, colon tumor, skin tumor, | |
| | | | brain tumor, melanoma | |
| TAN-1518A | Takeda Chemical Industries Ltd | JP 05306278 | carcinoma | Topoisomerase I inhibitor |
| NSC-675967 | National Cancer Institute | | carcinoma | Topoisomerase I inhibitor |
| DACA | University of Auckland | | solid tumor | Topoisomerase I inhibitor |
| julibrosides | Taisho Pharmaceutical Co Ltd | | carcinoma | Topoisomerase I inhibitor |
| A35566-A | Sankyo KK | JP 07316091 | neoplasm | Topoisomerase I inhibitor |
| CKD-602 | Chong Kun Dang Corp | WO 96/21666 | neoplasm | Topoisomerase I inhibitor |
| HAR-7 | Harrier Inc | | solid tumor | Topoisomerase I inhibitor |
| camptothecin analogs, RTI/BMS | Research Triangle Institute | | neoplasm | Topoisomerase I inhibitor |
| TAS-103 | Taiho Pharmaceutical Co Ltd | | lung tumor, neoplasm, stomach | Topoisomerase I inhibitor |
| | | | tumor | |
| camptothecin derivatives, | Pharmacia & Upjohn SpA | WO 96/37496 | neoplasm | Topoisomerase I inhibitor |
| Pharmacia | | | | |
| NU/ICRF-505 | Imperial Cancer Research | | neoplasm | Topoisomerase I inhibitor |
| | Technology Ltd | | | |
| MPI-5019 | Matrix Pharmaceutical Inc | | carcinoma | Topoisomerase I inhibitor |
| BNP-1350 | Bionumerik Pharmaceuticals Inc | | solid tumor | Topoisomerase I inhibitor |
| RFS-2000 | Stehlin Foundation For Cancer | | neoplasm, pancreas tumor, ovary | Topoisomerase I inhibitor |
| | Research | | tumor | |
| DMNQ derivatives, Chungnam | Chungnam University | | neoplasm | Topoisomerase I inhibitor |
| University | | | | |
| BN-80245 | Institut Henri Beaufour | | carcinoma | Topoisomerase I inhibitor |
| NSC-314622 | National Cancer Institute | | neoplasm | Topoisomerase I inhibitor |
| 10-hydroxycamptothecin | Chiba University | | solid tumor | Topoisomerase I inhibitor |
| derivatives, Chiba | | | | |
| NX-211 | Glaxo Wellcome plc | | neoplasm | Topoisomerase I inhibitor |
| irinotecan | Yakult Honsha KK | JP 60-019790 | carcinoma, lung tumor, colon | Topoisomerase I inhibitor |
| | | | tumor, neoplasm, uterus tumor, | |
| | | | ovary tumor, colorectal tumor, | |
| | | | stomach tumor, brain tumor, | |
| | | | non-Hodgkin's lymphoma, | |
| | | | uterine cervix tumor, pancreas | |
| | | | tumor | |
| DU-6596 | Daiichi Seiyaku Co Ltd | | carcinoma, neoplasm | Topoisomerase I inhibitor |
| DX-8951 | Daiichi Seiyaku Co Ltd | | neoplasm | Topoisomerase I inhibitor |
| NB-506 | Banyu Pharmaceutical Co Ltd | WO 93/11145 | neoplasm | Topoisomerase I inhibitor |
| SKF-108025 | SmithKline Beecham plc | | carcinoma | Topoisomerase I inhibitor |
| topoisomerase I inhibitors, | Glaxo Wellcome plc | | carcinoma | Topoisomerase I inhibitor |
| Glaxo | | | | |
| SKF-107874 | SmithKline Beecham plc | | carcinoma | Topoisomerase I inhibitor |
| AG-555 | Hebrew University of Jerusalem | | carcinoma | Topoisomerase I inhibitor |
| 9-aminocamptothecin | Research Triangle Institute | | bladder tumor, carcinoma, colon | Topoisomerase I inhibitor |
| | | | tumor, colorectal tumor, head & | |
| | | | neck tumor, lung tumor, | |
| | | | neoplasm, pancreas tumor, | |
| | | | prostate tumor, renal tumor, | |
| | | | solid tumor, stomach tumor | |
| lurtotecan | Glaxo Inc | EP 0 540 099 | neoplasm | Topoisomerase I inhibitor |
| TAN-1496 | Takeda Chemical Industries Ltd | JP 05301877 | carcinoma | Topoisomerase I inhibitor |
| topotecan | SmithKline Beecham plc | EP 0 321 122 | breast tumor, carcinoma, colon | Topoisomerase I inhibitor |
| | | | tumor, glioma, leukemia, lung | |
| | | | tumor, lymphoma, | |
| | | | myeloproliferative disorder, | |
| | | | ovary tumor | |
| JSKIV-47 | Rutgers University | U.S. Pat. No. | neoplasm | Topoisomerase I inhibitor |
| | | 5,767,142 | | |
| UCE-6 | Kyowa Hakko Kogyo Co Ltd | | neoplasm | Topoisomerase I modulator |
| TLC-D-99 | The Liposome Company Inc | | breast tumor, carcinoma, kaposis | Topoisomerase II inhibitor |
| | | | sarcoma | |
| intoplicine | Rhone-Poulenc Rorer Inc | EP 0 402 232 | solid tumor | Topoisomerase II inhibitor |
| doxorubicin (liposome- | NeoPharm Inc | | breast tumor, kaposis sarcoma, | Topoisomerase II inhibitor |
| encapsulated), NeoPharm | | | ovary tumor, prostate tumor, | |
| | | | solid tumor | |
| iododoxorubicin | Pharmacia & Upjohn AB | BE 0 892 943 | breast tumor, carcinoma, lung | Topoisomerase II inhibitor |
| | | | tumor | |
| teloxantrone | Parke-Davis & Co | | carcinoma, neoplasm | Topoisomerase II inhibitor |
| aclacinomycin | Il Dong Pharm Co Ltd | | carcinoma | Topoisomerase II inhibitor |
| Ro-23-7777 | Roche Holding AG | | carcinoma | Topoisomerase II inhibitor |
| NK-109 | Nippon Kayaku Co Ltd | EP 0 432 630 | neoplasm | Topoisomerase II inhibitor |
| 7U85 | Burroughs Wellcome Inc | WO 91/14688 | carcinoma | Topoisomerase II inhibitor |
| 773U82 | Burroughs Wellcome Inc | EP 0 125 702 | carcinoma, pancreas tumor | Topoisomerase II inhibitor |
| elsamitrucin | Bristol-Myers Squibb Co | BE 0 900 735 | carcinoma, neoplasm | Topoisomerase II inhibitor |
| nemorubicin | Pharmacia & Upjohn AB | BE 0 904 431 | carcinoma | Topoisomerase II inhibitor |
| losoxantrone | Parke-Davis & Co | EP 0 103 381 | breast tumor, neoplasm | Topoisomerase II inhibitor |
| TAS-103 | Taiho Pharmaceutical Co Ltd | | lung tumor, neoplasm, stomach | Topoisomerase II inhibitor |
| | | | tumor | |
| AD-312 | Anthra Pharmaceuticals | | carcinoma, neoplasm, solid | Topoisomerase II inhibitor |
| | | | tumor | |
| AD-347 | Pharmacia & Upjohn AB | | neoplasm | Topoisomerase II inhibitor |
| BBR-2778 | Boehringer Mannheim GmbH | | leukemia, lymphoma | Topoisomerase II inhibitor |
| WIN-33377 | Sterling Winthrop Products Inc | | solid tumor | Topoisomerase II inhibitor |
| NSC-655649 | University of Wisconsin, | | neoplasm | Topoisomerase II inhibitor |
| | Madison | | | |
| azatoxin | National Institutes of Health | | carcinoma | Topoisomerase II inhibitor |
| NSC-665517 | National Cancer Institute | | carcinoma | Topoisomerase II inhibitor |
| topoisomerase inhibitor, Daiichi | Daiichi Seiyaku Co Ltd | | carcinoma | Topoisomerase II inhibitor |
| anhydrous delivery system, | Matrix Pharmaceutical Inc | | carcinoma | Topoisomerase II inhibitor |
| Matrix | | | | |
| XR-5942 | Xenova Group plc | | neoplasm | Topoisomerase II inhibitor |
| BE-13793C | Banyu Pharmaceutical Co Ltd | EP 0 388 956 | carcinoma, neoplasm | Topoisomerase II inhibitor |
| TRK-710 | Toray Industries Inc | | neoplasm | Topoisomerase II inhibitor |
| XR-5000 | Cancer Research Campaign | | carcinoma, breast tumor, lung | Topoisomerase II inhibitor |
| | Technology Ltd | | tumor, colon tumor, skin tumor, | |
| | | | brain tumor, melanoma | |
| mitonafide | BASF AG | | carcinoma | Topoisomerase II inhibitor |
| pazelliptine | Elf Sanofi | DE 2815724 | carcinoma | Topoisomerase II inhibitor |
| AQ4N | De Montfort University | | neoplasm | Topoisomerase II inhibitor |
| A-65281 | Abbott Laboratories | | neoplasm | Topoisomerase II inhibitor |
| MPI-6003 | Matrix Pharmaceutical Inc | | carcinoma | Topoisomerase II inhibitor |
| piroxantrone | Parke-Davis & Co | EP 0 103 381 | carcinoma, melanoma, neoplasm | Topoisomerase II inhibitor |
| datelliptium chloride | Elf Sanofi | EP 0 209 511 | neoplasm, breast tumor | Topoisomerase II inhibitor |
| BBR-2828 | Boehringer Mannheim GmbH | | neoplasm | Topoisomerase II inhibitor |
| BO-2367 | Banyu Pharmaceutical Co Ltd | | carcinoma | Topoisomerase II inhibitor |
| NCA-0465 | Taisho Pharmaceutical Co Ltd | | neoplasm | Topoisomerase II inhibitor |
| sobuzoxane | Zenyaku Kogyo Co Ltd | | leukemia, non Hodgkin's | Topoisomerase II inhibitor |
| | | | lymphoma | |
| ER-37328 | Eisai Co Ltd | | neoplasm | Topoisomerase II inhibitor |
| CC-131 | Erasmus University | | renal tumor | Topoisomerase II inhibitor |
| ellipticine-estradiol conjugates | R W Johnson Pharmaceutical | | carcinoma | Topoisomerase II inhibitor |
| | Research Institute | | | |
| GR-122222X | Glaxo Wellcome plc | | neoplasm | Topoisomerase II inhibitor |
| ICRF-193 | Imperial Cancer Research | | neoplasm | Topoisomerase II inhibitor |
| | Technology Ltd | | | |
| morindone | Meiji Milk Products Co Ltd | | neoplasm | Topoisomerase II inhibitor |
| A-74932 | Abbott Laboratories | | carcinoma, lung tumor, | Topoisomerase II inhibitor |
| | | | melanoma, neoplasm | |
| BE-10988 | Banyu Pharmaceutical Co Ltd | JP 03197481 | carcinoma, neoplasm | Topoisomerase II inhibitor |
| Win-58161 | Sterling Winthrop Products Inc | | carcinoma | Topoisomerase II inhibitor |
| elinafide | Knoll AG | | neoplasm | Topoisomerase II inhibitor |
| GL-331 | University of North Carolina | | colorectal tumor, lung tumor | Topoisomerase II inhibitor |
| GI-149893 | Glaxo Inc | | neoplasm | Topoisomerase II inhibitor |
| CP-100964 | Pfizer Inc | | neoplasm | Topoisomerase II inhibitor |
| Win-64593 | Sterling Winthrop Products Inc | | carcinoma | Topoisomerase II inhibitor |
| WR-63320 | Elf Sanofi | | carcinoma | Topoisomerase II inhibitor |
| TOP-53 | Otsuka Pharmaceutical Co Ltd | | lung tumor | Topoisomerase II inhibitor |
| asulacrine | Auckland Division Cancer | EP 0 039 224 | breast tumor, lung tumor, | Topoisomerase II inhibitor |
| | Society of New Zealand Inc | | melanoma, solid tumor | |
| amrubicin | Sumitomo Pharmaceuticals Co | | lung tumor, neoplasm | Topoisomerase II inhibitor |
| | Ltd | | | |
| fostriecin | Parke-Davis & Co | EP 0 087 021 | neoplasm | Topoisomerase II inhibitor |
| fosquidone | Glaxo Wellcome plc | DE 3725185 | carcinoma, neoplasm | Topoisomerase II inhibitor |
| Win-63320 | Sterling Winthrop Products Inc | | neoplasm | Topoisomerase II inhibitor |
| NK-611 | Nippon Kayaku Co Ltd | EP 0 369 369 | neoplasm, solid tumor | Topoisomerase II inhibitor |
| Ro-46-7864 | Roche Holding AG | EP 0 433 648 | neoplasm | Topoisomerase II inhibitor |
| Ro-47-3359 | Roche Holding AG | | neoplasm | Topoisomerase II inhibitor |
| S-16020-2 | Servier | | carcinoma | Topoisomerase II inhibitor |
| clerocidin | Bristol-Myers Squibb Co | | neoplasm | Topoisomerase II inhibitor |
| merbarone | Uniroyal Chemical Co Inc | | neoplasm, uterine cervix tumor, | Topoisomerase II inhibitor |
| | | | pancreas tumor | |
| A-85226 | Abbott Laboratories | | neoplasm | Topoisomerase II inhibitor |
| BBR-2577 | Boehringer Mannheim GmbH | | lung tumor, viral infection | Topoisomerase II inhibitor |
| DNA topoisomerase 2 inhibitor, | Centre National de la Recherche | | carcinoma | Topoisomerase II inhibitor |
| CNRS | Scientifique (CNRS) | | | |
| BE-22179 | Banyu Pharmaceutical Co Ltd | | leukemia, neoplasm | Topoisomerase II inhibitor |
| W4R | Mediolanum Pharmaceuticals | | colon tumor | Topoisomerase II inhibitor |
| | Inc | | | |
| A-74932 derivatives, Abbott | Abbott Laboratories | | neoplasm | Topoisomerase II inhibitor |
| AP-4010 | ACCESS Pharmaceuticals Inc | | carcinoma | Topoisomerase II inhibitor |
| AHMA | Eli Lilly & Co | | leukemia, neoplasm | Topoisomerase II inhibitor |
| IST-622 | Ishihara Sangyo KK | | neoplasm | Topoisomerase II inhibitor |
| DACA | University of Auckland | | solid tumor | Topoisomerase II inhibitor |
| CP-115953 | Pfizer Inc | | neoplasm | Topoisomerase II modulator |
| AD-312 | Anthra Pharmaceuticals | | carcinoma, neoplasm, solid | Topoisomerase inhibitor |
| | | | tumor | |
| AD-347 | Pharmacia & Upjohn AB | | neoplasm | Topoisomerase inhibitor |
| CP-115953 | Pfizer Inc | | neoplasm | Topoisomerase inhibitor |
| anticancer, Biota/La Trobe | La Trobe University | | colon tumor, lung tumor, | Topoisomerase inhibitor |
| | | | stomach tumor | |
| ED-110 | Banyu Pharmaceutical Co Ltd | WO 91/18003 | carcinoma | Topoisomerase inhibitor |
| PD-115934 | Parke-Davis & Co | EP 0 138 302 | carcinoma | Topoisomerase inhibitor |
| LU-125950 | BASF Bioresearch Corp | | carcinoma | Topoisomerase inhibitor |
| PEG-camptothecin, Enzon | Enzon Inc | | carcinoma | Topoisomerase inhibitor |
| NC-190 | Taisho Pharmaceutical Co Ltd | | neoplasm | Topoisomerase inhibitor |
| azonafide | Research Corp Technologies Inc | | carcinoma, neoplasm | Topoisomerase inhibitor |
| anticancer (quinolone), II Dong | II Dong Pharm Co Ltd | | carcinoma | Topoisomerase inhibitor |
| topoisomerase inhibitors, Avax | Avax Technologies Inc | | neoplasm | Topoisomerase inhibitor |
| pazelliptine | Elf Sanofi | DE 2815724 | carcinoma | Topoisomerase inhibitor |
| zaragozic acid C | Merck & Co Inc | | carcinoma | Transferase inhibitor |
| zaragozic acid C | Merck & Co Inc | | carcinoma | Transferase inhibitor |
| LY-231514 | Eli Lilly & Co | EP 0 432 677 | breast tumor, carcinoma, | Transferase inhibitor |
| | | | colorectal tumor, lung tumor, | |
| | | | pancreas tumor | |
| minamestane | Pharmacia & Upjohn AB | DE 3604179 | carcinoma | Transferase inhibitor |
| LY-225693 | Eli Lilly & Co | | carcinoma | Transferase inhibitor |
| edatrexate | SRI International | FR 2 464 956 | carcinoma, lung tumor, | Transferase inhibitor |
| | | | neoplasm | |
| E-7010 | Eisai Co Ltd | EP 0 472 053 | carcinoma | Transferase inhibitor |
| lamivudine | BioChem Pharma Inc | EP 0 382 526 | | Transferase inhibitor |
| atamestane | Schering AG | DE 3322285 | carcinoma, neoplasm, breast | Transferase inhibitor |
| | | | tumor | |
| exemestane | Pharmacia & Upjohn AB | DE 3622841 | breast tumor | Transferase inhibitor |
| fadrozole hydrochloride | Novartis AG | U.S. Pat. No. | breast tumor, carcinoma | Transferase inhibitor |
| | | 4,588,732 | | |
| sparfosic acid | Warner-Lambert Co | U.S. Pat. No. | carcinoma, colorectal tumor, | Transferase inhibitor |
| | | 4,215,070 | neoplasm | |
| etanidazole | National Cancer Institute | | neoplasm | Transferase inhibitor |
| XR-3005 | Xenova Ltd | | colon tumor, pancreas tumor, | Transferase inhibitor |
| | | | solid tumor | |
| L-744832 | Merck & Co Inc | | neoplasm | Transferase inhibitor |
| letrozole | Novartis AG | EP 0 236 940 | breast tumor | Transferase inhibitor |
| AICARFT inhibitor, Agouron | Agouron Pharmaceuticals Inc | WO 94/13295 | neoplasm | Transferase inhibitor |
| GGTI-286 | University of Pittsburgh | | carcinoma | Transferase inhibitor |
| PD-128763 | Parke-Davis & Co | EP 0 355 750 | carcinoma | Transferase inhibitor |
| L-736728 | Merck & Co Inc | | neoplasm | Transferase inhibitor |
| BMS-182566 | Bristol-Myers Squibb AG | | neoplasm | Transferase inhibitor |
| mifepristone | Roussel Uclaf SA | FR 2 497 807 | breast tumor | Transferase stimulator |
| Humalog | Eli Lilly & Co | | diabetes mellitus | Transferase stimulator |
| taxol analogs, Stanford/Albert | Stanford University | | neoplasm | Tubulin agonist |
| Einstein | | | | |
| paclitaxel, NIH | National Institutes of Health | | ovary tumor, breast tumor, | Tubulin agonist |
| | | | restenosis, sarcoma, neoplasm, | |
| | | | head & neck tumor, lung tumor, | |
| | | | kaposis sarcoma, stomach tumor | |
| taxol analogs, Abbott | Abbott Laboratories | | carcinoma | Tubulin agonist |
| taxol analogs, Rhone Poulenc | Rhone-Poulenc Rorer Inc | | carcinoma | Tubulin agonist |
| Rorer | | | | |
| RPR-112378 | Rhone-Poulenc SA | | neoplasm | Tubulin antagonist |
| anticancers, University of North | University of North Carolina | | neoplasm | Tubulin antagonist |
| Carolina | | | | |
| anticancers, University of North | University of North Carolina | | neoplasm | Tubulin antagonist |
| Carolina | | | | |
| MPI-6003 | Matrix Pharmaceutical Inc | | carcinoma | Tubulin antagonist |
| azatoxin | National Institutes of Health | | carcinoma | Tubulin antagonist |
| spongistatins | Chiroscience Group plc | EP 0 608 111 | neoplasm | Tubulin antagonist |
| rhizoxin | Fujisawa Pharmaceutical Co Ltd | EP 0 132 772 | carcinoma, solid tumor, breast | Tubulin binding agent |
| | | | tumor, lung tumor, head & neck | |
| | | | tumor, melanoma, ovary tumor, | |
| | | | colorectal tumor, renal tumor | |
| PNU-156692 | Pharmacia & Upjohn Inc | | neoplasm | Tubulin binding agent |
| PNU-166087 | Pharmacia & Upjohn Inc | | neoplasm | Tubulin binding agent |
| PNU-156691 | Pharmacia & Upjohn Inc | | neoplasm | Tubulin binding agent |
| PNU-157548 | Pharmacia & Upjohn Inc | | neoplasm | Tubulin binding agent |
| palmitoylrhizoxin | Sankyo KK | | carcinoma | Tubulin binding agent |
| noscapine | Emory University | | neoplasm | Tubulin binding agent |
| anti-tubulin MAb, Allegheny | Allegheny University of the | | leukemia, neoplasm | Tubulin ligand |
| | Health Sciences | | | |
| farnesyl transferase inhibitors, | Pierre Fabre Participations SA | | neoplasm | Tubulin modulator |
| Pierre Fabre | | | | |
| cemadotin | Knoll AG | | neoplasm | Tubulin modulator |
| calcein/AM | Uppsala University | | carcinoma | Tubulin modulator |
| LL-15 | Pharma Mar SA | | neoplasm | Tubulin modulator |
| estratropones, | Allergan Inc | | angiogenesis disorder, neoplasm | Tubulin modulator |
| Allergan/University of Virginia | | | | |
| T-138068 | Tularik Inc | | neoplasm | Tubulin modulator |
| CV-6504 | Takeda Chemical Industries Ltd | U.S. Pat. No. | carcinoma | TXA2 antagonist |
| | | 4,851,413 | | |
| FCE-28718 | Pharmacia & Upjohn SpA | EP 0 755 931 | breast tumor, ovary tumor, | TXA2 synthesis inhibitor |
| | | | prostate tumor | |
| QX-101 | Taiho Pharmaceutical Co Ltd | | neoplasm | Tyrosinase inhibitor |
| SU-5271 | Zeneca Group Plc | | neoplasm | Tyrosine kinase inhibitor |
| flavopiridol | Hoechst AG | | breast tumor, lung tumor, | Tyrosine kinase inhibitor |
| | | | digestive system tumor, | |
| | | | neoplasma, lymphoma | |
| SU-101 | Sugen Inc | WO 96/33745 | neoplasm, solid tumor, ovary | Tyrosine kinase inhibitor |
| | | | tumor, glioma, kaposis sarcoma, | |
| | | | prostate tumor, lung tumor | |
| celastrol | Schering AG | | neoplasm | Tyrosine kinase inhibitor |
| CGP-52411 | Novartis AG | EP 0 516 588 | neoplasm | Tyrosine kinase inhibitor |
| anti-flk-1, ImClone systems Inc | Imclone Systems Inc | WO 95/21868 | angiogenesis disorder, | Tyrosine kinase inhibitor |
| | | | carcinoma | |
| CEP-2563 | Cephalon Inc | WO 96/31515 | prostate tumor | Tyrosine kinase inhibitor |
| HER-2 antagonist, Sugen/Asta | Sugen Inc | | breast tumor, lung tumor, ovary | Tyrosine kinase inhibitor |
| | | | tumor, prostate tumor, stomach | |
| | | | tumor | |
| NSC-675967 | National Cancer Institute | | carcinoma | Tyrosine kinase inhibitor |
| SU-5416 | Sugen Inc | | angiogenesis disorder, diabetic | Tyrosine kinase inhibitor |
| | | | retinopathy, neoplasm, solid | |
| | | | tumor | |
| FCE-26806 | Pharmacia & Upjohn SpA | | neoplasm | Tyrosine kinase inhibitor |
| DAB-720 | Mitsubishi Chemical Corp | | neoplasm | Tyrosine kinase inhibitor |
| CEP-751 | Cephalon Inc | | prostate tumor | Tyrosine kinase inhibitor |
| ZD-1838 | Zeneca Group Plc | WO 96/15118 | breast tumor, lung tumor | Tyrosine kinase inhibitor |
| tyrosine kinase inhibitor, Pfizer | Pfizer Inc | | neoplasm | Tyrosine kinase inhibitor |
| CGP-60261 | Novartis AG | | carcinoma | Tyrosine kinase inhibitor |
| EGF-RTK antagonist, Sugen | Sugen Inc | | brain tumor, breast tumor, head | Tyrosine kinase inhibitor |
| | | | & neck tumor, lung tumor, | |
| | | | stomach tumor | |
| ALL-TK antagonists, Sugen | Sugen Inc | | lymphoma, leukemia | Tyrosine kinase inhibitor |
| GRB2 antagonists, Sugen | Sugen Inc | | leukemia, neoplasm | Tyrosine kinase inhibitor |
| CGP-57148 | Novartis AG | | bone marrow transplantation, | Tyrosine kinase inhibitor |
| | | | myeloid leukemia, neoplasm | |
| ZD-1839 | Zeneca Group Plc | WO 96/33980 | carcinoma, solid tumor | Tyrosine kinase inhibitor |
| erbB-2 receptor inhibitors, SRI | Southern Research Inst | | neoplasm | Tyrosine kinase inhibitor |
| PD-158780 | Parke-Davis & Co Ltd | | carcinoma, neoplasm, breast | Tyrosine kinase inhibitor |
| | | | tumor | |
| benzothiazoles | University of Nottingham | | breast tumor | Tyrosine kinase inhibitor |
| PD-171026 | Parke-Davis & Co | | neoplasm | Tyrosine kinase inhibitor |
| BE-23372M derivatives, Banyu | Banyu Pharmaceutical Co Ltd | | neoplasm | Tyrosine kinase inhibitor |
| Met TK antagonist, Sugen | Sugen Inc | | stomach tumor, colorectal | Tyrosine kinase inhibitor |
| | | | tumor, lung tumor | |
| PD-159973 | Parke-Davis & Co | | carcinoma | Tyrosine kinase inhibitor |
| GW-282974 | Glaxo Wellcome plc | | breast tumor, lung tumor | Tyrosine kinase inhibitor |
| CP-292597 | Pfizer Central Research | | neoplasm | Tyrosine kinase inhibitor |
| ZM-105180 | Zeneca Pharmaceuticals | WO 96/15118 | neoplasm | Tyrosine kinase inhibitor |
| GW-7072X | Glaxo Wellcome plc | | neoplasm | Tyrosine kinase inhibitor |
| Lck tyrosine kinase inhibitors, | Bristol-Myers Squibb Co | | carcinoma | Tyrosine kinase inhibitor |
| BMS | | | | |
| PD-168393 | Parke-Davis & Co | | neoplasm | Tyrosine kinase inhibitor |
| PD-173956 | Parke-Davis & Co | | neoplasm | Tyrosine kinase inhibitor |
| tyrosine kinase inhibitors, | Novartis AG | | neoplasm | Tyrosine kinase inhibitor |
| Novartis | | | | |
| RG-14620 | Rhone-Poulenc Rorer Inc | WO 91/16051 | psoriasis, squamous cell | Tyrosine kinase inhibitor |
| | | | carcinoma | |
| CGP-59326 | Novartis AG | WO 96/10028 | neoplasm | Tyrosine kinase inhibitor |
| genistein | Yamanouchi Pharmaceutical Co | | carcinoma | Tyrosine kinase inhibitor |
| | Ltd | | | |
| FCE-27119 | Pharmacia & Upjohn SpA | | neoplasm | Tyrosine kinase inhibitor |
| RG-13022 | Rhone-Poulenc Rorer Inc | WO 91/16051 | breast tumor, squamous cell | Tyrosine kinase inhibitor |
| | | | carcinoma | |
| RG-50864 | Rhone-Poulenc SA | WO 91/16892 | neoplasm | Tyrosine kinase inhibitor |
| PD-154233 | Parke-Davis & Co | | neoplasm | Tyrosine kinase inhibitor |
| TT-232 | BioSignal Inc | | neoplasm | Tyrosine kinase inhibitor |
| AG-514 | Agouron Pharmaceuticals Inc | | neoplasm | Tyrosine kinase inhibitor |
| AG-568 | Agouron Pharmaceuticals Inc | | neoplasm | Tyrosine kinase inhibitor |
| PD-151514 | Parke-Davis & Co | | neoplasm | Tyrosine kinase inhibitor |
| BE-23372M | Banyu Pharmaceutical Co Ltd | JP 42-75284 | neoplasm | Tyrosine kinase inhibitor |
| KW-6151 | Kyowa Hakko Kogyo Co Ltd | | prostate tumor | Tyrosine kinase inhibitor |
| paeciloquinones | Novartis AG | | neoplasm | Tyrosine kinase inhibitor |
| PDGFrTK inhibitors, Sterling | Sterling Winthrop Group Ltd | | carcinoma | Tyrosine kinase inhibitor |
| Winthrop | | | | |
| SDZ-LAP-977 | Novartis AG | | melanoma, neoplasm | Tyrosine kinase inhibitor |
| CGP-53716 | Novartis AG | | neoplasm | Tyrosine kinase inhibitor |
| CGP-79787 | Novartis AG | | carcinoma | Tyrosine kinase inhibitor |
| B43-genistein | University of Minnesota | WO 96/06116 | leukemia | Tyrosine kinase inhibitor |
| tyrosine kinase inhibitors, Sugen | Sugen Inc | | carcinoma | Tyrosine kinase inhibitor |
| CGP-62706 | Novartis AG | | neoplasm | Tyrosine kinase inhibitor, |
| | | | | Anticancer |
| AG-957 | National Cancer Institute | | myeloid leukemia | Tyrosine kinase modulator |
| cdk4 inhibitor, Agouron | Agouron Pharmaceuticals Inc | | neoplasm | Unclassified enzyme inhibitor |
| CHIR-11509 | Chiron Corp | WO 96/40747 | neoplasm | Urokinase inhibitor |
| urokinase inhibitor, 3- | 3-Dimensional Pharmaceuticals | | metastasis | Urokinase inhibitor |
| Dimensional | Inc | | | |
| B-428 | Eisai Co Ltd | EP 0 568 289 | neoplasm | Urokinase inhibitor |
| B-623 | Eisai Co Ltd | | neoplasm | Urokinase inhibitor |
| p-aminobenzamidine | Pharmacia & Upjohn AB | | neoplasm | Urokinase inhibitor |
| uPAR antagonists, Glaxo | Glaxo Wellcome plc | | neoplasm | Urokinase modulator |
| Wellcome | | | | |
| DUROS (leuprolide) | Alza Corp | | prostate tumor | Vaccine |
| Provax, IDEC | IDEC Pharmaceuticals Corp | | carcinoma, vaccination | Vaccine |
| Il-2 gene therapy (cancer), | Sidney Kimmel Cancer Center | | brain tumor, colon tumor | Vaccine |
| Immune Response/SDRCC | | | | |
| HSPPC-96 | Mount Sinai School of Medicine | | carcinoma, colorectal tumor, | Vaccine |
| | | | imelanoma, neoplasm, pancreas | |
| | | | tumor, stomach tumor | |
| CERES-Vax vaccine delivery | Ceres Pharmaceuticals | | carcinoma | Vaccine |
| system | | | | |
| cancer vaccine, | Polymasc Pharmaceuticals plc | EP 0 727 438 | carcinoma | Vaccine |
| PolyMASC/Hydro Med | | | | |
| cancer vaccine, Cytel/Searle | Cytel Corp | | neoplasm | Vaccine |
| GVAX | Cell Genesys Inc | WO 92/05262 | colorectal tumor, lung tumor, | Vaccine |
| | | | melanoma, neoplasm, prostate | |
| | | | tumor, renal tumor | |
| B43.13, Biomira | Biomira Inc | | ovary tumor | Vaccine |
| B43.13, Biomira | Biomira Inc | | ovary tumor | Vaccine |
| interleukin-2 vaccine, ICR | Institute of Cancer Research, | | carcinoma | Vaccine |
| | UK | | | |
| interleukin-2 vaccine, ICR | Institute of Cancer Research, | | carcinoma | Vaccine |
| | UK | | | |
| Globo-H-KLH, Memorial Sloan- | Memorial Sloan-Kettering | | prostate tumor | Vaccine |
| Kettering | Cancer Center Institute | | | |
| DC-Cholesterol cationic lipid | RGene Therapeutics Inc | | vaccination, neoplasm | Vaccine |
| GM-CSF vaccine, University of | University of Wisconsin, | | melanoma | Vaccine |
| Wisconsin | Madison | | | |
| melanoma vaccine, Immunex | Immunex Corp | | melanoma | Vaccine |
| GM-CSF vaccine, Johns | Johns Hopkins University | | renal tumor | Vaccine |
| Hopkins | | | | |
| IL-4 gene therapy, Genetic | University of Pittsburgh | | breast tumor, colon tumor, | Vaccine |
| Therapy/Univ Pittsburgh | | | melanoma, renal tumor | |
| fucosyl-GM1-KLH, Sloan- | Memorial Sloan-Kettering | | lung tumor | Vaccine |
| Kettering | Cancer Center Institute | | | |
| GMK | Memorial Sloan-Kettering | | melanoma | Vaccine |
| | Cancer Center Institute | | | |
| TA-HPV | Cancer Research Campaign | | uterine cervix tumor | Vaccine |
| | Technology Ltd | | | |
| LP-2307 | Medical Biology Institute | WO 90/11085 | melanoma, neoplasm | Vaccine |
| vaccine (ras protein), IDEC | IDEC Pharmaceuticals Corp | | carcinoma | Vaccine |
| vaccine (cervical cancer), Johns | Johns Hopkins University | | uterine cervix tumor | Vaccine |
| Hopkins | | | | |
| MGV, Progenics | Progenics Pharmaceuticals Inc | | colorectal tumor, lung tumor, | Vaccine |
| | | | lymphoma, melanoma, | |
| | | | neoplasm, nervous system | |
| | | | tumor, sarcoma, stomach tumor | |
| HPV-16-E7, Loyola University | Loyola University of Chicago | | neoplasm | Vaccine |
| COLO-Vax | Avax Technologies Inc | | colorectal tumor | Vaccine |
| cancer vaccine, Geniva | PowderJect Vaccines | | melanoma, sarcoma, carcinoma, | Vaccine |
| | | | breast tumor | |
| hepatoma/B-cell fusion vaccine | InterCell Co | | liver tumor | Vaccine |
| UNIGEN technology, StressGen | StressGen Biotechnologies Corp | | lung tumor, neoplasm, uterine | Vaccine |
| | | | cervix tumor, vaccination + | |
| | | | d3143 | |
| BIWB-1 | Boehringer Ingelheim Corp | | melanoma | Vaccine |
| Genevax vaccine (lymphoma), | Apollon Inc | | non-Hodgkin's lymphoma | Vaccine |
| Apollon | | | | |
| gene therapy (vaccine), ICRT | Imperial Cancer Research | | neoplasm | Vaccine |
| | Technology Ltd | | | |
| melanoma vaccine, SB | SmithKline Beecham plc | | melanoma | Vaccine |
| papillomavirus vaccine, CSL | CSL Ltd | | uterine cervix tumor | Vaccine |
| vaccine (cancer), NCI | National Cancer Institute | | neoplasm, melanoma | Vaccine |
| OncoVAX | OncoTherapeutics Inc | | carcinoma, lymphoma, non- | Vaccine |
| | | | Hodgkin's lymphoma | |
| Theratope MUC-1 | Biomira Inc | | breast tumor, carcinoma | Vaccine |
| TA-CIN | Cantab Pharmaceuticals plc | | precancer, uterine cervix tumor | Vaccine |
| BP-24 | Biomira Inc | | carcinoma | Vaccine |
| vaccine (breast cancer), Austin | Austin Research Inst | | breast tumor | Vaccine |
| Research | | | | |
| Oncovax-P | Jenner Biotherapies Inc | | prostate tumor | Vaccine |
| gene therapy (HPV), Chiron | Chiron Viagene Inc | | papillomavirus infection, uterine | Vaccine |
| Viagene | | | cervix tumor | |
| vaccine (cancer), Virogenetics | Virogenetics Corp | | colon tumor, neoplasm | Vaccine |
| rV-CEA | National Cancer Institute | | neoplasm | Vaccine |
| p53 cancer vaccine, Virogenetics | Virogenetics Corp | | neoplasm | Vaccine |
| vaccine (B cell lymphoma), IRC | Immune Response Corp | | non-Hodgkin's lymphoma, | Vaccine |
| | | | vaccination | |
| vaccine (B-cell lymphoma), | Stanford University | | non-Hodgkin's lymphoma | Vaccine |
| Stanford University | | | | |
| GD3 ganglioside (vaccine 1), | Memorial Sloan-Kettering | | lung tumor | Vaccine |
| Sloan-Kettering Cancer Center | Cancer Center Institute | | | |
| vaccine (cancer), Jenner/Walter | Jenner Biotherapies Inc | | carcinoma | Vaccine |
| Reed | | | | |
| vaccine (genitourinary cancer), | Urovac Inc | | genitourinary tract tumor | Vaccine |
| Urovac | | | | |
| vaccine (melanoma)(2), Therion | Therion Biologics Corp | | melanoma, metastasis | Vaccine |
| RASVAC | Therion Biologics Corp | | colorectal tumor | Vaccine |
| TBC-NEU | Therion Biologics Corp | | breast tumor, ovary tumor | Vaccine |
| PROSTVAC | Therion Biologics Corp | | prostate tumor | Vaccine |
| MUVAC | Therion Biologics Corp | | breast tumor | Vaccine |
| vaccine (DNP-modified), | Thomas Jefferson University | | melanoma | Vaccine |
| Thomas Jefferson | | | | |
| gene therapy (cancer), | Medical Research Council | | carcinoma, lung tumor, | Vaccine |
| MRC/Stressgen | (MRC) | | melanoma | |
| melanoma vaccines, Univ of | University of Pittsburgh | | melanoma | Vaccine |
| cancer vaccine, MediGene | MediGene GmbH | | neoplasm | Vaccine |
| drug delivery (oral), | Massachusetts Institute of | | hormone replacement therapy, | Vaccine |
| MIT/Endorex | Technology | | neoplasm | |
| Gemvac | Titan Pharmaceuticals Inc | | melanoma, lung tumor | Vaccine |
| B cell lymphoma vaccine, | Vical Inc | | non-Hodgkin's lymphoma | Vaccine |
| Vical/Stanford | | | | |
| HPV vaccine, MediGene | MediGene GmbH | | neoplasm | Vaccine |
| vaccine (GI tumor), Wistar | Wistar Institute of Anatomy & | | colorectal tumor | Vaccine |
| | Biology | | | |
| Theradigm-p53 | Cytel Corp | | carcinoma, lung tumor | Vaccine |
| Theradigm-CEA | Cytel Corp | | carcinoma, colon tumor | Vaccine |
| Theradigm-Her-2 | Cytel Corp | | breast tumor, ovary tumor | Vaccine |
| gene therapy (cancer), | MediGene GmbH | | neoplasm | Vaccine |
| MediGene | | | | |
| vaccine [anticancer], Norsk | Norsk Hydro A/S | | carcinoma | Vaccine |
| Hydro | | | | |
| gp75 melanoma therapy, Sloan- | Memorial Sloan-Kettering | WO 91/14775 | melanoma | Vaccine |
| Kettering | Cancer Center Institute | | | |
| Large Multivalent Immunogen | Lidak Pharmaceuticals | | carcinoma | Vaccine |
| technology, Lidak | | | | |
| vaccine (angiogenesis), | Entremed Inc | | carcinoma | Vaccine |
| Entremed | | | | |
| MVA vector (melanoma), | Bavarian Nordic Research | | melanoma | Vaccine |
| Bavarian Nordic | Institute AS | | | |
| CTP-37 | Immunotherapy Corp | | neoplasm, colorectal tumor, | Vaccine |
| | | | pancreas tumor, breast tumor, | |
| | | | prostate tumor | |
| melanoma vaccine, Sloan- | Memorial Sloan-Kettering | | melanoma | Vaccine |
| Kettering | Cancer Center Institute | | | |
| idiotypic cancer vaccines, | National Cancer Institute | | non-Hodgkin's's lymphoma + | Vaccine |
| NCI/Genzyme Transgenics | | | d3187 | |
| M-Vax | Avax Technologies Inc | | melanoma | Vaccine |
| PJ-2204 | PowderJect Pharmaceuticals | | melanoma | Vaccine |
| PJ-3505 | PowderJect Pharmaceuticals | | neoplasm | Vaccine |
| Oncovax-CL | Jenner Biotherapies Inc | | colorectal tumor, lung tumor | Vaccine |
| 4B5 antibody, Novopharm | University of Alabama in | | melanoma, lung tumor, nervous | Vaccine |
| | Birmingham | | system tumor | |
| GeneVax vaccine (cancer), | Apollon Inc | | breast tumor, colorectal tumor, | Vaccine |
| Centocor | | | neoplasm, prostate tumor | |
| MAGE-3, Epimmune | Epimmune Inc | | neoplasm | Vaccine |
| gene therapy (cancer), | Vical Inc | | neoplasm | Vaccine |
| Vical/Centocor | | | | |
| cancer vaccine, Allegheny | Allegheny University of the | | colon tumor, breast tumor | Vaccine |
| | Health Sciences | | | |
| prostate cancer vaccine, ML | ML Laboratories plc | | prostate tumor | Vaccine |
| Labs | | | | |
| vaccine (breast cancer), AltaRex | AltaRex Corp | | breast tumor | Vaccine |
| nanoparticle technology, BA | Ben-Abraham Technologies Inc | | neoplasm + d3203 | Vaccine |
| Tech | | | | |
| vaccine (prostate), Pacific | Pacific Northwest Cancer | | prostate tumor | Vaccine |
| Northwest | Foundation | | | |
| BhCG vaccine (cancer), | University College London | | neoplasm, vaccination | Vaccine |
| UCL/Vaxcel | | | | |
| vaccines (cancer), Onyvax | Onyvax Ltd | | carcinoma | Vaccine |
| vaccine (cancer), Cytokine | Cytokine Networks Inc | | carcinoma | Vaccine |
| rMVA, NIH | National Institutes of Health | | neoplasm | Vaccine |
| vaccine (prostate tumor), | Corixa Corp | WO 97/08318 | prostate tumor | Vaccine |
| Corixa/SB Biologicals | | | | |
| melanoma-specific antigens, | Argonex Inc | | melanoma | Vaccine |
| Argonex | | | | |
| melanoma vaccine, Memorial | Memorial Sloan-Kettering | | melanoma | Vaccine |
| Sloan-Kettering | Cancer Center Institute | | | |
| melanoma vaccine, NYU | New York University | U.S. Pat. No. | melanoma | Vaccine |
| pTG-1031 | Transgene SA | 5,030,621 | breast tumor | Vaccine |
| vaccine (TCR), T Cell Sciences | T Cell Sciences Inc | | carcinoma, neoplasm | Vaccine |
| BEC-2 | Imclone Systems Inc | | carcinoma, d3205lung tumor, | Vaccine |
| | | | neoplasm | |
| SDZ-SCV-106 | Novartis AG | | carcinoma, neoplasm | Vaccine |
| Melacine | Ribi ImmunoChem Research Inc | | melanoma | Vaccine |
| gene therapy (gamma | Chiron Viagene Inc | | melanoma, neoplasm, nervous | Vaccine |
| interferon), Chiron Viagene | | | system tumor, renal tumor | |
| retroviral vectors, Chiron | Chiron Viagene Inc | | neoplasm | Vaccine |
| Viagene | | | | |
| peptic ulcer therapy, MicroCarb | Antex Biologics Inc | | stomach tumor | Vaccine |
| pox vector technology, Therion | Therion Biologics Corp | | neoplasm | Vaccine |
| MAGE-1, Somatix/Ludwig | Ludwig Institute for Cancer | WO 92/20356 | neoplasm, carcinoma | Vaccine |
| Institute | Research | | | |
| vaccine (B cell lymphoma), NIH | Trega Biosciences Inc | | lymphoma | Vaccine |
| 105AD7 | Cancer Research Campaign | | digestive system tumor | Vaccine |
| | (UK) | | | |
| Theratope STn-KLH | Biomira Inc | | breast tumor, colorectal tumor, | Vaccine |
| | | | ovary tumor, stomach tumor, | |
| | | | vaccination | |
| vaccine (cancer), Biochem | BioChem Pharma Inc | | bladder tumor, carcinoma, | Vaccine |
| Pharma | | | neoplasm | |
| Magevac | Therion Biologics Corp | | melanoma, breast tumor | Vaccine |
| TBC-CEA, Therion | Therion Biologics Corp | | colon tumor, breast tumor, lung | Vaccine |
| | | | tumor | |
| vaccine (cancer), MedImmune | MedImmune Inc | | carcinoma | Vaccine |
| MEDI-501 | MedImmune Inc | | uterine cervix tumor | Vaccine |
| vaccine (EBV), Bioresearch | BioResearch Ireland | | carcinoma | Vaccine |
| Ireland | | | | |
| vaccine (MUC-1), | Corixa Corp | | breast tumor, colon tumor, | Vaccine |
| Corixa/Vaxcel | | | pancreas tumor | |
| vaccine (Her-2/neu), | Corixa Corp | | breast tumor, ovary tumor | Vaccine |
| Corixa/Vaxcel | | | | |
| Chimeric Virus Particle (CVP) | Axis Genetics | | colon tumor, prostate tumor | Vaccine |
| technology, Axis Genetics | | | | |
| human papillomavirus vaccine, | Merck & Co Inc | | uterine cervix tumor | Vaccine |
| Merck | | | | |
| vaccine (colon cancer), | Immune Response Corp | | colon tumor | Vaccine |
| IRC/SKCC | | | | |
| HPV vaccine, UniQuest | UniQuest Ltd | | uterine cervix tumor | Vaccine |
| Optivax | Vaxcel Inc | | carcinoma, vaccination | Vaccine |
| vaccine (3H1 mAb), Kentucky | University of Kentucky | | colorectal tumor | Vaccine |
| Uni | | | | |
| Gastrimmune | Aphton Corp | | colon tumor, liver tumor, | Vaccine |
| | | | neoplasm, pancreas tumor, | |
| | | | stomach tumor | |
| recombinant vaccine (colon | National Institutes of Health | | colon tumor | Vaccine |
| cancer), National Institutes of | | | | |
| Health | | | | |
| BLP-25 | Biomira Inc | | carcinoma | Vaccine |
| melanoma vaccine, John Wayne | John Wayne Cancer Institute | WO 96/17614 | melanoma | Vaccine |
| BP1-7 | Biomira Inc | | neoplasm, breast tumor | Vaccine |
| O-Vax | Avax Technologies Inc | | ovary tumor | Vaccine |
| L-Vax | Avax Technologies Inc | | myeloid leukemia | Vaccine |
| NOVOVAC-M1 | Novopharm Biotech Inc | | melanoma | Vaccine |
| BP-16 | Biomira Inc | | breast tumor | Vaccine |
| GM-CSF tumor vaccine, | PowderJect Pharmaceuticals | | melanoma | Vaccine |
| PowderJect | | | | |
| TBC-1635 | Texas Biotechnology Corp | | angiogenesis disorder, solid | VEGF antagonist |
| | | | tumor | |
| ZD-4190 | Zeneca Group Plc | | solid tumor | VEGF antagonist |
| anti VEGF antibody, Toagosei | Toagosei Co Ltd | | neoplasm | VEGF antagonist |
| CI-935 | Parke-Davis & Co | | neoplasm | Viral replication inhibitor |
| MAP-30 | New York University | | neoplasm | Viral replication inhibitor |
| GAP-31 | New York University | | neoplasm | Viral replication inhibitor |
| tricibine | University of Michigan | | carcinoma | Viral replication inhibitor |
| mecobalamin | Eisai Co Ltd | | leukemia | Vitamin B12 agonist |
| EB-1089 | Leo Denmark | | breast tumor, colon tumor, | Vitamin D agonist |
| | | | neoplasm | |
| dihydroxycalcitriol | University of Pittsburgh | | neoplasm | Vitamin D agonist |
| EB-1089 | Leo Denmark | | breast tumor, colon tumor, | Vitamin D agonist |
| | | | neoplasm | |
| one-alpha-D2, Bone Care | Bone Care International Inc | WO 94/05630 | prostate tumor | Vitamin D2 agonist |
| (Lunar) | | | | |
| MC-1301 | Leo Denmark | | carcinoma | Vitamin D3 agonist |
| CB-1093 | Leo Pharmaceutical Products | | myeloid leukemia, carcinoma | Vitamin D3 agonist |
| | BV | | | |
| LR-103 | Bone Care International Inc | | breast tumor, colon tumor, | Vitamin D3 agonist |
| | | | prostate tumor, psoriasis | |
| CB-1267 | Leo Denmark | | carcinoma, prostate tumor | Vitamin D3 agonist |
| MC-1357 | Leo Denmark | WO 91/15475 | neoplasm | Vitamin D3 agonist |
| MC-1288 | Leo Denmark | | carcinoma | Vitamin D3 agonist |
| lexacalcitol | Leo Pharmaceutical Products Inc | | skin tumor, breast tumor | Vitamin D3 agonist |
| ATRISORB | Atrix Labs Inc | | neoplasm | |
| Spartaject | Sparta Pharmaceuticals Inc | | bone marrow transplantation, | |
| | | | breast tumor, lung tumor, ovary | |
| | | | tumor | |
| LADD technology, Sparta | Yale University | WO 92/20816 | breast tumor, colorectal tumor, | |
| | | | liver disease, liver tumor, solid | |
| | | | tumor | |
| beta-interferon, Schering AG | Schering AG | | breast tumor, prostate tumor, | |
| | | | carcinoma | |
| gene therapy (H-NUC TSG), | Canji Inc | | breast tumor | |
| Canji | | | | |
| oligonucleotides (CAPL), | Hybridon Inc | WO 96/25499 | neoplasm | |
| Hybridon | | | | |
| ImmuRAID-LL1 | Immunomedics Inc | EP 0 336 678 | solid tumor | |
| GLIOMAb-H | Novopharm Biotech | | malignant neoplastic disease, | |
| | | | melanoma, glioma | |
| gadoteric acid | Guerbet SA | | carcinoma | |
| RIGS/ACT, Neoprobe/Cellcor | Neoprobe Corp | | breast tumor, colorectal tumor, | |
| | | | pancreas tumor | |
| drug screening, Xenova | Xenova Ltd | | neoplasm | |
| CLN-IgG | Japan Pharmaceutical | JP 06141884 | uterus tumor, glioma | |
| | Development Co Ltd | | | |
| LymphoScan | Immunomedics Inc | EP 0 336 678 | non-Hodgkin's lymphoma | |
| AAV vectors, Theragen | Theragen Inc | WO 95/34671 | colon tumor | |
| 2B-1 | Chiron Corp | | breast tumor, colon tumor | |
| AMI-25 | Advanced Magnetics Inc | | liver tumor | |
| aristeromycin | Pharmacia & Upjohn Co | | neoplasm | |
| breast cancer gene therapy, | Canji Inc | | breast tumor | |
| Canji | | | | |
| MT2 | BioCure Ltd | | carcinoma, neoplasm | |
| BeneFin | Lane Laboratories | | prostate tumor, sarcoma | |
| CD5/CD8 cell therapy, Applied | Applied Immune Sciences Inc | | bone marrow transplantation, | |
| Immune Sciences | | | graft vs host disease | |
| CD8 TIL cell therapy, Applied | Applied Immune Sciences Inc | | renal tumor | |
| Immune Sciences | | | | |
| lamellarin-N-triacetate | Pharma Mar SA | | lung tumor | |
| palauamine | Pharma Mar SA | | lung tumor | |
| myriaporone | Pharma Mar SA | | leukemia | |
| isohomohalicondrin | Pharma Mar SA | | central nervous system tumor, | |
| | | | colon tumor, lung tumor, | |
| | | | melanoma, ovary tumor | |
| variolin B | Pharma Mar SA | | carcinoma, central nervous | |
| | | | system tumor, lung tumor, | |
| | | | melanoma, renal tumor | |
| oligonucleotides (telomerase), | Genta Inc | | neoplasm | |
| Genta/Geron | | | | |
| oligonucleotides (glioblastoma), | Genta Inc | | nervous system tumor | |
| Genta | | | | |
| creatine analogs, Repligen | RepliGen Corp | | neoplasm | |
| vitalethine | University of New Mexico | WO 92/00955 | neoplasm | |
| ER-34410 | Eisai Co Ltd | | carcinoma | |
| DMP-315 | DuPont Pharmaceuticals Co | | carcinoma | |
| CC-1065 analogs, Pharma Mar | Pharma Mar SA | | carcinoma | |
| HN-66000 | National Institutes of Health | | brain tumor, central nervous | |
| | | | system tumor, head & neck | |
| | | | tumor | |
| NC-100100 | Hafslund Nycomed A/S | | renal tumor | |
| haematopoietic growth factors, | AMRAD Corp | | carcinoma | |
| AMRAD | | | | |
| anticancer, BTG | British Technology Group Plc | | carcinoma | |
| PC-1MAb, Matritech | Matritech Inc | | prostate tumor | |
| Eovist | Schering AG | | liver tumor | |
| Magnetites | Schering AG | | liver tumor | |
| Cavisomes | Schering AG | | liver tumor | |
| blood substitute, Sonus | Sonus Pharmaceuticals Inc | | neoplasm | |
| anticancer, Sugen/Zeneca | Zeneca Group Plc | | neoplasm | |
| CTL gene therapy, Targeted | Targeted Genetics Corp | | melanoma | |
| Genetics | | | | |
| angiogenesis antibody, Antisoma | Antisoma plc | | angiogenesis disorders, | |
| | | | carcinoma | |
| creatine analogs, Repligen | RepliGen Corp | | neoplasm | |
| vitalethine | University of New Mexico | WO 92/00955 | neoplasm | |
| ER-34410 | Eisai Co Ltd | | carcinoma | |
| DMP-315 | DuPont Pharmaceuticals Co | | carcinoma | |
| CC-1065 analogs, Pharma Mar | Pharma Mar SA | | carcinoma | |
| HN-66000 | National Institutes of Health | | brain tumor, central nervous | |
| | | | system tumor, head & neck | |
| | | | tumor | |
| NC-100100 | Hafslund Nycomed A/S | | renal tumor | |
| haematopoietic growth factors, | AMRAD Corp | | carcinoma | |
| AMRAD | | | | |
| anticancer, BTG | British Technology Group Plc | | carcinoma | |
| PC-1MAb, Matritech | Matritech Inc | | prostate tumor | |
| Eovist | Schering AG | | liver tumor | |
| Magnetites | Schering AG | | liver tumor | |
| Cavisomes | Schering AG | | liver tumor | |
| blood substitute, Sonus | Sonus Pharmaceuticals Inc | | neoplasm | |
| anticancer, Sugen/Zeneca | Zeneca Group Plc | | neoplasm | |
| CTL gene therapy, Targeted | Targeted Genetics Corp | | melanoma | |
| Genetics | | | | |
| angiogenesis antibody, Antisoma | Antisoma plc | | angiogenesis disorders, | |
| | | | carcinoma | |
| CTL (cancer), Targeted Genetics | Targeted Genetics Corp | | renal tumor | |
| orphan receptor program, Karo | Karo Bio AB | | carcinoma | |
| Bio/Tripos | | | | |
| VincaXome | NeXstar Pharmaceuticals Inc | | carcinoma | |
| TSARs, Cytogen/Elan | CYTOGEN Corp | | neoplasm | |
| WAF1, PharmaGenics | Genzyme Molecular Oncology | | neoplasm | |
| DCC, Genzyme Mol Oncology | Genzyme Molecular Oncology | | neoplasm | |
| SMART anti-B cell lymphoma | Protein Design Labs Inc | | non-Hodgkin's lymphoma | |
| PM-92100 | Universidad Complutense de | | colon tumor, lung tumor, | |
| | Madrid | | melanoma, ovary tumor | |
| DepoFoam | DepoTech Corp | | neoplasm | |
| SR-4554 | SRI International | | neoplasm | |
| RS7-3G11 | University of Medicine and | | neoplasm | |
| | Dentistry of New Jersey | | | |
| monoclonals (bladder cancer), | AMRAD Corp | | bladder tumor | |
| AMRAD | | | | |
| antineoplastic, Dr Reddys Res | Dr Reddy's Research | | neoplasm | |
| Found | Foundation | | | |
| Osteomark | Ostex International Inc | | Paget's disease, bone tumor | |
| delivery system (AVE), | Advanced Therapies Inc | | carcinoma | |
| Advanced Therapies | | | | |
| DP-003 | Daikin Industries Ltd | | carcinoma, colon tumor | |
| cancer diagnostic test, EntreMed | Entremed Inc | | neoplasm | |
| anti-Ptk, Theratechnologies | Theratechnologies Inc | | breast tumor, prostate tumor | |
| intracellular proteolysis agents, | Mitotix Inc | | neoplasm, uterine cervix tumor | |
| Mitotix | | | | |
| colchicine analogs, BioSpecifics | National Institutes of Health | | carcinoma | |
| antibody therapeutics (cancer), | MorphoSys GmbH | | carcinoma | |
| MorphoSys/Micromet | | | | |
| andrographolide | Paracelsian Inc | | neoplasm | |
| monoclonal (squamous cell | Queensland Institute of Medical | | squamous cell carcinoma | |
| carcinoma), QIMR | Research | | | |
| MSI-238 | Magainin Pharmaceuticals Inc | | ovary tumor | |
| SMART bispecific mAb, Protein | Protein Design Labs Inc | | skin tumor | |
| Design Labs | | | | |
| Oncozole | ICN Pharmaceuticals Inc | | solid tumor | |
| antiprostate MAb, NIH | National Institutes of Health | | prostate tumor | |
| OncoCELL | OncoTherapeutics Inc | | carcinoma, renal tumor | |
| TF inhibitors (TNF), Tularik | Tularik Inc | | neoplasm | |
| signal transduction modulator, | Vertex Pharmaceuticals Inc | | neoplasm | |
| Vertex | | | | |
| Nuclear Matrix Proteins (colon | Matritech Inc | WO 94/00573 | colon tumor | |
| cancer), Matritech | | | | |
| ferrixan | Schering AG | | carcinoma, liver tumor | |
| MS-264 | EPIX Medical Inc | | hepatobiliary system tumor | |
| gadobutrol | Schering AG | | brain tumor | |
| prostatic inhibin peptide, | Procyon Biopharma | | prostate tumor | |
| Procyon Biopharma | | | | |
| Trimera XTL | XTL Biopharmaceutical Ltd | | carcinoma | |
| CJM-216 | Max-Delbrueck-Centrum fuer | | lung tumor, ovary tumor, breast | |
| | Molekulare Medizin | | tumor | |
| GS-2888 | Gilead Sciences Inc | | neoplasm | |
| synthetic p16, Dundee | University of Dundee | WO 97/11174 | neoplasm | |
| TLC-ELL-12 | The Liposome Company Inc | | lung tumor, | |
| | | | melanoma, neoplasm, prostate | |
| | | | tumor | |
| gene therapy (herpes simplex | Progenitor Inc | | neoplasm | |
| thymidine kinase), Progenitor | | | | |
| TEI-9826 | Teijin Ltd | | neoplasm | |
| SH-L-545 | Schering AG | | carcinoma | |
| transcript imaging technology, | Sugen Inc | | carcinoma | |
| Sugen/NCI | | | | |
| MS-136 | EPIX Medical Inc | | breast tumor, liver tumor | |
| MS-325 | EPIX Medical Inc | | breast tumor | |
| bph treatment, Zonagen | Zonagen Inc | | prostate tumor | |
| balsalazide | Salix Pharmaceuticals Inc | | colon tumor, intestine tumor | |
| Recolin | NPO Vector | | neoplasm | |
| rheumatoid arthritis therapeutics, | Phytera Inc | | neoplasm | |
| Phytera/Tsumura | | | | |
| oligonucleotide CML, Lynx | Lynx Therapeutics Inc | | myeloid leukemia | |
| YM-534 | Yamanouchi Pharmaceutical Co | | carcinoma | |
| | Ltd | | | |
| Y-25510 | Yoshitomi Pharmaceutical | | carcinoma | |
| | Industries Ltd | | | |
| signal transduction inhibitors, | Sugen Inc | | neoplasm | |
| SUGEN/Chinese Academy | | | | |
| RIGScan CR49 | Neoprobe Corp | | breast tumor, colorectal tumor, | |
| | | | ovary tumor, pancreas tumor, | |
| | | | stomach tumor | |
| optical imaging agents, | Mallinckrodt Medical | | neoplasm, breast tumor | |
| Mallinckrodt/Optimedx | | | | |
| rhenium-186 etidronate | Mallinckrodt Medical | | bone tumor, pain | |
| imaging agent, | AltaRex Corp | | carcinoma | |
| AltaRex/Resolution Pharm | | | | |
| NM-324 | University of Michigan | | solid tumor | |
| paclitaxel, Cytoclonal | Cytoclonal Pharmaceuticals Inc | | carcinoma | |
| patched gene, Ontogeny | Stanford University | | skin tumor, carcinoma | |
| intrabody, ITI/RPR | IntraImmune Therapies Inc | | neoplasm | |
| fusion proteins, | Techniclone Corp | | solid tumor | |
| Techniclone/USC | | | | |
| hyaluronan (cancer) | Hyal Pharmaceutical Corp | | breast tumor, carcinoma, colon | |
| | | | tumor, lung tumor | |
| ICN-70 | ICN Pharmaceuticals Inc | | breast tumor, melanoma, | |
| | | | prostate tumor | |
| ICN-107 | ICN Pharmaceuticals Inc | | breast tumor, lung tumor, | |
| | | | melanoma, prostate tumor | |
| ICN-240 | ICN Pharmaceuticals Inc | | breast tumor, lung tumor, | |
| | | | melanoma, prostate tumor | |
| 3-deazaguanine | ICN Pharmaceuticals Inc | | carcinoma | |
| delivery system [doxorubicin], | Supratek Pharma Inc | | carcinoma | |
| Supratek | | | | |
| V-489 | Uniroyal Chemical Co Inc | | carcinoma | |
| immunoconjugates (cancer), | Immunomedics Inc | WO 93/23062 | carcinoma | |
| Immunomedics | | | | |
| cancer genetics, | Sequana Therapeutics | | prostate tumor, breast tumor, | |
| Sequana/Memorial Sloan | | | colon tumor | |
| Kettering | | | | |
| Mab-170 | Biomira Inc | | breast tumor | |
| ribozymes (cancer), Ribozyme | Ribozyme Pharmaceuticals Inc | | leukemia | |
| MADR2 gene | Hospital for Sick Children | | colon tumor | |
| busulfan, Spartaject | Sparta Pharmaceuticals Inc | | bone marrow transplantation, | |
| | | | carcinoma | |
| taxanes, Spartaject | Sparta Pharmaceuticals Inc | | carcinoma | |
| D-22631 | ASTA Medica AG | | carcinoma | |
| etoposide, Spartaject | Sparta Pharmaceuticals Inc | | carcinoma | |
| camptothecin, Spartaject | Sparta Pharmaceuticals Inc | | carcinoma, colon tumor, lung | |
| | | | tumor | |
| DU-86 | Kyowa Hakko Kogyo Co Ltd | | carcinoma | |
| TXS-0202 | Cobra Therapeutics | | head & neck tumor, prostate | |
| | | | tumor, liver tumor | |
| D2AT21 | Demeter Biotechnologies Ltd | | carcinoma, neoplasm, prostate | |
| | | | tumor | |
| ellagic acid analogs, Bowling | Bowling Green State University, | | neoplasm | |
| Green | USA | | | |
| prohibitin, NIH | National Institutes of Health | | carcinoma | |
| Apigenin | Kyowa Hakko Kogyo Co Ltd | | carcinoma | |
| CancerVax-M | CancerVax Inc | | melanoma | |
| GT-1106 | Genset | WO 96/12803 | myeloid leukemia | |
| Transferrin CRM-107 | Hafslund Nycomed A/S | | brain tumor | |
| Prostatec | Targon Corp | | prostate tumor | |
| Oncotec | Targon Corp | | breast tumor | |
| Panoject | Elan Corp Plc | | neoplasm, pain | |
| anti-estrogens, BioNumerik | Bionumerik Pharmaceuticals Inc | | breast tumor | |
| platinum compounds, | Bionumerik Pharmaceuticals Inc | | solid tumor | |
| BioNumerik | | | | |
| synthetic p53, BioNumerik | Bionumerik Pharmaceuticals Inc | | solid tumor | |
| anti-MDR, BioNumerik | Bionumerik Pharmaceuticals Inc | | solid tumor | |
| leukemia therapy, OSI | OSI Pharmaceuticals Inc | | myeloid leukemia | |
| Pharmaceuticals | | | | |
| PH45 | Pherin Corp | | prostate tumor | |
| Medipad | Elan Corp Plc | | neoplasm, pain | |
| polyorthoester DDS (cancer) | Advanced Polymer Systems | | breast tumor | |
| bioerodible DDS (vaccines) | Advanced Polymer Systems | | vaccination, neoplasm | |
| CZ-112 | Stehlin Foundation For Cancer | | carcinoma, neoplasm | |
| | Research | | | |
| leukemia gene, | Myriad Genetics Inc | | leukemia | |
| Myriad/Anderson | | | | |
| BRCA2 gene, Myriad | Myriad Genetics Inc | | breast tumor | |
| vomeropherin [breast cancer], | Pherin Corp | | breast tumor | |
| Pherin | | | | |
| SB-T-1102 | Rhone-Poulenc Rorer Ltd | | carcinoma | |
| SB-T-1213 | Rhone-Poulenc Rorer Ltd | | carcinoma | |
| SB-T-1214 | Rhone-Poulenc Rorer Ltd | | carcinoma | |
| SB-T-12162 | Rhone-Poulenc Rorer Ltd | | carcinoma | |
| melanoma gene, Sequana | Sequana Therapeutics | | melanoma | |
| EK-5504 | Schering AG | | carcinoma | |
| NMP-22 | Matritech Inc | | bladder tumor | |
| CD-Tagging | Vyrex Corp | | carcinoma | |
| manzamines | Meiji Seika Kaisha Ltd | | carcinoma | |
| KR-2827 and derivatives, Kirin | Kirin Brewery Co Ltd | | carcinoma | |
| FR-182877 | Fujisawa Pharmaceutical Co Ltd | WO 96/32402 | carcinoma | |
| photodynamic Abs, | Bioenhancements Ltd | WO 96/34892 | neoplasm | |
| BioEnhancements | | | | |
| RIDD therapy, PNRF | Pacific Northwest Research | | breast tumor | |
| | Foundation | | | |
| TriAB | Trilex Pharmaceuticals Inc | | breast tumor | |
| anti-4Dc antibody, Trilex | Trilex Pharmaceuticals Inc | | non-Hodgkin's lymphoma | |
| anti-NHL antibody, | Immunomedics Inc | | non-Hodgkin's lymphoma | |
| Immunomedics | | | | |
| delivery system [fluorouracil], | Matrix Pharmaceutical Inc | | carcinoma | |
| Matrix | | | | |
| MMAC1 gene, Myriad/MD | Myriad Genetics Inc | | breast tumor, glioma, neoplasm, | |
| Anderson | | | prostate tumor, renal tumor, skin | |
| | | | tumor | |
| LXSN-BRCA1 gene therapy, | University of Tennessee, | | prostate tumor | |
| UT | Knoxville | | | |
| gene therapy (melanoma), | Genzyme Molecular Oncology | | melanoma | |
| Genzyme Molecular/NCI | | | | |
| ras inhibitors, Proscript | ProScript Inc | | carcinoma | |
| prostate cancer genes, Gene | Baylor College of Medicine | | prostate tumor | |
| Logic/Baylor College | | | | |
| CHK gene, Beth Israel | Beth Israel Hospital Association | | breast tumor | |
| gene discovery (prostate cancer), | Mercator Genetics Inc | | prostate tumor | |
| Mercator | | | | |
| combinatorial chemistry, Trega | Trega Biosciences Inc | | carcinoma | |
| Atrigel | Atrix Labs Inc | | carcinoma, prostate tumor | |
| Notch signaling cascade, | Exelixis Pharmaceuticals Inc | | neoplasm | |
| Exelixis | | | | |
| Pseudomonas exotoxin, John | John Wayne Cancer Institute | | brain tumor | |
| Wayne | | | | |
| anti-FAK oligonucleotides, | Genta Inc | | neoplasm | |
| Genta | | | | |
| Cytoporter-CP | AVI BioPharma | | neoplasm | |
| Adrenomedullin peptides | US Department of Health & | WO 97/07214 | neoplasm | |
| | Human Services | | | |
| APC gene therapy, Onyx | ONYX Pharmaceuticals Inc | | neoplasm | |
| B7-1 gene therapy, University of | University of Wisconsin, | | melanoma | |
| Wisconsin | Madison | | | |
| UCH-15 | Kyowa Hakko Kogyo Co Ltd | WO 97/10208 | neoplasm | |
| TI-356 | Taisho Pharmaceutical Co Ltd | WO 97/10264 | neoplasm | |
| Pyrrolosporin A | Bristol-Myers Squibb Co | | leukemia | |
| LymphoCide | Immunomedics Inc | | lymphoma, non-Hodgkin's | |
| | | | lymphoma | |
| PNU-153429 | Pharmacia & Upjohn Inc | | neoplasm | |
| PTL-03001 | Peptech Ltd | | prostate tumor | |
| Chimeric MAb 31.1 | International Bioimmune | | colon tumor | |
| | Systems Inc | | | |
| BST-1004 | BioStratum Inc | | lung tumor | |
| BST-1005 | BioStratum Inc | | bladder tumor | |
| p53 modulators, Genzyme | Genzyme Molecular Oncology | | neoplasm | |
| Molecular Oncology/Xenova | | | | |
| ARF-p19 | St Jude Childrens Hospital | WO 97/12060 | neoplasm | |
| gene therapy, RPR/Stanford | Stanford University | | neoplasm | |
| Prognox | Amersham International plc | | solid tumor | |
| cancer gene therapy, | Prizm Pharmaceuticals Inc | | neoplasm | |
| Prizm/Chiron | | | | |
| melanoma gene therapy, | Megabios Corp | | melanoma, solid tumor | |
| Megabios | | | | |
| APC gene theapy, Genzyme | Genzyme Molecular Oncology | | colon tumor | |
| melanoma therapy, Cytel/Baxter | Cytel Corp | | melanoma | |
| gene therapy (liver disease), | Genzyme Corp | | liver tumor | |
| Genzyme | | | | |
| AN-207 | ASTA Medica AG | | neoplasm | |
| D-23980 | ASTA Medica AG | | neoplasm | |
| p53 gene therapy, Sidney | Sidney Kimmel Cancer Center | | neoplasm, glioma | |
| Kimmel Cancer Center | | | | |
| p53 gene therapy, NCI | National Cancer Institute | | glioma, neoplasm | |
| gene therapy (glioma), Dana | Dana Farber Cancer Institute Inc | | glioma | |
| Farber | | | | |
| ribozyme (glioma), University of | University of Pittsburgh | | glioma | |
| Pittsburgh | | | | |
| reconstitutable formulation | Bioglan Pharma Plc | | carcinoma | |
| system, Bioglan | | | | |
| Tc-HL-91 | Warwick University | | solid tumor | |
| dendritic cell cancer therapy, | Cellpro Inc | | neoplasm | |
| CellPro | | | | |
| sandramycin analogs, Scripps | Scripps Research Institute | | neoplasm | |
| colorectal tumor therapy, | Nycomed ASA | | colorectal tumor | |
| Nycomed/TDT | | | | |
| antivirals, RiboGene/Trega | Ribogene Inc | | carcinoma | |
| D-21621 | ASTA Medica AG | | neoplasm | |
| LY-312340 | Oxford University | | prostate tumor, breast tumor | |
| estradiol analogs, Pharma-Eco | Pharm-Eco Laboratories Inc | | neoplasm | |
| gene therapy (DNA repair), | Lexicon Genetics Inc | | neoplasm | |
| Lexicon | | | | |
| nanoerythrosomes | DiagnoCure Inc | | neoplasm | |
| cytovaricin B, Tokyo University | Tokyo University | | neoplasm | |
| drug discovery, BioChem | BioChem Therapeutic Inc | | hepatitis c virus infection, | |
| Therapeutics/Structural | | | neoplasm | |
| Bioinformatics | | | | |
| anticancer agents, Tokyo | Tokyo University | | neoplasm | |
| University/Shionogi/NCI | | | | |
| PEG technology, OXIS | OXIS International Inc | | neoplasm | |
| conopeptides (neoplasm), | Cognetix Inc | | lung tumor | |
| Cognetix | | | | |
| B-0983 | Yissum Research Development | | metastasis | |
| | Co of the Hebrew University of | | | |
| | Jerusalem | | | |
| anticancer agents, Sandoz | Sandoz Pharmaceuticals Corp | | neoplasm | |
| growth factor complex, IPR | IPR-Institute for Pharmaceutical | | skin tumor | |
| | Research Riehen AG | | | |
| macrophage gene therapy, | University of Sheffield | | solid tumor | |
| Oxford Biomedica | | | | |
| TheraCIM | York Medical Inc | | breast tumor, lung tumor, head | |
| | | | & neck tumor | |
| RNA vaccine (cancer), Duke | Duke University | | breast tumor, colorectal tumor, | |
| University | | | lung tumor | |
| HSR-3/A9 | Biotest Pharma GmbH | | Hodgkin's disease, glioma | |
| microalgal therapeutics, | InflaZyme Pharmaceuticals Ltd | | neoplasm | |
| Aquasearch/Inflazyme | | | | |
| lentiviral vectors, Cell Genesys | The Salk Institute | | neoplasm | |
| ISIS-3466 | ISIS Pharmaceuticals Inc | | neoplasm | |
| oligonucleotide (leukemia)(2), | University of Pennsylvania | | myeloid leukemia, neoplasm | |
| University of Pennsylvania | | | | |
| SB-RA-4102 | Stony Brook University | | carcinoma | |
| oligonucleotide (IL-1r), ISIS | ISIS Pharmaceuticals Inc | | neoplasm | |
| Pharmaceuticals | | | | |
| oligonucleotide (IGF-1R), Lynx | Lynx Therapeutics Inc | | neoplasm, brain tumor, ovary | |
| Therapeutics | | | tumor | |
| cancer therapeutics, Agouron | Agouron Pharmaceuticals Inc | | neoplasm | |
| CGP-62360 | Novartis AG | | melanoma | |
| INGN-401 | Introgen Therapeutics Inc | | neoplasm | |
| MR-566A | Korea Institute of Bioscience | | carcinoma | |
| | and Biotechnology | | | |
| oligonucleotide (Rel-A), | Hoffmann-La Roche Inc | | neoplasm | |
| Hoffmann-La Roche | | | | |
| genomics agreement, | Reprogen Inc | | genital tract tumor | |
| Reprogen/Genzyme | | | | |
| gene therapy (brain disorders), | St Jude Childrens Hospital | | neoplasm | |
| St Jude Childrens Hospital | | | | |
| taxuspines, Hokkaido University | Hokkaido University | | carcinoma | |
| leptofuranin A | Tokyo University | | neoplasm | |
| Sch-202596 | Schering-Plough Corp | | carcinoma | |
| BM-920700 | Boehringer Mannheim GmbH | | neoplasm | |
| CZ-105 | Stehlin Foundation For Cancer | | carcinoma | |
| | Research | | | |
| NSC-652287 | MD Anderson Cancer Center | | carcinoma | |
| KB-R8498 | Kanebo KK | | carcinoma | |
| SC-101i | Scotia Pharmaceuticals | | bladder tumor | |
| TAS-101 | Taiho Pharmaceutical Co Ltd | | carcinoma | |
| gene therapy | University of Alabama in | | neoplasm | |
| (cholangiocarcinoma), | Birmingham | | | |
| University of Alabama | | | | |
| gene therapy (gastric tumor), | Tokyo University | | stomach tumor | |
| Tokyo University | | | | |
| oligonucleotides (HPV), | University of Pittsburgh | | uterine cervix tumor | |
| University of Pittsburgh | | | | |
| MDI-301 | Molecular Design International | | neoplasm | |
| anti-VEGF mAb, Mitsui | Mitsui Toatsu Chemicals Inc | EP 0 787 742 | neoplasm | |
| oligonucleotide (Ha-ras), Osaka | Osaka University | | liver tumor | |
| University | | | | |
| gene therapy (p16), | National Institute for | | neoplasm | |
| Physiological Sciences Institute | Physiological Sciences | | | |
| SKI-2054R | Sunkyong Industries Co Ltd | | neoplasm | |
| anticancers, | Axiom Biotechnologies Inc | | neoplasm | |
| Axiom/Zaiya/Nippon Kayaku | | | | |
| drug screening, Genzyme | Genzyme Corp | | neoplasm | |
| Molecular/Parke-Davis | | | | |
| drug discovery (cancer), | Ventiv BioGroup | | myeloproliferative disorder, non- | |
| VIMRx/Columbia University | | | Hodgkin's lymphoma | |
| ES-921 | Sankyo KK | | carcinoma | |
| SN-7167 | University of Auckland | | carcinoma | |
| ribozyme (bcl-2), Columbia | Columbia University | | prostate tumor | |
| University | | | | |
| Plat-23 | The Liposome Company Inc | | neoplasm | |
| aminothiadiazole | National Cancer Institute | | neoplasm | |
| DNA immunization therapy, | Dynavax Technologies Corp | | neoplasm | |
| Dynavax | | | | |
| levofolinate | Lederle (Japan) Ltd | | neoplasm | |
| Therapore | Harvard University | | neoplasm | |
| L-amino oxidase, Cornell | Cornell Research Foundation Inc | | neoplasm | |
| SH-920132 | Dong-Wha Pharmaceutical | | neoplasm | |
| | Industry Co Ltd | | | |
| ara-C derivatives, Boryung | Boryung Pharm Co Ltd | | neoplasm | |
| ara-C derivatives, Shinpoong | Shinpoong | | neoplasm | |
| CRD-602 | Chong Kun Dang Corp | | neoplasm | |
| KCRI-128-2 | Kolon Pharmaceuticals Inc | | neoplasm | |
| Xavedos | Pharmacia & Upjohn Inc | | leukemia | |
| Somatoscan | Draximage | | neoplasm | |
| 5-FU enhancer, Glaxo Wellcome | Glaxo Wellcome plc | | colorectal tumor | |
| antibiotics, | Micrologix Biotech Inc | | carcinoma | |
| Micrologix/PENCE/Alberta | | | | |
| hemochromatosis gene, | Progenitor Inc | | neoplasm | |
| Progenitor | | | | |
| facilitating cell technology | Chimeric Therapies Inc | | leukemia, lymphoma | |
| AIT (prostate cancer), AltaRex | AltaRex Corp | | prostate tumor | |
| Optimark | Mallinckrodt Inc | | brain tumor, spinal cord tumor, | |
| | | | liver tumor | |
| Cytocaps | Andaris Ltd | | neoplasm | |
| drug discovery, ArQule/Curagen | ArQule Inc | | carcinoma | |
| NX-1838 | NeXstar Pharmaceuticals Inc | | neoplasm | |
| prostate cancer therapy, UroGen | UroGen Corp | | prostate tumor | |
| leptin receptor technology | Progenitor Inc | | neoplasm | |
| PZ-301 | Prizm Pharmaceuticals Inc | | solid tumor | |
| (99m)technetium mAb- | CYTOGEN Corp | | breast tumor | |
| 17OH.82, CYTOGEN | | | | |
| endothelial cell vectors, | Neurotech SA | | glioma | |
| Neurotech/Kennedy Krieger | | | | |
| MIBG | Free University of Amsterdam | | leukemia | |
| etoposide analogs | IGT Pharma Inc | | carcinoma | |
| LMB-9 | National Cancer Institute | | carcinoma | |
| progression-elevation genes, | GenQuest Inc | | neoplasm | |
| PD-169540 | Parke-Davis & Co | | carcinoma | |
| cancer monoclonals, BMS | Bristol-Myers Squibb Co | | carcinoma | |
| 3-oxauracil | Walter Reed Army Institute of | | neoplasm | |
| | Research | | | |
| gene therapy (cancer), | GenVec Inc | | neoplasm | |
| GenVec/Fuso | | | | |
| multiplex screening, | Genometrix Inc | | neoplasm | |
| Genometrix/GeneMedicine | | | | |
| radiopharmaceuticals, | Mallinckrodt Inc | | breast tumor, colon tumor, lung | |
| Mallinckrodt | | | tumor, pancreas tumor, prostate | |
| | | | tumor, skin tumor | |
| mammastatin | Biotherapies Inc | WO 98/14577 | breast tumor | |
| Taxoprexin | Neuromedica Inc | | neoplasm | |
| CP-255 | Cell Pathways Inc | | neoplasm | |
| HYB-190 | Hybridon Inc | | neoplasm | |
| RENs/RENt analogs, NABI | University of Maryland | | neoplasm | |
| DIRECT technology, Argonex | Argonex Inc | | colorectal tumor, lung tumor, | |
| | | | melanoma, ovary tumor, prostate | |
| | | | tumor | |
| testisin | Queensland Institute of Medical | | testis tumor | |
| | Research | | | |
| BCH-2537 | BioChem Therapeutic Inc | | neoplasm | |
| G250 | Daniel den Hoed Cancer Center | | renal tumor | |
| GD-0039 | Glycodesign Inc | | breast tumor, colorectal tumor, | |
| | | | lung tumor, neoplasm, renal | |
| | | | tumor | |
| antinuclear autoantibodies, | Procyon Biopharma | | neoplasm | |
| Procyon | | | | |
| CART, Arena Pharmaceuticals | Arena Pharmaceuticals | | breast tumor, neoplasm | |
| drug targeting (angiogenesis), | Duke University | | angiogenesis disorder, neoplasm | |
| Duke | | | | |
| TX3.833, Beth Israel | Beth Israel Deaconess Medical | | lung tumor | |
| | Center | | | |
| bispecific antibody (cancer), | IBC Pharmaceuticals LLC | | neoplasm | |
| IBC | | | | |
| DTctGM-CSF | Wayne Hughes Institute | | leukemia | |
| LT gene, Targeted Genetics | Targeted Genetics Corp | | neoplasm | |
| flavone antitumor agents, | Kyowa Hakko Kogyo Co Ltd | | neoplasm | |
| Kyowa | | | | |
| gene vector (HSV), University | Tel Aviv University | | lymphoma | |
| of Tel-Aviv | | | | |
| gene vector (HHV-6), University | Tel Aviv University | | lymphoma | |
| of Tel-Aviv | | | | |
| Tamplicon-7, Univ of Tel-Aviv | Tel Aviv University | | lymphoma | |
| gene therapy (cancer), | Princeton University | | neoplasm | |
| Princeton/Gen | | | | |
| adenoviral gene therapy, | UroGen Corp | | neoplasm | |
| UroGen/Baxter | | | | |
| C5-OHP-Cl | Kanazawa University | | neoplasm | |
| LTKOSN.1, Human Gene | Human Gene Therapy Research | | neoplasm | |
| Therapy Research Institute | Institute | | | |
| NSC-161128 | University of Kansas | | prostate tumor | |
| DF-203 | University of Nottingham | | breast tumor, colon tumor, ovary | |
| | | | tumor | |
| AMI-227 | Advanced Magnetics Inc | | liver tumor, lymphoma | |
| argimesna | Schering AG | EP 0 198 542 | carcinoma, neoplasm | |
| BE-12406A | Banyu Pharmaceutical Co Ltd | EP 0 381 138 | carcinoma, neoplasm | |
| CP-79328 | Pfizer Inc | | carcinoma, neoplasm | |
| desmethoxystreptonigrin | Bristol-Myers Squibb Co | | carcinoma, neoplasm | |
| dinaline | Parke-Davis & Co | | carcinoma, neoplasm | |
| EG-6 | Takeda Chemical Industries Ltd | JP 01019048 | carcinoma | |
| gadodiamide | Hafslund Nycomed A/S | WO 86/02841 | central nervous system disease | |
| GTC, Pfizer | Pfizer Inc | | carcinoma | |
| OncoTrac NSCLC, NeoRx | NeoRx Corp | | lung tumor | |
| R-26390 | Janssen Pharmaceutica NV | | carcinoma | |
| 28A32 | Akzo Nobel NV | | carcinoma | |
| RG-83852 | Rhone-Poulenc SA | | carcinoma | |
| SMART ABL-364 | Novartis AG | | breast tumor, colon tumor, | |
| | | | colorectal tumor, lung tumor, | |
| | | | neoplasm, ovary tumor, pancreas | |
| | | | tumor | |
| sperabillin A | Takeda Chemical Industries Ltd | U.S. Pat. No. | neoplasm | |
| | | 4,839,351 | | |
| SR-26050 | Sanofi Recherche SA | | carcinoma | |
| tetrazomine | Yamanouchi Pharmaceutical Co | JP 02218684 | carcinoma | |
| | Ltd | | | |
| theonelladin A | Mitsubishi Chemical Corp | JP 03017060 | carcinoma | |
| U-77863 | Pharmacia & Upjohn Co | | carcinoma | |
| VSA-671 | Medivir AB | | carcinoma | |
| Zyn-linkers technology, Zynaxis | Zynaxis Inc | WO 93/11120 | neoplasm | |
| E-5166 | Eisai Co Ltd | | carcinoma | |
| A-62176 | Abbott Laboratories | | neoplasm | |
| EchoGen | Sonus Pharmaceuticals Inc | | prostate tumor | |
| vaccine (cancer), Sandoz/Wistar | Wistar Institute of Anatomy & | | neoplasm | |
| | Biology | | | |
| BCH-730 | BioChem Pharma Inc | | neoplasm | |
| BCH-671 | BioChem Pharma Inc | | neoplasm | |
| BCH-670 | BioChem Pharma Inc | | neoplasm | |
| UCF-1C | Kyowa Hakko Kogyo Co Ltd | | neoplasm | |
| MEN-10561 | A Menarini Ind Farm Riunite | | carcinoma, neoplasm | |
| | SrL | | | |
| RG-50860 | Elf Sanofi | | neoplasm | |
| RG-50872 | Elf Sanofi | | neoplasm | |
| RG-50875 | Elf Sanofi | | neoplasm | |
| PD-141076 | Parke-Davis & Co | | neoplasm | |
| PD-141703 | Parke-Davis & Co | | neoplasm | |
| socorromycin | Abbott Laboratories | | neoplasm | |
| Goe-4902 | Goedecke AG | U.S. Pat. No. | neoplasm | |
| | | 4,933,368 | | |
| acivicin | Pharmacia & Upjohn Co | | neoplasm | |
| steffimycin B | Pharmacia & Upjohn Co | U.S. Pat. No. | neoplasm | |
| | | 3,794,721 | | |
| U-77848 | Pharmacia & Upjohn Co | | neoplasm | |
| prednimustine | Leo AB | DE 2001305 | carcinoma | |
| BU-4704 | Bristol-Myers Squibb Co | | carcinoma, melanoma | |
| lentinan sulphate, Yamanouchi | Ajinomoto Co Inc | | carcinoma | |
| NSC-357704 | Pharmacia & Upjohn AB | | carcinoma, neoplasm | |
| SM-11355 | Sumitomo Pharmaceuticals Co | WO 94/14470 | neoplasm | |
| | Ltd | | | |
| WS-1279 | Fujisawa Pharmaceutical Co Ltd | JP 04046194 | neoplasm | |
| sultriecin | Bristol-Myers Squibb Co | U.S. Pat. No. | carcinoma, melanoma | |
| | | 4,952,709 | | |
| verucopeptin | Bristol-Myers Squibb Co | | neoplasm | |
| gallium nitrate | Fujisawa Pharmaceutical Co Ltd | | bone tumor, hypercalcemia | |
| mitoflaxone | Merck KGaA | | carcinoma, neoplasm | |
| adenosine nucleosides, Du Pont | DuPont Pharmaceuticals Co | | neoplasm | |
| Merck | | | | |
| cytosine nucleosides, Taiho | Taiho Pharmaceutical Co Ltd | | neoplasm | |
| DX-52-1 | Kyowa Hakko Kogyo Co Ltd | | melanoma, neoplasm | |
| KW-2152 | Kyowa Hakko Kogyo Co Ltd | | melanoma, neoplasm | |
| U-891 | Pharmacia & Upjohn Co | | neoplasm | |
| BMY-27557 | Bristol-Myers Squibb Co | | neoplasm | |
| angelmicin | Bristol-Myers Squibb Co | | neoplasm | |
| BCH-1128 | BioChem Pharma Inc | | neoplasm | |
| MSI-511 | Magainin Pharmaceuticals Inc | | melanoma, neoplasm | |
| AD-198 | Anthra Pharmaceuticals | | lung tumor, neoplasm | |
| XK-469 | DuPont Pharmaceuticals Co | | neoplasm | |
| miltefosine | ASTA Medica Inc | | breast tumor, neoplasm, skin | |
| | | | tumor | |
| MDR-1 gene therapy, Genetic | Genetic Therapy Inc | U.S. Pat. No. | breast tumor | |
| Therapy | | 5,399,346 | | |
| 3F8 | Genetics Institute Inc | | nervous system tumor, | |
| | | | melanoma, neoplasm | |
| GNI-250 | Genetics Institute Inc | | neoplasm | |
| capromab | CYTOGEN Corp | | prostate tumor | |
| CYT-103-Y90 | CYTOGEN Corp | | carcinoma, colorectal tumor, | |
| | | | ovary tumor | |
| monoclonals (cancer), BTG | British Technology Group Plc | | bladder tumor | |
| ICAM-3 antibodies, ICOS | Icos Corp | U.S. Pat. No. | neoplasm, nervous system tumor | |
| | | 5,525,487 | | |
| oligonucleotides (ras), Genta | Genta Inc | | neoplasm | |
| oligonucleotides | Genta Inc | | squamous cell carcinoma | |
| (thrombospondin), Genta | | | | |
| oligonucleotide (myc), Genta | Genta Inc | | leukemia | |
| Zyn-Linker oligonucleotides, | Genta Inc | | neoplasm | |
| Genta/Zynaxis | | | | |
| oligonucleotide (interleukin-1), | Genta Inc | | leukemia | |
| Genta | | | | |
| fosteabine | Nippon Kayaku Co Ltd | | leukemia, liver tumor, neoplasm | |
| CD5 monoclonals/RIPs, | Italfarmaco SpA | | neoplasm | |
| Italfarmaco | | | | |
| P-67, Immunex | Immunex Corp | | neoplasm | |
| pEEDCK | Hafslund Nycomed A/S | | neoplasm | |
| Versaluma | NeoRx Corp | | lung tumor | |
| MAb PR1, NIH | National Institutes of Health | | carcinoma | |
| monoclonal-porphyrins, Quadra | QLT PhotoTherapeutics Inc | | neoplasm | |
| Logic | | | | |
| CD4 fusion toxin, Seragen | Seragen Inc | | neoplasm | |
| interleukin-6 fusion toxin, | Seragen Inc | | kaposis sarcoma, | |
| Seragen | | | myeloproliferative disorder | |
| MSH fusion toxin, Seragen | Seragen Inc | | melanoma | |
| XomaZyme-791 | XOMA Corp | | carcinoma, neoplasm | |
| Tru-Scint | Biomira Inc | | carcinoma, breast tumor | |
| Pepscan | Antisoma plc | | carcinoma, brain tumor | |
| MAbs (solid tumors), BMS | Bristol-Myers Squibb Co | | carcinoma, lung tumor, | |
| | | | melanoma | |
| CDP-833 | Celltech Group plc | | colon tumor, colorectal tumor | |
| BABS proteins, Creative | Creative Biomolecules Inc | | neoplasm, breast tumor | |
| BioMol | | | | |
| stem cells, Progenitor | Interneuron Pharmaceuticals Inc | | bone marrow transplantation, | |
| | | | lymphoma, neoplasm | |
| NG-1 | Novopharm Biotech Inc | | carcinoma, neoplasm | |
| MDX-11 | Medarex Inc | | carcinoma, myeloid leukemia | |
| MPC-467 | Microprobe Corp | | carcinoma, colon tumor, liver | |
| | | | tumor, lymphoma | |
| gene isolation process, | Transkaryotic Therapies Inc | | melanoma | |
| Transkaryotic Ther | | | | |
| gene therapy, Transkaryotic | Transkaryotic Therapies Inc | | melanoma, neoplasm | |
| Therapies | | | | |
| gene targeting technology, | Transkaryotic Therapies Inc | | melanoma | |
| Transkaryotic Ther | | | | |
| epidermal negative growth | Yissum Research Development | | neoplasm | |
| factor, Yissum | Co of the Hebrew University of | | | |
| | Jerusalem | | | |
| 6-azacytidine analogs | National Academy of Sciences | | neoplasm | |
| | of Ukraine | | | |
| Ro-44-5912 | Roche Holding AG | | neoplasm | |
| 9187 | Baxter International Inc | | breast tumor, neoplasm | |
| DAB389-hGMCSF | Hafslund Nycomed A/S | | neoplasm | |
| DAB389-hGCSF | Hafslund Nycomed A/S | | neoplasm | |
| CRL-1336 | CytRx Corp | | leukemia | |
| 99mTc P587 | Diatide Inc | | neoplasm | |
| carbetimer | G D Searle & Co | | colorectal tumor, melanoma, | |
| | | | neoplasm | |
| cytoros | Health Research Inc | | neoplasm | |
| ProGRP(31-98), Tonen | Tonen Corp | | carcinoma | |
| CGP-55398 | Novartis AG | | carcinoma, neoplasm | |
| autolymphocyte therapy (RCC), | Cellcor Inc | | melanoma, prostate tumor, renal | |
| Cellcor | | | tumor | |
| autologous T cells, Somatix | Somatix Therapy Corp | | neoplasm | |
| FJ-776 | Fuji Chemical Industries Co Ltd | | neoplasm | |
| AP-633 | Ariad Pharmaceuticals Inc | | neoplasm | |
| AP-656 | Ariad Pharmaceuticals Inc | | neoplasm | |
| OCTR lymphocytes, Centocor | Centocor Inc | | neoplasm | |
| EF-13 | Efamol | | brain tumor, breast tumor, colon | |
| | | | tumor, lung tumor, melanoma, | |
| | | | neoplasm, pancreas tumor | |
| mitomycin C derivative, Kyowa | Kyowa Yakuhin Kogyo Co Ltd | | neoplasm | |
| BBR-2889 | Boehringer Mannheim GmbH | | leukemia | |
| BBR-2382 | Boehringer Mannheim GmbH | | lung tumor | |
| BCH-1167 | BioChem Pharma Inc | | neoplasm | |
| BCH-1184 | BioChem Pharma Inc | | neoplasm | |
| BCH-2005 | BioChem Pharma Inc | | neoplasm | |
| BCH-2050 | BioChem Pharma Inc | | neoplasm | |
| interleukin-13, Schering-Plough | Schering-Plough Corp | | leukemia | |
| AdoHcy hydrolase inhibitor, | Rega Institute for Biomedical | | carcinoma | |
| Rega Institute | Research | | | |
| IL-2 antibody (anti-tumor), | Hybritech Inc | | neoplasm | |
| Hybritech | | | | |
| SF-2738 | Meiji Seika Kaisha Ltd | | carcinoma | |
| himastatin | Bristol-Myers Squibb | | leukemia, melanoma | |
| | Pharmaceuticals Ltd | | | |
| retinoblastoma gene therapy, | Canji Inc | | bladder tumor, bone tumor, | |
| Canji | | | breast tumor, lung tumor, | |
| | | | prostate tumor | |
| NCU-304 | Japanese Cancer Institute | | carcinoma | |
| hCTMO1 | Celltech Group plc | | breast tumor | |
| CT-3532 | Cell Therapeutics Inc | | carcinoma | |
| MA-5000 | Merck & Co Inc | | carcinoma | |
| irsogladine | Nippon Shinyaku Co Ltd | | metastasis | |
| OctreoScan | NeXstar Pharmaceuticals Inc | | neoplasm | |
| gene therapy (HSV-tk/GCV), | Genopoietic | | glioma, melanoma | |
| RPR/Genopoietic | | | | |
| MDX-22 | Medarex Inc | | myeloid leukemia | |
| CRL-1337 | CytRx Corp | | leukemia | |
| ZYN-162 | Zynaxis Inc | | ovary tumor | |
| NeuGene | AVI BioPharma | | neoplasm | |
| azaanthraquinones, Pharma Mar | Pharma Mar SA | | carcinoma | |
| mycaperoxide B | Pharma Mar SA | | carcinoma, lung tumor, ovary | |
| | | | tumor | |
| kahalalide F | Pharma Mar SA | | carcinoma, colon tumor, prostate | |
| | | | tumor | |
| PM-92114 | Pharma Mar SA | | melanoma | |
| crambescidia-816 | Pharma Mar SA | | melanoma | |
| thiocoraline | Pharma Mar SA | | breast tumor, melanoma | |
| AR-726 | Aronex Pharmaceuticals Inc | | neoplasm | |
| D-20566 | ASTA Medica Arzneimittel Ges | | neoplasm | |
| | mbH | | | |
| lytic peptides, Proteus | Proteus Molecular Design Ltd | | carcinoma | |
| RGA-1512 | RGene Therapeutics Inc | | leukemia, myeloid leukemia | |
| GS-438 | Gilead Sciences Inc | | neoplasm | |
| AMP-53 | NeXstar Pharmaceuticals Inc | | neoplasm | |
| tetrofosmin | Amersham International plc | | breast tumor | |
| iobitridol | Guerbet SA | | carcinoma | |
| 99mTc P829 | Diatide Inc | | carcinoma, lung tumor, | |
| | | | melanoma, metastasis, | |
| | | | neuroendocrine tumor | |
| Sn-117m DTPA | Diatide Inc | | carcinoma | |
| KNK-41 | Kuraray Co Ltd | | carcinoma | |
| anti-ovary cancer, MAb | Medarex Inc | | carcinoma | |
| DC-92-B | Kyowa Hakko Kogyo Co Ltd | | carcinoma | |
| IPAB, RCT | Research Corp Technologies Inc | | carcinoma | |
| CEPRATE, CellPro | Cellpro Inc | | bone marrow transplantation, | |
| | | | lung tumor, myeloproliferative | |
| | | | disorder, neoplasm | |
| BHC-AC | Asahi Chemical Industry Co Ltd | | leukemia | |
| spirogermanium hydrochloride | Unimed Pharmaceuticals Inc | | neoplasm | |
| bullatacin | Upjohn Holding Co | | carcinoma | |
| B2D, SRI International | SRI International | | carcinoma | |
| AC-9401 | Anticancer Inc | | lung tumor, neoplasm | |
| peptide-T, Peptech | Peptech (UK) Ltd | | carcinoma | |
| jasplakinolide | National Institutes of Health | | breast tumor | |
| DNA-binding drugs, Progene | Progene Partners | | carcinoma | |
| Aastrom Cell Production System | Aastrom Biosciences Inc | | bone marrow transplantation, | |
| | | | carcinoma | |
| boronated nucleic acids, BBI | Boron Biologicals Inc | | neoplasm | |
| DNA binding agents, Proteus | Progene Partners | | carcinoma | |
| micelle platinum complexes, | Roswell Park Cancer Institute | | neoplasm | |
| Roswell | | | | |
| UK-21 | Gifu Pharmaceutical University | | neoplasm | |
| icodextrin | ML Laboratories plc | | neoplasm, ovary tumor, | |
| | | | colorectal tumor, intestine tumor | |
| HSCs, SyStemix | SyStemix Inc | | non-Hodgkin's lymphoma, | |
| | | | breast tumor, carcinoma, | |
| | | | myeloproliferative disorder | |
| SC-101a | Scotia Holdings plc | | brain tumor, metastasis, | |
| | | | neoplasm, prostate tumor | |
| COL-1 | National Cancer Institute | | pancreas tumor, stomach tumor, | |
| | | | intestine tumor, breast tumor, | |
| | | | lung tumor | |
| cell cycle regulators, | ONYX Pharmaceuticals Inc | | neoplasm | |
| Onyx/Parke-Davis | | | | |
| IDEC-In2B8 | IDEC Pharmaceuticals Corp | WO 94/11026 | non-Hodgkin's lymphoma | |
| Gadolinium-Bopta | Bracco Industria Chimica SpA | | neoplasm | |
| gene therapy (breast cancer), | Imperial Cancer Research | | breast tumor, neoplasm | |
| ICRF | Technology Ltd | | | |
| minichromosome, Canji | American Gene Therapy Inc | | neoplasm | |
| anticancers, Signal | Signal Pharmaceuticals Inc | | lung tumor, breast tumor, ovary | |
| | | | tumor, myeloproliferative | |
| | | | disorder, leukemia | |
| Dy-Tex | Pharmacyclics Inc | | neoplasm | |
| Prosorba | Cypress Bioscience Inc | | neoplasm, breast tumor | |
| cell therapy, X-Cell Biotech | X-Cell Biotech | | neoplasm | |
| Eukaryotic Layered Vector | Chiron Viagene Inc | | neoplasm | |
| System | | | | |
| hIgG antibodies, GenPharm | Genpharm International Inc | | neoplasm | |
| recombinant adenoviral vectors, | Institut Gustave Roussy | | lung tumor | |
| Gustave Roussy | | | | |
| salcatonin (pulmonary), Inhale | Inhale Therapeutic Systems | | Paget's disease, hypercalcemia | |
| gene discovery [prostate], | Genset | | prostate tumor | |
| Genset/Synthelabo/SB/HGS | | | | |
| TAPET, Vion | Vion Pharmaceuticals Inc | | neoplasm | |
| Nuclear Matrix Proteins | Matritech Inc | | uterine cervix tumor | |
| (cervical cancer), Matritech | | | | |
| U-Fucoidan | Takara Shuzo Co Ltd | | carcinoma | |
| translation inhibitors, RiboGene | Ribogene Inc | | carcinoma | |
| Theriform | Therics Inc | | neoplasm, hormone replacement | |
| | | | therapy | |
| drug delivery system | Cellegy Pharmaceuticals Inc | | skin tumor | |
| (transdermal), Cellegy | | | | |
| SPI-49 | Sequus Pharmaceuticals Inc | | carcinoma | |
| therapeutics, | Pharmacopeia Inc | | neoplasm | |
| Pharmacopeia/Schering-Plough | | | | |
| ribozymes (IGF-1), Ribozyme | Ribozyme Pharmaceuticals Inc | | neoplasm | |
| III-121C | Keio University | | carcinoma | |
| E2F inhibitors, Prolifix/Chugai | Prolifix Ltd | | neoplasm | |
| gene vectors (HSV), | NeuroVir | | neoplasm | |
| NeuroVir/Aviron | | | | |
| delivery system (p53), | Inex Pharmaceuticals Corp | | neoplasm | |
| Inex/Schering-Plough | | | | |
| colon specific DDS (budesonide) | Advanced Polymer Systems | | colon tumor | |
| chimeraplasty | Kimeragen, Inc | | leukemia | |
| RB-94 | Ingenex | | solid tumor | |
| p53 reactivation therapy, | Cyclacel Ltd | | head & neck tumor, neoplasm | |
| Cyclacel | | | | |
| Eu-Tex | Pharmacyclics Inc | | neoplasm | |
| TriGem | Trilex Pharmaceuticals Inc | | neoplasm, melanoma | |
| gene therapy (p16/p27), Mitotix | Mitotix Inc | | neoplasm | |
| gene therapy (interleukin-2), | Imperial Cancer Research | | melanoma, neoplasm | |
| ICRF | Technology Ltd | | | |
| leukemia therapy, University of | University of Pennsylvania | | myeloid leukemia | |
| Pennsylvania | | | | |
| diabetes therapy, | Telik Inc | | breast tumor | |
| Terrapin/Sanwa | | | | |
| OntoScreen | Ontogeny Inc | | neoplasm | |
| Vascular Targeting Agents, | Peregrine Pharmaceuticals Inc | | solid tumor | |
| Techniclone | | | | |
| cell therapy (leukemia), | Chiron Corp | | leukemia | |
| Chiron/Baxter | | | | |
| Taxol Transport, Enzon | Enzon Inc | | neoplasm | |
| Hycamptin Transport, Enzon | Enzon Inc | | neoplasm | |
| 105A5 | Universitat Tubingen | WO 97/07204 | neoplasm | |
| ribozymes, Ribozyme/Berlex | Ribozyme Pharmaceuticals Inc | | neoplasm | |
| cancer therapeutics, Schering- | Schering-Plough Corp | | prostate tumor, neoplasm | |
| Plough/Myriad Genetics | | | | |
| apoptosis inhibitors, | LXR Biotechnology Inc | | neoplasm | |
| LXR/Oxford Asymmetry | | | | |
| immunotherapy (neoplasm), IBS | International Bioimmune | | colorectal tumor, lung tumor | |
| | Systems Inc | | | |
| dendritic cell therapy, | University of California San | | prostate tumor, non-Hodgkin's | |
| UCSF/Activated Cell Therapy | Francisco | | lymphoma, myeloproliferative | |
| | | | disorder | |
| MB-300 series | Megabios Corp | | neoplasm | |
| MFPD | Nippon Kayaku Co Ltd | | neoplasm | |
| D-24241 | ASTA Medica AG | | neoplasm | |
| PTEN gene, ICRF | The UK Imperial Cancer | | brain tumor, glioma, neoplasm | |
| | Research Fund | | | |
| vascular targeting technology, | Corvas International Inc | | carcinoma | |
| Corvas | | | | |
| age-related disease therapy, | Geron Corp | | neoplasm | |
| Geron/Darwin | | | | |
| Adeno-Quest | Quantum Biotechnologies Inc | | neoplasm | |
| gene therapy (cancer), IDUN | IDUN Pharmaceuticals Inc | | neoplasm | |
| NA-22598-A1 | Nippon Kayaku Co Ltd | | neoplasm | |
| NSC-330507 | University of Maryland | | neoplasm | |
| PFP-6, University of Vienna | University of Vienna | | neoplasm | |
| SA-47 | The Salk Institute for Biological | | lymphoma, leukemia | |
| | Studies | | | |
| SA-450 | The Salk Institute for Biological | | leukemia, lymphoma | |
| | Studies | | | |
| IgA receptor bispecifics, | Medarex Inc | | neoplasm | |
| Medarex | | | | |
| PHP-CT | Ajinomoto Co Inc | | solid tumor | |
| K-ras ribozyme therapy (cancer), | Schering AG | | bladder tumor, prostate tumor, | |
| Schering | | | skin tumor, lung tumor | |
| fusogenic lipids, Liposome | The Liposome Company Inc | | neoplasm | |
| Company | | | | |
| gene therapy, Chugai | Chugai Research Institute for | | neoplasm | |
| | Molecular Medicine Inc | | | |
| p53 gene therapy, | Transgene SA | | neoplasm | |
| Transgene/Schering-Plough | | | | |
| cell surface MAb, Cambridge | Cambridge Antibody | | neoplasm | |
| Antibody/Mitsubishi | Technology Ltd | | | |
| LentiPak | Genetix Pharmaceuticals | | neoplasm | |
| gene therapy (cancer), NIH | National Institutes of Health | | brain tumor, neoplasm | |
| Alzheimers disease therapy, | University of Pittsburgh | | neoplasm | |
| Pittsburgh | | | | |
| prostate tumor-inducing genes, | GenQuest Inc | | prostate tumor | |
| GenQuest | | | | |
| prostate carcinoma tumor | GenQuest Inc | | prostate tumor | |
| antigens, GenQuest | | | | |
| gene therapy (cancer) | GenVec Inc | | neoplasm | |
| GenVec/Varian | | | | |
| MB-400 | Megabios Corp | WO 96/40963 | neoplasm | |
| chemotherapy (cancer), Enzacta | Enzacta Ltd | | neoplasm | |
| gene therapy (liver cancer), | Nagoya University | | liver tumor | |
| Nagoya University | | | | |
| tumor-activated prodrugs, | ImmunoGen Inc | | neoplasm | |
| ImmunoGen | | | | |
| BEPH, HMR | Hoechst Marion Roussel Inc | EP 0 277 635 | carcinoma | |
| BMS-181321 | Bristol-Myers Squibb Co | | solid tumor | |
| gene transfer therapy, Sandoz | Novartis AG | | neoplasm | |
| tumor necrosis therapy, | Techniclone Corp | | solid tumor, prostate tumor, | |
| Techniclone | | | brain tumor, glioma | |
| AO-82 | Novartis AG | | neoplasm | |
| TLC I-16 | The Liposome Company Inc | | liver tumor | |
| zinostatin stimalamer | Yamanouchi Pharmaceutical Co | EP 0 136 791 | liver tumor, brain tumor | |
| | Ltd | | | |
| KBS | Yamanouchi Pharmaceutical Co | | carcinoma | |
| | Ltd | | | |
| PAM4 | Merck & Co Inc | | neoplasm | |
| oncostatins | Bristol-Myers Squibb Co | WO 94/04190 | carcinoma | |
| Cardiolite | DuPont Pharmaceuticals Co | EP 0 233 368 | breast tumor | |
| OncoScint CR372 | CYTOGEN Corp | | neoplasm | |
| casein kinase 1 inhibitors, ICOS | Icos Corp | | neoplasm | |
| ERIC-1, ICRT | Imperial Cancer Research | | neoplasm | |
| | Technology Ltd | | | |
| gene therapeutics technology, | Vical Inc | | neoplasm | |
| Vical | | | | |
| AAV vectors, Avigen | Research Corp Technologies Inc | | liver tumor, neoplasm | |
| radiation therapy, GenVec | GenVec Inc | | neoplasm | |
| EGS technology, Innovir | Yale University | | leukemia, neoplasm | |
| oligonucleotide AML, Lynx | Lynx Therapeutics Inc | | leukemia | |
| p53 gene therapy, Genzyme | Genzyme Molecular Oncology | EP 0 518 650 | neoplasm | |
| Molecular Oncology | | | | |
| vector technology, Theragen | Theragen Inc | | head & neck tumor | |
| doxorubicin prodrugs, Hoechst | Hoechst AG | | lung tumor, breast tumor, | |
| | | | digestive system tumor | |
| AR-623 | Aronex Pharmaceuticals Inc | | leukemia, kaposis sarcoma | |
| MSI-103 | Magainin Pharmaceuticals Inc | | carcinoma | |
| MAb32, Peptide Technology | Peptech Ltd | | carcinoma | |
| Monopharm-C | Novophram Biotech Inc | | breast tumor, colon tumor, lung | |
| | | | tumor, pancreas tumor, prostate | |
| | | | tumor, stomach tumor | |
| gene therapy (cancer), Darwin | Darwin Molecular Corp | | neoplasm | |
| Molecular | | | | |
| TJ-9 | Tsumura & Co Ltd | | carcinoma, liver tumor | |
| PLATAR, Tanox | Tanox Biosystems Inc | | infection, neoplasm | |
| gene therapy technology, | Progenitor Inc | | neoplasm | |
| Progenitor | | | | |
| IntraDose-CDDP | Matrix Pharmaceutical Inc | | breast tumor, esophagus tumor, | |
| | | | head & neck tumor, liver tumor, | |
| | | | lung tumor, melanoma, | |
| | | | neoplasm, prostate tumor, rectal | |
| | | | tumor | |

In one embodiment of the present invention, a combination comprising a Cox-2 inhibitor and an antineoplastic agent is administered to a subject by a standard route of drug delivery, such standard routes being well known to one of ordinary skill in the art.

Either or both of the Cox-2 inhibitor and the antineoplastic agent can optionally be supplied in the form of a pharmaceutically active salt, a prodrug, an isomer, a racemic mixture, or in any other chemical form or combination.

Illustrative pharmaceutically acceptable salts are prepared from formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, cyclohexylaminosulfonic, algenic, β-hydroxybutyric, galactaric and galacturonic acids.

Suitable pharmaceutically-acceptable base addition salts include metallic ion salts and organic ion salts. Metallic ion salts include, but are not limited to, appropriate alkali metal (group Ia) salts, alkaline earth metal (group IIa) salts and other physiologically acceptable metal ions. Such salts can be made from the ions of aluminum, calcium, lithium, magnesium, potassium, sodium and zinc. Organic salts can be made from tertiary amines and quaternary ammonium salts, including in part, trimethylamine, diethylamine, N,N′-dibenzylethylenediamine, chloroprocaine, choline, diethanolamine, ethylenediamine, meglumine (N-methylglucamine) and procaine. All of the above salts can be prepared by those skilled in the art by conventional means from the corresponding compound.

A combination of a Cox-2 inhibitor and an antineoplastic agent can be provided in a pharmaceutically acceptable carrier or excipient to form a pharmaceutical composition. Pharmaceutical compositions can also include stabilizers, antioxidants, colorants and diluents. Pharmaceutically acceptable carriers and additives are chosen such that side effects from the pharmaceutical compound are minimized and the performance of the compound is not canceled or inhibited to such an extent that treatment is ineffective. In one embodiment, a Cox-2 inhibitor and an antineoplastic agent are administered to a subject together in one pharmaceutical carrier. In another embodiment, they are administered separately.

The pharmaceutical compositions may be administered enterally and/or parenterally. Oral (intra-gastric) is a typical route of administration. Pharmaceutically acceptable carriers can be in solid dosage forms, including tablets, capsules, pills and granules, which can be prepared with coatings and shells, such as enteric coatings and others well known in the art. Liquid dosage forms for oral administration include pharmaceutically acceptable emulsions, solutions, suspensions, syrups and elixirs.

Parenteral administration includes subcutaneous, intramuscular, intradermal, intramammary, intravenous, and other routes known in the art. Enteral administration includes solution, tablets, sustained release capsules, enteric coated capsules, and syrups. When administered, the pharmaceutical composition can be at or near body temperature.

Compositions intended for oral use can be prepared according to any method known in the art for the manufacture of pharmaceutical compositions and such compositions can contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients, which are suitable for the manufacture of tablets. These excipients may be, for example, inert diluents, such as calcium carbonate, sodium carbonate, lactose, calcium phosphate or sodium phosphate, granulating and disintegrating agents, for example, maize starch, or alginic acid, binding agents, for example starch, gelatin or acacia, and lubricating agents, for example magnesium stearate, stearic acid, or talc. Tablets can be uncoated or they can be coated by known techniques, for example to delay disintegration and absorption in the gastrointestinal tract and thereby provide sustained action over a longer period. For example, a time delay material such as glyceryl monostearate or glyceryl distearate can be employed.

Formulations for oral use can also be presented as hard gelatin capsules wherein the active ingredients are mixed with an inert solid diluent, for example, calcium carbonate, calcium phosphate or kaolin, or as soft gelatin capsules wherein the active ingredients are present as such, or mixed with water or an oil medium, for example, peanut oil, liquid paraffin or olive oil.

Aqueous suspensions can be produced that contain the active materials in a mixture with excipients suitable for the manufacture of aqueous suspensions. Such excipients include suspending agents, for example, sodium carboxymethylcellulose, methylcellulose, hydroxypropylmethylcellulose, sodium alginate, polyvinylpyrrolidone, gum tragacanth and gum acacia; dispersing or wetting agents can be naturally-occurring phosphatides, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyoxyethylene sorbitan monooleate.

Aqueous suspensions can also contain one or more preservatives, for example, ethyl or n-propyl p-hydroxybenzoate, one or more coloring agents, one or more flavoring agents, or one or more sweetening agents, such as sucrose or saccharin.

Oily suspensions may be formulated by suspending the active ingredients in an omega-3 fatty acid, a vegetable oil, for example, arachis oil, olive oil, sesame oil or coconut oil, or in a mineral oil such as liquid paraffin. The oily suspensions can contain a thickening agent, for example beeswax, hard paraffin or cetyl alcohol.

Sweetening agents, such as those set forth above, and flavoring agents can be added to provide a palatable oral preparation. These compositions can be preserved by addition of an antioxidant such as ascorbic acid.

Dispersible powders and granules suitable for preparation of an aqueous suspension by addition of water provide the active ingredient in admixture with a dispersing or wetting agent, a suspending agent and one or more preservatives. Suitable dispersing or wetting agents and suspending agents are exemplified by those already mentioned above. Additional excipients, for example sweetening, flavoring and coloring agents, can also be present.

Syrups and elixirs containing a Cox-2 inhibitor and/or an antineoplastic agent can be formulated with sweetening agents, for example glycerol, sorbitol, or sucrose. Such formulations can also contain a demulcent, a preservative and flavoring and coloring agents.

A Cox-2 inhibitor and an antineoplastic agent can be administered parenterally, for example subcutaneously, intravenously, intramuscularly or intrasternally, or by infusion techniques, in the form of sterile injectable aqueous or oleaginous suspensions. Such suspensions can be formulated according to known art using suitable dispersing or wetting agents and suspending agents such as those mentioned above or other acceptable agents. A sterile injectable preparation can be a sterile injectable solution or suspension in a non-toxic parenterally acceptable diluent or solvent, for example a solution in 1,3-butanediol. Among acceptable vehicles and solvents that can be employed are water, Ringer's solution and isotonic sodium chloride solution. In addition, sterile fixed oils are conventionally employed as a solvent or suspending medium. For this purpose, any bland fixed oil may be employed, including synthetic mono- or diglycerides. In addition, omega-3 polyunsaturated fatty acids can find use in preparation of injectables.

Administration can also be by inhalation, in the form of aerosols or solutions for nebulizers, or rectally, in the form of suppositories prepared by mixing the drug with a suitable non-irritating excipient which is solid at ordinary temperature, but liquid at rectal temperature and will therefore, melt in the rectum to release the drug. Such materials are cocoa butter and polyethylene glycols.

Also encompassed by the present invention is buccal and sub-lingual administration, including administration in the form of lozenges, pastilles or a chewable gum comprising the compounds set forth herein. The compounds can be deposited in a flavored base, usually sucrose, and acacia or tragacanth.

Other methods for administration of the Cox-2 inhibitor and the antineoplastic agent include dermal patches that release the medicaments directly into and/or through a subject's skin.

Topical delivery systems are also encompassed by the present invention and include ointments, powders, sprays, creams, jellies, collyriums, solutions or suspensions.

Powders have the advantage of sticking to moist surfaces, and consequently, can remain active for longer periods. Therefore, powders are especially attractive for treating neoplasms in, for example, the otic canal. For much the same reason, creams are also effective pharmaceutically acceptable carriers.

Compositions of the present invention can optionally be supplemented with additional agents such as, for example, viscosity enhancers, preservatives, surfactants and penetration enhancers.

Viscosity-building agents include, for example, polyvinyl alcohol, polyvinyl pyrrolidone, methylcellulose, hydroxypropylmethylcellulose, hydroxyethylcellulose, carboxymethylcellulose, hydroxypropylcellulose or other agents known to those skilled in the art. Such agents are typically employed at a level of about 0.01% to about 2% by weight of a pharmaceutical composition.

Preservatives are optionally employed to prevent microbial growth prior to or during use. Suitable preservatives include polyquaternium-1, benzalkonium chloride, thimerosal, chlorobutanol, methylparaben, propylparaben, phenylethyl alcohol, edetate disodium, sorbic acid, or other agents known to those skilled in the art. Typically, such preservatives are employed at a level of about 0.001% to about 1.0% by weight of a pharmaceutical composition.

Solubility of components of the present compositions can be enhanced by a surfactant or other appropriate cosolvent in the composition. Such cosolvents include polysorbates 20, 60 and 80, polyoxyethylene/polyoxypropylene surfactants (e.g., Pluronic™ F-68, F-84 and P-103), cyclodextrin, or other agents known to those skilled in the art. Typically, such cosolvents are employed at a level of about 0.01% to about 2% by weight of a pharmaceutical composition.

Pharmaceutically acceptable excipients and carriers encompass all the foregoing and the like. The above considerations concerning effective formulations and administration procedures are well known in the art and are described in standard textbooks. See, e.g., Remington: The Science and Practice of Pharmacy, 20th Edition, (Lippincott, Williams and Wilkins), 2000; Lieberman et al., ed., Pharmaceutical Dosage Forms, Marcel Decker, New York, N.Y., 1980; and Kibbe et al., ed., Handbook of Pharmaceutical Excipients (3rd Edition), American Pharmaceutical Association, Washington, 1999.

For purposes of the present invention, where a Cox-2 inhibitor and an antineoplastic agent are used in a combination therapy, the amount of the Cox-2 inhibitor and the amount of the antineoplastic agent should comprise an effective amount of the combination of the two treatment agents.

Thus, the present invention encompasses a method of treating or preventing neoplasia or a neoplasia-related disorder in a subject in need of such treatment or prevention, the method comprising administering a first amount of a Cox-2 inhibitor in combination with a second amount of an antineoplastic agent, wherein the amount of the combination, i.e., the total of said first and second amounts, is therapeutically effective for such treatment or prevention.

In determining an effective amount or dose, a number of factors are considered by the attending physician, including, but not limited to, the potency and duration of action of the compounds used, the nature and severity of the illness to be treated, as well as the sex, age, weight, general health and individual responsiveness of the patient to be treated, and other relevant circumstances. Those skilled in the art will appreciate that dosages can also be determined with guidance from Goodman & Goldman's The Pharmacological Basis of Therapeutics, Ninth Edition (1996), Appendix II, pp. 1707-1711.

It will be appreciated that the amount of the combination comprising a Cox-2 inhibitor and an antineoplastic agent required for use in the treatment or prevention of neoplasia and neoplasia-related disorders will vary within wide limits and will be adjusted to the individual requirements in each particular case. In general, for administration to adults, an appropriate daily dosage is described herein, although the limits that are identified as being preferred can be exceeded if expedient. The daily dosage can be administered as a single dosage or in divided dosages.

The dosage level of an antineoplastic agent will necessarily depend on the particular agent that is used. Appropriate dosages can be readily determined by one of skill in the art based upon the present specification and published information on the agent in question, available for example on the Internet. However, an appropriate dosage level of an antineoplastic agent is generally from about 0.0001 mg/kg to about 200 mg/kg subject body weight per day, administered in single or multiple doses. More typically, the dosage level is about 0.1 mg/kg to about 25 mg/kg per day.

A combination therapy comprising a Cox-2 inhibitor and an antineoplastic agent has an appropriate dosage level of the Cox-2 inhibitor that is generally from about 0.01 mg/kg to about 140 mg/kg subject body weight per day, administered in single or multiple doses. More typically, the dosage level is about 0.01 mg/kg to about 50 mg/kg per day, for example about 0.1 mg/kg to about 25 mg/kg per day, about 0.1 mg/kg to about 10 mg/kg per day, or about 0.5 mg/kg to about 10 mg/kg per day.

In larger mammals, for example humans, a typical indicated dose for the Cox-2 inhibitor is about 0.5 mg to about 7 grams orally per day. A compound can be administered on a regimen of several times per day, for example 1 to about 4 times per day, preferably once or twice per day.

The amount of the Cox-2 inhibitor that can be combined with carrier materials to produce a single dosage form varies depending upon the subject to be treated and the particular mode of administration. For example, a formulation intended for oral administration to humans can contain about 0.5 mg to about 7 g of active agent compounded optionally with an appropriate and convenient amount of carrier material which can vary from about 5 to about 95 percent of the total composition. Dosage unit forms for the Cox-2 inhibitor generally contain about 1 mg to about 500 mg of the active ingredient, for example 5 mg, 10 mg, 20 mg, 25 mg, 50 mg, 100 mg, 200 mg, 300 mg, 400 mg, 500 mg, 600 mg, 800 mg or 1000 mg.

The exact dosage and regimen for administering a Cox-2 inhibitor in combination with an antineoplastic agent will necessarily depend upon the potency and duration of action of the compounds used, the nature and severity of the illness to be treated, as well as the sex, age, weight, general health and individual responsiveness of the patient to be treated, and other relevant circumstances. Those skilled in the art will appreciate that dosages may also be determined with guidance from Goodman & Goldman's The Pharmacological Basis of Therapeutics, Ninth Edition (1996), Appendix II, pp. 1707-1711.

The effectiveness of a particular dosage of a combination therapy comprising a Cox-2 inhibitor and an antineoplastic agent can be determined by monitoring the effect of a given dosage on the progression of the disorder or prevention of a neoplasia disorder.

In one embodiment, the effectiveness of a particular dosage is determined by staging the disorder at multiple points during a subject's treatment. For example, once a histological diagnosis is made, staging (i.e., determination of the extent of disease) helps determine treatment decisions and prognosis. Clinical staging uses data from the patient's history, physical examination, and noninvasive studies. Pathologic staging requires tissue specimens.

Pathological staging is performed by obtaining a biopsy of the neoplasm or tumor. A biopsy is performed by obtaining a tissue specimen of the tumor and examining the cells microscopically. A bone marrow biopsy is especially useful in determining metastases from malignant lymphoma and small cell lung cancer. Marrow biopsy will be positive in 50 to 70% of patients with malignant lymphoma (low and intermediate grade) and in 15 to 18% of patients with small cell lung cancer at diagnosis. See The Merck Manual of Diagnosis & Therapy, 17th edition (1999), Sec. 11, Chapter 84, Hematology and Oncology, Overview of Cancer.

Determination of serum chemistries and enzyme levels can also help staging. Elevation of liver enzymes (alkaline phosphatase, LDH and ALT) suggests presence of liver metastases. Elevated alkaline phosphatase and serum Ca may be the first evidence of bone metastases. Elevated acid phosphatase (tartrate inhibited) suggests extracapsular extension of prostate cancer. Fasting hypoglycemia may indicate an insulinoma, hepatocellular carcinoma, or retroperitoneal sarcoma. Elevated BUN or creatinine levels may indicate an obstructive uropathy secondary to a pelvic mass, intrarenal obstruction from tubular precipitation of myeloma protein, or uric acid nephropathy from lymphoma or other cancers. Elevated uric acid levels often occur in myeloproliferative and lymphoproliferative disorders, α-Fetoprotein may be elevated in hepatocellular carcinoma and testicular carcinomas, carcinoembryonic antigen-S in colon cancer, human chorionic gonadotropin in choriocarcinoma and testicular carcinoma, serum immunoglobulins in multiple myeloma, and DNA probes (bcr probe to identify the chromosome 22 change) in CML.

Tumors may synthesize proteins that produce no clinical symptoms, e.g., human chorionic gonadotropin, α-fetoprotein, carcinoembryonic antigen, CA 125, and CA 153. These protein products can be used as tumor markers in serial evaluation of patients for determining disease recurrence or response to therapy. Thus, monitoring a subject for these tumor markers is indicative of progress of a neoplasia disorder. Such monitoring is also indicative of how well the methods, combinations and compositions of the present invention are treating or preventing a neoplasia disorder. Likewise, tumor marker monitoring is effective to determine appropriate dosages of a combination or composition of the present invention for treating neoplasia.

Other techniques include mediastinoscopy, which is especially valuable in the staging of non-small cell lung cancer. If mediastinoscopy shows mediastinal lymph node involvement, then the subject would not usually benefit from a thoracotomy and lung resection. Imaging studies, especially CT and MRI, can detect metastases to brain, lung, spinal cord, or abdominal viscera, including the adrenal glands, retroperitoneal lymph nodes, liver, and spleen. MRI (with gadolinium) is the procedure of choice for recognition and evaluation of brain tumors.

Ultrasonography can be used to study orbital, thyroid, cardiac, pericardial, hepatic, pancreatic, renal, and retroperitoneal areas. It may guide percutaneous biopsies and differentiate renal cell carcinoma from a benign renal cyst. Lymphangiography reveals enlarged pelvic and low lumbar lymph nodes and is useful in the clinical staging of patients with Hodgkin's disease, but it has generally been replaced by CT.

Liver-spleen scans can identify liver metastases and splenomegaly. Bone scans are sensitive in identifying metastases before they are evident on x-ray. Because a positive scan requires new bony formation (i.e., osteoblastic activity), this technique is useless in neoplasms that are purely lytic (e.g., multiple myeloma); routine bone x-rays are the study of choice in such diseases. Gallium scans can help in staging lymphoid neoplasms. Radiolabeled monoclonal antibodies (e.g., to carcinoembryonic antigen, small cell lung cancer cells) provide important staging data in various neoplasms (e.g., colon cancer, small cell lung cancer). See The Merck Manual of Diagnosis & Therapy, 17th edition (1999), Sec. 11, Chapter 84, Hematology and Oncology, Overview of Cancer.

As used herein, the term “subject” for purposes of treatment is one that is in need of the treatment of neoplasia or a neoplasia-related disorder. For purposes of prevention, the subject is one that is at risk for, or is predisposed to, developing neoplasia or a neoplasia-related disorder, including relapse of a previously occurring neoplasia or neoplasia-related disorder.

As used herein, the phrase “subject in need of” includes any subject that is suffering from or is predisposed to neoplasia or any neoplasia-related disorder described herein. The phrase “subject in need of” also includes any subject that requires a lower dose of conventional neoplasia treatment agents. In addition, a “subject in need of” includes any subject that requires a reduction in the side-effects of a conventional treatment agent. Furthermore, a “subject in need of” includes any subject that requires improved tolerability to any conventional treatment agent for a neoplasia disorder therapy.

The subject is an animal, typically a mammal, including humans, domestic and farm animals, zoo, sports and pet animals, such as dogs, horses, cats, cattle, etc. The subject is most typically a human subject.

The methods, combinations and compositions of the present invention can be used for treatment or prevention of several neoplasia disorders and neoplasia-related disorders including, but are not limited to, acral lentiginous melanoma, actinic keratosis, adenocarcinoma, adenoid cystic carcinoma, adenoma, adenosarcoma, adenosquamous carcinoma, adrenocortical carcinoma, AIDS-related lymphoma, anal cancer, astrocytic tumors, bartholin gland carcinoma, basal cell carcinoma, bile duct cancer, bladder cancer, brain stem glioma, brain tumor, breast cancer, bronchial gland carcinoma, capillary carcinoma, carcinoids, carcinoma, carcinosarcoma, cavernous cell carcinoma, central nervous system lymphoma, cerebral astrocytoma, childhood cancers, cholangiocarcinoma, chondrosarcoma, chorioid plexus papilloma and carcinoma, clear cell carcinoma, colon cancer, colorectal cancer, cutaneous T-cell lymphoma, cystadenoma, endodermal sinus tumor, endometrial hyperplasia, endometrial stromal sarcoma, endometrioid adenocarcinoma, ependymal cancer, epithelioid carcinoma, esophageal cancer, Ewing's sarcoma, extragonadal germ cell tumor, fibrolamellar carcinoma, focal nodular hyperplasia, gallbladder cancer, gastrinoma, germ cell tumors, gestational trophoblastic tumor, glioblastoma, glioma, glucagonoma, hemangioblastoma, hemangioendothelioma, hemangioma, hepatic adenoma, hepatic adenomatosis, hepatocellular carcinoma, Hodgkin's lymphoma, hypopharyngeal cancer, hypothalamic and visual pathway glioma, insulinoma, interepithelial squamous cell neoplasia, intraepithelial neoplasia, intraocular melanoma, invasive squamous cell carcinoma, islet cell carcinoma, Kaposi's sarcoma, kidney cancer, large cell carcinoma, laryngeal cancer, leiomyosarcoma, lentigo maligna melanoma, leukemia-related disorders, lip and oral cavity cancer, liver cancer, lung cancer, lymphoma, malignant mesothelial tumors, malignant thymoma, medulloblastoma, medulloepithelioma, melanoma, meningeal carcinoma, merkel cell carcinoma, mesothelial carcinoma, metastatic carcinoma, mucoepidermoid carcinoma, multiple myeloma/plasma cell neoplasm, mycosis fungoides, myelodysplastic syndrome, myeloproliferative disorders, nasal cavity and paranasal sinus cancer, nasopharyngeal cancer, neuroblastoma, neuroepithelial adenocarcinoma, nodular melanoma, non-Hodgkin's lymphoma, oat cell carcinoma, oligodendroglial carcinoma, oral cancer, oropharyngeal cancer, osteosarcoma, ovarian cancer, ovarian germ cell tumor, pancreatic cancer, papillary serous adenocarcinoma, parathyroid cancer, penile cancer, pheochromocytoma, pineal and supratentorial primitive neuroectodermal tumors, pineal cell carcinoma, pituitary tumors, plasma cell neoplasm, plasmacytoma, pleuropulmonary blastoma, prostate cancer, pseudosarcoma, pulmonary blastoma, rectal cancer, renal cell carcinoma, retinoblastoma, rhabdomyosarcoma, sarcoma, serous carcinoma, small cell carcinoma, small intestine cancer, soft tissue carcinomas, somatostatin-secreting tumor, squamous cell carcinoma, submesothelial carcinoma, superficial spreading melanoma, thyroid cancer, undifferentiated carcinoma, urethral cancer, uterine sarcoma, uveal melanoma, vaginal cancer, verrucous carcinoma, vipoma, vulvar cancer, Waldenstrom's macroglobulinemia, well differentiated carcinoma, and Wilm's tumor.

All references cited in this specification are incorporated by reference into this specification in their entireties. Discussion of any reference herein is intended merely to summarize statements made by its authors and no admission is made as to accuracy, pertinence or status as prior art of any reference. Applicant reserves the right to challenge the accuracy and pertinence of the cited references.

In view of the above, it will be seen that several advantages of the invention are achieved and other advantageous results obtained.

As various changes could be made in the above methods and compositions without departing from the scope of the invention, it is intended that all matter contained in the above detailed description shall be interpreted as illustrative and not in a limiting sense.

1. A combination comprising a Cox-2 inhibitor and an antineoplastic agent in amounts effective when used in combination therapy for treatment or prevention of neoplasia or a neoplasia-related disorder; wherein the antineoplastic agent is selected from the group consisting of
(1) polyglutamic acid-paclitaxel;
(2) BMS-184476;
(3) Paclimer microspheres with encapsulated paclitaxel;
(4) taxane (IV) of Bayer;
(5) BMS-188797;
(6) epothilone B and analogs thereof including BMS-247550;
(7) ILX-651;
(8) N-[3-[(aminocarbonyl)amino]-4-methoxyphenyl]-2,3,4,5,6-pentafluorobenzenesulfonamide;
(9) T-900607;
(10) BAY 59-8862;
(11) T-138067;
(12) N,N-dimethyl-L-valyl-L-valyl-N-methyl-L-valyl-L-prolyl-N-(1,1-dimethylethyl)-L-prolinamide;
(13) benzoylphenylurea;
(14) trimetrexate glucuronate;
(15) 5-aza-2′-deoxycytidine;
(16) tocladesine;
(17) imatinib;
(18) PTK-787;
(19) BAY-439006;
(20) N-[4-[(3-chloro-4-fluorophenyl)amino]-7-[3-(4-morpholinyl)propoxy]-6-quinazolinyl]-2-propenamide;
(21) GW-572016;
(22) EKB-569;
(23) CP 609754;
(24) CI-1033;
(25) CCI-779;
(26) BMS-214662;
(27) (R)-2,3,4,5-tetrahydro-1-(1H-imidazol-4-ylmethyl)-3-(phenylmethyl)-4-(2-thienylsulfonyl)-1H-1,4-benzodiazepine-7-carbonitrile;
(28) cilengitide;
(29) bevacizumab;
(30) PK-412;
(31) IMC-1C11;
(32) 1-(2-chloroethyl)-2-[(methylamino)]carbonyl}-2-(methylsulfonyl) hydrazide;
(33) VNP-40101M;
(34) camptothecin glycoconjugate;
(35) liposome lurtotecan;
(36) gallium maltolate;
(37) N-[(3S,4E)-3-hydroxy-7-mercapto-1-oxo-4-heptenyl]-D-valyl-D-cysteinyl-(2Z)-2-amino-2-butenoyl-L-valine (4-1)-lactone cyclic (1-2) disulfide;
(38) buthionine sulfoximine;
(39) BMS-275291;
(40) phenylacetate;
(41) MS-275;
(42) chloroquinoxaline sulfonamide;
(43) INX-3280;
(44) phosphorothioate antisense oligonucleotide;
(45) GTI-2501;
(46) GTI-2040;
(47) K-ras protein vaccine;
(48) K-ras antisense oligonucleotide;
(49) MG-98;
(50) liposome C-raf antisense oligonucleotide;
(51) liposome raf-1 antisense oligonucleotide;
(52) SPD-424;
(53) Abarelix-depot;
(54) ERA-923;
(55) GTx-006;
(56) ILX 23-7553;
(57) 2B1 bispecific MAb;
(58) 3A1 MAb;
(59) SS1(dsFv)-PE38;
(60) chimeric TNT 1/B labeled with I-131;
(61) MAb Hum291;
(62) MEDI-507;
(63) HumaRad-HN;
(64) HumaRad-OV;
(65) MAb humanized CD3;
(66) Mylotarg;
(67) MAb-CTLA-4;
(68) cetuximab;
(69) BEC2;
(70) chimeric MAb 14.18;
(71) anti-transferrin receptor MAb;
(72) epratuzumab;
(73) MGS rCEA;
(74) INGN-241;
(75) CV-787;
(76) peripheral blood lymphocytes transduced with a gene encoding a chimeric T-cell receptor;
(77) BCI Immune Activator;
(78) Interferon-alpha gene therapy;
(79) Xcellerate;
(80) interleukin-2+staphylococcal enterotoxin B;
(81) NBI-3001;
(82) beta-alethine;
(83) APC-8020;
(84) interleukin-2/superantigen B gene combination;
(85) Melacine vaccine;
(86) SD/01;
(87) ALVAC B7.1 vaccine;
(88) APC-8024;
(89) GnRH Pharmaccine vaccine;
(90) rV-MUC-1;
(91) HPV 16 E6 and E7 peptide vaccine;
(92) allogeneic colon cancer vaccine;
(93) allogeneic glioma vaccine;
(94) autologous vaccine;
(95) VHL peptide vaccine;
(96) myeloma-derived idiotypic antigen vaccine;
(97) CaPVax;
(98) idiotype KLH lymphoma vaccine;
(99) LHRH immunotherapeutic (synthetic peptide vaccine);
(100) MAGE-12:170-178 peptide vaccine;
(101) MART-1 melanoma vaccine;
(102) MART-1 with gp100;
(103) rF-tyrosine vaccine;
(104) ESO-1:157-165 peptide vaccine;
(105) fowlpox-CEA(6D) tricom and vaccinia-CEA(6D) tricom vaccine;
(106) fowlpox gp100:ES 209-217 (2m) vaccine;
(107) RAS 5-17 peptide vaccine;
(108) proteinase-3 peptide vaccine;
(109) canarypox CEA;
(110) Helicobacter pylori vaccine;
(111) P53 and RAS vaccine;
(112) BAM-002;
(113) MedPulser in combination with bleomycin;
(114) lasofoxifene;
(115) Filmix;
(116) L-377202;
(117) T4N5 Liposome Lotion;
(118) Egr-1+TNF-alpha;
(119) aprepitant;
(120) skeletal targeted radiotherapy;
(121) combretastatin;
(122) CDC-501;
(123) taurolidine;
(124) Oramed;
(125) nystatin;
(126) Dynepo gene activated EPO;
(127) NC-100150;
(128) NC-100100;
(129) CDC-801;
(130) atrasentan;
(131) Aranesp;
(132) RK-0202;
(133) SB-251353;
(134) rasburicase;
(135) AFP-scan;
(136) Lymphoscan;
(137) ADL 8-2698;
(138) carboxypeptidase G2;
(139) metoclopromide nasal;
(140) dalteparin;
(141) MK-869;
(142) monomethyl arginine;
(143) repifermin;
(144) rH TPO;
(145) SR-29142;
(146) ancestin;
(147) CP-461;
(148) Bexxar;
and combinations thereof.

2. The combination of claim 1 wherein the Cox-2 inhibitor is a Cox-2 selective inhibitor.

3. The combination of claim 2 wherein the Cox-2 selective inhibitor provides a Cox-1 IC₅₀/Cox-2 IC₅₀ ratio of at least about 10.

4. The combination of claim 2 wherein the Cox-2 selective inhibitor provides a Cox-1 IC₅₀/Cox-2 IC₅₀ ratio of at least about 100.

5. The combination of claim 2 wherein the Cox-2 selective inhibitor is a tricyclic compound, a substituted benzopyran derivative or a phenylacetic acid derivative.

6. The combination of claim 2 wherein the Cox-2 selective inhibitor is selected from the group consisting of celecoxib, valdecoxib, parecoxib, rofecoxib, etoricoxib, lumiracoxib and pharmaceutically acceptable salts thereof.

7. The combination of claim 2 wherein the Cox-2 selective inhibitor is parecoxib sodium.

8. A method of treating or preventing neoplasia or a neoplasia-related disorder in a subject, the method comprising administering in combination therapy to the subject a Cox-2 inhibitor and an antineoplastic agent in amounts effective when used in said combination therapy for treatment or prevention of neoplasia or a neoplasia-related disorder; wherein the antineoplastic agent is selected from the group consisting of
(1) polyglutamic acid-paclitaxel;
(2) BMS-184476;
(3) Paclimer microspheres with encapsulated paclitaxel;
(4) taxane (IV) of Bayer;
(5) BMS-188797;
(6) epothilone B and analogs thereof including BMS-247550;
(7) ILX-651;
(8) N-[3-[(aminocarbonyl)amino]-4-methoxyphenyl]-2,3,4,5,6-pentafluorobenzenesulfonamide;
(9) T-900607;
(10) BAY 59-8862;
(11) T-138067;
(12) N,N-dimethyl-L-valyl-L-valyl-N-methyl-L-valyl-L-prolyl-N-(1,1-dimethylethyl)-L-prolinamide;
(13) benzoylphenylurea;
(14) trimetrexate glucuronate;
(15) 5-aza-2′-deoxycytidine;
(16) tocladesine;
(17) imatinib;
(18) PTK-787;
(19) BAY-439006;
(20) N-[4-[(3-chloro-4-fluorophenyl)amino]-7-[3-(4-morpholinyl)propoxy]-6-quinazolinyl]-2-propenamide;
(21) GW-572016;
(22) EKB-569;
(23) CP 609754;
(24) CI-1033;
(25) CCI-779;
(26) BMS-214662;
(27) (R)-2,3,4,5-tetrahydro-1-(1H-imidazol-4-ylmethyl)-3-(phenylmethyl)-4-(2-thienylsulfonyl)-1H-1,4-benzodiazepine-7-carbonitrile;
(28) cilengitide;
(29) bevacizumab;
(30) PK-412;
(31) IMC-1C11;
(32) 1-(2-chloroethyl)-2-[(methylamino)]carbonyl}-2-(methylsulfonyl) hydrazide;
(33) VNP-40101M;
(34) camptothecin glycoconjugate;
(35) liposome lurtotecan;
(36) gallium maltolate;
(37) N-[(3S,4E)-3-hydroxy-7-mercapto-1-oxo-4-heptenyl]-D-valyl-D-cysteinyl-(2Z)-2-amino-2-butenoyl-L-valine (4-1)-lactone cyclic (1-2) disulfide;
(38) buthionine sulfoximine;
(39) BMS-275291;
(40) phenylacetate;
(41) MS-275;
(42) chloroquinoxaline sulfonamide;
(43) INX-3280;
(44) phosphorothioate antisense oligonucleotide;
(45) GTI-2501;
(46) GTI-2040;
(47) K-ras protein vaccine;
(48) K-ras antisense oligonucleotide;
(49) MG-98;
(50) liposome C-raf antisense oligonucleotide;
(51) liposome raf-1 antisense oligonucleotide;
(52) SPD-424;
(53) Abarelix-depot;
(54) ERA-923;
(55) GTx-006;
(56) ILX 23-7553;
(57) 2B1 bispecific MAb;
(58) 3A1 MAb;
(59) SS1(dsFv)-PE38;
(60) chimeric TNT 1/B labeled with I-131;
(61) MAb Hum291;
(62) MEDI-507;
(63) HumaRad-HN;
(64) HumaRad-OV;
(65) MAb humanized CD3;
(66) Mylotarg;
(67) MAb-CTLA-4;
(68) cetuximab;
(69) BEC2;
(70) chimeric MAb 14.18;
(71) anti-transferrin receptor MAb;
(72) epratuzumab;
(73) MGS rCEA;
(74) INGN-241;
(75) CV-787;
(76) peripheral blood lymphocytes transduced with a gene encoding a chimeric T-cell receptor;
(77) BCI Immune Activator;
(78) Interferon-alpha gene therapy;
(79) Xcellerate;
(80) interleukin-2+staphylococcal enterotoxin B;
(81) NBI-3001;
(82) beta-alethine;
(83) APC-8020;
(84) interleukin-2/superantigen B gene combination;
(85) Melacine vaccine;
(86) SD/01;
(87) ALVAC B7.1 vaccine;
(88) APC-8024;
(89) GnRH Pharmaccine vaccine;
(90) rV-MUC-1;
(91) HPV 16 E6 and E7 peptide vaccine;
(92) allogeneic colon cancer vaccine;
(93) allogeneic glioma vaccine;
(94) autologous vaccine;
(95) VHL peptide vaccine;
(96) myeloma-derived idiotypic antigen vaccine;
(97) CaPVax;
(98) idiotype KLH lymphoma vaccine;
(99) LHRH immunotherapeutic (synthetic peptide vaccine);
(100) MAGE-12:170-178 peptide vaccine;
(101) MART-1 melanoma vaccine;
(102) MART-1 with gp100;
(103) rF-tyrosine vaccine;
(104) ESO-1:157-165 peptide vaccine;
(105) fowlpox-CEA(6D) tricom and vaccinia-CEA(6D) tricom vaccine;
(106) fowlpox gp100:ES 209-217 (2m) vaccine;
(107) RAS 5-17 peptide vaccine;
(108) proteinase-3 peptide vaccine;
(109) canarypox CEA;
(110) Helicobacter pylori vaccine;
(111) P53 and RAS vaccine;
(112) BAM-002;
(113) MedPulser in combination with bleomycin;
(114) lasofoxifene;
(115) Filmix;
(116) L-377202;
(117) T4N5 Liposome Lotion;
(118) Egr-1+TNF-alpha;
(119) aprepitant;
(120) skeletal targeted radiotherapy;
(121) combretastatin;
(122) CDC-501;
(123) taurolidine;
(124) Oramed;
(125) nystatin;
(126) Dynepo gene activated EPO;
(127) NC-100150;
(128) NC-100100;
(129) CDC-801;
(130) atrasentan;
(131) Aranesp;
(132) RK-0202;
(133) SB-251353;
(134) rasburicase;
(135) AFP-scan;
(136) Lymphoscan;
(137) ADL 8-2698;
(138) carboxypeptidase G2;
(139) metoclopromide nasal;
(140) dalteparin;
(141) MK-869;
(142) monomethyl arginine;
(143) repifermin;
(144) rH TPO;
(145) SR-29142;
(146) ancestin;
(147) CP-461;
(148) Bexxar;
and combinations thereof.

9. The method of claim 8 wherein the Cox-2 inhibitor is a Cox-2 selective inhibitor.

10. The method of claim 9 wherein the Cox-2 selective inhibitor provides a Cox-1 IC₅₀/Cox-2 IC₅₀ ratio of at least about 10.

11. The method of claim 9 wherein the Cox-2 selective inhibitor provides a Cox-1 IC₅₀/Cox-2 IC₅₀ ratio of at least about 100.

12. The method of claim 9 wherein the Cox-2 selective inhibitor is a tricyclic compound, a substituted benzopyran derivative or a phenylacetic acid derivative.

13. The method of claim 9 wherein the Cox-2 selective inhibitor is selected from the group consisting of celecoxib, valdecoxib, parecoxib, rofecoxib, etoricoxib, lumiracoxib and pharmaceutically acceptable salts thereof.

14. The method of claim 9 wherein the Cox-2 selective inhibitor is parecoxib sodium.

15. The method of claim 8 wherein the Cox-2 inhibitor and the antineoplastic agent are administered sequentially.

16. The method of claim 8 wherein the Cox-2 inhibitor and the antineoplastic agent are administered substantially simultaneously.

17. The method of claim 8 wherein the neoplasia is selected from the group consisting of acral lentiginous melanoma, actinic keratosis, adenocarcinoma, adenoid cystic carcinoma, adenoma, adenosarcoma, adenosquamous carcinoma, adrenocortical carcinoma, AIDS-related lymphoma, anal cancer, astrocytic tumors, bartholin gland carcinoma, basal cell carcinoma, bile duct cancer, bladder cancer, brain stem glioma, brain tumor, breast cancer, bronchial gland carcinoma, capillary carcinoma, carcinoids, carcinoma, carcinosarcoma, cavernous cell carcinoma, central nervous system lymphoma, cerebral astrocytoma, childhood cancers, cholangiocarcinoma, chondrosarcoma, chorioid plexus papilloma and carcinoma, clear cell carcinoma, colon cancer, colorectal cancer, cutaneous T-cell lymphoma, cystadenoma, endodermal sinus tumor, endometrial hyperplasia, endometrial stromal sarcoma, endometrioid adenocarcinoma, ependymal cancer, epithelioid carcinoma, esophageal cancer, Ewing's sarcoma, extragonadal germ cell tumor, fibrolamellar carcinoma, focal nodular hyperplasia, gallbladder cancer, gastrinoma, germ cell tumors, gestational trophoblastic tumor, glioblastoma, glioma, glucagonoma, hemangioblastoma, hemangioendothelioma, hemangioma, hepatic adenoma, hepatic adenomatosis, hepatocellular carcinoma, Hodgkin's lymphoma, hypopharyngeal cancer, hypothalamic and visual pathway glioma, insulinoma, interepithelial squamous cell neoplasia, intraepithelial neoplasia, intraocular melanoma, invasive squamous cell carcinoma, islet cell carcinoma, Kaposi's sarcoma, kidney cancer, large cell carcinoma, laryngeal cancer, leiomyosarcoma, lentigo maligna melanoma, leukemia-related disorders, lip and oral cavity cancer, liver cancer, lung cancer, lymphoma, malignant mesothelial tumors, malignant thymoma, medulloblastoma, medulloepithelioma, melanoma, meningeal carcinoma, merkel cell carcinoma, mesothelial carcinoma, metastatic carcinoma, mucoepidermoid carcinoma, multiple myeloma/plasma cell neoplasm, mycosis fungoides, myelodysplastic syndrome, myeloproliferative disorders, nasal cavity and paranasal sinus cancer, nasopharyngeal cancer, neuroblastoma, neuroepithelial adenocarcinoma, nodular melanoma, non-Hodgkin's lymphoma, oat cell carcinoma, oligodendroglial carcinoma, oral cancer, oropharyngeal cancer, osteosarcoma, ovarian cancer, ovarian germ cell tumor, pancreatic cancer, papillary serous adenocarcinoma, parathyroid cancer, penile cancer, pheochromocytoma, pineal and supratentorial primitive neuroectodermal tumors, pineal cell carcinoma, pituitary tumors, plasma cell neoplasm, plasmacytoma, pleuropulmonary blastoma, prostate cancer, pseudosarcoma, pulmonary blastoma, rectal cancer, renal cell carcinoma, retinoblastoma, rhabdomyosarcoma, sarcoma, serous carcinoma, small cell carcinoma, small intestine cancer, soft tissue carcinomas, somatostatin-secreting tumor, squamous cell carcinoma, submesothelial carcinoma, superficial spreading melanoma, thyroid cancer, undifferentiated carcinoma, urethral cancer, uterine sarcoma, uveal melanoma, vaginal cancer, verrucous carcinoma, vipoma, vulvar cancer, Waldenstrom's macroglobulinemia, well differentiated carcinoma, and Wilm's tumor.

18. The method of claim 21, further comprising radiation therapy administered in combination with administration of the Cox-2 inhibitor and the antineoplastic agent.

19. A pharmaceutical composition comprising the combination of claim 1 and a pharmaceutically acceptable carrier.

20. A kit comprising a first dosage form that comprises an Cox-2 inhibitor in a first amount and a second dosage form that comprises an antineoplastic agent in a second amount; wherein said first and second amounts are effective when used in combination therapy for treating or preventing neoplasia or a neoplasia-related disorder; and wherein the antineoplastic agent is selected from the group consisting of
(1) polyglutamic acid-paclitaxel;
(2) BMS-184476;
(3) Paclimer microspheres with encapsulated paclitaxel;
(4) taxane (IV) of Bayer;
(5) BMS-188797;
(6) epothilone B and analogs thereof including BMS-247550;
(7) ILX-651;
(8) N-[3-[(aminocarbonyl)amino]-4-methoxyphenyl]-2,3,4,5,6-pentafluorobenzenesulfonamide;
(9) T-900607;
(10) BAY 59-8862;
(11) T-138067;
(12) N,N-dimethyl-L-valyl-L-valyl-N-methyl-L-valyl-L-prolyl-N-(1,1-dimethylethyl)-L-prolinamide;
(13) benzoylphenylurea;
(14) trimetrexate glucuronate;
(15) 5-aza-2′-deoxycytidine;
(16) tocladesine;
(17) imatinib;
(18) PTK-787;
(19) BAY-439006;
(20) N-[4-[(3-chloro-4-fluorophenyl)amino]-7-[3-(4-morpholinyl)propoxy]-6-quinazolinyl]-2-propenamide;
(21) GW-572016;
(22) EKB-569;
(23) CP 609754;
(24) CI-1033;
(25) CCI-779;
(26) BMS-214662;
(27) (R)-2,3,4,5-tetrahydro-1-(1H-imidazol-4-ylmethyl)-3-(phenylmethyl)-4-(2-thienylsulfonyl)-1H-1,4-benzodiazepine-7-carbonitrile;
(28) cilengitide;
(29) bevacizumab;
(30) PK-412;
(31) IMC-1C11;
(32) 1-(2-chloroethyl)-2-[(methylamino)]carbonyl}-2-(methylsulfonyl) hydrazide;
(33) VNP-40101M;
(34) camptothecin glycoconjugate;
(35) liposome lurtotecan;
(36) gallium maltolate;
(37) N-[(3S,4E)-3-hydroxy-7-mercapto-1-oxo-4-heptenyl]-D-valyl-D-cysteinyl-(2Z)-2-amino-2-butenoyl-L-valine (4-1)-lactone cyclic (1-2) disulfide;
(38) buthionine sulfoximine;
(39) BMS-275291;
(40) phenylacetate;
(41) MS-275;
(42) chloroquinoxaline sulfonamide;
(43) INX-3280;
(44) phosphorothioate antisense oligonucleotide;
(45) GTI-2501;
(46) GTI-2040;
(47) K-ras protein vaccine;
(48) K-ras antisense oligonucleotide;
(49) MG-98;
(50) liposome C-raf antisense oligonucleotide;
(51) liposome raf-1 antisense oligonucleotide;
(52) SPD-424;
(53) Abarelix-depot;
(54) ERA-923;
(55) GTx-006;
(56) ILX 23-7553;
(57) 2B1 bispecific MAb;
(58) 3A1 MAb;
(59) SS1(dsFv)-PE38;
(60) chimeric TNT 1/B labeled with I-131;
(61) MAb Hum291;
(62) MEDI-507;
(63) HumaRad-HN;
(64) HumaRad-OV;
(65) MAb humanized CD3;
(66) Mylotarg;
(67) MAb-CTLA-4;
(68) cetuximab;
(69) BEC2;
(70) chimeric MAb 14.18;
(71) anti-transferrin receptor MAb;
(72) epratuzumab;
(73) MGS rCEA;
(74) INGN-241;
(75) CV-787;
(76) peripheral blood lymphocytes transduced with a gene encoding a chimeric T-cell receptor;
(77) BCI Immune Activator;
(78) Interferon-alpha gene therapy;
(79) Xcellerate;
(80) interleukin-2+staphylococcal enterotoxin B;
(81) NBI-3001;
(82) beta-alethine;
(83) APC-8020;
(84) interleukin-2/superantigen B gene combination;
(85) Melacine vaccine;
(86) SD/01;
(87) ALVAC B7.1 vaccine;
(88) APC-8024;
(89) GnRH Pharmaccine vaccine;
(90) rV-MUC-1;
(91) HPV 16 E6 and E7 peptide vaccine;
(92) allogeneic colon cancer vaccine;
(93) allogeneic glioma vaccine;
(94) autologous vaccine;
(95) VHL peptide vaccine;
(96) myeloma-derived idiotypic antigen vaccine;
(97) CaPVax;
(98) idiotype KLH lymphoma vaccine;
(99) LHRH immunotherapeutic (synthetic peptide vaccine);
(100) MAGE-12:170-178 peptide vaccine;
(101) MART-1 melanoma vaccine;
(102) MART-1 with gp100;
(103) rF-tyrosine vaccine;
(104) ESO-1:157-165 peptide vaccine;
(105) fowlpox-CEA(6D) tricom and vaccinia-CEA(6D) tricom vaccine;
(106) fowlpox gp100:ES 209-217 (2m) vaccine;
(107) RAS 5-17 peptide vaccine;
(108) proteinase-3 peptide vaccine;
(109) canarypox CEA;
(110) Helicobacter pylori vaccine;
(111) P53 and RAS vaccine;
(112) BAM-002;
(113) MedPulser in combination with bleomycin;
(114) lasofoxifene;
(115) Filmix;
(116) L-377202;
(117) T4N5 Liposome Lotion;
(118) Egr-1+TNF-alpha;
(119) aprepitant;
(120) skeletal targeted radiotherapy;
(121) combretastatin;
(122) CDC-501;
(123) taurolidine;
(124) Oramed;
(125) nystatin;
(126) Dynepo gene activated EPO;
(127) NC-100150;
(128) NC-100100;
(129) CDC-801;
(130) atrasentan;
(131) Aranesp;
(132) RK-0202;
(133) SB-251353;
(134) rasburicase;
(135) AFP-scan;
(136) Lymphoscan;
(137) ADL 8-2698;
(138) carboxypeptidase G2;
(139) metoclopromide nasal;
(140) dalteparin;
(141) MK-869;
(142) monomethyl arginine;
(143) repifermin;
(144) rH TPO;
(145) SR-29142;
(146) ancestin;
(147) CP-461;
(148) Bexxar;
and combinations thereof.
